(19) United States
(12) Patent Application Publication
(10) Pub. No.: US 2025/0084044 A1
(43) Pub. Date: Mar. 13, 2025

(54) PROTOPORPHYRINOGEN OXIDASE INHIBITORS

(71) Applicant: Enko Chem, Inc., Mystic, CT (US)

(72) Inventors: Neville John ANTHONY, Northborough, MA (US); Paul GALATSIS, Newton, MA (US); David Jeffrey LAUFFER, Stow, MA (US); Peter STCHUR, III, Waterford, CT (US)

(73) Assignee: Enko Chem, Inc., Mystic, CT (US)

(21) Appl. No.: 18/727,309

(22) PCT Filed: Jan. 11, 2023
(86) PCT No.: PCT/US2023/060448

§ 371 Date: Jul. 8, 2024

Related U.S. Application Data
(60) Provisional application No. 63/299,862, filed on Jan. 14, 2022.

(51) Int. Cl. C07D 265/36; A01N 43/84; A01P 13/00

(52) U.S. Cl. CPC C07D 265/36; A01N 43/84; A01P 13/00

(57) ABSTRACT

Tire present invention relates to protopoiphyrinogen oxidase inhibitors of the general formula (I) where the variables are defined herein. The invention features processes and intermediates for preparing the compounds of formula (I), compositions comprising them, and their use as herbicides i.e. for controlling undesired vegetation. The invention also features methods for controlling unwanted vegetation comprising allowing an herbicidal effective amount of at least one benzoxazinone of formula (I) to act on plants, their seed, and/or their environment.

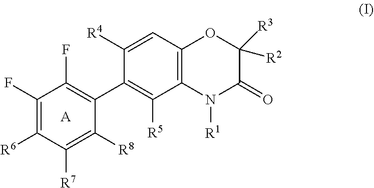

### CROSS-REFERENCE TO RELATED APPLICATIONS

This application claims priority to U.S. Provisional Patent Application No. 63/299,862, filed Jan. 14, 2022, which is incorporated herein by reference in its entirety.

### FIELD

The present invention relates to protoporphyrinogen IX oxidase (PPO) inhibitors useful as herbicides. In particular, the present invention relates to certain fluorinated biphenyl compounds, compositions comprising such compounds, processes for making such compounds and compositions, and methods for using such compounds for crop protection and to control unwanted vegetation.

### BACKGROUND

Herbicides that inhibit protoporphyrinogen oxidase (hereinafter referred to as Protox or PPO; EC: 1.3.3.4), a key enzyme in the biosynthesis of protoporphyrin IX, have been used for selective weed control since the 1960s. PPO catalyzes the last common step in chlorophyll and heme biosynthesis, which is the oxidation of protoporphyrinogen IX to protoporphyrin IX [Matringe M. et al., Protoporphyrinogen oxidase as a molecular target for diphenyl ether herbicides, Biochemistry Journal (1989) 260: 231-235]. Application of PPO-inhibiting herbicides results in the accumulation of protoporphyrinogen IX in the chloroplast and mitochondria, which is believed to leak into the cytosol where it is oxidized by a peroxidase. When exposed to light, protoporphyrin IX causes formation of singlet oxygen in the cytosol and the formation of other reactive oxygen species, which can cause lipid peroxidation and membrane disruption leading to rapid cell death [Lee H. J. et al., Cellular localization of protoporphyrinogen-oxidizing activities of etiolated barley leaves, Plant Physiology (1993) 102: 881].

To date, thousands of PPO inhibitors have been reported in the literature, with about 30 currently used as herbicides to decimate weeds in fields [Hao, G. F., et al., Protoporphyrinogen oxidase inhibitor: an ideal target for herbicide discovery, Chimia (2011) 65, 961-969]. PPO-inhibiting herbicides include many different structural classes of molecules, including diphenyl ethers (e.g. lactofen, acifluorfen, acifluorfen methyl ester, or oxyfluorfen); oxadiazoles (e.g. oxadiazon); cyclic imides [e.g. S-23142, N-(4-chloro-2-fluoro-5-propargyloxyphenyl)-3,4,5,6-tetrahydrophthalimide, chlorophthalim, N-(4-chlorophenyl)-3,4,5,6-tetrahydrophthalimide)]; phenyl pyrazoles (e.g. TNPP-ethyl, ethyl 2-[1-(2,3,4-trichlorophenyl)-4-nitropyrazolyl-5-oxy]propionate, M&B 39279); pyridine derivatives (e.g. LS 82-556); and phenopylate and its O-phenylpyrrolidino- and piperidinocarbamate analogs (Kramer W., ed., Modern Crop Protection Compounds, 2^{nd} Ed Vol 1: Herbicides, (2012) Wiley-VCH. Weinheim, Germany). Many of these compounds competitively inhibit the normal reaction catalyzed by the enzyme, apparently acting as substrate analogs.

The herbicidal properties of these known compounds towards harmful plants, however, are not always entirely satisfactory. Herbicide resistant weeds present a serious problem for efficient weed control because such resistant weeds are increasingly widespread and thus weed control by the application of herbicides is no longer effective, causing a huge problem to farmers. Resistance to PPO herbicides has been slow to evolve (about four decades from first commercialization), and to date has been confirmed in 13 weed species [Heap 1, The International Survey of Herbicide Resistant Weeds. Available online: http:/hvww.wcedscience.org/(October 2019)]. The first weed to evolve resistance to PPO herbicides was waterhemp (Amaranthus tuberculatus) in 2001 [Shoup D. E., et al., Common waterhemp (Amaranthus rudis) resistance to protoporphyrinogen oxidase-inhibiting herbicides Weed Sci. (2003) 51:145-150]. Resistance to PPO herbicides in weedy species has been attributed to target-site mutation in the PPX2 gene. For example, a unique target-site amino acid deletion (Gly₂₁₀) and Arg98Leu substitution confer PPO resistance in waterhemp [Patzoldt W. L., et al., A codon deletion confers resistance to herbicides inhibiting protoporphyrinogen oxidase. Proc. Natl. Acad Sci. USA (2006) 103:12329-12334] and common ragweed [Rousonelos, et al., Characterization of a common ragweed (Ambrosia artemisiijblia) population resistant to ALS- and PPO-inhibiting herbicides, Weed Sci. (2012) 60:335-344], respectively.

Thus, there is a need for novel methods to effectively control weeds, including herbicide resistant weeds and in particular PPO resistant weeds, which at the same time is tolerated by the useful plants (crops) in question.

### BRIEF SUMMARY

Accordingly, in one aspect, provided are benzoxazinones having formula (I):

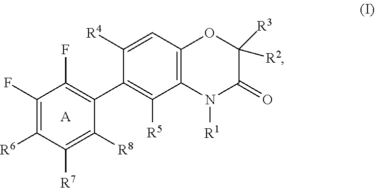

or a salt thereof, wherein Ring A and R¹-R⁸ are as defined herein. In some embodiments, Ring A contains at least 4 F atom substituents.

In certain embodiments, provided are benzoxazinones having formula (II):

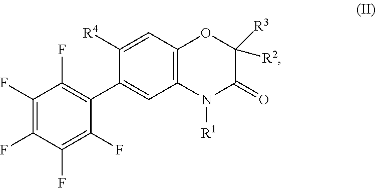

or a salt thereof, wherein R¹-R⁴ are as defined elsewhere herein.

In certain embodiments, provided are benzoxazinones having formula (III):

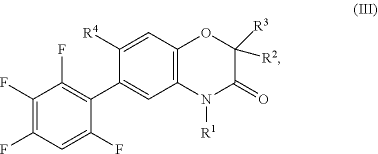

or a salt thereof, wherein R¹-R⁴ are as defined elsewhere herein.

In certain embodiments, provided are benzoxazinones having formula (IV):

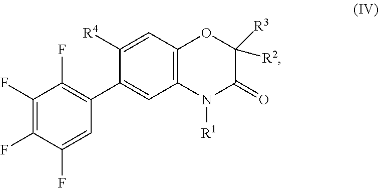

or a salt thereof, wherein R¹-R⁴ are as defined elsewhere herein.

In certain embodiments, provided are benzoxazinones having formula (V):

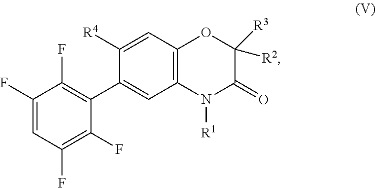

or a salt thereof, wherein R¹-R⁴ are as defined elsewhere herein

In other aspects, provided is also an agricultural composition (including, in some variations, herbicidal compositions) that includes a compound of formula (I), (II), (III), (IV) or (V), or a salt thereof, in a herbicidally effective amount and at least one component selected from the group consisting of surfactants, solid diluents and liquid diluents (e.g., formulations). In some variations, the salt is an agriculturally suitable salt. In some embodiments, the composition optionally further includes at least one additional active ingredient. In one variation, the additional active ingredient may be an herbicide and/or herbicide safener.

In yet another aspect, provided are also processes for making the above-identified compounds, salts, and compositions.

In certain aspects, provided are compounds that are intermediates for making one or more compounds of the invention, including one or more compounds of Table 1, or a salt thereof.

In yet other aspects, provided are also methods for controlling the growth of undesired vegetation comprising contacting the vegetation or its environment with a herbicidally effective amount of a compound of the invention, its salt, or a composition that includes a compound of the invention as described herein.

### DETAILED DESCRIPTION

### Benzoxazinone Compounds

In one aspect, provided are benzoxazinones having formula (I):

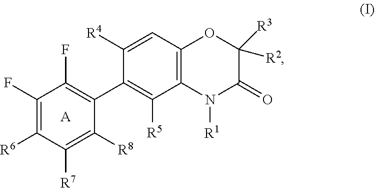

or suitable salt thereof, wherein: R¹ is C₁₋₆alkyl, C₃₋₇alkenyl, C₃₋₇alkynyl, cyclopropyl, CH₂C₃₋₆cycloalkyl, phenyl or C₁₋₂alkyl-phenyl, each substituted with C(O)R^{1a} or CH₂C(O)R^{1a} and each optionally substituted with up to 3 F or Cl atoms, wherein each C₁₋₆ alkyl is also optionally substituted with —OR^{1b},
R^{1a} is OR^{1b}, CH₂OC(O)C₁₋₄alkyl, C(O)OR^{1b}, N(R^{1b})(R^{1c}), ON(R^{1b})(R^{1c}), NHN(R^{1b})(R^{1c}), NHS(O)₂N(R^{1b})₂, NHS(O)₂C₁₋₄alkyl, or NHOR^{1b};
each R^{1b} is, independently, H, C₃₋₆cycloalkyl, CH₂phenyl, or C₁₋₄alkyl optionally substituted with up to 3 F or Cl atoms,
R^{1c} is H or C₁₋₄alkyl optionally substituted with C(O)OR^{1b} or R^{1b} and R^{1c} together with an intervening nitrogen atom form a 4 to 6 membered heterocyclic ring, optionally containing an additional atom or group selected from N, O, S, S(O)₂ and optionally substituted with one or more groups selected from —C(O)OR^{1b} and —C(O)R^{1b};
each of R² and R³ is H, F, or R² and R³ together with the intervening carbon is cyclopropyl;
R⁴ is H, F, CH₃, or Cl;
R⁵ is H or F;
each of R⁶ and R⁷ is, independently, F, H, CH₃, CF₃, or OCH₃;
R⁸ is H or F; and
wherein Ring A contains at least 4 F atom substituents.

In another aspect, provided are benzoxazinones having formula (I):

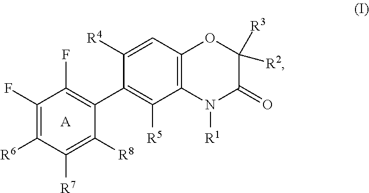

or suitable salt thereof, wherein
R¹ is alkyl, alkenyl, alkynyl, cycloalkyl, CH₂cycloalkyl, phenyl or alkyl-phenyl, each substituted with C(O)R^{1a} or CH₂C(O)R^{1a} and each optionally substituted with up to 3 F or Cl atoms, wherein each alkyl is also optionally substituted with —OR^{1b};
R^{1a} is OR^{1b}, CH₂OC(O)alkyl, C(O)OR^{1b}, N(R^{1b})(R^{1c}), ON(R^{1b})(R^{1c}), NHN(R^{1b})(R^{1c}), NHS(O)₂N(R^{1b})₂, NHS(O)₂alkyl, or NHOR^{1b},
each R^{1b} is, independently, H, cycloalkyl, CH₂phenyl, or alkyl optionally substituted with up to 3 F or Cl atoms;
R^{1c} is H or alkyl optionally substituted with C(O)OR^{1b} or R^{1b} and R^{1c} together with an intervening nitrogen atom form a 4 to 6 membered heterocyclic ring, optionally containing an additional atom or group selected from N, O, S, S(O)₂ and optionally substituted with one or more groups selected from —C(O)OR^{1b} and —C(O)R^{1b};
each of R² and R³ is H, F, or R² and R³ together with the intervening carbon is cyclopropyl;
R⁴ is H, F, CH₃, or Cl;
R⁵ is H or F;
each of R⁶ and R⁷ is, independently, F, H, CH₃, CF₃, or OCH₃;
R⁸ is H or F; and
wherein Ring A contains at least 4 F atom substituents.

In another aspect, provided are benzoxazinones having formula (I):

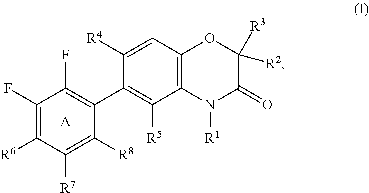

or suitable salt thereof, wherein
R¹ is C₁₋₆alkyl, C₃₋₄alkenyl, C₃₋₄alkynyl, cyclopropyl, phenyl or C₁₋₂alkyl-phenyl, each substituted with —C(O)R^{1a} or —CH₂C(O)R^{1a}, each optionally substituted with up to 3 halogens, and each C₁₋₆alkyl optionally substituted with OR^{1b};
R^{1a} is —OR^{1b}, —C(O)R^{1b}, CH₂OC(O)C₁₋₄alkyl, CF₃, —N(R^{1b})(R^{1c}), —ON(R^{1b})(R^{1c}), —NHN(R^{1b})(R^{1c}), NHS(O)₂N(R^{1b})₂, NHS(O)₂C₁₋₄alkyl, or —NHOR^{1b};
each R^{1b} is, independently, H, cyclopropyl, or C₁₋₄alkyl optionally substituted with up to 3 halogens;
R^{1c} is H or C₁₋₄alkyl optionally substituted with —C(O)OR^{1b} or R^{1b} and R^{1c} together with an intervening nitrogen atom form a 4 to 6 membered heterocyclic ring optionally containing an additional heteroatom selected from N, O, or S and optionally substituted with one or more groups selected from —C(O)OR^{1b} and —C(O)R^{1b};
each of R² and R³ is H, F, Cl, CH₃, or R² and R³ together with the intervening carbon is cyclopropyl;
R⁴ is H, F, or Cl;
R⁵ is H or F;
each of R⁶ and R⁷ is, independently, F, H, C₁₋₂alkyl, alkenyl, CF₃, —OH, —OC₁₋₂alkyl, or —SCH₃;
R⁸ is H or F; and
wherein Ring A contains at least 4 F atom substituents.

In one embodiment, each of R², R³, and R⁴ is F.

In another embodiment, each of R² and R³ is H and R⁴ is F.

In another embodiment, R¹ is C₁₋₆alkyl substituted with —C(O)R^{1a}, wherein R^{1a} is —OR^{1b} or —N(R^{1b})(R^{1c}). In one embodiment, R¹ is C₁₋₂alkyl substituted with —C(O)R^{1a}, wherein R^{1a} is —OR^{1b} or —N(R^{1b})(R^{1c}).

In another embodiment, R¹ is C₁₋₆alkyl substituted with —C(O)R^{1a}, wherein R^{1a} is —OR^{1b}. In another embodiment, R¹ is —CH(CH₃)C(O)R^{1a}, wherein R^{1a} is —OR^{1b}.

In one embodiment, R¹ is C₁₋₆alkyl substituted with —C(O)OH. In one embodiment, R¹ is C₁₋₆alkyl substituted with —C(O)OC₁₋₄alkyl. In one embodiment, R¹ is C₁₋₆alkyl substituted with —C(O)OCH₃. In one embodiment R¹ is

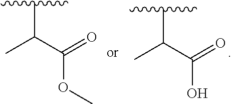

In another embodiment, R¹ is C₁₋₆alkyl substituted with —C(O)R^{1a}, wherein R^{1a} is —N(R^{1b})(R^{1c}). In one embodiment, R¹ is methyl substituted with —C(O)R^{1a}, wherein R^{1a} is

In one embodiment. R¹ is C₁₋₆alkyl substituted with —C(O)N(R^{1b})(R^{1c}), wherein R^{1b} is C₁₋₄alkyl and R^{1c} is C₁₋₄alkyl optionally substituted with C(O)OR^{1b}. In one embodiment, R¹ is

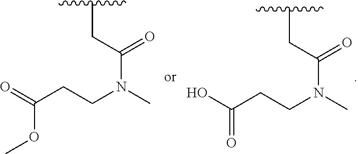 [SMILES: *CC(=O)N(C)CCC(=O)OC ; *CC(=O)N(C)CCC(=O)O]

In one embodiment, R¹ is C₁₋₆alkyl substituted with —C(O)N(R^{1b})(R^{1c}), wherein R^{1b} and R^{1c} together with an intervening nitrogen atom form a 4 to 6 membered heterocyclic ring, optionally containing an additional atom or group selected from N, O, S, S(O)₂ and optionally substituted with one or more groups selected from —C(O)OR^{1b} and —C(O)R^{1b}. In one embodiment, R¹ is C₁₋₆alkyl substituted with —C(O)N(R^{1b})(R^{1c}), wherein R^{1b} and R^{1c} together with the intervening nitrogen atom form an azetidine ring, which is optionally substituted with one or more groups selected from —C(O)OR^{1b} and —C(O)R^{1b}. In one embodiment, R¹ is

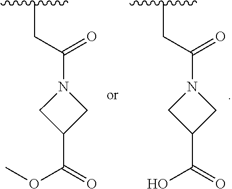 [SMILES: *CC(=O)N1CC(C(=O)OC)C1 ; *CC(=O)N1CC(C(=O)O)C1]

In one embodiment, the invention features benzoxazinones having formula (II):

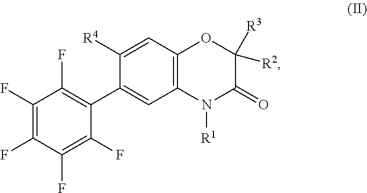

or a salt thereof (including an agriculturally suitable salt thereof).

In a further embodiment, each of R², R³, and R⁴ is F.

In another further embodiment, each of R², R¹, and R⁴ is F.

In one embodiment, the invention features benzoxazinones having formula (III):

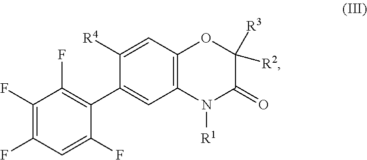

or salt thereof (including an agriculturally suitable salt thereof).

In a further embodiment, each of R², R³, and R⁴ is F.

In another further embodiment, each of R², R³, and R⁴ is F.

In one embodiment, the invention features benzoxazinones having formula (IV):

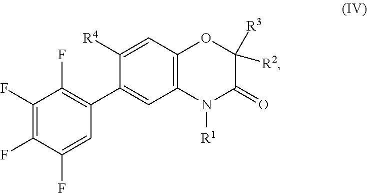

or salt thereof (including an agriculturally suitable salt thereof).

In a further embodiment, each of R², R³, and R⁴ is F.

In another further embodiment, each of R², R¹, and R⁴ is F.

In one embodiment, the invention features benzoxazinones having formula (V):

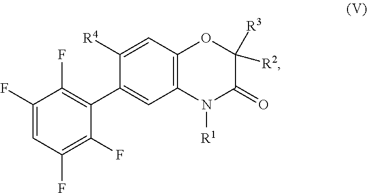

or salt thereof (including an agriculturally suitable salt thereof).

In a further embodiment, each of R², R³, and R⁴ is F.

In another further embodiment, each of R², R³, and R⁴ is F.

In some variations of the foregoing, the salt may be an agriculturally suitable salt.

In certain variations, the agriculturally suitable salt is a salt that exhibits herbicidal activity, or that is or can be converted in plants, water, or soil into a compound or salt with herbicidal activity.

In some aspects, provided is a compound selected from the compounds listed in Table 1 below, or a salt thereof (including an agriculturally suitable salt thereof).

TABLE 1
Exemplary Compounds

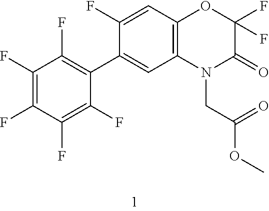 [SMILES: COC(=O)CN1C(=O)C(F)(F)Oc2cc(F)c(-c3c(F)c(F)c(F)c(F)c3F)cc21]

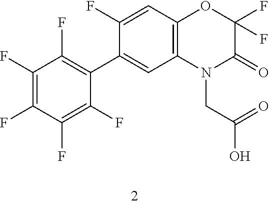 [SMILES: O=C(O)CN1C(=O)C(F)(F)Oc2cc(F)c(-c3c(F)c(F)c(F)c(F)c3F)cc21]

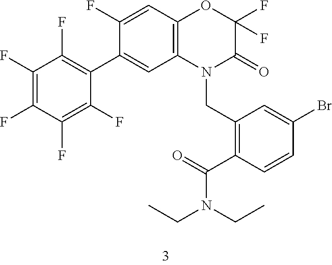 [SMILES: CCN(CC)C(=O)c1ccc(Br)cc1CN1C(=O)C(F)(F)Oc2cc(F)c(-c3c(F)c(F)c(F)c(F)c3F)cc21]

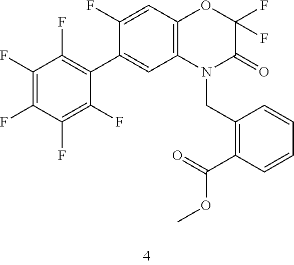 [SMILES: COC(=O)c1ccccc1CN1C(=O)C(F)(F)Oc2cc(F)c(-c3c(F)c(F)c(F)c(F)c3F)cc21]

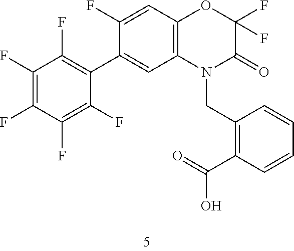 [SMILES: O=C(O)c1ccccc1CN1C(=O)C(F)(F)Oc2cc(F)c(-c3c(F)c(F)c(F)c(F)c3F)cc21]

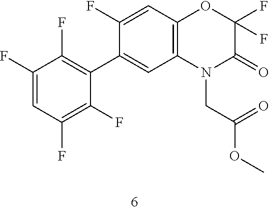 [SMILES: COC(=O)CN1C(=O)C(F)(F)Oc2cc(F)c(-c3c(F)c(F)cc(F)c3F)cc21]

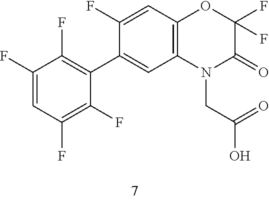 [SMILES: O=C(O)CN1C(=O)C(F)(F)Oc2cc(F)c(-c3c(F)c(F)cc(F)c3F)cc21]

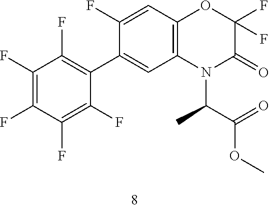 [SMILES: COC(=O)[C@@H](C)N1C(=O)C(F)(F)Oc2cc(F)c(-c3c(F)c(F)c(F)c(F)c3F)cc21]

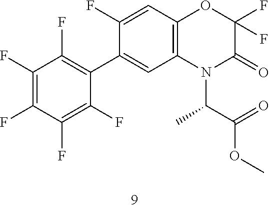 [SMILES: COC(=O)[C@H](C)N1C(=O)C(F)(F)Oc2cc(F)c(-c3c(F)c(F)c(F)c(F)c3F)cc21]

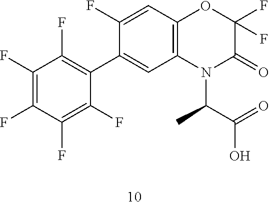 [SMILES: C[C@H](C(=O)O)N1C(=O)C(F)(F)Oc2cc(F)c(-c3c(F)c(F)c(F)c(F)c3F)cc21]

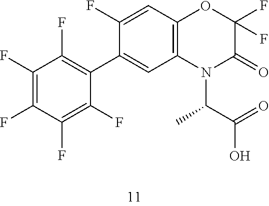 [SMILES: C[C@@H](C(=O)O)N1C(=O)C(F)(F)Oc2cc(F)c(-c3c(F)c(F)c(F)c(F)c3F)cc21]

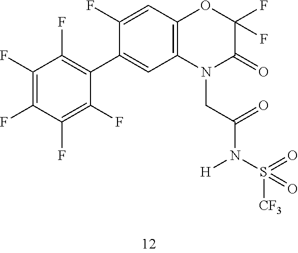 [SMILES: O=C(CN1C(=O)C(F)(F)Oc2cc(F)c(-c3c(F)c(F)c(F)c(F)c3F)cc21)NS(=O)(=O)C(F)(F)F]

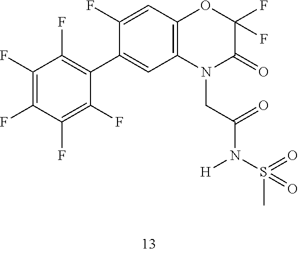 [SMILES: CS(=O)(=O)NC(=O)CN1C(=O)C(F)(F)Oc2cc(F)c(-c3c(F)c(F)c(F)c(F)c3F)cc21]

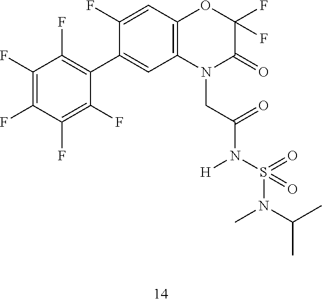 [SMILES: CC(C)N(C)S(=O)(=O)NC(=O)CN1C(=O)C(F)(F)Oc2cc(F)c(-c3c(F)c(F)c(F)c(F)c3F)cc21]

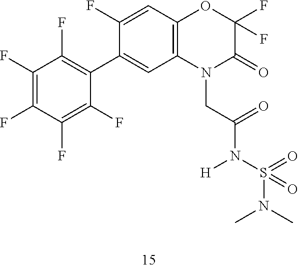 [SMILES: CN(C)S(=O)(=O)NC(=O)CN1C(=O)C(F)(F)Oc2cc(F)c(-c3c(F)c(F)c(F)c(F)c3F)cc21]

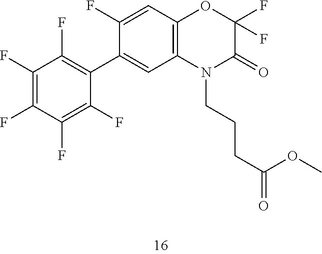 [SMILES: COC(=O)CCCN1C(=O)C(F)(F)Oc2cc(F)c(-c3c(F)c(F)c(F)c(F)c3F)cc21]

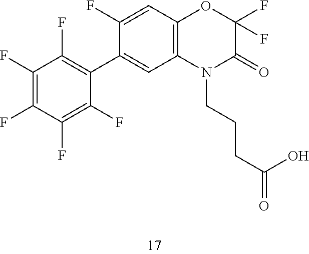 [SMILES: O=C(O)CCCN1C(=O)C(F)(F)Oc2cc(F)c(-c3c(F)c(F)c(F)c(F)c3F)cc21]

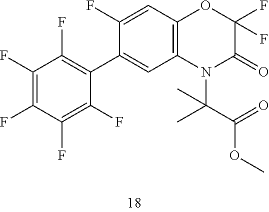 [SMILES: COC(=O)C(C)(C)N1C(=O)C(F)(F)Oc2cc(F)c(-c3c(F)c(F)c(F)c(F)c3F)cc21]

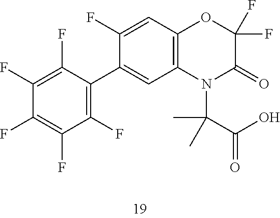 [SMILES: CC(C)(C(=O)O)N1C(=O)C(F)(F)Oc2cc(F)c(-c3c(F)c(F)c(F)c(F)c3F)cc21]

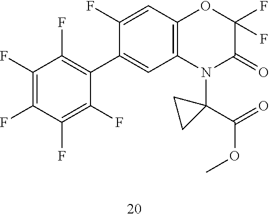 [SMILES: COC(=O)C1(N2C(=O)C(F)(F)Oc3cc(F)c(-c4c(F)c(F)c(F)c(F)c4F)cc32)CC1]

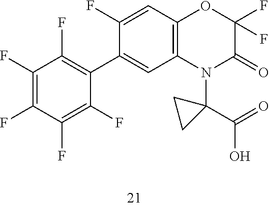 [SMILES: O=C1N(C2(C(=O)O)CC2)c2cc(-c3c(F)c(F)c(F)c(F)c3F)c(F)cc2OC1(F)F]

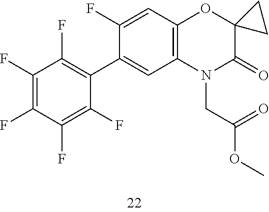 [SMILES: COC(=O)CN1C(=O)C2(CC2)Oc2cc(F)c(-c3c(F)c(F)c(F)c(F)c3F)cc21]

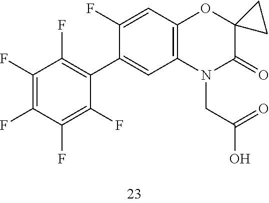 [SMILES: O=C(O)CN1C(=O)C2(CC2)Oc2cc(F)c(-c3c(F)c(F)c(F)c(F)c3F)cc21]

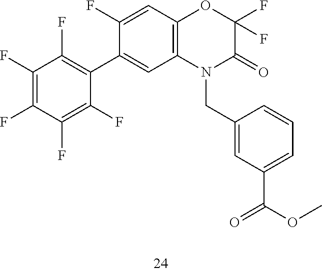 [SMILES: COC(=O)c1cccc(CN2C(=O)C(F)(F)Oc3cc(F)c(-c4c(F)c(F)c(F)c(F)c4F)cc32)c1]

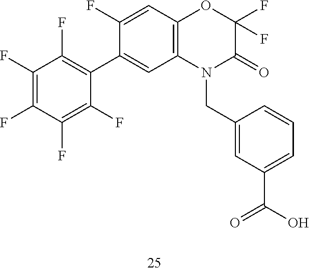 [SMILES: O=C(O)c1cccc(CN2C(=O)C(F)(F)Oc3cc(F)c(-c4c(F)c(F)c(F)c(F)c4F)cc32)c1]

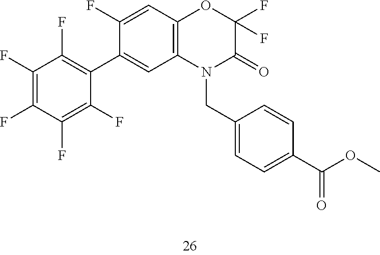 [SMILES: COC(=O)c1ccc(CN2C(=O)C(F)(F)Oc3cc(F)c(-c4c(F)c(F)c(F)c(F)c4F)cc32)cc1]

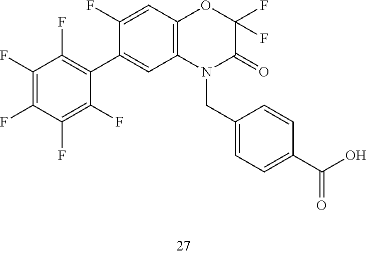 [SMILES: O=C(O)c1ccc(CN2C(=O)C(F)(F)Oc3cc(F)c(-c4c(F)c(F)c(F)c(F)c4F)cc32)cc1]

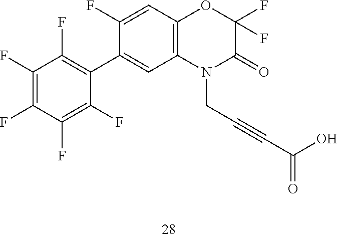 [SMILES: O=C(O)C#CCN1C(=O)C(F)(F)Oc2cc(F)c(-c3c(F)c(F)c(F)c(F)c3F)cc21]

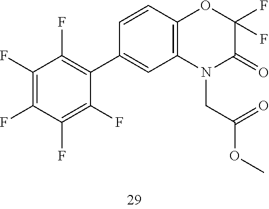 [SMILES: COC(=O)CN1C(=O)C(F)(F)Oc2ccc(-c3c(F)c(F)c(F)c(F)c3F)cc21]

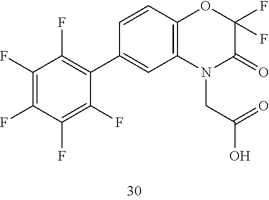 [SMILES: O=C(O)CN1C(=O)C(F)(F)Oc2ccc(-c3c(F)c(F)c(F)c(F)c3F)cc21]

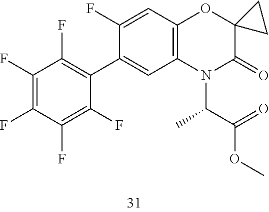 [SMILES: COC(=O)[C@H](C)N1C(=O)C2(CC2)Oc2cc(F)c(-c3c(F)c(F)c(F)c(F)c3F)cc21]

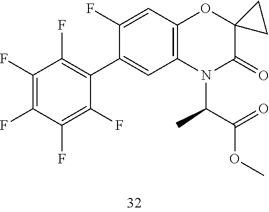 [SMILES: COC(=O)[C@@H](C)N1C(=O)C2(CC2)Oc2cc(F)c(-c3c(F)c(F)c(F)c(F)c3F)cc21]

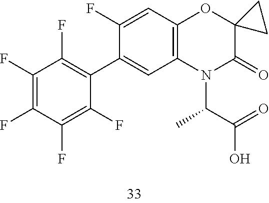 [SMILES: C[C@@H](C(=O)O)N1C(=O)C2(CC2)Oc2cc(F)c(-c3c(F)c(F)c(F)c(F)c3F)cc21]

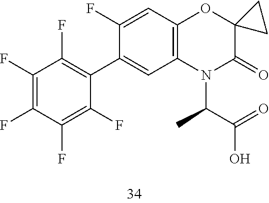 [SMILES: C[C@H](C(=O)O)N1C(=O)C2(CC2)Oc2cc(F)c(-c3c(F)c(F)c(F)c(F)c3F)cc21]

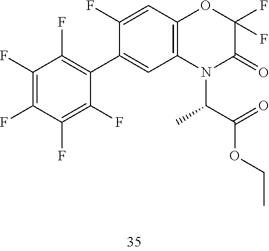 [SMILES: CCOC(=O)[C@H](C)N1C(=O)C(F)(F)Oc2cc(F)c(-c3c(F)c(F)c(F)c(F)c3F)cc21]

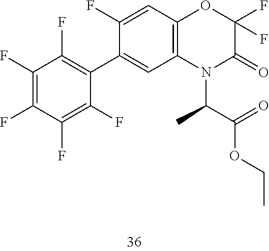 [SMILES: CCOC(=O)[C@@H](C)N1C(=O)C(F)(F)Oc2cc(F)c(-c3c(F)c(F)c(F)c(F)c3F)cc21]

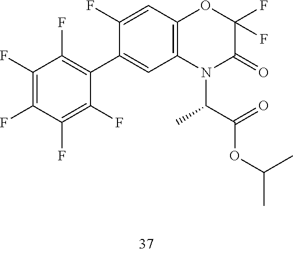 [SMILES: CC(C)OC(=O)[C@H](C)N1C(=O)C(F)(F)Oc2cc(F)c(-c3c(F)c(F)c(F)c(F)c3F)cc21]

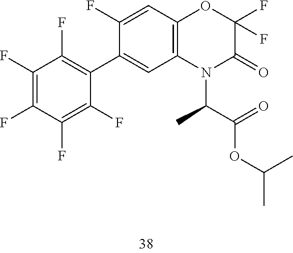 [SMILES: CC(C)OC(=O)[C@@H](C)N1C(=O)C(F)(F)Oc2cc(F)c(-c3c(F)c(F)c(F)c(F)c3F)cc21]

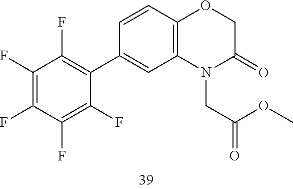 [SMILES: COC(=O)CN1C(=O)COc2ccc(-c3c(F)c(F)c(F)c(F)c3F)cc21]

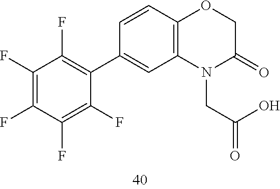 [SMILES: O=C(O)CN1C(=O)COc2ccc(-c3c(F)c(F)c(F)c(F)c3F)cc21]

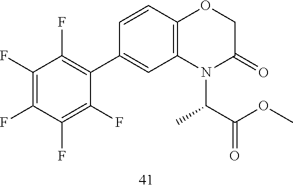 [SMILES: COC(=O)[C@H](C)N1C(=O)COc2ccc(-c3c(F)c(F)c(F)c(F)c3F)cc21]

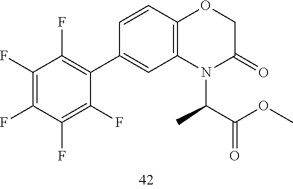 [SMILES: COC(=O)[C@@H](C)N1C(=O)COc2ccc(-c3c(F)c(F)c(F)c(F)c3F)cc21]

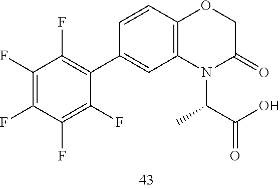 [SMILES: C[C@@H](C(=O)O)N1C(=O)COc2ccc(-c3c(F)c(F)c(F)c(F)c3F)cc21]

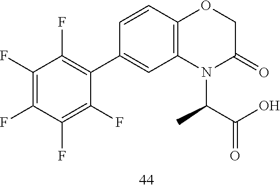 [SMILES: C[C@H](C(=O)O)N1C(=O)COc2ccc(-c3c(F)c(F)c(F)c(F)c3F)cc21]

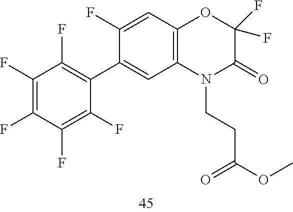 [SMILES: COC(=O)CCN1C(=O)C(F)(F)Oc2cc(F)c(-c3c(F)c(F)c(F)c(F)c3F)cc21]

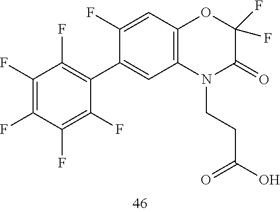 [SMILES: O=C(O)CCN1C(=O)C(F)(F)Oc2cc(F)c(-c3c(F)c(F)c(F)c(F)c3F)cc21]

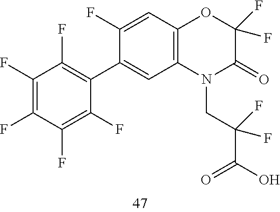 [SMILES: O=C(O)C(F)(F)CN1C(=O)C(F)(F)Oc2cc(F)c(-c3c(F)c(F)c(F)c(F)c3F)cc21]

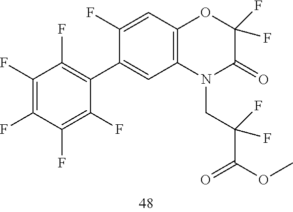 [SMILES: COC(=O)C(F)(F)CN1C(=O)C(F)(F)Oc2cc(F)c(-c3c(F)c(F)c(F)c(F)c3F)cc21]

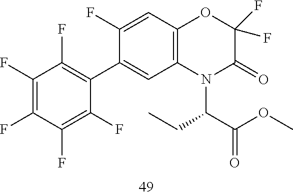 [SMILES: CC[C@@H](C(=O)OC)N1C(=O)C(F)(F)Oc2cc(F)c(-c3c(F)c(F)c(F)c(F)c3F)cc21]

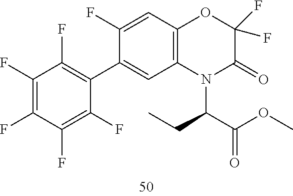 [SMILES: CC[C@H](C(=O)OC)N1C(=O)C(F)(F)Oc2cc(F)c(-c3c(F)c(F)c(F)c(F)c3F)cc21]

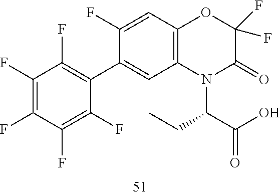 [SMILES: CC[C@@H](C(=O)O)N1C(=O)C(F)(F)Oc2cc(F)c(-c3c(F)c(F)c(F)c(F)c3F)cc21]

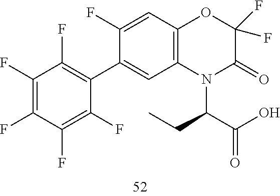 [SMILES: CC[C@H](C(=O)O)N1C(=O)C(F)(F)Oc2cc(F)c(-c3c(F)c(F)c(F)c(F)c3F)cc21]

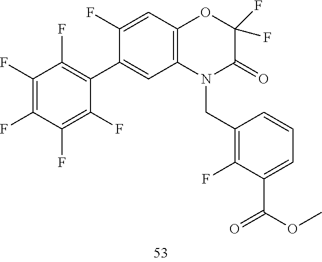 [SMILES: COC(=O)c1cccc(CN2C(=O)C(F)(F)Oc3cc(F)c(-c4c(F)c(F)c(F)c(F)c4F)cc32)c1F]

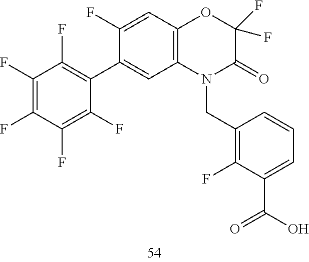 [SMILES: O=C(O)c1cccc(CN2C(=O)C(F)(F)Oc3cc(F)c(-c4c(F)c(F)c(F)c(F)c4F)cc32)c1F]

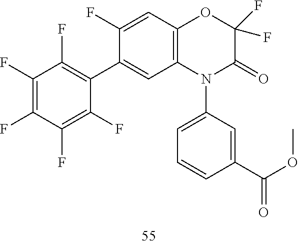 [SMILES: COC(=O)c1cccc(N2C(=O)C(F)(F)Oc3cc(F)c(-c4c(F)c(F)c(F)c(F)c4F)cc32)c1]

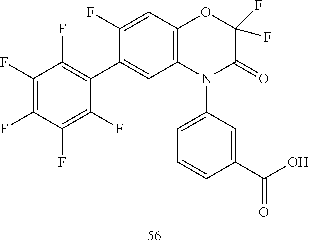 [SMILES: O=C(O)c1cccc(N2C(=O)C(F)(F)Oc3cc(F)c(-c4c(F)c(F)c(F)c(F)c4F)cc32)c1]

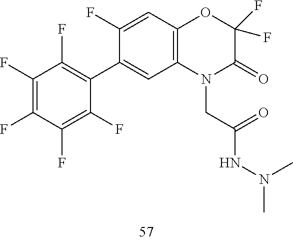 [SMILES: CN(C)NC(=O)CN1C(=O)C(F)(F)Oc2cc(F)c(-c3c(F)c(F)c(F)c(F)c3F)cc21]

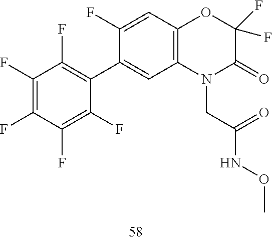 [SMILES: CONC(=O)CN1C(=O)C(F)(F)Oc2cc(F)c(-c3c(F)c(F)c(F)c(F)c3F)cc21]

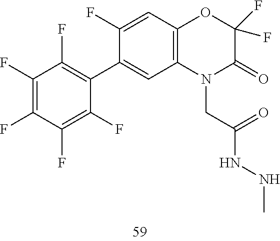 [SMILES: CNNC(=O)CN1C(=O)C(F)(F)Oc2cc(F)c(-c3c(F)c(F)c(F)c(F)c3F)cc21]

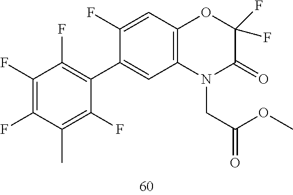 [SMILES: COC(=O)CN1C(=O)C(F)(F)Oc2cc(F)c(-c3c(F)c(C)c(F)c(F)c3F)cc21]

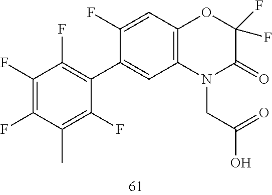 [SMILES: Cc1c(F)c(F)c(F)c(-c2cc3c(cc2F)OC(F)(F)C(=O)N3CC(=O)O)c1F]

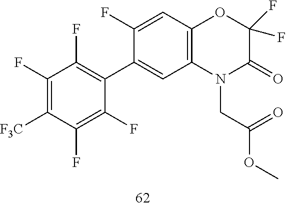 [SMILES: COC(=O)CN1C(=O)C(F)(F)Oc2cc(F)c(-c3c(F)c(F)c(C(F)(F)F)c(F)c3F)cc21]

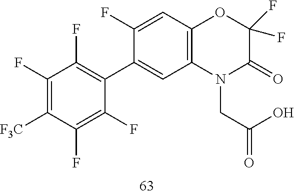 [SMILES: O=C(O)CN1C(=O)C(F)(F)Oc2cc(F)c(-c3c(F)c(F)c(C(F)(F)F)c(F)c3F)cc21]

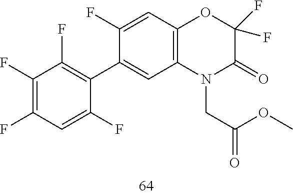 [SMILES: COC(=O)CN1C(=O)C(F)(F)Oc2cc(F)c(-c3c(F)cc(F)c(F)c3F)cc21]

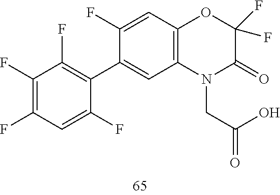 [SMILES: O=C(O)CN1C(=O)C(F)(F)Oc2cc(F)c(-c3c(F)cc(F)c(F)c3F)cc21]

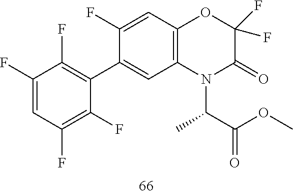 [SMILES: COC(=O)[C@H](C)N1C(=O)C(F)(F)Oc2cc(F)c(-c3c(F)c(F)cc(F)c3F)cc21]

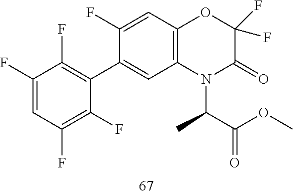 [SMILES: COC(=O)[C@@H](C)N1C(=O)C(F)(F)Oc2cc(F)c(-c3c(F)c(F)cc(F)c3F)cc21]

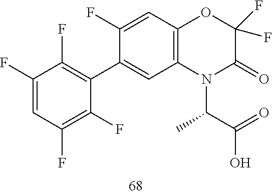 [SMILES: C[C@@H](C(=O)O)N1C(=O)C(F)(F)Oc2cc(F)c(-c3c(F)c(F)cc(F)c3F)cc21]

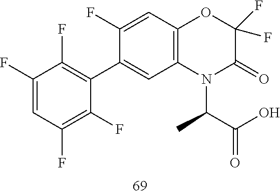 [SMILES: C[C@H](C(=O)O)N1C(=O)C(F)(F)Oc2cc(F)c(-c3c(F)c(F)cc(F)c3F)cc21]

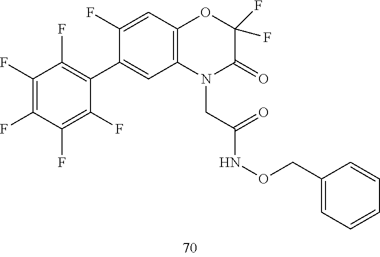 [SMILES: O=C(CN1C(=O)C(F)(F)Oc2cc(F)c(-c3c(F)c(F)c(F)c(F)c3F)cc21)NOCc1ccccc1]

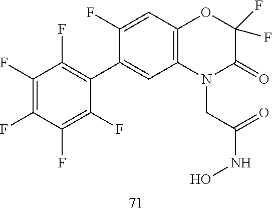 [SMILES: O=C(CN1C(=O)C(F)(F)Oc2cc(F)c(-c3c(F)c(F)c(F)c(F)c3F)cc21)NO]

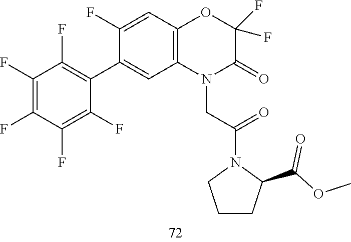 [SMILES: COC(=O)[C@H]1CCCN1C(=O)CN1C(=O)C(F)(F)Oc2cc(F)c(-c3c(F)c(F)c(F)c(F)c3F)cc21]

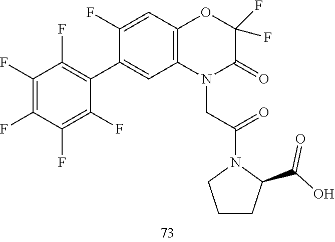 [SMILES: O=C(O)[C@H]1CCCN1C(=O)CN1C(=O)C(F)(F)Oc2cc(F)c(-c3c(F)c(F)c(F)c(F)c3F)cc21]

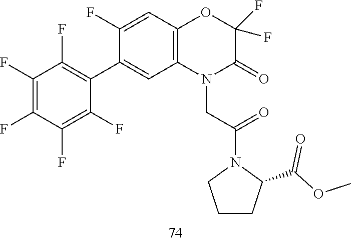 [SMILES: COC(=O)[C@@H]1CCCN1C(=O)CN1C(=O)C(F)(F)Oc2cc(F)c(-c3c(F)c(F)c(F)c(F)c3F)cc21]

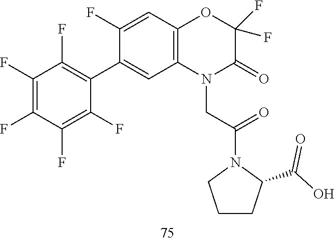 [SMILES: O=C(O)[C@@H]1CCCN1C(=O)CN1C(=O)C(F)(F)Oc2cc(F)c(-c3c(F)c(F)c(F)c(F)c3F)cc21]

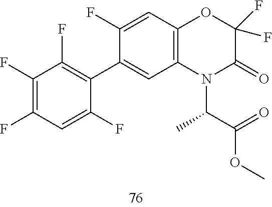 [SMILES: COC(=O)[C@H](C)N1C(=O)C(F)(F)Oc2cc(F)c(-c3c(F)cc(F)c(F)c3F)cc21]

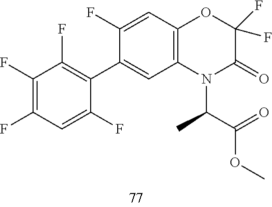 [SMILES: COC(=O)[C@@H](C)N1C(=O)C(F)(F)Oc2cc(F)c(-c3c(F)cc(F)c(F)c3F)cc21]

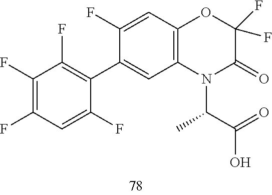 [SMILES: C[C@@H](C(=O)O)N1C(=O)C(F)(F)Oc2cc(F)c(-c3c(F)cc(F)c(F)c3F)cc21]

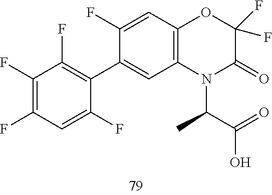 [SMILES: C[C@H](C(=O)O)N1C(=O)C(F)(F)Oc2cc(F)c(-c3c(F)cc(F)c(F)c3F)cc21]

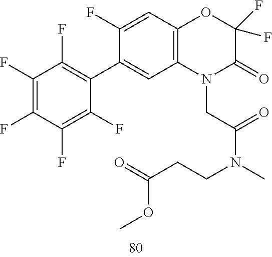 [SMILES: COC(=O)CCN(C)C(=O)CN1C(=O)C(F)(F)Oc2cc(F)c(-c3c(F)c(F)c(F)c(F)c3F)cc21]

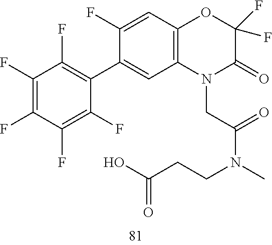 [SMILES: CN(CCC(=O)O)C(=O)CN1C(=O)C(F)(F)Oc2cc(F)c(-c3c(F)c(F)c(F)c(F)c3F)cc21]

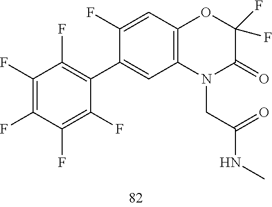 [SMILES: CNC(=O)CN1C(=O)C(F)(F)Oc2cc(F)c(-c3c(F)c(F)c(F)c(F)c3F)cc21]

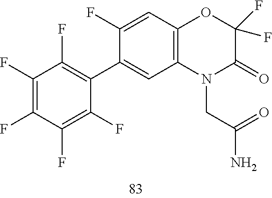 [SMILES: NC(=O)CN1C(=O)C(F)(F)Oc2cc(F)c(-c3c(F)c(F)c(F)c(F)c3F)cc21]

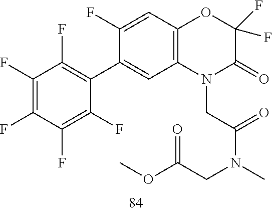 [SMILES: COC(=O)CN(C)C(=O)CN1C(=O)C(F)(F)Oc2cc(F)c(-c3c(F)c(F)c(F)c(F)c3F)cc21]

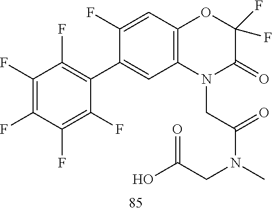 [SMILES: CN(CC(=O)O)C(=O)CN1C(=O)C(F)(F)Oc2cc(F)c(-c3c(F)c(F)c(F)c(F)c3F)cc21]

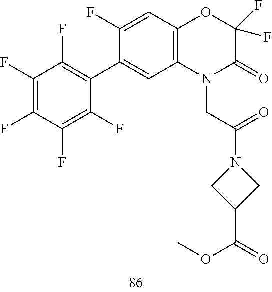 [SMILES: COC(=O)C1CN(C(=O)CN2C(=O)C(F)(F)Oc3cc(F)c(-c4c(F)c(F)c(F)c(F)c4F)cc32)C1]

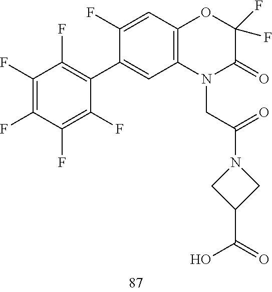 [SMILES: O=C(O)C1CN(C(=O)CN2C(=O)C(F)(F)Oc3cc(F)c(-c4c(F)c(F)c(F)c(F)c4F)cc32)C1]

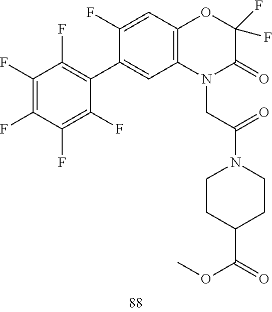 [SMILES: COC(=O)C1CCN(C(=O)CN2C(=O)C(F)(F)Oc3cc(F)c(-c4c(F)c(F)c(F)c(F)c4F)cc32)CC1]

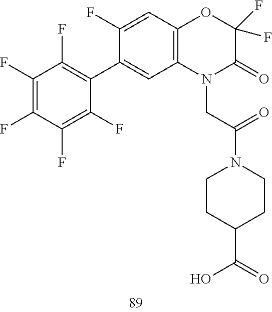 [SMILES: O=C(O)C1CCN(C(=O)CN2C(=O)C(F)(F)Oc3cc(F)c(-c4c(F)c(F)c(F)c(F)c4F)cc32)CC1]

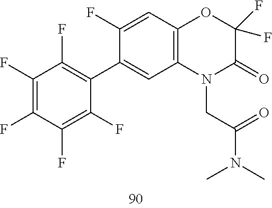 [SMILES: CN(C)C(=O)CN1C(=O)C(F)(F)Oc2cc(F)c(-c3c(F)c(F)c(F)c(F)c3F)cc21]

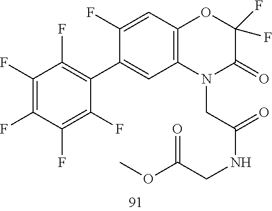 [SMILES: COC(=O)CNC(=O)CN1C(=O)C(F)(F)Oc2cc(F)c(-c3c(F)c(F)c(F)c(F)c3F)cc21]

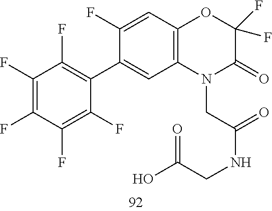 [SMILES: O=C(O)CNC(=O)CN1C(=O)C(F)(F)Oc2cc(F)c(-c3c(F)c(F)c(F)c(F)c3F)cc21]

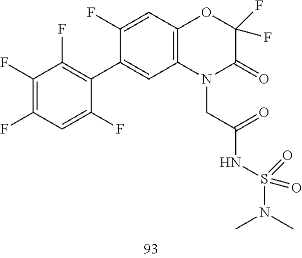 [SMILES: CN(C)S(=O)(=O)NC(=O)CN1C(=O)C(F)(F)Oc2cc(F)c(-c3c(F)cc(F)c(F)c3F)cc21]

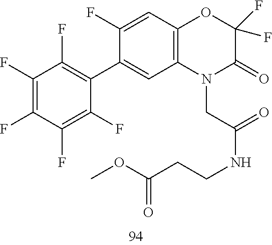 [SMILES: COC(=O)CCNC(=O)CN1C(=O)C(F)(F)Oc2cc(F)c(-c3c(F)c(F)c(F)c(F)c3F)cc21]

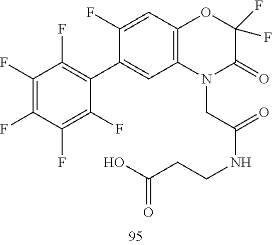 [SMILES: O=C(O)CCNC(=O)CN1C(=O)C(F)(F)Oc2cc(F)c(-c3c(F)c(F)c(F)c(F)c3F)cc21]

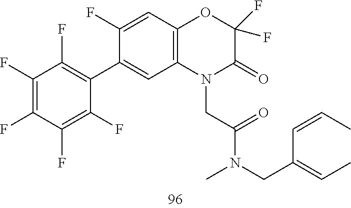 [SMILES: C/C=C\C(=C/C)CN(C)C(=O)CN1C(=O)C(F)(F)Oc2cc(F)c(-c3c(F)c(F)c(F)c(F)c3F)cc21]

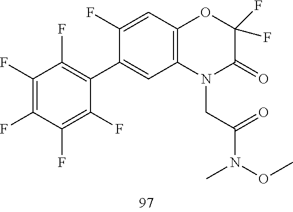 [SMILES: CON(C)C(=O)CN1C(=O)C(F)(F)Oc2cc(F)c(-c3c(F)c(F)c(F)c(F)c3F)cc21]

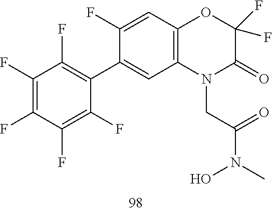 [SMILES: CN(O)C(=O)CN1C(=O)C(F)(F)Oc2cc(F)c(-c3c(F)c(F)c(F)c(F)c3F)cc21]

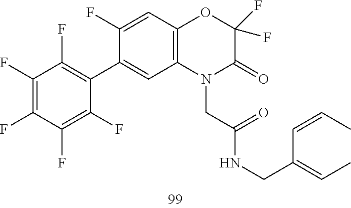 [SMILES: C/C=C\C(=C/C)CNC(=O)CN1C(=O)C(F)(F)Oc2cc(F)c(-c3c(F)c(F)c(F)c(F)c3F)cc21]

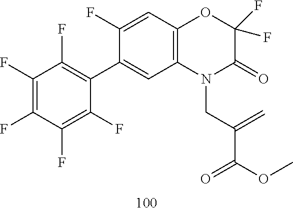 [SMILES: C=C(CN1C(=O)C(F)(F)Oc2cc(F)c(-c3c(F)c(F)c(F)c(F)c3F)cc21)C(=O)OC]

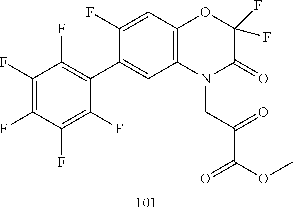 [SMILES: COC(=O)C(=O)CN1C(=O)C(F)(F)Oc2cc(F)c(-c3c(F)c(F)c(F)c(F)c3F)cc21]

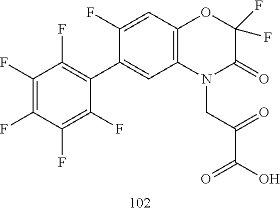 [SMILES: O=C(O)C(=O)CN1C(=O)C(F)(F)Oc2cc(F)c(-c3c(F)c(F)c(F)c(F)c3F)cc21]

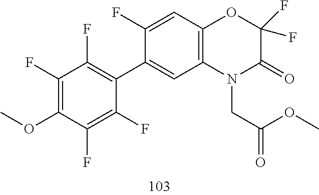

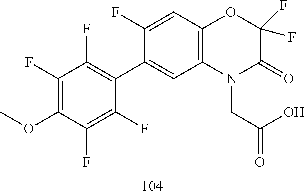 [SMILES: COc1c(F)c(F)c(-c2cc3c(cc2F)OC(F)(F)C(=O)N3CC(=O)O)c(F)c1F]

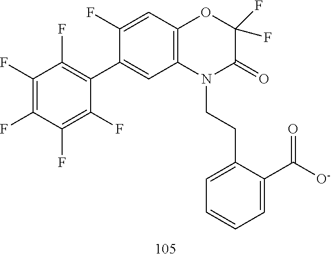 [SMILES: O=C([O-])c1ccccc1CCN1C(=O)C(F)(F)Oc2cc(F)c(-c3c(F)c(F)c(F)c(F)c3F)cc21]

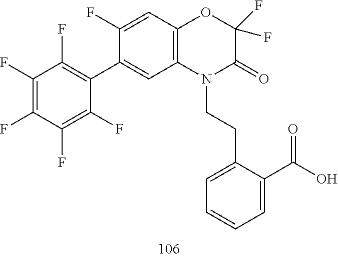 [SMILES: O=C(O)c1ccccc1CCN1C(=O)C(F)(F)Oc2cc(F)c(-c3c(F)c(F)c(F)c(F)c3F)cc21]

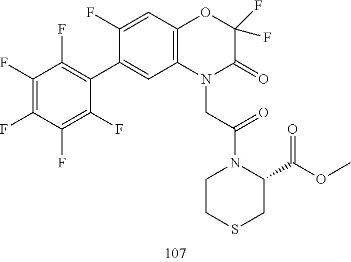 [SMILES: COC(=O)[C@@H]1CSCCN1C(=O)CN1C(=O)C(F)(F)Oc2cc(F)c(-c3c(F)c(F)c(F)c(F)c3F)cc21]

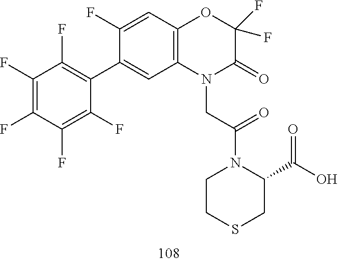 [SMILES: O=C(O)[C@@H]1CSCCN1C(=O)CN1C(=O)C(F)(F)Oc2cc(F)c(-c3c(F)c(F)c(F)c(F)c3F)cc21]

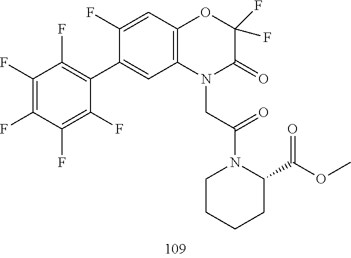 [SMILES: COC(=O)[C@@H]1CCCCN1C(=O)CN1C(=O)C(F)(F)Oc2cc(F)c(-c3c(F)c(F)c(F)c(F)c3F)cc21]

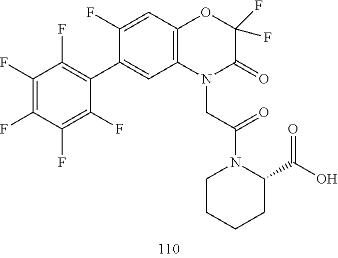 [SMILES: O=C(O)[C@@H]1CCCCN1C(=O)CN1C(=O)C(F)(F)Oc2cc(F)c(-c3c(F)c(F)c(F)c(F)c3F)cc21]

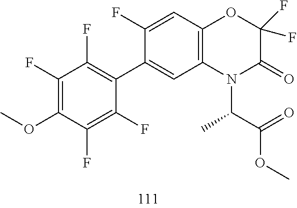 [SMILES: COC(=O)[C@H](C)N1C(=O)C(F)(F)Oc2cc(F)c(-c3c(F)c(F)c(OC)c(F)c3F)cc21]

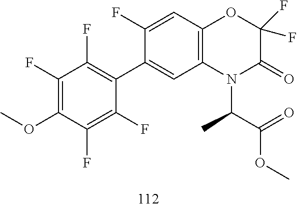 [SMILES: COC(=O)[C@@H](C)N1C(=O)C(F)(F)Oc2cc(F)c(-c3c(F)c(F)c(OC)c(F)c3F)cc21]

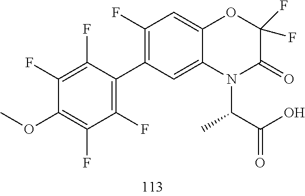 [SMILES: COc1c(F)c(F)c(-c2cc3c(cc2F)OC(F)(F)C(=O)N3[C@@H](C)C(=O)O)c(F)c1F]

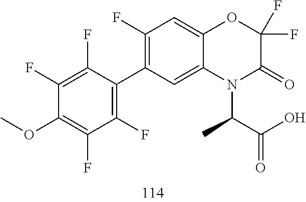 [SMILES: COc1c(F)c(F)c(-c2cc3c(cc2F)OC(F)(F)C(=O)N3[C@H](C)C(=O)O)c(F)c1F]

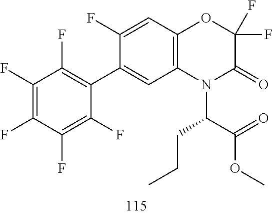 [SMILES: CCC[C@@H](C(=O)OC)N1C(=O)C(F)(F)Oc2cc(F)c(-c3c(F)c(F)c(F)c(F)c3F)cc21]

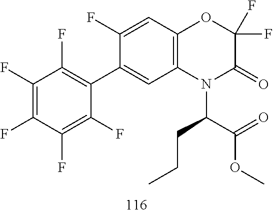 [SMILES: CCC[C@H](C(=O)OC)N1C(=O)C(F)(F)Oc2cc(F)c(-c3c(F)c(F)c(F)c(F)c3F)cc21]

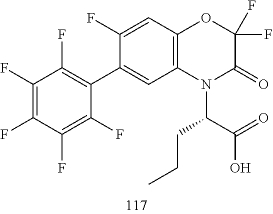 [SMILES: CCC[C@@H](C(=O)O)N1C(=O)C(F)(F)Oc2cc(F)c(-c3c(F)c(F)c(F)c(F)c3F)cc21]

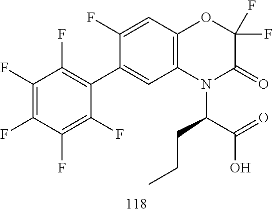 [SMILES: CCC[C@H](C(=O)O)N1C(=O)C(F)(F)Oc2cc(F)c(-c3c(F)c(F)c(F)c(F)c3F)cc21]

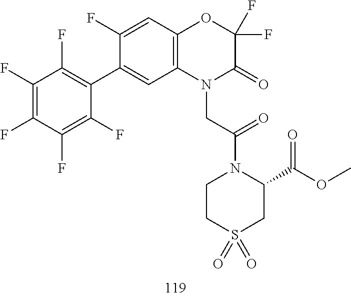 [SMILES: COC(=O)[C@@H]1CS(=O)(=O)CCN1C(=O)CN1C(=O)C(F)(F)Oc2cc(F)c(-c3c(F)c(F)c(F)c(F)c3F)cc21]

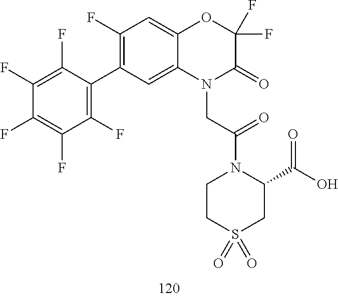 [SMILES: O=C(O)[C@@H]1CS(=O)(=O)CCN1C(=O)CN1C(=O)C(F)(F)Oc2cc(F)c(-c3c(F)c(F)c(F)c(F)c3F)cc21]

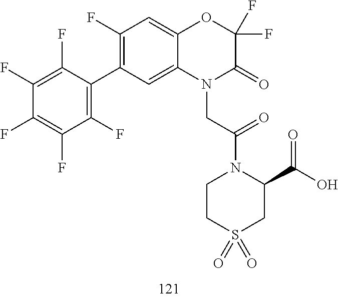 [SMILES: O=C(O)[C@H]1CS(=O)(=O)CCN1C(=O)CN1C(=O)C(F)(F)Oc2cc(F)c(-c3c(F)c(F)c(F)c(F)c3F)cc21]

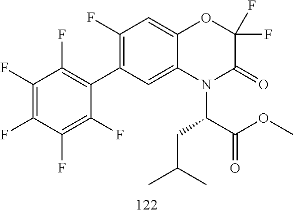 [SMILES: COC(=O)[C@H](CC(C)C)N1C(=O)C(F)(F)Oc2cc(F)c(-c3c(F)c(F)c(F)c(F)c3F)cc21]

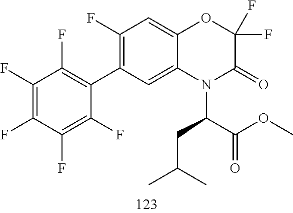 [SMILES: COC(=O)[C@@H](CC(C)C)N1C(=O)C(F)(F)Oc2cc(F)c(-c3c(F)c(F)c(F)c(F)c3F)cc21]

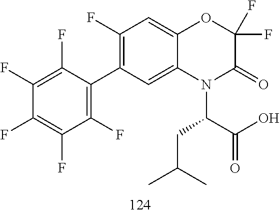 [SMILES: CC(C)C[C@@H](C(=O)O)N1C(=O)C(F)(F)Oc2cc(F)c(-c3c(F)c(F)c(F)c(F)c3F)cc21]

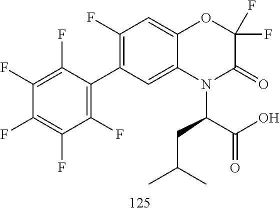 [SMILES: CC(C)C[C@H](C(=O)O)N1C(=O)C(F)(F)Oc2cc(F)c(-c3c(F)c(F)c(F)c(F)c3F)cc21]

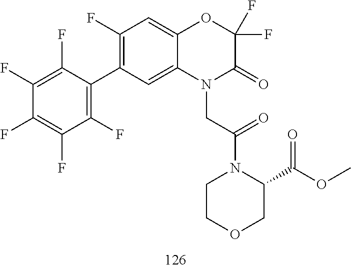 [SMILES: COC(=O)[C@@H]1COCCN1C(=O)CN1C(=O)C(F)(F)Oc2cc(F)c(-c3c(F)c(F)c(F)c(F)c3F)cc21]

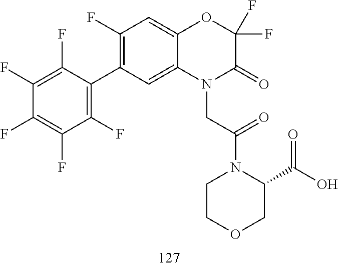 [SMILES: O=C(O)[C@@H]1COCCN1C(=O)CN1C(=O)C(F)(F)Oc2cc(F)c(-c3c(F)c(F)c(F)c(F)c3F)cc21]

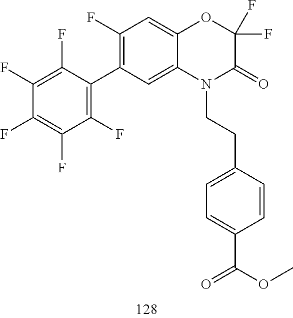 [SMILES: COC(=O)c1ccc(CCN2C(=O)C(F)(F)Oc3cc(F)c(-c4c(F)c(F)c(F)c(F)c4F)cc32)cc1]

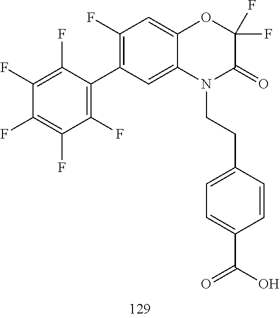 [SMILES: O=C(O)c1ccc(CCN2C(=O)C(F)(F)Oc3cc(F)c(-c4c(F)c(F)c(F)c(F)c4F)cc32)cc1]

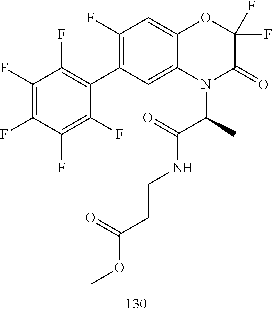 [SMILES: COC(=O)CCNC(=O)[C@H](C)N1C(=O)C(F)(F)Oc2cc(F)c(-c3c(F)c(F)c(F)c(F)c3F)cc21]

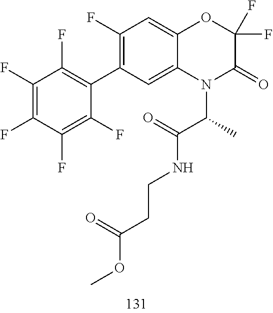 [SMILES: COC(=O)CCNC(=O)[C@@H](C)N1C(=O)C(F)(F)Oc2cc(F)c(-c3c(F)c(F)c(F)c(F)c3F)cc21]

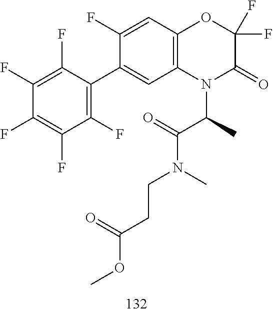 [SMILES: COC(=O)CCN(C)C(=O)[C@H](C)N1C(=O)C(F)(F)Oc2cc(F)c(-c3c(F)c(F)c(F)c(F)c3F)cc21]

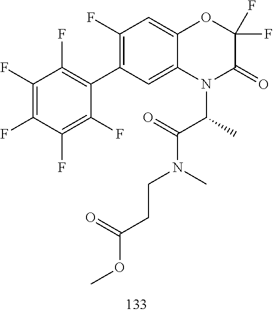 [SMILES: COC(=O)CCN(C)C(=O)[C@@H](C)N1C(=O)C(F)(F)Oc2cc(F)c(-c3c(F)c(F)c(F)c(F)c3F)cc21]

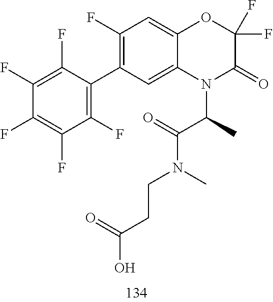 [SMILES: C[C@@H](C(=O)N(C)CCC(=O)O)N1C(=O)C(F)(F)Oc2cc(F)c(-c3c(F)c(F)c(F)c(F)c3F)cc21]

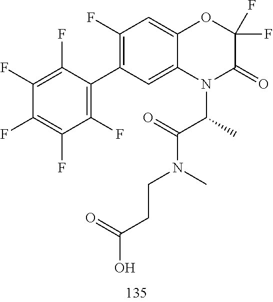 [SMILES: C[C@H](C(=O)N(C)CCC(=O)O)N1C(=O)C(F)(F)Oc2cc(F)c(-c3c(F)c(F)c(F)c(F)c3F)cc21]

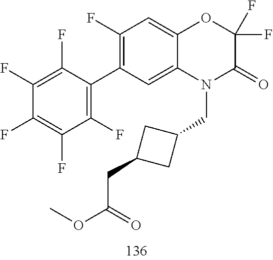 [SMILES: COC(=O)C[C@H]1C[C@H](CN2C(=O)C(F)(F)Oc3cc(F)c(-c4c(F)c(F)c(F)c(F)c4F)cc32)C1]

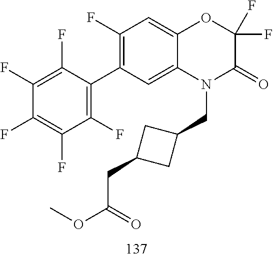 [SMILES: COC(=O)C[C@H]1C[C@@H](CN2C(=O)C(F)(F)Oc3cc(F)c(-c4c(F)c(F)c(F)c(F)c4F)cc32)C1]

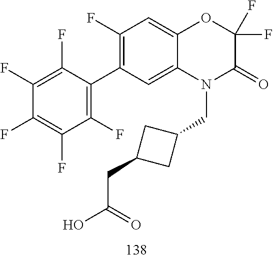 [SMILES: O=C(O)C[C@H]1C[C@H](CN2C(=O)C(F)(F)Oc3cc(F)c(-c4c(F)c(F)c(F)c(F)c4F)cc32)C1]

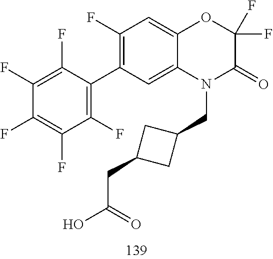 [SMILES: O=C(O)C[C@H]1C[C@@H](CN2C(=O)C(F)(F)Oc3cc(F)c(-c4c(F)c(F)c(F)c(F)c4F)cc32)C1]

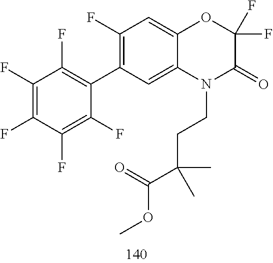 [SMILES: COC(=O)C(C)(C)CCN1C(=O)C(F)(F)Oc2cc(F)c(-c3c(F)c(F)c(F)c(F)c3F)cc21]

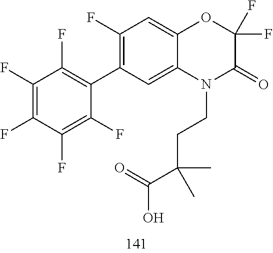 [SMILES: CC(C)(CCN1C(=O)C(F)(F)Oc2cc(F)c(-c3c(F)c(F)c(F)c(F)c3F)cc21)C(=O)O]

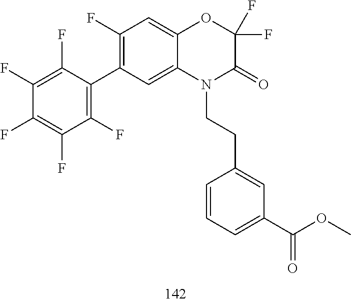 [SMILES: COC(=O)c1cccc(CCN2C(=O)C(F)(F)Oc3cc(F)c(-c4c(F)c(F)c(F)c(F)c4F)cc32)c1]

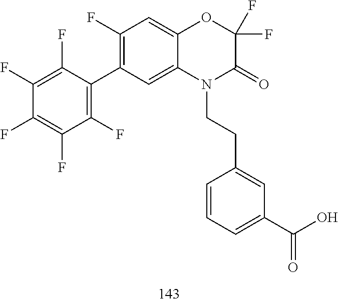 [SMILES: O=C(O)c1cccc(CCN2C(=O)C(F)(F)Oc3cc(F)c(-c4c(F)c(F)c(F)c(F)c4F)cc32)c1]

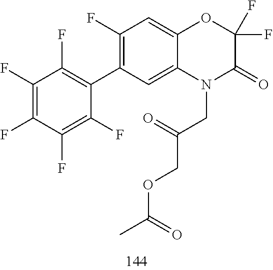 [SMILES: CC(=O)OCC(=O)CN1C(=O)C(F)(F)Oc2cc(F)c(-c3c(F)c(F)c(F)c(F)c3F)cc21]

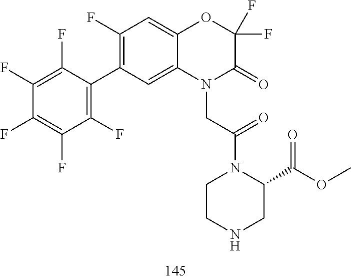 [SMILES: COC(=O)[C@@H]1CNCCN1C(=O)CN1C(=O)C(F)(F)Oc2cc(F)c(-c3c(F)c(F)c(F)c(F)c3F)cc21]

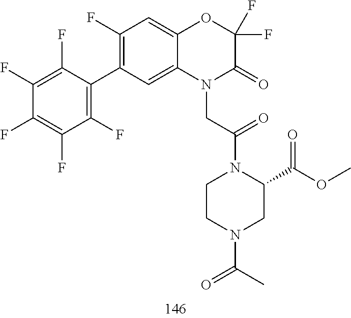 [SMILES: COC(=O)[C@@H]1CN(C(C)=O)CCN1C(=O)CN1C(=O)C(F)(F)Oc2cc(F)c(-c3c(F)c(F)c(F)c(F)c3F)cc21]

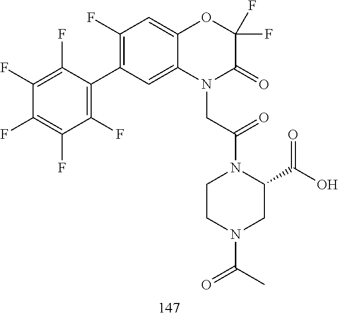 [SMILES: CC(=O)N1CCN(C(=O)CN2C(=O)C(F)(F)Oc3cc(F)c(-c4c(F)c(F)c(F)c(F)c4F)cc32)[C@H](C(=O)O)C1]

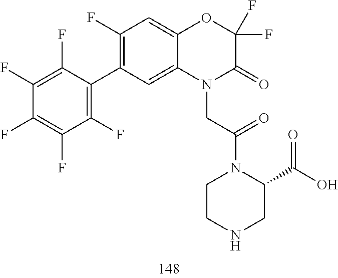 [SMILES: O=C(O)[C@@H]1CNCCN1C(=O)CN1C(=O)C(F)(F)Oc2cc(F)c(-c3c(F)c(F)c(F)c(F)c3F)cc21]

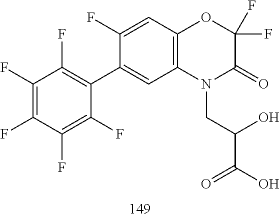 [SMILES: O=C(O)C(O)CN1C(=O)C(F)(F)Oc2cc(F)c(-c3c(F)c(F)c(F)c(F)c3F)cc21]

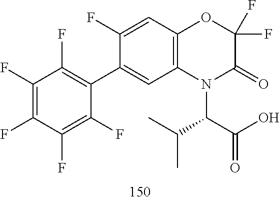 [SMILES: CC(C)[C@@H](C(=O)O)N1C(=O)C(F)(F)Oc2cc(F)c(-c3c(F)c(F)c(F)c(F)c3F)cc21]

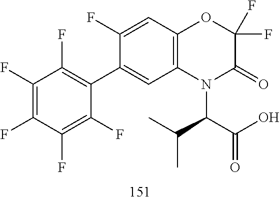 [SMILES: CC(C)[C@H](C(=O)O)N1C(=O)C(F)(F)Oc2cc(F)c(-c3c(F)c(F)c(F)c(F)c3F)cc21]

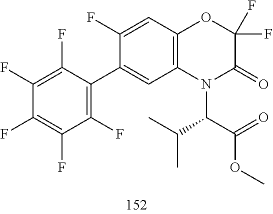 [SMILES: COC(=O)[C@H](C(C)C)N1C(=O)C(F)(F)Oc2cc(F)c(-c3c(F)c(F)c(F)c(F)c3F)cc21]

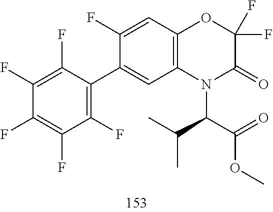 [SMILES: COC(=O)[C@@H](C(C)C)N1C(=O)C(F)(F)Oc2cc(F)c(-c3c(F)c(F)c(F)c(F)c3F)cc21]

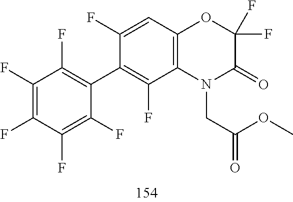 [SMILES: COC(=O)CN1C(=O)C(F)(F)Oc2cc(F)c(-c3c(F)c(F)c(F)c(F)c3F)c(F)c21]

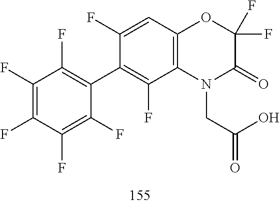 [SMILES: O=C(O)CN1C(=O)C(F)(F)Oc2cc(F)c(-c3c(F)c(F)c(F)c(F)c3F)c(F)c21]

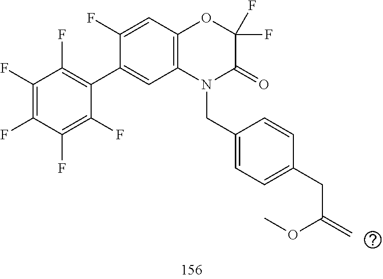 [SMILES: C=C(Cc1ccc(CN2C(=O)C(F)(F)Oc3cc(F)c(-c4c(F)c(F)c(F)c(F)c4F)cc32)cc1)OC]

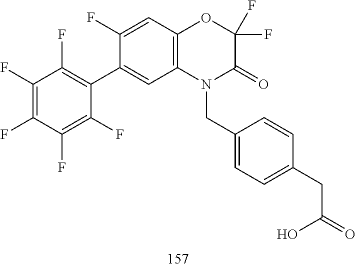 [SMILES: O=C(O)Cc1ccc(CN2C(=O)C(F)(F)Oc3cc(F)c(-c4c(F)c(F)c(F)c(F)c4F)cc32)cc1]

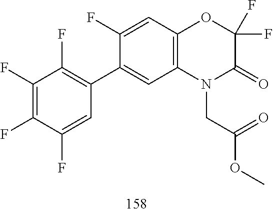 [SMILES: COC(=O)CN1C(=O)C(F)(F)Oc2cc(F)c(-c3cc(F)c(F)c(F)c3F)cc21]

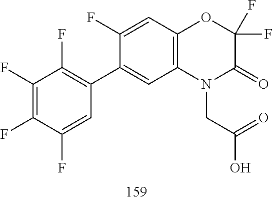 [SMILES: O=C(O)CN1C(=O)C(F)(F)Oc2cc(F)c(-c3cc(F)c(F)c(F)c3F)cc21]

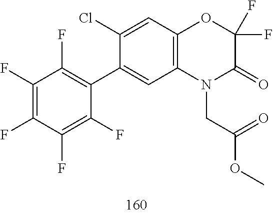 [SMILES: COC(=O)CN1C(=O)C(F)(F)Oc2cc(Cl)c(-c3c(F)c(F)c(F)c(F)c3F)cc21]

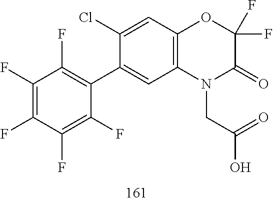 [SMILES: O=C(O)CN1C(=O)C(F)(F)Oc2cc(Cl)c(-c3c(F)c(F)c(F)c(F)c3F)cc21]

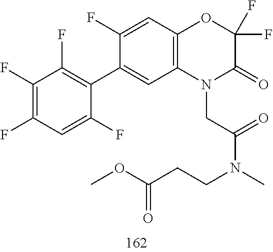 [SMILES: COC(=O)CCN(C)C(=O)CN1C(=O)C(F)(F)Oc2cc(F)c(-c3c(F)cc(F)c(F)c3F)cc21]

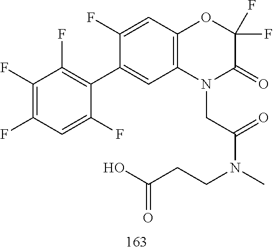 [SMILES: CN(CCC(=O)O)C(=O)CN1C(=O)C(F)(F)Oc2cc(F)c(-c3c(F)cc(F)c(F)c3F)cc21]

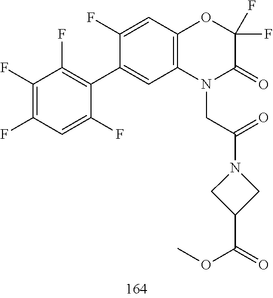 [SMILES: COC(=O)C1CN(C(=O)CN2C(=O)C(F)(F)Oc3cc(F)c(-c4c(F)cc(F)c(F)c4F)cc32)C1]

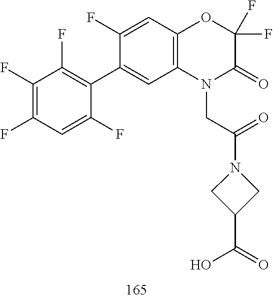 [SMILES: O=C(O)C1CN(C(=O)CN2C(=O)C(F)(F)Oc3cc(F)c(-c4c(F)cc(F)c(F)c4F)cc32)C1]

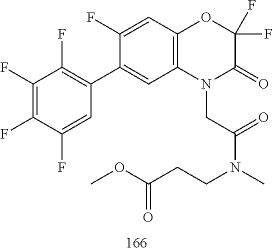 [SMILES: COC(=O)CCN(C)C(=O)CN1C(=O)C(F)(F)Oc2cc(F)c(-c3cc(F)c(F)c(F)c3F)cc21]

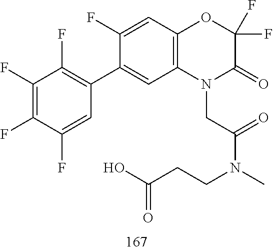 [SMILES: CN(CCC(=O)O)C(=O)CN1C(=O)C(F)(F)Oc2cc(F)c(-c3cc(F)c(F)c(F)c3F)cc21]

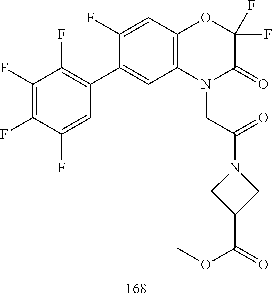 [SMILES: COC(=O)C1CN(C(=O)CN2C(=O)C(F)(F)Oc3cc(F)c(-c4cc(F)c(F)c(F)c4F)cc32)C1]

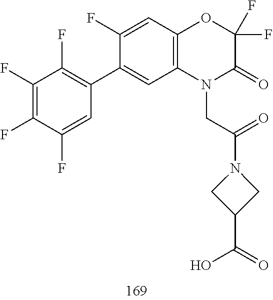 [SMILES: O=C(O)C1CN(C(=O)CN2C(=O)C(F)(F)Oc3cc(F)c(-c4cc(F)c(F)c(F)c4F)cc32)C1]

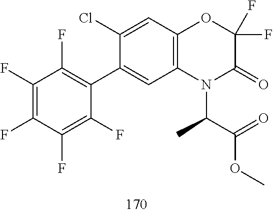 [SMILES: COC(=O)[C@@H](C)N1C(=O)C(F)(F)Oc2cc(Cl)c(-c3c(F)c(F)c(F)c(F)c3F)cc21]

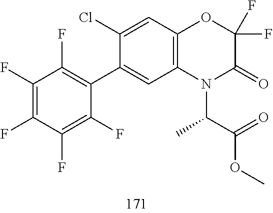 [SMILES: COC(=O)[C@H](C)N1C(=O)C(F)(F)Oc2cc(Cl)c(-c3c(F)c(F)c(F)c(F)c3F)cc21]

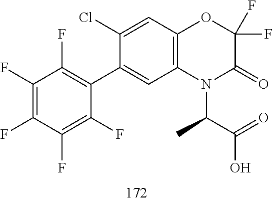 [SMILES: C[C@H](C(=O)O)N1C(=O)C(F)(F)Oc2cc(Cl)c(-c3c(F)c(F)c(F)c(F)c3F)cc21]

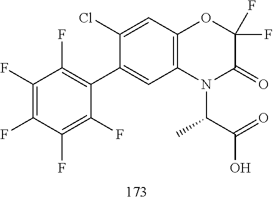 [SMILES: C[C@@H](C(=O)O)N1C(=O)C(F)(F)Oc2cc(Cl)c(-c3c(F)c(F)c(F)c(F)c3F)cc21]

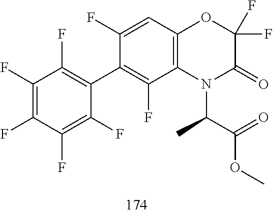 [SMILES: COC(=O)[C@@H](C)N1C(=O)C(F)(F)Oc2cc(F)c(-c3c(F)c(F)c(F)c(F)c3F)c(F)c21]

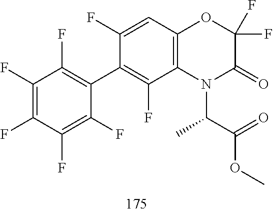 [SMILES: COC(=O)[C@H](C)N1C(=O)C(F)(F)Oc2cc(F)c(-c3c(F)c(F)c(F)c(F)c3F)c(F)c21]

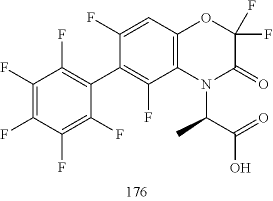 [SMILES: C[C@H](C(=O)O)N1C(=O)C(F)(F)Oc2cc(F)c(-c3c(F)c(F)c(F)c(F)c3F)c(F)c21]

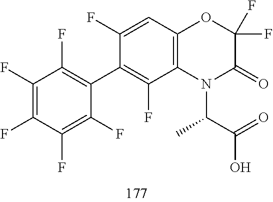 [SMILES: C[C@@H](C(=O)O)N1C(=O)C(F)(F)Oc2cc(F)c(-c3c(F)c(F)c(F)c(F)c3F)c(F)c21]

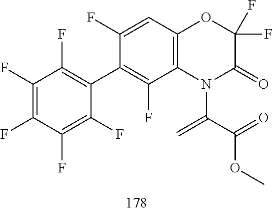 [SMILES: C=C(C(=O)OC)N1C(=O)C(F)(F)Oc2cc(F)c(-c3c(F)c(F)c(F)c(F)c3F)c(F)c21]

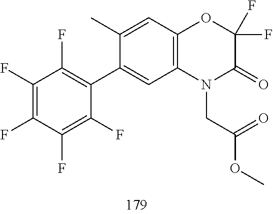 [SMILES: COC(=O)CN1C(=O)C(F)(F)Oc2cc(C)c(-c3c(F)c(F)c(F)c(F)c3F)cc21]

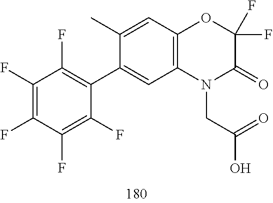 [SMILES: Cc1cc2c(cc1-c1c(F)c(F)c(F)c(F)c1F)N(CC(=O)O)C(=O)C(F)(F)O2]

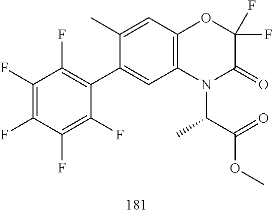 [SMILES: COC(=O)[C@H](C)N1C(=O)C(F)(F)Oc2cc(C)c(-c3c(F)c(F)c(F)c(F)c3F)cc21]

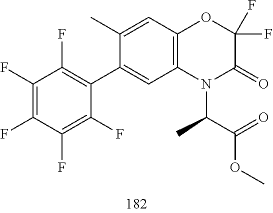 [SMILES: COC(=O)[C@@H](C)N1C(=O)C(F)(F)Oc2cc(C)c(-c3c(F)c(F)c(F)c(F)c3F)cc21]

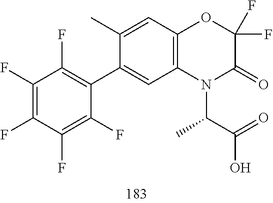 [SMILES: Cc1cc2c(cc1-c1c(F)c(F)c(F)c(F)c1F)N([C@@H](C)C(=O)O)C(=O)C(F)(F)O2]

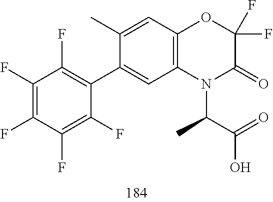 [SMILES: Cc1cc2c(cc1-c1c(F)c(F)c(F)c(F)c1F)N([C@H](C)C(=O)O)C(=O)C(F)(F)O2]

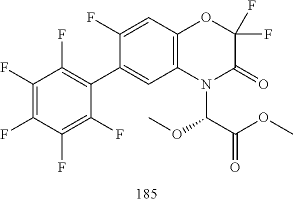 [SMILES: COC(=O)[C@H](OC)N1C(=O)C(F)(F)Oc2cc(F)c(-c3c(F)c(F)c(F)c(F)c3F)cc21]

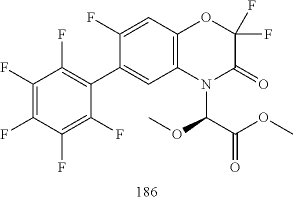 [SMILES: COC(=O)[C@@H](OC)N1C(=O)C(F)(F)Oc2cc(F)c(-c3c(F)c(F)c(F)c(F)c3F)cc21]

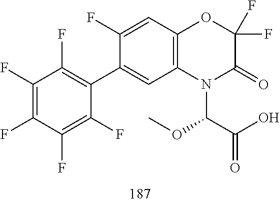 [SMILES: CO[C@@H](C(=O)O)N1C(=O)C(F)(F)Oc2cc(F)c(-c3c(F)c(F)c(F)c(F)c3F)cc21]

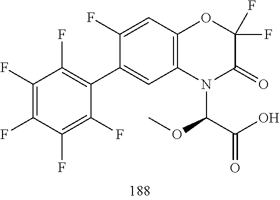 [SMILES: CO[C@H](C(=O)O)N1C(=O)C(F)(F)Oc2cc(F)c(-c3c(F)c(F)c(F)c(F)c3F)cc21]

In some variations, provided is Compound 8 to 11, or a salt thereof (including an agriculturally suitable salt thereof). In some variations, provided is Compound 76 to 79, or a salt thereof (including an agriculturally suitable salt thereof). In some variations, provided is Compound 80 to 81, or a salt thereof (including an agriculturally suitable salt thereof). In some variations, provided is Compound 86 to 87, or a salt thereof (including an agriculturally suitable salt thereof). In some variations, provided is Compound 162 to 165, or a salt thereof (including an agriculturally suitable salt thereof).

In another aspect, the invention features an agricultural composition comprising a compound of of the invention, or a salt thereof, and at least one additional component that serves as a carrier.

In one embodiment, at least one additional component of the agricultural composition is a surfactant or a diluent.

In another embodiment, the composition is an herbicidal composition.

In another aspect, the invention features a method of controlling undesired vegetation, the method comprising contacting the vegetation or its environment with an herbicidally effective amount of a compound of the invention, or agriculturally acceptable salt thereof.

In one embodiment, the undesired vegetation includes weeds. In a further embodiment, the undesired vegetation includes protoporphyrinogen IX oxidase (PPO) inhibitor-resistant weeds. In yet another further embodiment, the PPO inhibitor-resistant weeds have a dG210 mutation.

In one embodiment, a compound or composition of the invention is applied at a rate of 1 to 100 g per 10,000 m².

In one embodiment, contacting the undesired vegetation or its environment with a compound or composition of the invention leads to post-emergence control of the undesired vegetation. In a further embodiment, the undesired vegetation is at least 60% controlled. In another embodiment the undesired vegetation is at least 80% controlled. In yet another embodiment, the undesired vegetation is at least 90% controlled.

In one embodiment, contacting the undesired vegetation or its environment with a compound or composition of the invention leads to pre-emergence control of the undesired vegetation. In a further embodiment, the undesired vegetation is at least 60% controlled. In another embodiment the undesired vegetation is at least 80% controlled. In yet another embodiment, the undesired vegetation is at least 90% controlled.

### Definitions

As used herein, the terms “comprises,” “comprising,” “includes,” “including,” “has,” “having,” “contains,” “containing,” “characterized by,” or any other variation thereof, are intended to cover a non-exclusive inclusion, subject to any limitation explicitly indicated. For example, a composition, mixture, process, or method that includes or comprises a list of elements is not necessarily limited to only those elements but may include other elements not expressly listed or inherent to such composition, mixture, process, or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

Further, unless expressly stated to the contrary, “or” refers to an inclusive ‘or’ and not to an exclusive ‘or.’ For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling,” used either alone or in a combination of words means a young plant developing from the embryo of a seed.

As referred to herein, the term “broadleaf,” used either alone or in terms such as “broadleaf weed” means dicot or dicotyledon, a term used to describe a group of angiosperms characterized by embryos having two cotyledons.

In the above recitations, the term “alkyl,” used either alone or in compound words such as “alkylthio” or “haloalkyl” includes straight-chain or branched alkyl, such as, methyl, ethyl, n-propyl, i-propyl, or the different butyl, pentyl, or hexyl isomers. “Alkenyl” includes straight-chain or branched alkenes such as ethenyl, 1-propenyl, 2-propenyl, and the different butenyl, pentenyl, and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl, and the different butynyl, pentynyl, and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy, and hexyloxy isomers.

“Cycloalkyl” includes, for example, cyclopropyl, cyclobutyl, cyclopentyl, and cyclohexyl.

The term “halogen” or “halo” either alone or in compound words such as “haloalkyl,” or when used in descriptions such as “alkyl substituted with halogen” includes fluorine, chlorine, bromine, or iodine.

The total number of carbon atoms in a substituent group is indicated by the “Cᵢ-Cⱼ” or “Cᵢ₋ⱼ” prefix, where i and j are numbers from 1 to 10. For example, C₁₋₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂—, or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a compound is substituted with a substituent bearing a subscript that indicates the number of said substituents can exceed 1, the substituents (when they exceed 1) are independently selected from the group of defined substituents, e.g., (R¹)ₘ, where m is 0, 1, 2 or 3. Further, when the subscript indicates a range, e.g. (R)ᵢ₋ⱼ, then the number of substituents may be selected from the integers between ‘i’ and ‘j’ inclusive. When a group contains a substituent, which can be hydrogen (H), for example, then when this substituent is taken as hydrogen, it is recognized that this is equivalent to the group being unsubstituted. When a variable group is shown to be optionally attached to a position, then hydrogen may be at the position even if not recited in the variable group definition. When one or more positions on a group are said to be “not substituted” or “unsubstituted,” then hydrogen atoms are attached to take up any free valency.

“Aromatic” indicates that each of the ring atoms is essentially in the same plane and has a p-orbital perpendicular to the ring plane, and that (4n+2) π electrons, where n is a positive integer, are associated with the ring to comply with Hückel's rule. The term “aromatic ring system” denotes a carbocyclic or heterocyclic ring system in which at least one ring of the ring system is aromatic.

The term “nonaromatic ring system” denotes a carbocyclic or heterocyclic ring system that may be fully saturated, as well as partially or fully unsaturated, provided that none of the rings in the ring system are aromatic.

The term “optionally substituted” in connection with the heterocyclic rings refers to groups which are unsubstituted or have at least one non-hydrogen substituent that does not extinguish the biological activity possessed by the unsubstituted analog. As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

The term “acceptable salt” or “salt” when related to a compound of the invention includes cations or anions. Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms axe replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl, or benzyl—preferably ammonium, methylammonium, isopropylammonium, dimethylammonium, diethylammonium, diisopropylammonium, trimethylammonium, triethylammonium, tris(isopropyl)ammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy)eth-1-ylammonium (diglycolamine salt), di(2-hydroxyeth-1-yl)ammonium (diolamine salt), tris(2-hydroxyethyl)ammonium (trolamine salt), tris(2-hydroxypropyl)ammonium, benzylthmethylammonium, benzyltriethylammonium, N,N,N-trimethylethanolammonium (choline salt), furthermore phosphonium ions, sulfonium ions, preferably tri(C₁-C₄-alkyl)sulfonium, such as trimethylsulfonium, and sulfoxonium ions, preferably tri(C₁-C₄-alkyl)sulfoxonium, and finally the salts of polybasic amines such as N,N-bis-(3-aminopropyl)methylamine, and diethylenetriamine.

Anions of useful acid addition salts are primarily chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate, and also the anions of C₁-C₄-alkanoic acids—preferably formate, acetate, propionate, and butyrate.

As used herein, the terms “undesired vegetation” and “harmful plants” are synonyms.

### Preparation of Compounds of the Invention

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve-volume set of Comprehensive Heterocyclic Chemistry II, A. R. Katritzky, C. W. Rees and E. F. V. Scriven editors-in-chief, Pergamon Press, Oxford, 1996.

Compounds of the invention can exist as one or more stereoisomers. The various stereoisomers include enantiomers, diastereomers, atropisomers, and geometric isomers. Stereolsomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form. For a comprehensive discussion of all aspects of stereoisomerism, see Ernest L. Eliel and Samuel H. Stereochemistny of Organic Compounds, John Wiley & Sons, New York, 1994. Compounds of the invention typically exist in more than one form, and the formulas of the invention thus include all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e., different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical, and biological properties as crystal shape, density, hardness, color, chemical stability, melting point, hygroscopicity, suspensibility, dissolution rate, and biological availability. One skilled in the art will appreciate that a polymorph of a compound of the invention can exhibit beneficial effects (e.g., suitability for preparation of useful formulations, improved biological performance) relative to another polymorph or a mixture of polymorphs of the same compound. Preparation and isolation of a particular polymorph of a compound of a compound of the invention can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinhcim, 2006.

One skilled in the art recognizes that because in the environment and under physiological conditions salts of chemical compounds are in equilibrium with their corresponding nonsalt forms, salts share the biological utility of the nonsalt forms. Thus, a wide variety of salts of compounds of the invention are useful for control of undesired vegetation (i.e., are agriculturally suitable). The salts of compounds of the invention include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic, or valeric acids. When a compound of the invention contains an acidic moiety such as a carboxylic acid or phenol, salts also include those formed with organic or inorganic bases such as pyridine, triethylamine, or ammonia, or amides, hydrides, hydroxides or carbonates of sodium, potassium, lithium, calcium, magnesium, or barium.

Moreover, the invention features processes and intermediates for preparing compounds of the invention. These compounds can be prepared by general methods known in the art of synthetic organic chemistry. One or more of the following methods and variations as described in Schemes 1 & 2 can be used.

In one general example, the compounds of formula (I) can be prepared as shown in Scheme 1.

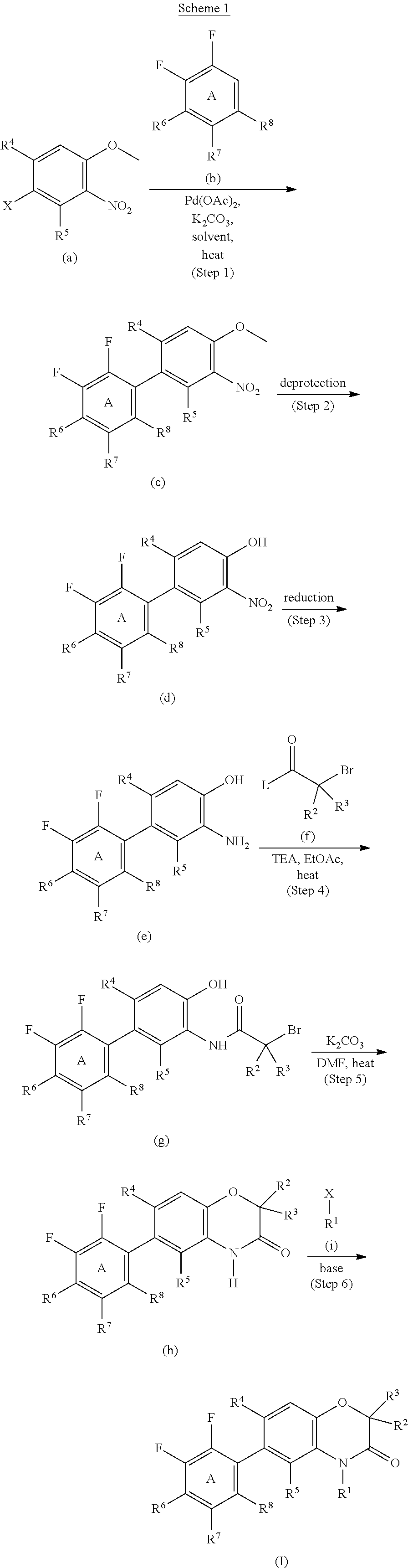

Accordingly, as shown in Step 1 of Scheme 1, compounds of formula (c) can be prepared by reaction of a compound of formula (a), where X is a leaving group such as Br, I, or OTf, with a substituted phenyl of formula (b) using cross-coupling reaction conditions with the aid of a metal catalyst. Suitable catalysts include palladium catalysts, such as Pd(OAc)₂ combined with 2-dicyclohcxylphosphino-2′,6′-dimethoxybiphenyl (SPhos) or chloro[(diadamantan-1-yl)(n-butyl)phosphino][2-amino-1,1-biphenyl-2-yl]palladium(II), and/or bis(adamantan-1-yl)(butyl)phosphane. As shown in Step 2 of Scheme 1, compounds of formula (d) can be prepared by demethylation of the aryl methyl ether of a compound of formula (c) under acidic conditions. In one example, a Lewis acid such as boron tribromide can be used. As shown in Step 3 of Scheme 1, compounds of formula (e) can be prepared by reduction of the nitro group of a compound of formula (d). Several methods for this are known to those skilled in the art, including the use of catalytic hydrogenation, zinc metal, or sodium hydrosulfite. As shown in Step 4 of Scheme 1, compounds of formula (g) can be prepared by condensing the amino group of a compound of formula (e) with a compound of formula (f) under basic conditions in an organic solvent, where L is a suitable leaving group such as an alkoxy group or a halogen atom. In one example, the base is a non-nucleophic base such as triethylamine or diisopropylethylamine. As shown in Step 5 of Scheme 1, benzoxazinones of formula (h) can be prepared via intramolecular ring closure between the phenolic hydroxyl group and N-acyl halide of a compound of formula (g) in a suitable polar organic solvent such as DMF or DMSO. As shown in Step 6 of Scheme 1, a compound of formula (I) (wherein R¹ is, for example, a C₁₋₄alkyl substituted with —C(O)R^{1a}) can be formed by reacting the benzoxazinone amino group of a compound of formula (h) with an alkyl or aryl halide of formula (i) under conditions suitable for bond formation. Compounds of formula (I), where R^{1a} is OH, can be further elaborated into other compounds of formula (I) (where, for example, R^{1a} is, —N(R^{1b})(R^{1c}), —NHN(R^{1b})(R^{1c}), —NHS(O)₂N(R^{1b})₂, or —NHOR^{1b}) by condensation methods known to those skilled in the art.

In another general example, the compounds of formula (I) can also be prepared as shown in Scheme 2.

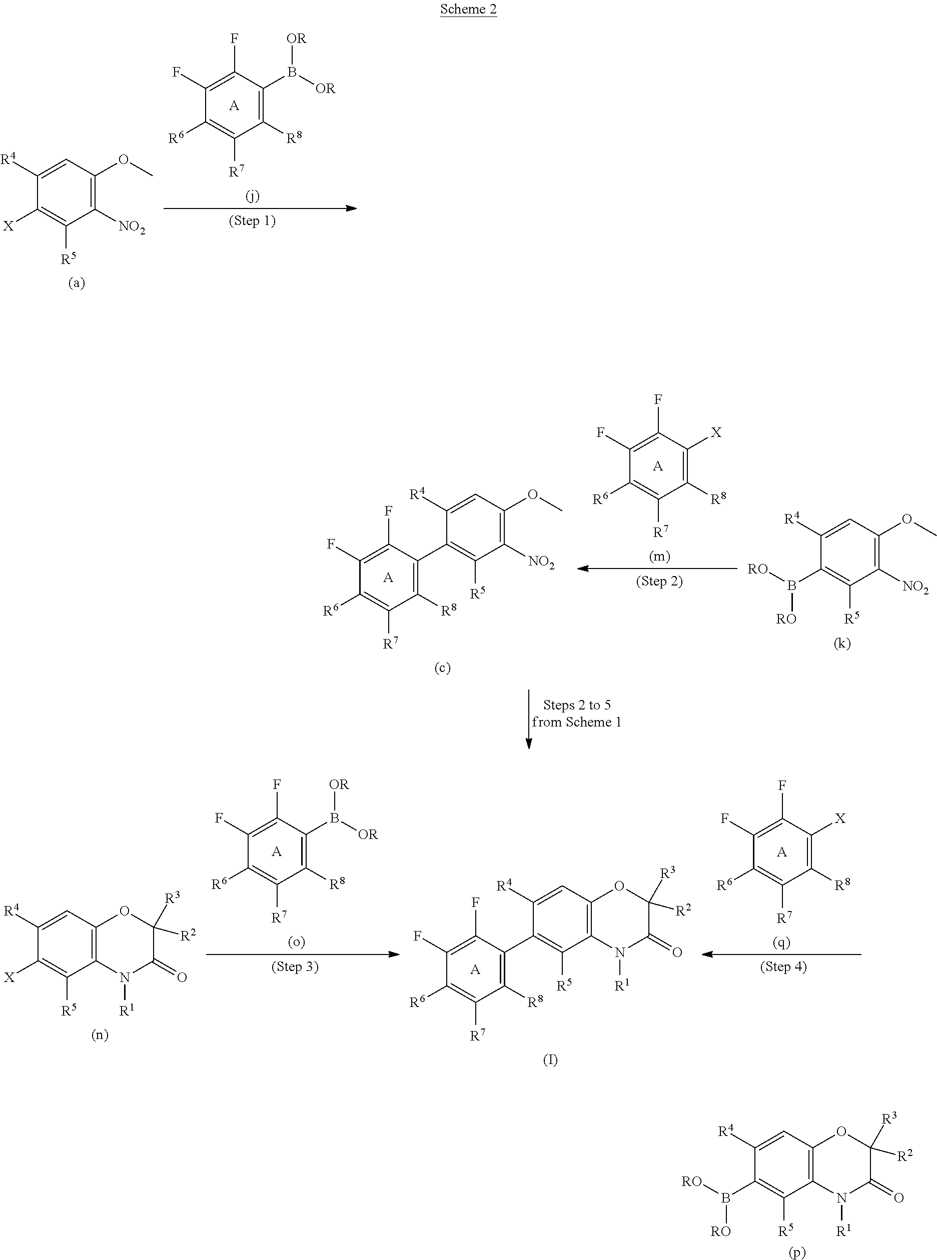

Accordingly, as shown in Step 1 of Scheme 2, phenyl boronic acids (where R=H) or phenyl boronates (e.g., where —B(OR)₂ represents a pinacol ester) of formula j can be coupled to a suitably substituted phenyl bromide or iodide of formula (a), where X is Br or I, in a Suzuki-Miyaura-type reaction using a suitable metal catalyst to produce a compound of formula (c) (Step 1). This can also be accomplished under similar conditions by reacting a compound for formula (k) with a compound (m) (Step 2). Employing steps analogous to Steps 2 to 5 as described in Scheme I can then be used to produce compounds of formula (I). Alternatively, a compound of formula (n) can be reacted with a compound of formula (o) (Step 3) or a compound of formula (p) can be reacted with a compound of formula (q) (Step 4) under Suzuki conditions to produce a compound of formula (I).

In one aspect, provided is a method of preparing a compound of formula (I) as described herein, or a salt thereof, comprising:
reacting a protected phenyl bromide or iodide of formula (a) with a fluorinated phenyl of formula (b), using the catalytic assistance of a metal such as palladium, to produce a biphenyl of formula (c), where R⁴ to R⁸ are as described elsewhere herein and Ring A contains 4 or 5 fluorine atoms,

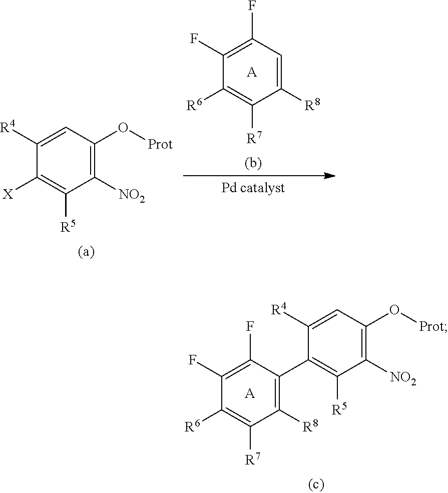

removing the protecting group of a biphenyl compound of formula (c) produce a phenol of formula (d),

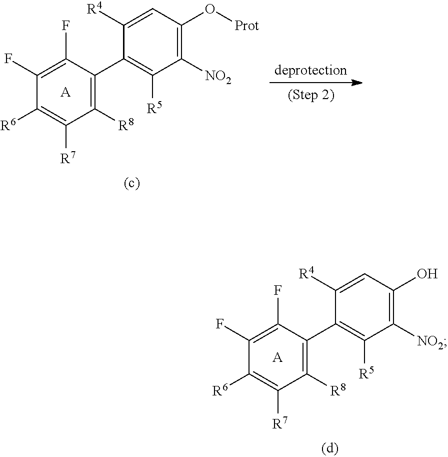

reducing the nitro group of a compound of formula (d) to produce an amine of formula (e),

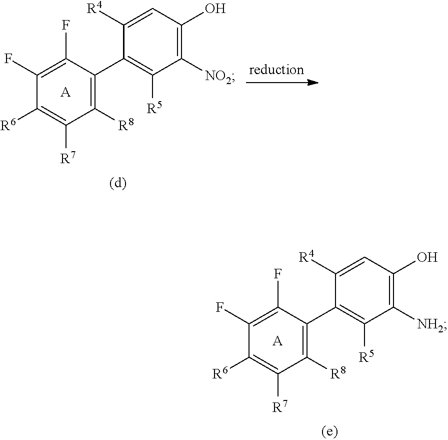

alkylating the amine of formula (e) with a compound of formula (f) using a non-nucleophilic base, where L is a suitable leaving group such as alkoxy or halogen atom, to produce a compound of formula (g),

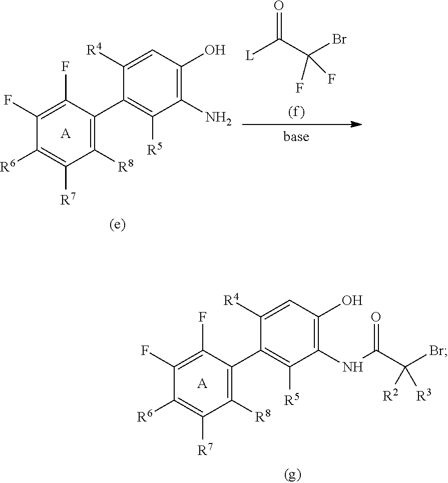

treating a compound of formula (g) with a suitable base, such as K₂CO₃, with the use of heat to produce a benzoxazinone of formula (h),

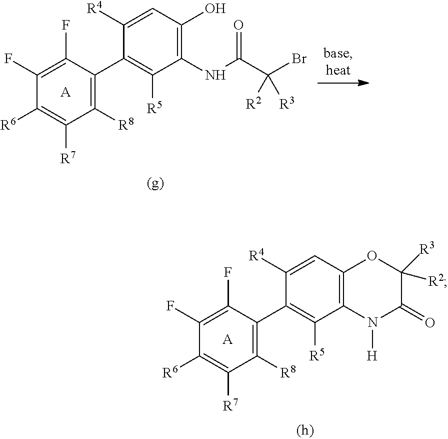

and
alkylating the amide of a benzoxazinone of formula (h) to produce a compound of the invention of formula (I),

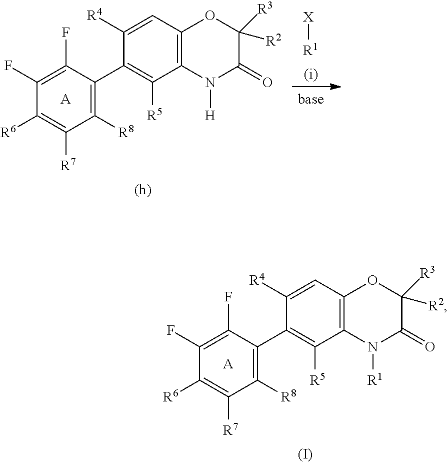

wherein R² and R³ are H or F atoms and R¹, R⁴, R⁵, R⁶, R⁷, and R⁸ are as described elsewhere herein.

In one embodiment, a compound of formula (c) is formed in a Suzuki-Miyaura-type reaction using a suitable metal catalyst by reaction a compound of formula (a) with a boronate or boronic acid of formula (j);

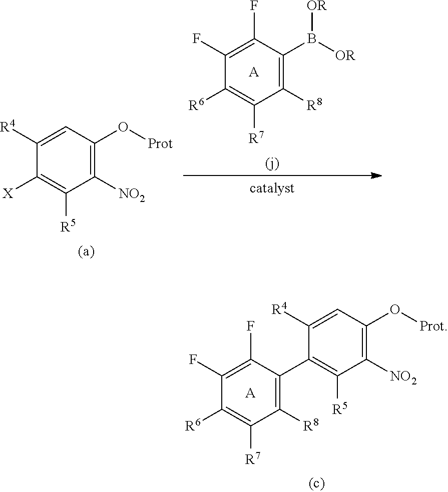

In another embodiment, a compound of formula (c) is formed in a Suzuki-Miyaura-type reaction using a suitable metal catalyst by reaction a boronate or boronic acid of formula (m) with a fluorinated phenyl bromide or iodide of formula (m);

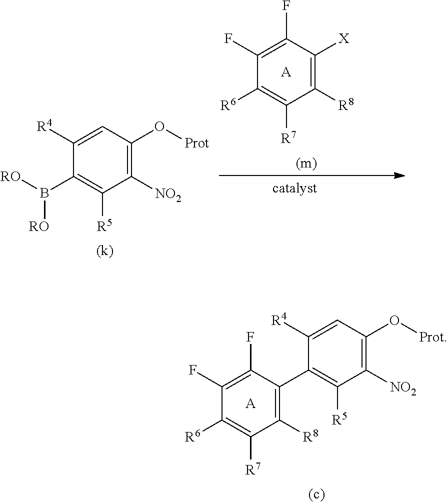

In another aspect, provided is a method of preparing a compound of formula (I) as described herein, or a salt thereof, wherein:
a compound of formula (n), where X is a leaving group such as a bromide, iodide, or tosylate, is reacted with a fluorinated phenyl boronate or boronic acid in a Suzuki-Miyaura-type reaction using a suitable metal catalyst;

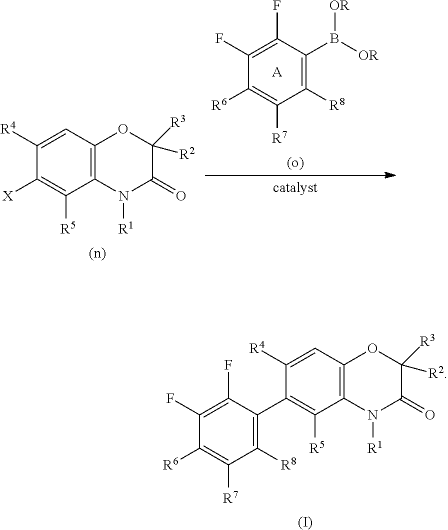

In yet another aspect, provided is a method of preparing a compound of formula (I) as described herein, or a salt thereof wherein:
a fluorinated phenyl compound of formula (q), where X is a leaving group such as a bromide, iodide, or tosylate, is reacted with a boronate or boronic acid of formula (p) in a Suzuki-Miyaura-type reaction using a suitable metal catalyst;

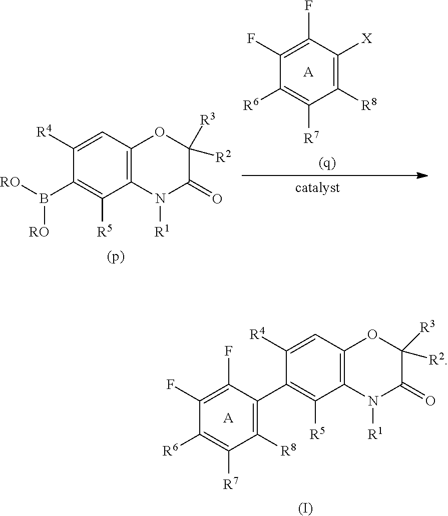

Any of the embodiments and variations described herein for compounds of formula (I) also applies to intermediates of formulas (c), (d), (e), (g), (h), (n), or (p). In some aspects, provided is a compound of formula (c), (d), (e), (g), (h), (n), or (p):

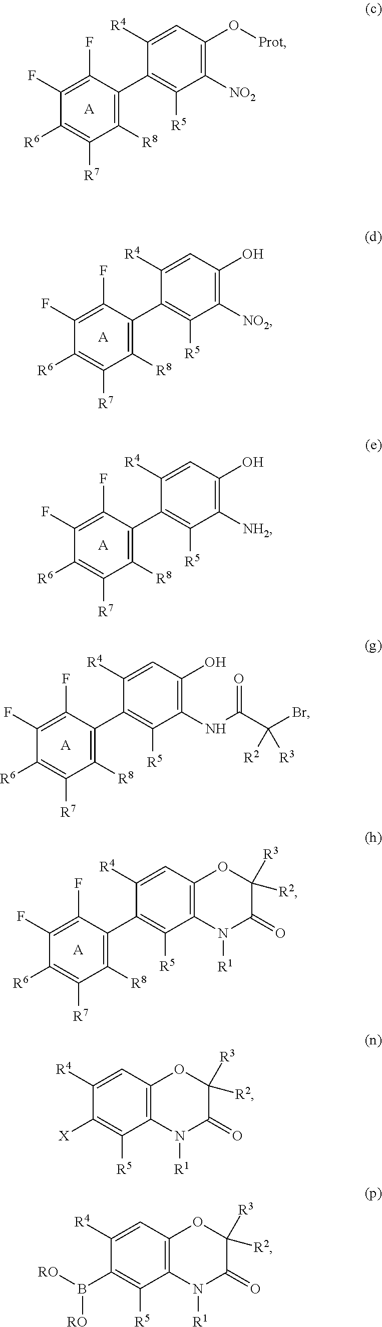

or a salt thereof (including an agriculturally suitable salt thereof).

It is recognized by one skilled in the art that various functional groups can be converted into others to provide different compounds of the invention. For a valuable resource that illustrates the interconversion of functional groups in a simple and straightforward fashion, see Larock, R. C, Comprehensive Organic Transformations: A Guide to Functional Group Preparations, 2^{nd} Ed., Wiley—VCH, New York, 1999.

It is also recognized that some reagents and reaction conditions described above for preparing compounds of the invention may not be compatible with certain functionalitics present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M., Protective Groups in Organic Synthesis, 2nd ed.; Wiley: New York, 1991). One skilled in the art will recognize that, in some cases, after the introduction of a given reagent as depicted in any individual scheme, it may be necessary to perform additional routine synthetic steps not described in detail to complete the synthesis of compounds of the invention. One skilled in the art will also recognize that it may be necessary to perform a combination of the steps illustrated in the above schemes in an order other than that implied by the particulars presented to prepare the compounds of the invention.

One skilled in the art will also recognize that compounds of the invention and the intermediates described herein can be subjected to various electrophilic, nucleophilic, radical, organometallic, oxidation, and reduction reactions to add substituents or modify existing substituents.

### Compositions

In certain aspects, a compound of this disclosure, including an agriculturally suitable salt thereof, may be used as an herbicidal active ingredient in a formulation, with at least one additional component selected from the group consisting of surfactants, solid diluents, and liquid diluents, which serves as a carrier. The formulation ingredients are selected to be consistent with the physical properties of the active ingredient, mode of application, and environmental factors such as soil type, moisture, and temperature.

In some variations, the compositions provided here are herbicides. In some variations, the compositions comprise a compound of this disclosure that controls or modifies the growth of plants. In certain variations, the compositions comprise a herbicidally effective amount of the compound, such that the quantity of such compound is capable of producing a controlling or modifying effect on the growth of plants. Controlling or modifying effects include all deviation from natural development, for example killing, retardation, leaf burn, albinism, dwarfing and the like.

Liquid formulations include solutions (including emulsifiable concentrates), suspensions, emulsions (including microemulsions, oil-in-water emulsions, flowable concentrates and/or suspoemulsions), and the like, which optionally can be thickened into gels. The general types of aqueous liquid formulations are soluble concentrate, suspension concentrate, capsule suspension, concentrated emulsion, microcmulsion, oil-in-water emulsion, flowable concentrate, and suspoemulsion. The general types of nonaqueous liquid formulations are emulsifiable concentrate, microemulsifiable concentrate, dispersible concentrate, and oil dispersion.

The general types of solid formulations are dusts, powders, granules, pellets, prills, pastilles, tablets, filled films (including seed coatings), and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation. Alternatively, the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength formulations are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant.

Liquid and dry formulations can be metered directly into drip irrigation systems or metered into the furrow during planting.

The formulations will typically contain effective amounts of active ingredient, diluent, and surfactant within the following approximate ranges, shown in Table 2, which add up to 100 percent by weight.

TABLE 2
Formulation Ratios

| | Weight Percent | | |
|---|---|---|---|
| | Active | | |
| | Ingredient | Diluent | Surfactant |
| Water-Dispersible and | 0.001-90 | 0-99.999 | 0-15 |
| Water-Soluble Granules, | | | |
| Tablets, and Powders | | | |
| Oil Dispersions, Suspensions, | 1-50 | 40-99 | 0-50 |
| Emulsions Solutions (including | | | |
| emulsifiable Concentrates) | | | |
| Dusts | 1-25 | 70-99 | 0-5 |
| Granules and Pellets | 0.001-99 | 5-99.999 | 0-15 |
| High Strength Formulations | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water; N,N-dimethylalkanamides (e.g., N,N-dimethylformamide); limonene; dimethyl sulfoxide; N-alkylpyrrolidones (e.g., N-methylpyrrolidinone); alkyl phosphates (e.g., triethyl phosphate); ethylene glycol; triethylene glycol; propylene glycol; dipropylene glycol; polypropylene glycol; propylene carbonate; butylene carbonate; paraffins (e.g., white mineral oils, normal paraffins, isoparaffins); alkylbenzenes; alkylnaphthalenes; glycerine; glycerol triacetate; sorbitol; aromatic hydrocarbons; dearomatized aliphatics; alkylbenzenes; alkylnaphthalenes; ketones such as cyclohexanone, 2-heptanone, isophorone, and 4-hydroxy-4-methyl-2-pentanone; acetates such as isoamyl acetate, hexyl acetate, heptyl acetate, octyl acetate, nonyl acetate, tridecyl acetate, and isobornyl acetate; other esters such as alkylated lactate esters, dibasic esters, alkyl and aryl benzoates, and 7-butyrolactone; and alcohols, which can be linear, branched, saturated or unsaturated, such as methanol, ethanol, n-propanol, isopropyl alcohol, n-butanol, isobutyl alcohol, n-hexanol, 2-ethvlhcxanol, n-octanol, decanol, isodecyl alcohol, isooctadecanol, cetyl alcohol, laurel alcohol, tridecyl alcohol, oleyl alcohol, cyclohexanol, tetrahydrofurfuryl alcohol, diacetone alcohol, cresol, and benzyl alcohol. Liquid diluents also include glycerol esters of saturated and unsaturated fatty acids (typically C₆-C₂₂) such as plant seed and fruit oils (e.g., oils of olive, castor, linseed, sesame, corn (maize), peanut, sunflower, grapeseed, safflower, cottonseed, soybean, rapeseed, coconut, and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources and can be purified by distillation. Typical liquid diluents are described in C. Marsden & S. Mann, Solvents Guide, Cleaver-Hume Press, London, 1963.

Surfactants can be classified as nonionic, anionic, or cationic. Nonionic surfactants useful for the present formulations include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides, and ethoxylated alkanolamides; alkoxylated triglycerides such as ethoxylated soybean, castor, and rapeseed oils; alkylphenol alkoxylates such as octylphenol ethoxylates, nonylphenol ethoxylates, dinonyl phenol ethoxylates, and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide, or mixtures thereof); fatty acid esters, glycerol esters, lanolin-based derivatives, polyethoxylate esters such as polyethoxylated sorbitan fatty acid esters, polyethoxylated sorbitol fatty acid esters, and polyethoxylated glycerol fatty acid esters; other sorbitan derivatives such as sorbitan esters; polymeric surfactants such as random copolymers, block copolymers, alkyd PEG (polyethylene glycol) resins, graft or comb polymers and star polymers; polyethylene glycols (PEGS); polyethylene glycol fatty acid esters; silicone-based surfactants; and sugar-derivatives such as sucrose esters, alkyl polyglycosidcs, and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines, and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts, and diquatemary salts; and amine oxides such as alkyldimethylamine oxides and bis-(2-hydroxyethyl)-alkylamine oxides.

Also useful for the present formulations are mixtures of nonionic and anionic surfactants or mixtures of nonionic and cationic surfactants. Nonionic, anionic, and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood. Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Formulations of the present invention may also contain formulation auxiliaries and additives, known to those skilled in the art as formulation aids (some of which may be considered to also function as solid diluents, liquid diluents, or surfactants). Such formulation auxiliaries and additives may control the following: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers, and waxes. Examples of formulation auxiliaries and additives include those listed in McCutcheon's Volume 2: Functional Materials, annual International and North American editions published by McCutcheon's Division, The Manufacturing Confectioner Publishing Co., and PCT Publication WO 03/024222.

The compounds of the invention and any other active ingredients are typically incorporated into the present formulations by dissolving the active ingredient in a solvent or by grinding in a liquid or dry diluent. Solutions, including emulsifiable concentrates, can be prepared by simply mixing the ingredients. If the solvent of a liquid formulations intended for use as an emulsifiable concentrate is water-immiscible, an emulsifier is typically added to emulsify the active-containing solvent upon dilution with water. Active ingredient slurries, with particle diameters of up to 2,000 microns can be wet milled using media mills to obtain particles with average diameters below 3 microns. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 micron to 10 micron range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration,” Chemical Engineering, Dec. 4, 1967, pp 147-48, Perry's Chemical Engineer's Handbook, 4th Ed., McGraw-Hill, New York, 1963, pages 8-57 and following, and PCT Publication WO 91/13546. Pellets can be prepared as described in U.S. Pat. No. 4,172,714. Water-dispersible and water-soluble granules can be prepared as taught in U.S. Pat. Nos. 4,144,050 and 3,920,442 and German Pat. No. 3,246,493. Tablets can be prepared as taught in U.S. Pat. Nos. 5,180,587, 5,232,701, and 5,208,030. Films can be prepared as taught in Great Britain Pat. No. 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4, Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

### Biological Activity

Test results indicate that the compounds of the present invention are highly active preemergent and/or postemergent herbicides and/or plant growth regulants. The compounds of the invention generally show highest activity for postemergence weed control (e.g., applied after weed seedlings emerge from the soil) and preemergence weed control (e.g., applied before weed seedlings emerge from the soil). Many of them have utility for broad-spectrum pre- and/or postemergence weed control in areas where complete control of all vegetation is desired such as around fuel storage tanks, industrial storage areas, parking lots, drive-in theaters, airfields, riverbanks, irrigation, and other waterways, around billboards and highway and railroad structures. Many of the compounds of this disclosure, by virtue of selective metabolism in crops versus weeds, or by selective activity at the locus of physiological inhibition in crops and weeds, or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays.

In some variations, provided herein is a method of controlling undesired vegetation, comprising applying a compound of formula (I), (II), (III), (IV), or (V), or a salt thereof (including an agriculturally suitable salt thereof). In some variations, the compound is applied at low application rates. In certain variations, the compound is applied at a rate of 1 to 10,000 g per 10.000 m², 2 to 5,000 g per 10,000 m², 5 to 2,000 g per 10,000 m², 1 to 1000 g per 10,000 m², 1 to 500 g per 10,000 m², 1 to 100 g per 10,000 m², 1 to 75 g per 10,000 m², 15 to 1000 g per 10,000 m², 15 to 100 g per 10,000 m²15 to 75 g per 10,000 m², or 15 to 60 g per 10,000 m². In certain variations of the foregoing, the application of the compound at the aforementioned application rates leads to postemergence control of the undesired vegetation and/or preemergence control of the undesired vegetation.

In certain variations, the application of the compound, including at the aforementioned application rate, leads to burndown. In one variation, burndown refers to when an herbicide is used to reduce weed presence at the time of treatment. Burndown is often used in minimum or no-till fields because the weeds cannot be managed by tilling the soil. The burndown application may be used post-harvest and/or prior to crop emergence. Burndown may be useful against weeds that emerge between growing seasons.

In certain variations, the application of the compound, including at the aforementioned application rate, imparts residual control. The compounds described herein may be used as pre-emergence herbicides, which may be applied after crop planting, but prior to crop and/or weed emergence. Herbicides considered pre-emergence also may be referred to as those imparting “residual control.” and provide extended control of germinating or newly emerged weeds.

In one variation, the undesired vegetation is at least 50%, at least 55%, at least 60%, at least 65%, at least 70%, at least 75%, at least 80%, at least 85%, at least 90%, at least 95%, at least 96%, at least 97%, at least 98%, at least 99%, or about 100% controlled. In some variations of the foregoing, the undesired vegetation is a weed. In one variation, the undesired vegetation is a PPO inhibitor-resistant weed.

Examples of crop fields treated by the compounds in the present invention include edible crop fields such as peanut fields, soybean fields, corn fields, and wheat fields, feed crop fields such as sorghum fields and oat fields, industrial crop fields such as cotton fields and rape fields, and sugar crop fields such as sugarcane fields and sugar beet fields. In one variation, crop fields treated by the compounds herein include corn, soybean, wheat, and cotton fields.

Examples of vegetable fields treated by the compounds in the present invention include fields for cultivation of solanaceous vegetables (eggplants, tomatoes, bell peppers, capsicums, potatoes, and the like), fields for cultivation of cucurbitaceous vegetables (cucumbers, pumpkins, zucchini, watermelons, melons, and the like), fields for cultivation of cruciferous vegetables (radishes, turnips, horseradishes, kohlrabies, Chinese cabbages, cabbages, mustard, broccolis, cauliflowers, and the like), fields for cultivation of asteraceous vegetables (burdocks, garland chrysanthemums, artichokes, lettuces, and the like), fields for cultivation of liliaceous vegetables (leeks, onions, garlics, and asparagus), fields for cultivation of apiaceous vegetables (carrots, parsley, celery, parsnips, and the like), fields for cultivation of chenopodiaceous vegetables (spinach, chards, and the like), fields for cultivation of lamiaceous vegetables (perilla, mint, basil, and lavender), strawberry fields, sweet potato fields, yam fields, and taro fields.

Examples of the land under perennial crops in the present invention include orchards, tea fields, mulberry fields, coffee fields, banana fields, palm fields, flowering tree firms, flowering tree fields, planting stock fields, nursery fields, forest lands, and gardens. Examples of the orchard trees in the present invention include pomaceous fruits (apples, pears, Japanese pears, Chinese quinces, quinces, and the like), stone fruits (peaches, plums, nectarines, Japanese apricots, cherries, apricots, prunes, and the like), citrus fruits (Citrus unshiu, oranges, lemons, limes, grapefruits, and the like), nut trees (chestnuts, walnuts, hazelnut trees, almonds, pistachios, cashew nut trees, macadamia nut trees, and the like), berry fruits (grapes, blueberries, cranberries, blackberries, raspberries, and the like), Japanese persimmons, olives, and loquats.

Examples of the non-crop land in the present invention include athletic fields, empty lots, railroad edges, parks, parking lots, road edges, dry riverbeds, lands under a power line, residential lands, and factory sites.

The crop cultivated in the crop field in the present invention is not limited as long as the crop is a variety generally cultivated as a crop.

The plant of the above-mentioned variety may be a plant that can be produced by natural crossing, a plant that can be generated by mutation, an F1 hybrid plant, or a transgenic plant (also referred to as a genetically-modified plant). The plant generally has properties such as obtaining of the tolerance to an herbicide, accumulation of a toxic substance against a pest, suppression of the susceptibility to a disease, increase in the yield potential, improvement in the tolerance to a biotic and an abiotic stressors, accumulation of a substance, and improvement in the preservability and the processability.

An F1 hybrid plant is a first-generation hybrid obtained by crossing varieties of two different strains, and generally has a heterotic property with a trait superior to that of either of the parents. A transgenic plant has a foreign gene introduced from another organism or the like such as a microorganism and has a property that cannot be easily obtained by cross breeding, mutagenesis, or natural recombination in a natural environment.

Examples of the techniques for producing the above-mentioned plants include conventional breeding techniques; genetic engineering techniques; genome breeding techniques; new breeding techniques; and genome editing techniques. Conventional breeding techniques are for obtaining a plant having a desirable property by mutation or crossing. Genetic engineering techniques include techniques for imparting a new property to a target organism by extracting a target gene (DNA) from another organism (for example, a microorganism) and introducing the target gene into the genome of the target organism. Genetic engineering techniques also include antisense techniques or RNA interference techniques for imparting a new or improved property by silencing another gene present in the plant. Genome breeding techniques are for improving breeding efficiency using genomic information, and examples of the genome breeding techniques include DNA marker (also called genomic marker or genetic marker) breeding techniques and genomic selection. For example, DNA marker breeding is a method in which a progeny having a target useful trait gene is selected from a large number of crossed progenies using a DNA marker that is a DNA sequence that serves as a marker of the location of the specific useful trait gene on the genome. In the method, the crossed progeny is analyzed when it is an infant plant using a DNA marker to effectively shorten the time required for the breeding.

Genomic selection is a technique in which a prediction formula is created from a phenotype and genomic information obtained in advance to predict the property from the prediction formula and the genomic information without evaluating the phenotype and is a technique that can contribute to improving breeding efficiency. The term “new breeding techniques” is a general term for breed improvement (breeding) techniques that combine molecular biological techniques. Examples of the new breeding techniques include cisgenesis/intragenesis, oligonucleotide-directed mutagenesis, RNA-dependent DNA methvlation, genome editing, grafting on a GM rootstock or a scion, reverse breeding, agminfiltration, and seed production technology (SPT). The genome editing technique is for converting genetic information in a sequence-specific manner, and it is possible to delete a base sequence, substitute an amino acid sequence, introduce a foreign gene, and the like using the technique. Examples of the tool include sequence-specific genome modification techniques such as a zinc finger nuclease capable of sequence-specific DNA cleavage (Zinc-Finger, ZFN). TALEN, CRISPR-Cas9, CRISPER-Cpf1, Meganuclease, and CAS9 Nickase and Target-AID created by modifying the aforementioned tools.

Examples of the above-mentioned plants include plants listed in the database of the registered genetically-modified crops (GM Approval Database) in the electronic information site of International Service for the Acquisition of Agri-biotech Applications (ISAAA) (http://www.isaaa.org/). More specific examples are herbicide-tolerant plants, pest-resistant plants, disease-resistant plants, plants modified in the quality (for example, with increase or decrease in the content or change in the composition) of the products (for example, starch, amino acids, and fatty acids), fertility trait-modified plants, abiotic stress-tolerant plants, and plants modified in the trait related to the growth or the yield.

Mechanisms of obtaining herbicide tolerance include reduction in the affinity between the agent and its target, rapid metabolism (decomposition, modification, and the like) of the agent by an expressed enzyme that inactivates the agent, or inhibition of incorporation or translocation of the agent in the plant body. Examples of the plants to which herbicide tolerance has been imparted by genetic engineering technique include plants to which tolerance has been imparted to 4-hydroxyphenylpyruvate dioxygenase (hereinafter abbreviated as HPPD) inhibitors such as isoxaflutole and mesotrione, acetolactate synthase (hereinafter abbreviated as ALS) inhibitors such as imidazolinone herbicides containing imazethapyr and sulfonylurca herbicides containing thifensulfuron-methyl, 5-enolpyruvylshikimate-3-phosphate synthase (hereinafter abbreviated as EPSP) inhibitors such as glyphosate, glutamine synthase inhibitors such as glufosinate, auxin herbicides such as 2,4-D and dicamba, and oxynyl herbicides containing bromoxynil. Preferable herbicide-tolerant transgenic plants treated by the combinations of the invention are cereals such as wheat, barley, rye, and oats, canola, sorghum, soybeans, rice, rape, sugar beet, sugar cane, grapes, lentils, sunflowers, alfalfa, pomaceous fruits, drupes, coffee, tea, strawberries, lawn grass, tomatoes, potatoes, cucumbers, and vegetables such as lettuces, and more preferable herbicide-tolerant transgenic plants are cereals such as wheat, barley, rye, and oats, soybeans, rice, vines, tomatoes, potatoes, and pomaceous fruits.

In one example, in order to obtain the glyphosate herbicide-tolerant plants one or more genes are introduced from: a glyphosate-tolerant EPSPS gene (CP4 epsps) from Agrobacterium tumefaciens strain CP4; a glyphosate metabolizing enzyme gene (gat4601, gat4621) in which the metabolic activity of the glyphosate metabolizing enzyme (glyphosate N-acetyltransferase) gene from Bacillus licheniformis is enhanced by a shuffling technique; a glyphosate metabolizing enzyme (glyphosate oxidase gene, goxy247) from Ochrobacterum anthropi strain LBAA; and EPSPS genes from maize having a glyphosate-tolerant mutation (mepsps, 2mepsps). Main examples of the plants are alfalfa (Medicago saliva), Argentine canola (Brassica napes), cotton (Gossypium hirsutum L.), creeping bentgrass (Agrostis stolonifera), maize (Zea mays L.), polish canola (Brassica rapa), potato (Solanum tuberosum L.), soybean (Glycine max L.), sugar beet (Beta vulgaris), and wheat (Triticum aestivum). Some glyphosate-tolerant transgenic plants are commercially available. For example, the genetically-modified plant in which the glyphosate-tolerant EPSPS from the Agrobacterium is expressed is commercially available with a trade name such as “Roundup Ready;” the genetically-modified plant in which the glyphosate metabolizing enzyme that is from Bacillus and has the metabolic activity enhanced by a shuffling technique is expressed is commercially available with a trade name such as “Optimum® GAT®, or “Optimum® Gly canola”, and the genetically-modified plant in which the EPSPS that is from maize and has glvphosate-tolerant mutation is expressed is commercially available with the trade name “GlyTol®”.

In another example, in order to obtain the glufosinate herbicide-tolerant plants one or more genes are introduced from: a phosphinothricin N-acetyltransferase (PAT) gene (bar) that is a glufosinate metabolizing enzyme from Streptomyces hygroscopicus; a phosphinothricin N-acetyltransferase (PAT) enzyme gene (pat) that is a glufosinate metabolizing enzyme from Streptomyces viridochromogenes; and a synthesized pat gene (pat syn) from Streptomyces viridochromogenes strain Tu494. Main examples of the plants include Argentine canola (Brassica napus), chicory (Cichorium intybus), cotton (Gossypium hirsutum L.), maize (Zea mans L.), polish canola (Brassica rapa), rice (Oryza sativa L.), soybean (Glycine max L.), and sugar beet (Beta vulgaris). Some glufosinate-tolerant genetically-modified plants are commercially available. For example, a genetically-modified plant from a glufosinate metabolizing enzyme (bar) from Streptomyces hygroscopicus and from Streptomyces viridochromogenes is commercially available with trade names such as “LibertvLink®”, “InVigorz®”, or “WideStrike®”.

In another example, oxynil herbicide-tolerant plants are known. For example, bromoxynil-tolerant transgenic plants into which a nitrilase gene (bxn) is introduced from an ox nil herbicide metabolizing enzyme from Klebsiella pneumoniae subsp. ozaenae. Main examples of the plants are Argentine canola (Brassica napus), cotton (Gossypium hirsutum L.), and tobacco (Nicotiana tabacum L.). The plants are commercially available with a trade name such as “Navigator® canola” or “BXN®”.

ALS herbicide-tolerant plants are also known. Examples include carnations (Dianthus caryophyllus), which are obtained by introduction of an ALS herbicide-tolerant ALS gene (surB) as a selection marker from tobacco (Nicotiana tabacum) and are commercially available with the trade names “Moondust®”, “Moonshadow®”, “Moonshade®”, “Moonlite®”, “Moonaqua®”, “Moonvista®”, “Moonique®”, “Moonpearl®”, “Moonberry®”, and “Moonvelvet®”; flax (Linum usitatissimum L.), into which an ALS herbicide-tolerant ALS gene (als) from Arabidopsis thaliana is introduced is commercially available with the trade name “CDC Triffid Flax”; sulfonylurea herbicide-tolerant and an imidazolinone herbicide-tolerant maize (Zea mays L.) into which an ALS herbicide-tolerant ALS gene (zm-hra) from maize is introduced is commercially available with the trade name “Optimum® GAT”; an imidazolinone herbicide-tolerant soybean into which an ALS herbicide-tolerant ALS gene (csr1-2) from Arabidopsis thaliana is introduced is commercially available with the trade name “Cultivance®”; and sulfonylurea herbicide-tolerant soybeans into which an ALS herbicide-tolerant ALS gene (gm-hra) from a soybean (Glycine max) is introduced are commercially available with the trade names “Treus®”, “Plenish®”, and “Optimum® GAT™”. There is also cotton into which an ALS herbicide-tolerant ALS gene (S4-HrA) from tobacco (Nicotiana tabacum cv. Xanthi) is introduced.

HPPD herbicide-tolerant plants are also known. In one example, a soybean into which a mesotrione-tolerant HPPD gene (avhppd-03) from an oat (Avena sativa) and a phinothricin N-acetyltransferase (PAT) enzyme gene (pat) are simultaneously introduced. In another example, a soybean tolerant to mesotrione into which a glufosinate metabolizing enzyme from Streptomyces viridochromogenes is introduced is commercially available.

In another example, 2,4-D-tolerant plants include: maize into which an aryloxyalkanoate dioxygenase gene (aad-1) for a 2,4-D metabolizing enzyme from Sphingobium herbicidovorans is introduced is commercially available with the trade name “Enlist® Maize”, and soybean and cotton into which an aryloxyalkanoate dioxygenase gene (aad-12) for a 2,4-D metabolizing enzyme from Delftia acidovorans is introduced is commercially available with the trade name “Enlist® Soybean”.

In another example. Dicamba-tolerant plants include: soybean and cotton into which a dicamba monooxygenase gene (dmo) having a dicamba metabolizing enzyme from Stenotrophomonas maltophilia strain DI-6 is introduced; and a soybean (Glycine max L.) into which a glyphosate-tolerant EPSPS gene (CP4 epsps) from Agrobacterium tumeficiens strain CP4 is introduced simultaneously with the above-mentioned gene is commercially available with the trade name Genuity® Roundup Ready 2 Xtend®”.

Further examples of the commercially available transgenic plants to which herbicide tolerance has been imparted include: the glyphosate-tolerant maize “Roundup Ready® Corn”, “Roundup Ready® 2”, “Agrisure® GT”, “Agrisure® GT/CB/LL”, “Agrisure® GT/RW”, “Agrisure® 3000GT”, “YieldGard™ VT™ Rootworm/RR2”, and “YieldGardi™ VT™ Triple”; the glyphosate-tolerant soybeans “Roundup Ready® Soybean” and “Optimum® GAT”; the glyphosate-tolerant cotton “Roundup Ready® Cotton” and “Roundup Ready® Flex”; the glyphosate-tolerant canola “Roundup Ready® Canola”; the glyphosate-tolerant alfalfa “Roundup Ready® Alfalfa”, the glyphosate-tolerant rice “Roundup Ready® Rice”; the glufosinate-tolerant maize “Roundup Ready® 2”, “LibertyLink®”, “Herculex®® 1”, “Herculex®® RW”, “Herculex®”, “Xtra”, “Agrisure® GT/CB/LL”, “Agrisurel® CB/LL/RW”, and “Bt10”, the glufosinate-tolerant cotton “FiberMax™ LibertyLink™”; the glufosinate-tolerant canola “InVigor®”; the glufosinate-tolerant rice “LibertyLink Rice” (manufactured by Bayer AG); the bromoxynil-tolerant cotton “BXN”; the bromoxynil-tolerant canola “Navigator®” and “Compass®”; and the glufosinate-tolerant canola “InVigor®”. Additional plants modified with respect to a herbicide are widely known, and the examples of the plants include alfalfa, apples, barley, eucalyptuses, flax, grapes, lentils, rape, peas, potatoes, rice, sugar beet, sunflowers, tobacco, tomato, turfgrass, and wheat that are tolerant to glyphosate (see, for example, U.S. Pat. Nos. 5,188,642, 4,940,835, 5,633,435, 5,804,425, and 5,627,061); beans, cotton, soybeans, peas, potatoes, sunflowers, tomatoes, tobacco, maize, sorghum, and sugar cane that are tolerant to dicamba (see, for example, WO2008051633, U.S. Pat. Nos. 7,105,724, and 5,670,454); soybeans, sugar beet, potatoes, tomatoes, and tobacco that are tolerant to glufosinate (see, for example, U.S. Pat. Nos. 6,376,754, 5,646,024, and 5,561,236); cotton, peppers, apples, tomatoes, sunflowers, tobacco, potatoes, maize, cucumbers, wheat, soybeans, sorghum, and cereals that are tolerant to 2,4-D (see, for example, U.S. Pat. Nos. 6,153,401, 6,100,446, WO2005107437, U.S. Pat. Nos. 5,608,147, and 5,670,454); and canola, maize, millet, barley, cotton, mustard, lettuces, lentils, melons, millet, oats, sword beans, potatoes, rice, rye, sorghum, soybeans, sugar beet, sunflowers, tobacco, tomatoes, and wheat that are tolerant to acetolactate synthase (ALS) inhibitor herbicide (for example, a sulfonylurea herbicide and an imidazolinone herbicide) (see, for example, U.S. Pat. No. 5,013,659, WO2006060634, U.S. Pat. Nos. 4,761,373, 5,304,732, 6,211,438, 6,211,439, and 6,222,100). The rice tolerant to an imidazolinone herbicide is especially known, and examples of the rice include rice having specific mutation (for example, S653N, S654K, A122T, S653(At)N, S654(At)K. and A 122(At)T) in the acetolactate synthase gene (acetohydroxyacid synthase gene) (see, for example, US 2003/0217381, and WO200520673); and the examples include barley, sugar cane, rice, maize, tobacco, soybeans, cotton, rape, sugar beet, wheat, and potatoes that are tolerant to an HPPD inhibitor herbicide (for example, an isoxazole herbicide such as isoxaflutole, a triketone herbicide such as sulcotrionc or mesotrionc, a pyrazole herbicide such as pyrazolynate, or diketonitrile that is a decomposition product of isoxaflutole) (see, for example, WO2004/055191, WO199638567, WO1997049816, and U.S. Pat. No. 6,791,014).

Examples of the plants to which herbicide tolerance has been imparted by a classical technique or a genome breeding technique include the rice “Clearfield® Rice”, the wheat “Clearfield® Wheat”, the sunflower “Clearfield®® Sunflower”, the lentil “Clearfield®® lentils”, and the canola “Clearfield® canola” (manufactured by BASF SE) that are tolerant to an imidazolinone-based ALS inhibitor herbicide such as imazethapyr or imazamox; the soybean “STS) soybean” that is tolerant to a sulfonvl-based ALS inhibitor herbicide such as thifensulfuron-methyl; the sethoxydim-tolerant maize “SR® corn” and “Poast Protected® corn” that are tolerant to an acetyl-CoA carboxylase inhibitor such as a trionoxime herbicide or an aryloxy phenoxypropionic acid herbicide; the sunflower “ExpressSun®” that is tolerant to a sulfonylurea herbicide such as tribenuron; the rice “Provisiem Rice” that is tolerant to an acetyl-CoA carboxylase inhibitor such as quizalofop; and the canola “Triazine Tolerant Canola” that is tolerant to a PSII inhibitor.

Examples of the plants to which herbicide tolerance has been imparted by a genome editing technique include the canola “SU Canola®” tolerant to a sulfonylurea herbicide in which a rapid variety development technique (Rapid Trait Development System, RTDS,) is used. RTDS) corresponds to oligonucleotide-directed mutagenesis of the genome editing technique, and by RTDS, it is possible to introduce mutation in a DNA in a plant via Gene Repair Oligonucleotide (GRON), that is, a chimeric oligonucleotide of the DNA and the RNA without cutting the DNA. In addition, examples of the plants include maize in which herbicide tolerance and phytic acid content have been reduced by deleting the endogenous gene IPK1 using zinc finger nuclease (see, for example, Nature 459, 437-441 2009); and rice to which herbicide tolerance has been imparted using CRISPR-Cas9 (see, for example, Rice, 7, 5 2014).

In the present invention, examples of the crop tolerant to a specific PPO inhibitor include crops to which PPO having a reduced affinity for the inhibitor is imparted by a genetic engineering technique. Alternatively, the crop may have a substance that detoxifies and decomposes the PPO inhibitor by cytochrome P450 monooxygenase alone or in combination with the above-mentioned PPO. The tolerant crops are described in, for example, patent documents such as WO2011085221, WO2012080975, WO2014030090, WO2015022640, WO2015022636, WO2015022639, WO2015092706, WO2016203377, WO2017198859, WO2018019860, WO2018022777, WO2017112589, WO2017087672. WO2017039969, and WO2017023778, and non-patent document Li & Nicholl in Pest Management Science (2005), Vol. 61, pgs. 277-285.

Examples of the plants to which herbicide tolerance has been imparted by a new breeding technique in which the property of a GM rootstock is imparted to a scion by a breeding technique in which grafting is used include the non-transgenic soybean scion to which glyphosate tolerance is imparted using the glyphosate-tolerant soybean Roundup Ready® as a rootstock (see Jiang, et al., in Weed Technology (2013) Vol. 27, pgs. 412-416).

The above-mentioned plants include strains to which two or more traits are imparted among abiotic stress tolerance, disease resistance, herbicide tolerance, pest resistance, a growth trait, a yield trait, nutrient uptake, product quality, a fertility trait, and the like as described above using a genetic engineering technique, a classical breeding technique, a genome breeding technique, a new breeding technique, a genome editing technique, or the like, and strains to which two or more of the properties of the parent strains are imparted by crossing plants having the same or different properties.

Examples of the commercially available plants to which tolerance to two or more herbicides are imparted include the cotton “GlyTol™ LibertyLink®” and “GlyTol™ LibertyLink®” that are tolerant to glyphosate and glufosinate; the maize “Roundup Ready™ LibertyLink™ Maize” that is tolerant to glyphosate and glufosinate; the soybean “Enlist™ Soybean” that is tolerant to glufosinate and 2,4-D; the soybean “Genuity® Roundup Ready (trademark) 2 Xtend (trademark)” that is tolerant to glyphosate and dicamba; the maize and the soybean “OptimumGAT®” that are tolerant to glyphosate and an ALS inhibitor; the genetically modified soybeans “Enlist E3®” and “Enlist™ Roundup Ready® 2 Yield” that are tolerant to three herbicides of glyphosate, glufosinate, and 2,4-D; the genetically modified maize “Enlist™ Roundup Ready® Corn 2” that is tolerant to glyphosate, 2,4-D, and an aryloxyphenoxypropionate (FOPs) herbicide; the genetically modified maize “Enlist™ Roundup Ready® Corn 2” that is tolerant to glyphosate, 2,4-D, and an aryloxyphenoxypropionate (FOPs) herbicide; the genetically modified cotton “Bollgard II®, XtendFlex™ Cotton” that is tolerant to dicamba, glyphosate, and glufosinate; and the genetically modified cotton “Enlist™ Cotton” that is tolerant to three herbicides of glyphosate, glufosinate, and 2,4-D. In addition, the cotton tolerant to glufosinate and 2,4-D, the cotton tolerant to both glufosinate and dicamba, the maize tolerant to both glyphosate and 2,4-D, the soybean tolerant to both glyphosate and an HPPD herbicide, and the genetically modified maize tolerant to glyphosate, glufosinate, 2,4-D, an aryloxyphenoxypropionate (FOPs) herbicide, and a cyclohexanedione (DIMs) herbicide have been also developed.

Examples of the commercially available plants to which herbicide tolerance and pest resistance are imparted include the maize “YieldGard Roundup Ready®” and “YieldGard Roundup Ready®2” that are tolerant to glyphosate and resistant to a corn borer; the maize “Agrisurec® CB/LL” that is tolerant to glufosinate and resistant to a corn borer; the maize “Yield Gard® VT Root worm/RR2” that is tolerant to glyphosate and resistant to a corn rootworm; the maize “Yield Gard® VT Triple” that is tolerant to glyphosate and resistant to a corn rootworm and a corn borer; the maize “Herculext® 1” that is tolerant to glufosinate and resistant to a lepidopteran maize pest (Cry1F) (for example, resistance to a western bean cutworm, a corn borer, a black cutworm, and a fall armyworm); the maize “YieldGanl® Corn Rootworm/Roundup Ready® 2” that is tolerant to glyphosate and resistant to a corn rootworm; the maize “Agrisure, GT/RW” that is tolerant to glufosinate and resistant to a Coleoptera maize pest (Cry3A) (for example, resistant to a western corn rootworm, a northern corn rootworm, and a Mexican corn rootworm); the maize “Herculex® RW” that is tolerant to glufosinate and resistant to a Coleoptera maize pest (Cry34/35Abl) (for example, resistant to a western corn rootworm, a northern corn rootworm, and a Mexican corn rootworm); the maize “Yield Gard® VT Root worm/RR2” that is tolerant to glyphosate and resistant to a corn rootworm; and the cotton “Bollgard 3A XtendFlext” that is tolerant to dicamba, glyphosate, and glufosinate and resistant to a lepidopteran cotton pest (for example, resistant to bollworms, a tobacco budworm, and armyworms).

In the present invention, a composition of the invention is applied to a place where weeds are growing or likely to grow. Examples of the method of applying the present composition include a method of spraying the present composition on soil and a method of spraying the present composition on weeds.

In some variations, the application rate of a composition of the invention is generally 1 to 10,000 g per 10,000 m², 2 to 5,000 g per 10,000 m², 5 to 2,000 g per 10,000 m², 1 to 1000 g per 10,000 m², 1 to 500 g per 10,000 m², 1 to 100 g per 10,000 m², 1 to 75 g per 10,000 m², 15 to 1000 g per 10,000 m², 15 to 100 g per 10,000 m², 15 to 75 g per 10,000 m², or 15 to 60 g per 10,000 m², in terms of the total amount of a compound of formula (I), (II), (III), (IV), or (V) or a salt thereof (including an agriculturally suitable salt thereof).

In one variation, the application rate of a composition of the invention is generally 1 to 10,000 g per 10,000 m², 2 to 5,000 g per 10,000 m², 5 to 2,000 g per 10,000 m², 1 to 1000 g per 10,000 m², 1 to 500 g per 10,000 m², 1 to 100 g per 10,000 m², 1 to 75 g per 10,000 m², 15 to 1000 g per 10,000 m², 15 to 100 g per 10,000 m², 15 to 75 g per 10,000 m², or 15 to 60 g per 10,000 m², in terms of the total amount of a compound of formula (I), (II), (III), (IV), or (V) and the at least one compound selected from the group consisting of the herbicide compound group B and the safener group C.

In the present method, an adjuvant may be mixed in a composition of the invention, followed by application. The type of the adjuvant is not particularly limited, and examples of the adjuvant include oil-based adjuvants such as Agri-Dext and methylated seed oil (MSO), non-ions (esters or ethers of polyoxyethylene) such as Induce, anions (substituted sulfonates) such as Gramine S, cations (polyoxyethylene amines) such as Genamint T 200BM, and organic silicons such as Silwet®) L77.

The pH and the hardness of the spray liquid prepared when a composition of the invention is applied are not particularly limited, and the pH is usually in the range of 5 to 9, and the hardness is usually in the range of 0 to 500.

The time period for applying a composition of the invention is not particularly limited, and is usually in the range of 5:00 AM to 9:00 PM, and the photon flux density is usually 10 to 2,500 μmol/m²/s.

When a composition of the invention is applied to a crop field, it may be applied before sowing a crop seed, simultaneously with sowing a crop seed, and/or after sowing a crop seed. That is, the frequency of the application of a composition of the invention is once before, simultaneously with, or after sowing a crop seed, twice excluding before the sowing, excluding simultaneously with the sowing, or excluding after the sowing, or three times at all the timing.

When a composition of the invention is applied before sowing a crop seed, it is applied from 50 days before to immediately before the sowing, preferably from 30 days before to immediately before the sowing, more preferably from 20 days before to immediately before the sowing, and still more preferably from 10 days before to immediately before the sowing.

When a composition of the invention is applied after sowing a crop seed, it is usually applied from immediately after the sowing to before flowering. The composition is more preferably applied from immediately after the sowing to before the emergence, or from 1 to 6 leaf stages of the crop. The case where a composition of the invention is applied simultaneously with sowing a crop seed is the case where a sowing machine and a sprayer are integrated with each other.

In the step of applying a composition of the invention in a cultivation area, a compound of formula (I), (II), (III), (IV), or (V) or the compound and at least one additional compound selected from the group consisting of the herbicide compound group B and the safener group C are usually mixed with a carrier such as a solid carrier or a liquid carrier, and an auxiliary agent for formulation such as a surfactant is added if necessary to prepare a formulation. Preferable formulation types is aqueous liquid suspension formulations, oil-based suspension formulations, wettable powders, water dispersible granules, granules, water-based emulsions, oil-based emulsions, and emulsifiable concentrates, and more preferable formulation type is emulsifiable concentrates. Furthermore, a formulation containing a compound of formula (I), (II), (III), (IV), or (V) alone as an active ingredient and a formulation containing the at least one compound selected from the group consisting of the herbicide compound group B and the safener group C as an active ingredient may be used in combination. Furthermore, a formulation containing the present composition as active ingredients and a formulation containing another herbicide as an active ingredient may be used in combination.

Examples of the method of applying a composition of the invention in a cultivation area include a method of spraying it on the soil in the cultivation area and a method of spraying the present composition on a weeds that are growing. The composition is usually diluted with water, followed by spraying. The spray volume is not particularly limited, and is usually 50 to 1,000 L/ha, preferably 100 to 500 L/ha, and more preferably 140 to 300 L/ha.

Specific examples of the weed species to be controlled by the present composition include, but are not limited to, the weed species described below.

Urticaceae weeds to be controlled include Urtica urens.

Polygonaceae weeds to be controlled include Polygonum convolvulus, Polygonum lapathifolium, Polygonum pensylvanicunm, Polygonum persicaria, Polygonum longisetum, Polygonum aviculare, Polygonum arenastrum, Polygonum cuspidatum, Rumex japonicus, Rumex crispus, Rumex obtusifolius, and Rumex acetosa.

Portulacaceae weeds to be controlled include Portulaca oleracea.

Caryophyllaceae weeds to be controlled include Stellaria media, Stellaria aquatica, Cerashum holosteoides, Cerastium glomeratum, Spergula arvensis, and Silene gallica.

Molluginaceae weeds to be controlled include Mollugo verticillate.

Chenopodiaceae weeds to be controlled include Chenopodium album, Chenopodium ambrosioides, Kochia scoparia, Salsola kali, and Atriplex spp.

Amaranthaceae weeds to be controlled include Amaranthus retroflexus, Amaranthus viridis, Amaranthus lividus, Amaranthus spinosus, Amaranthus hybridus, Amaranthus palmeri, Amaranthus patulus, Waterhemp (Amaranthus tuberculatus, Amaranthus rudis, or Amaranthus tamariscinus), Amaranthus blitoides, Amaranthus deflexus, Amaranthus quitensis, Alternanthera philoxeroides, Alternanthera sessilis, and Alternanthera tenella.

Papaveraceae weeds to be controlled include Papaver rhoeas, Papaver dubium, and Argemone Mexicana.

Brassicaceae weeds to be controlled include Raphanus raphanistrum, Raphanus sativus, Sinapis arvensis, Capsella bursa-pastoris, Brassica juncea, Brassica napus, Descutrainia pinnata, Rorippa islandica, Rorippa sylvestris, Thlaspi arvense, Mvagrum rugosum, Lepidium virginicum, and Coronopus didymus.

Capparaceae weeds to be controlled include Cleome affinis.

Fabaceae weeds to be controlled include Aeschynomene indica, Aeschynomene rudis, Sesbania exaltata, Cassia obtusifolia, Cassia occidentalis. Desmodium tortuosum, Desmodium adscendens, Desmodium illinoense, Trifolium repens, Pueraria lobata, Vicia angustifolia, Indigofera hirsuta, Indigofera truxillensis, and Vigna sinensis.

Oxalidaceae weeds to be controlled include Oxalis corniculata, Oxalis strica, and Oxalis oxyptera.

Geraniaceae weeds to be controlled include Geranium carolinense and Erodium cicutarium.

Euphorbiaceae weeds to be controlled include Euphorbia helioscopia, Euphorbia maculata, Euphorbia humistrata, Euphorbia esula, Euphorbia heterophylla, Euphorbia brasiliensis, Acalpha australis, Croton glandulosus, Croton lobatus, Phyllanthus corcovadensis, and Ricinus communis.

Malvaceae weeds to be controlled include Abutilon theophrasti, Sida rhombiforia, Sida cordifolia, Sida spinosa, Sida glaziovii, Sida santaremnensis, Hibiscus trionum, Anoda cristata, and Malvastrum coromandelianum.

Onagraceae weeds to be controlled include Ludwigia epilobioides, Ludwigia octovalvis, Ludwigia decurre, Oenothera biennis, and Oenothera laciniata.

Sterculiaceae weeds to be controlled include Waltheria indica.

Violaccac weeds to be controlled include Viola arvensis and Viola tricolor.

Cucurbitaceae weeds to be controlled include Sikos angulatus, Echinocystis lobata, and Momordica charantia.

Lythraceae weeds to be controlled include Ammannia multiflora, Ammannia auriculata, Ammannia coccinea, Lythrum salicaria, and Rotala indica.

Elatinaccae weeds to be controlled include Elatine triandra and Elatine californica.

Apiaceae weeds to be controlled include Oenanthe javanica, Daucus carota, and Conium maculatum.

Ceratophyllaceae weeds to be controlled include Ceratophyllum demersum.

Cabombaceae weeds to be controlled include Cabomba carohniana.

Haloragaceae weeds to be controlled include Myriophyllum aquaticum, Myriophyllum verticillatum, Afyriophyllum spicatum, and Afyriophyllum heterophyllum.

Sapindaccae weeds to be controlled include Cardiospermum halicacabum.

Primulaceae weeds to be controlled include Anagallis arvensis.

Asclepiadaceae weeds to be controlled include Asclepias syriaca, and Ampelamus albidus.

Rubiaceae weeds to be controlled include Gahum aparine, Galium spurium var. echinospermon, Spermacoce latifolia, Richardia brasiliensis, and Borreria alata.

Convolvulaceae weeds to be controlled include Ipomoea nil, Ipomoea hederacea, Ipomoea purpurea, Ipomoea hederacea var. integriuscula, Ipomoea lacunosa, Ipomoea triloba, Ipomoea acuminata, Ipomoea hederifolia, Ipomoea coccinea, Ipomoea quamoclit, Ipomoea grandifolia, Ipomoea aristolochiafolia, Ipomoea cairica, Convolvulus arvensis, Calystegia hederacea, Calystegia japonica, Merremia hedeacea, Merremia aegptia, Merremia cissoides, and Jacquemontia tamnifolia.

Boraginaceae weeds to be controlled include Myosotis arvensis.

Lamiaceae weeds to be controlled include Lamium purpureum, Lamium amplexicaule, Leonotis nepetaejolia, Hyptis suaveolens, Hyptis lophanta, Leonurus sibiricus, and Stachys arvensis.

Solanaceae weeds to be controlled include Datura stramonium, Solanum nigrum, Solanum americanum, Solanum ptycanthum, Solanum sarrachoides, Solanum rostratum, Solanum aculeatissimum, Solanum sisymbriifolium, Solanum carolinense, Physalis angulata, Physalis subglabrata, and Nicandra physaloides.

Scrophulariaceae weeds to be controlled include Veronica hederaefolia, Veronica persica, Veronica arvensis, Lindernia procumbens, Lindernia dubia, Lindernia angustifolia, Bacopa rorundifolia, Dopatrium junceum, and Gratiola japonica.

Plantaginaceae weeds to be controlled include Plantago asiatica, Plantago lanceolata, Plantago major, and Callitriche palustris.

Asteraceae weeds to be controlled include Xanthium pensylvanicum, Xanthium occidentale, Xanthium italicum, Helianthus annuus, Marricaria chamomilla, Matricaria perforata, Chrysanthemum segetum, Matricarra matricarioides. Artemisia princeps, Artemisia vulgaris, Artemisia verlotorum, Solidago altissima, Taraxacum officinale, Galinsoga ciliata, Galinsoga parvillora, Senecio vulgaris, Senecio brasiliensis, Senecio grisebachii, Conza bonariensis, Conyza smatrensis, Conpza canadensis, Ambrosia artemisraefoha, Ambrosia trifida, Bidens tripartita, Bidens pilosa, Bidens frondosa, Bidens subalternans, Cirsium arvense, Cirsium vulgare, Silybum marianum, Carduus nutans, Lactuca serriola, Sonchus oleraceus, Sonchus asper, Wedelia glauca, Melampodium perfoliatum, Emilia sonchifolia, Tagetes minuta, Blainvillea latifolia, Tridax procumbens, Porophyllum ruderale, Acanthospermum australe, Acanthospermum hispidum, Cardiospermum halicacabum, Ageratum conyzoides, Eupatorium perfoliatum, Eclipta alba, Erechtites hieracifolia, Gamochaela spicata, Gnaphalium spicatum, Jaegeria hirta, Parthenium hyterophorus, Siegesbeckia orientalis, Soliva sessilis, Eclipta prostrata, Eclipta alba, and Centipeda minima.

Alismataceae weeds to be controlled include Sagittaria pygmaea, Sagittaria trifiolia, Sagittaria sagittifolia, Sagittaria montevidensis, Sgittaria aginashi, Alisma canaliculatum, and Alisma plantago-aquatica.

Limnocharitaceac weeds to be controlled include Limnocharis flava.

Hydrocharitaceae weeds to be controlled include Limnobium spongia, Hydrilla verticillata, and Najas guadalupensis.

Araceae weeds to be controlled include Pistia stratiotes.

Lemnaceae weeds to be controlled include Lemna aoukikusa, Spirodela polyrhiza, and Wolffia spp.

Potamogetonaceae to be controlled include Potamogeton distinctus, Potamogeton crispus, Potamogeton illinoensis, and Stuckenia pectinata.

Liliaceae weeds to be controlled include Allium canadense, Allium vineale, and Allium macrostemon.

Pontederiaceae weeds to be controlled include Eichhornia crassipes, Heteranthera limosa, Monochoria korsakowii, and Monochoria vaginalis.

Commelinaceae weeds to be controlled include Commelina communis, Commelina bengharensis, Commelina erecta, and Murdannia keisak.

Poaceae weeds to be controlled include Echinochloa crus-galli, Echinochloa oryzicola, Echinochloa crus-galli var formosensis, Echinochloa oryzoides, Echinochloa colona, Echinochloa crus-pavonis, Setaria viridis, Setaria faberi, Setaria glauca, Selaria geniculata, Digitaria ciliaris, Digitaria sangunals, Digitaria horizontalis, Digitaria insularis, Eleusine indica, Poa annua, Poa rivialis, Poa pratensis, Alospecurus aequalis, Alopecurus myosuroides, Avena fatua, Sorghum halepense, Sorghum vulgare, Agropvron repens, Lolium multijlorum, Lolium perenne, Lolium rigidum, Bromus catharticus, Bromus sterilis, Bromus japonicus, Bromus secalinus, Bromus tectorum, Hordeum jubatum, Aegilops cylindrica, Phalaris arundinacea, Phalaris minor, Apera spica-venti, Panicum dichotomiflorum, Panicum texanum, Panicum maximum, Brachiaria platyphylla, Brachiaria ruziziensis, Brachiaria plantaginea, Brachiaria decumbens, Brachiaria brizantha, Brachiaria humidicola, Cenchrus echinatus, Cenchrus pauciflorus, Eriochloa villosa, Pennisetum setosum, Chions gayana, Chlorisvirgata, Eragrostis pilosa, Rhynchehtrum repens, Dactyloctenium aegyptium, Ischaemum rugosum, Isachne globosa, Oryza sativa, Paspalum notatum, Paspalum maritimum, Payalum distichum, Pennisetum clandestinum, Pennisetum setosum, Rottboellia cochinchinensis, Leptochloa chinensis, Leptochloa fascicilaris, Leptochloa fliformis, Leptochloa panicoides, Leersia japonica, Leersia sayanuka, Leersia orvzoides, Glyeria leptorrhiza, Glyceria acutiflora, Glvceria maxima, Agrostis gigantea, Agrostis stolonifera, Cynodon dactylon, Dactylis glomerata, Eremochloa ophiuroides, Festuca arundinacea, Festuca rubra, Imperata cylindrica, Miscanthus sinensis, Panicum virgatum, and Zoysia japonica.

Cyperaceae weeds to be controlled include Cyperus microiria, Cuperus iria, Cyperus compressus, Cyperus diformis, Cyperus flaccidus, Cyperus globosus, Cyperus nipponics, Cyperus odoratus, Cyperus serorinus, Cyperus rotundus, Cyperus escdentus, Kyllinga gracillima, Kyllinga brevifoha, Fimbristlis miliacea, Fimbristylis dichotoma, Eleocharis acicularis, Eleocharis kuroguwai, Schoenoplectiella hotarui, Schoenoplectiella juncoides, Schoenoplectiella wallichii, Schoenoplecriella mucronatus, Schoenoplecriella triangulatus, Schoenoplectiella nipponicus, Schoenoplectiella triqueter, Bolboschoenus koshevnikovii, and Bolboschoenus fluviatilis.

Equisetaceae weeds to be controlled include Equisetum arvense, and Equisetum palustre.

Salviniaceae weeds to be controlled include Salvinia natans.

Azollaceae weeds to be controlled include Azolla japonica and Azolla imbricata.

Marsileaceae weeds to be controlled include Marsilea quadrifolia.

Other weeds to be controlled include Pithophora, Cladophora, Bryophvta, Marchantiophyta, Anthocerotophyta, Cvanobacteria, Pteridophyta, sucker of perennial crops (pomaceous fruits, nut trees, citruses, Humulus lupulus, grapes, and the like).

In the above-mentioned weeds to be controlled, mutations within the species are not particularly limited. That is, the weeds include weeds having reduced sensitivity to a specific herbicide. The reduced sensitivity may be attributed to a mutation at a target site (target site mutation) or may be attributed to any factors other than the target site mutation (non-target site mutation). Examples of the factor of the reduced sensitivity due to a non-target site mutation include increased metabolism, malabsorption, translocation dysfunction, and excretion to out of system. Examples of the factor of the increased metabolism include the enhanced activity of a metabolizing enzyme such as cytochrome P450 monooxygenase, aryl acylamidase, esterase, or glutathione S-transferase. Examples of the excretion to out of system include transport to the vacuole by an ABC transporter. Examples of the weeds having reduced sensitivity due to a target site mutation include weeds having any one of or two or more of the following amino acid substitutions in the ALS gene: Ala122Thr, Ala122Val, Ala122Tyr, Pro197Ser, Pro197His, Pro197Thr, Pro197Arg, Pro197Leu, Pro197Gln, Pro197Ala, Pro197Ile, Ala205Val, Ala205Phe, Asp376Glu, Arg377His, Trp574Leu, Trp574Gly, Trp574Met, Ser653Thr, Ser653Thr, Ser653Asn, Ser635Ile, Gly654Glu, and Gly645Asp. Similarly, examples of the weeds having reduced sensitivity due to a target site mutation include weeds having any one of or two or more of the following amino acid substitutions in the ACCase gene: Ile1781Leu, Ile1781Val, Ile1781Thr, Trp1999Cys, Trp1999Leu, Ala2004Val, Trp2027Cys, Ile2041Asn, Ile2041 Val, Asp2078Gly, Cys2088Arg, Gly2096Ala, and Gly2096Ser.

Similarly, as an example of the weeds having reduced sensitivity due to a target site mutation. PPO inhibitor-resistant weeds having one or more mutations selected from an Arg128Leu mutation, an Arg128Met mutation, an Arg128Gly mutation, an Arg128His mutation, a Gly210 deletion mutation, and a Gly399Ala mutation in PPO. The word “PPO” means protoporphyrinogen oxidase. Weeds usually have PPO1 and PPO2 in PPO, and the above-mentioned mutations may be present in either PPO1 or PPO2 or in both. The case where weeds have the mutations in PPO2 is preferable. For example, the word “Arg128Met” means that the mutation is present in the 128th (the number is standardized with PPO2 of Amaranthus palmeri) amino acid. In PPO2 of Ambrosia artemisiaefolia, the mutation corresponds to a mutation in the 98th amino acid (Rousonelos, et al., Weed Science (2012) Vol. 60, pgs. 335-344) and is known as Arg98Leu. In this case, Arg98 is equivalent to Arg128 according to the present invention. The Arg128Met mutation and the Arg128Gly mutation in the PPO of the weed to be controlled in the present invention are known in Amaranthus palmeri (Giacomini, et al., Pest Management Science (2017) Vol. 73, pgs. 1559-1563), the Arg128His mutation is known in Lolium rigidum (Fernandez-Moreno, et al., Weed Science Society of America (WSSA) annual meeting, 2018), and the Gly399Ala mutation is known in Amaranthus palmeri (Rangani, et al., WSSA annual meeting, 2018). In the present invention, the above-mentioned reported resistant weeds are particularly effectively controlled, but particularly effectively controlled weeds are not limited thereto. That is, other weeds having the amino acid mutation are similarly controlled. Not only Amaranthus palmeri having an Arg128Leu mutation, an Arg128Met mutation, an Arg128Gly mutation, an Arg128His mutation, a Gly210 deletion mutation, or a Gly399Ala mutation, but also, for example, waterhemp having the above-mentioned mutation, Ambrosia artemisiaefolia having the above-mentioned mutation. Lolium rigidum having the above-mentioned mutation, Lolium multiflorum having the above-mentioned mutation, and Euphorbia heterophylla having the above-mentioned mutation are effectively controlled.

Similarly, examples of the weeds having reduced sensitivity due to a target site mutation include weeds having an amino acid substitution such as Thr102Ile, Pro106Ser, Pro106Ala, or Pro106Leu in the EPSP gene. In particular, Eleusine indica, Lolium multiflorum, Lolium rigichum, Digitaria insularis, waterhemp, Echinochloa colona, and the like which are resistant to glyphosate and have one or both of the mutations are effectively controlled. Similarly, examples of the weeds having reduced sensitivity due to a target site include weeds having increased copies of the EPSP gene and Amaranthus palmed, waterhemp. Kochia scoparia, and the like which are resistant to glyphosate and have the mutation are particularly effectively controlled. Conyza canadensis, Conyza smatrensis, and Conyza bonariensis which are resistant to glyphosate in which an ABC transporter is involved are also effectively controlled.

In the cultivation of a crop according to the present invention, plant nutritional management in general cultivation of a crop can be performed. The fertilization system may be based on Precision Agriculture or may be conventionally uniform one. In addition, a nitrogen-fixing bacterium or a mycorrhizal fungus can be inoculated in combination with seed treatment.

### Combinations

In certain aspects, controlling effect on weeds is exhibited by using a compound of formula (I), (II), (III), (IV), or (V) and a specific compound in combination.

Accordingly, the present invention features—(i) A herbicidal composition including a compound of formula (I), (II), (III), (IV), or (V) and at least one compound selected from the group consisting of a herbicide compound group B and a safener group C, wherein a weight ratio of a compound of formula (I), (II), (III), (IV), or (V) to the at least one compound selected from the group consisting of the herbicide compound group B and the safener group C is 1:0.1 to 1:50, and the herbicide compound group B is a group consisting of the following B-1 to B-12:
B-1 acetolactate synthase inhibitors;
B-2 acetyl-CoA carboxylase inhibitors;
B-3 protoporphyrinogen IX oxidase inhibitors;
B-4 4-hydrophenylpyruvate dioxygenase inhibitors;
B-5 phytoene desaturase inhibitors;
B-6 photosystem II inhibitors;
B-7 very long chain fatty acid synthesis inhibitors;
B-8 microtubule formation inhibitors;
B-9 auxin herbicides;
B-10 enolpyruvylshikimate 3-phosphate synthase inhibitors,
B-11 glutamine synthase inhibitors; and
B-12 other herbicides (including agriculturally acceptable salts or derivatives for each of B-1 to B-12)

The present invention also features—(ii) the herbicidal composition according to (i), wherein:
the B-1 is a group consisting of pyrithiobac, pyrithiobac-sodium salt, pyriminobac, pyriminobac-methyl, bispyribac, bispyribac-sodium salt, pyribenzoxim, pyrimisulfan, pyriftalid, triafamone, amidosulfuron, azimsulfuron, bensulfuron, bensulfuron-methyl, chlorimuron, chlorimuron-ethyl, cyclosulfamuron, ethoxysulfuron, flazasulfuron, flucetosulfuron, flupyrsulfuron, flupyrsulfuron-methyl-sodium, foramsulfuron, halosulfuron, halosulfuron-methyl, imazosulfuron, mesosulfuron, mesosulfuron-methyl, metazosulfuron, nicosulfuron, orthosulfamuron, oxasulfuron, primisulfuron, primisulfuron-methyl, propyrisulfuron, pyrazosulfuron, pyrazosulfuron-ethyl, rimsulfuron, sulfometuron, sulfometuron-methyl, sulfosulfuron, trifloxysulfuron, trifloxysulfuron-sodium salt, chlorsulfuron, cinosulfuron, ethametsulfuron, ethametsulfuron-methyl, iodosulfuron, iodosulfuron-methyl-sodium, iofensulfuron, iofensulfuron-sodium, metsulfuron, metsulfuron-methyl, prosulfuron, thifensulfuron, thifensulfuron-methyl, triasulfuron, tribenuron, tribenuron-methyl, triflusulfuron, triflusulfuron-methyl, tritosulfuron, bencarbazone, flucarbazone, flucarbazone-sodium salt, propoxycarbazone, propoxycarbazone-sodium salt, thiencarbazone, thiencarbazone-methyl, cloransulam, cloransulam-methyl, diclosulam, florasulam, flumetsulam, metosulam, penoxsulam, pyroxsulam, imazamethabenz, imazamethabenz-methyl, imazamox, imazamox-ammonium salt, imazapic, imazapic-ammonium salt, imazapyr, imazapyr-isopropylammonium salt, imazaquin, imazaquin-ammonium, imazethapyr, and imazethapyr-ammonium salt (including agriculturally acceptable salts and derivatives thereof for each);
the B-2 is a group consisting of clodinafop, clodinafop-propargyl, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, propaquizafop, quizalofop, quizalofop-ethyl, quizalofop-P, quizalofop-P-ethyl, alloxydim, clethodim, sethoxydim, tepraloxydim, tralkoxydim, and pinoxaden (including agriculturally acceptable salts and derivatives thereof for each);
the B-3 is a group consisting of azafenidin, oxadiazon, oxadiargyl, carfentrazone, carfentrazone-ethyl, saflufenacil, cinidon, cinidon-ethyl, sulfentrazone, pyraclonil, pyraflufen, pyraflufen-ethyl, butafenacil, fluazolate, fluthiacet, fluthiacet-methyl, flufenpyr, flufenpyr-ethyl, flumiclorac, flumiclorac-pentyl, flumioxazin, pentoxazone, oxyfluorfen, acifluorfen, acifluorfen-sodium salt, aclonifen, chlormethoxymil, chlornitrofen, nitrofen, bifenox, fluoroglycofen, fluoroglycofen-ethyl, fomesafen, fomesafen-sodium salt, lactofen, tiafenacil, and ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (including agriculturally acceptable salts and derivatives thereof for each);
the B-4 is a group consisting of benzobicyclon, bicyclopyrone, mesotrione, sulcotrione, tefuryltrione, tembotrione, isoxachlortole, isoxaflutole, benzofenap, pyrasulfotole, pyrazolynate, pyrazoxyfen, fenquinotrione, topramezone, tolpyralate, lancotrione, lancotrione-sodium salt, 2-methyl-N-(5-methyl-1,3,4-oxadiazol-2-yl)-3-(methylsulfonyl)-4-(trifluoromethyl)benzamide (CAS Registry Number: 1400904-50-8), 2-chloro-N-(1-methyl-1H-tetrazol-5-yl)-3-(methylthio)-4-(trifluoromethyl)-benzamide (CAS Registry Number: 1361139-71-0), and 4-(4-fluorophenyl)-6-[(2-hydroxy-6-oxo-1-cyclohexene-1-yl)carbonyl]-2-methyl-1,2,4-triazine-3,5(2H,4H)-dione (CAS Registry Number: 1353870-34-4) (including agriculturally acceptable salts and derivatives thereof for each);
the B-5 is a group consisting of diflufenican, picolinafen, beflubutamid, norflurazon, fluridonem, flurochloridone, and flurtamone (including agriculturally acceptable salts and derivatives thereof for each);
the B-6 is a group consisting of ioxynil, ioxynil-octanoate, bentazone, pyridate, bromoxynil, bromoxynil-octanoate, chlorotoluron, dimefuron, diuron, linuron, fluometuron, isoproturon, isouron, tebuthiuron, benzthiazuron, methabenzthiazuron, propanil, metobromuron, metoxuron, monolinuron, siduron, simazine, atrazine, propazine, cyanazine, ametryn, simetryn, dimethametryn, prometryn, terbumeton, terbuthylazine, terbutryn, trietazine, hexazinone, metamitron, metribuzin, amicarbazone, bromacil, lenacil, terbacil, chloridazon, desmedipham, and phenmedipham (including agriculturally acceptable salts and derivatives thereof for each),
the B-7 is a group consisting of propachlor, metazachlor, alachlor, acetochlor, metolachlor, S-metolachlor, butachlor, pretilachlor, thenylchlor, indanofan, cafenstrole, fentrazamide, dimethenamid, dimethenamid-P, mefenacet, pyroxasulfone, fenoxasulfone, naproanilide, napropamide, anilofos, flufenacet, and ipfencarbazone (including agriculturally acceptable salts and derivatives thereof for each);
the B-8 is a group consisting of trifluralin, pendimethalin, ethalfluralin, benfluralin, oryzalin, prodiamine, butamifos, dithiopyr, and thiazopyr (including agriculturally acceptable salts and derivatives thereof for each);
the B-9 is a group consisting of 2,4-DB [4-(2,4-dichlorophenoxy)butyric acid] and its salts or esters (dimethylammonium salt, isooctyl ester, and choline salt), MCPA and its salts or esters (dimethylammonium salt, 2-ethylhexyl ester, isooctyl ester, sodium salt, and choline salt), MCPB, mecoprop and its salts or esters (dimethylammonium salt, dioramine salt, ethadyl ester, 2-ethylhexyl ester, isooctyl ester, methyl ester, potassium salt, sodium salt, trolamine salt, and choline salt), mecoprop-P and its salts or esters (dimethylammonium salt, 2-ethylhexyl ester, isobutyl salt, potassium salt, and choline salt), dichlorprop and its salt or ester (butotyl ester, dimethylammonium salt, 2-ethylhexyl ester, isooctyl ester, methyl ester, potassium salt, sodium salt, and choline salt), dichlorprop-P, dichlorprop-P dimethylammonium, triclopyr and its salts or esters (butotyl ester, and triethylammonium salt), fluroxypyr, fluroxypyr-meptyl, picloram and its salts (potassium salt, tris(2-hydroxypropyl)ammonium salt, and choline salt), quinclorac, quinmerac, aminopyralid and its salts (potassium salt, tris(2-hydroxypropyl)ammonium salt, and choline salt), clopyralid and its salts (olamine salt, potassium salt, triethylammonium salt, and choline salt), clomeprop, aminocyclopyrachlor, halauxifen, halauxifen-methyl, florpyrauxifen, and florpyrauxifen-benzyl (including agriculturally acceptable salts and derivatives thereof for each);
the B-10 is a group consisting of glyphosate, glyphosate-isopropylammonium salt, glyphosate-trimesium salt, glyphosate-ammonium salt, glyphosate-diammonium salt, glyphosate-dimethylammonium salt, glyphosate-monoethanolamine salt, glyphosate-sodium salt, glyphosate-potassium salt, and glyphosate-guanidine salt (including agriculturally acceptable salts and derivatives thereof for each);
B-11 is a group consisting of glufosinate, glufosinate-ammonium salt, glufosinate-P, glufosinate-P-sodium salt, and bialaphos (including agriculturally acceptable salts and derivatives thereof for each), and
the B-12 is a group consisting of isoxaben, dichlobenil, methiozolin, diallate, butylate, triallate, chlorpropham, asulam, phenisopham, benthiocarb, molinate, esprocarb, pyributicarb, prosulfocarb, orbencarb, EPIC, dimepiperate, swep, difenoxuron, methyldymron, bromobutide, daimuron, cumyluron, diflufenzopyr, diflufenzopyr-sodium salt, etobenzanid, tridiphane, amitrole, clomazone, 2-[(2,4-dichlorophenyl)methyl]-4,4-dimethylisoxazolidin-3-one (CAS Registry Number: 81777-95-9), (3S,4S)-N-(2-fluorophenyl)-1-methyl-2-oxo-4-[3-(trifluoromethyl)phenyl]-3-pynolidinecarboxamide (CAS Registry Number: 2053901-33-8), maleic hydrazide, oxaziclomefone, cinmethylin, benfuresate, ACN, dalapon, chlorthiamid, flupoxain, bensulide, paraquat, paraquat-dichloride, diquat, diquat-dibromide, MSMA, indaziflam, and triaziflam (including agriculturally acceptable salts and derivatives thereof for each).

The present invention also features—(iii) the herbicidal composition according to (i) or (ii), wherein the safener group C is a group consisting of benoxacor, cloquintocet, cyometrinil, cyprosulfamide, dichlormid, dicyclonone, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane, 2,2,5-trimethyl-3-(dichloroacetyl)-1,3-oxazolidine, and N-(2-methoxybenzoyl)-4-[(methylaminocarbonyl)amino]benzenesulfon-amide.

in one embodiment, the present invention includes—(iv) the herbicidal composition according to (i), wherein B-1 is a group consisting of pyrithiobac, pyrithiobac-sodium salt, chlorimuron-ethyl, foramsulfuron, halosulfuron-methyl, nicosulfuron, primisulfuron-methyl, rimsulfuron, trifloxysulfuron-sodium salt, chlorsulfuron, iodosulfuron-methyl-sodium, iofensulfuron sodium, metsulfuron-methyl, prosulfuron, thifensulfuron-methyl, tribenuron-methyl, thiencarbazone-methyl, cloransulam-methyl, flumetsulam, imazamethabenz-methyl, imazamox-ammonium salt, imazapic-ammonium salt, imazapyr-isopropylammonium, imazaquin-ammonium salt, and imazethapyr-ammonium salt (including agriculturally acceptable salts and derivatives thereof for each).

In another embodiment, the present invention includes—(v) the herbicidal composition according to (i), wherein B-2 is a group consisting of fenoxaprop-ethyl, fenoxaprop-P-ethyl, fluazifop-butyl, fluazifop-P-butyl, quizalofop-ethyl, quizalofop-P-ethyl, clethodim, and sethoxydim (including agriculturally acceptable salts and derivatives thereof for each).

In another embodiment, the present invention includes—[6] the herbicidal composition according to [1], wherein B-3 is a group consisting of carfentrazone-ethyl, saflufenacil, sulfentrazone, pyraflufen-ethyl, fluthiacet-methyl, flufenpyr-ethyl, flumiclorac-pentyl, flumioxazin, oxyfluorfen, acifluorfen-sodium salt, fomesafen-sodium salt, lactofen, tiafenacil, and ethyl [(3-{2-chloro-4-fluoro-5-[3-methyl-4-(trifluoromethyl)-2,6-dioxo-1,2,3,6-tetrahydropyrimidine-1-yl]phenoxy}pyridin-2-yl)oxy]acetate (including agriculturally acceptable salts and derivatives thereof for each).

In another embodiment, the present invention includes—[7] the herbicidal composition according to [1], wherein B-4 is a group consisting of bicyclopyrone, mesotrione, tembotrione, isoxaflutole, fenquinotrione, topramezone, tolpyralate, lancotrione-sodium salt, 2-methyl-N-(5-methyl-1,3,4-oxadiazol-2-yl)-3-(methylsulfonyl)-4-(trifluoromethyl)benzamide (CAS Registry Number 1400904-50-8), 2-chloro-N(1-methyl-1H-tetrazol-5-yl)-3-(methylthio)-4-(trifluoromethyl)-benzamide (CAS Registry Number 1361139-71-0), and 4-(4-fluorophenyl)-6-[(2-hydroxy-6-oxo-1-cyclohexene-1-yl)carbonyl]-2-methyl-1,2,4-triazine-3,5-(2H,4H)-dione (CAS Registry Number 1353870-34-4) (including agriculturally acceptable salts and derivatives thereof for each).

In another embodiment, the present invention includes—[8] the herbicidal composition according to [1], wherein B-5 is a group consisting of norflurazon and fluridone (including agriculturally acceptable salts and derivatives thereof for each).

In another embodiment, the present invention includes—[9] the herbicidal composition according to [1], wherein B-6 is a group consisting of bentazone, bromoxynil octanoate, diuron, linuron, fluometuron, simazine, atrazine, ametryn, prometryn, and metribuzin (including agriculturally acceptable salts and derivatives thereof for each).

In another embodiment, the present invention includes—[10] the herbicidal composition according to [1], wherein B-7 is a group consisting of alachlor, acetochlor, metolachlor. S-metolachlor, dimethenamid, dimethenamid-P, pyroxasulfone, and flufenacet (including agriculturally acceptable salts and derivatives thereof for each).

In another embodiment, the present invention includes—[II] the herbicidal composition according to [1], wherein B-8 is a group consisting of trifluralin, pendimethalin, and ethalfluralin (including agriculturally acceptable salts and derivatives thereof for each).

In another embodiment, the present invention includes—[12] the herbicidal composition according to [1], wherein B-9 is a group consisting of 2,4-DB, fluroxypyr, flumxypyr-meptyl, clopyralid-olamine salt, clopyralid-potassium salt, clopyralid-triethylammonium salt, halauxifen, halauxifen-methyl, florpyrauxifen, and florpyrauxifen-benzyl (including agriculturally acceptable salts and derivatives thereof for each).

In another embodiment, the present invention includes—[13] the herbicidal composition according to [1], wherein B-10 is a group consisting of a combination of two or more of glyphosate, glvphosate-isopropylammonium salt, glyphosate-ammonium salt, glyphosate-dimethylamine salt, glyphosate-monoethanolamine salt, glyphosate-potassium salt, and glyphosate-guanidine salt (including agriculturally acceptable salts and derivatives thereof for each).

In another embodiment, the present invention includes—[14] the herbicidal composition according to [1], wherein the B-11 is a group consisting of glufosinate, glufosinate-ammonium salt, glufosinate-P, and glufosinate-P-sodium salt (including agriculturally acceptable salts and derivatives thereof for each).

In another embodiment, the present invention includes—[15] the herbicidal composition according to [1], wherein the B-12 is a group consisting of EPTC, diflufenzopyr, diflufenzopyr-sodium salt, clomazone, 2-[(2,4-dichlorophenyl)methyl]-4,4-dimethylisoxazolidin-3-one (CAS Registry Number: 81777-95-9), (3S,4S)—N-(2-fluorophenyl)-1-methyl-2-oxo-4-[3-(trifluoromethyl)phenyl]-3-pyrrolidinecarboxamide (CAS Registry Number: 2053901-33-8), cinmethylin, MSMA, paraquat, paraquat dichloride, diquat, and diquat dibromide (including agriculturally acceptable salts and derivatives thereof for each).

In another embodiment, the present invention includes—[16] The herbicidal composition according to [1], wherein the safener group C is a group consisting of benoxacor, cyprosulfamide, and isoxadifen-ethyl (including agriculturally acceptable salts and derivatives thereof for each).

The present invention also features—[18] A method for controlling weeds, the method including a step of applying a compound of formula (I), (II), (III), (IV), or (V) and at least one compound selected from the group consisting of the herbicide compound group B and the safener group C simultaneously or sequentially to a place where weeds are growing or to grow.

In one embodiment, the present invention includes—[19] The method according to [18], wherein a compound of formula (I), (II), (III), (IV), or (V) and the at least one compound selected from the group consisting of the herbicide compound group B and the safener group C are used at a weight ratio of 1:0.1 to 1:50.

In another embodiment, the present invention includes—[20] The method according to 1181 or 1191, wherein the place where weeds are growing or to grow is a crop field.

The present invention also features—[21] A use of the herbicidal composition according to any one of [1] to [16], for controlling weeds.

Herbicidal compositions according to the present invention also include a compound of formula (I), (II), (III), (IV), or (V) and at least one compound selected from the group consisting of an herbicide compound group B and a safener group C.

The method for controlling weeds according to the present invention (hereinafter referred to as “present method”) includes the step of applying the present composition to a place where weeds are growing or likely to grow in a crop field, a vegetable field, a land under perennial crops, a non-crop land, or the like. In a crop field and a vegetable field, the present composition may be applied before, simultaneously with, and/or after sowing a crop seed.

The present method includes the step of applying a compound of formula (I), (II), (III), (IV), or (V) and at least one compound selected from the group consisting of the herbicide compound group B and the safener group C simultaneously or sequentially to a place where weeds are growing or likely to grow. In the case of the sequential application, the order of the application is not particularly limited.

The present composition is usually a formulation prepared by mixing a compound of formula (I), (II), (III), (IV), or (V) and at least one compound selected from the group consisting of the herbicide compound group B and the safener group C with a carrier such as a solid carrier or a liquid carrier and adding an auxiliary agent for formulation such as a surfactant if necessary. Preferable formulation types of such a formulation are aqueous liquid suspension concentrates, wettable powders, water dispersible granules, granules, and emulsifiable concentrates. The present composition may be used in combination with a formulation containing another herbicide as an active ingredient.

The total content of a compound of formula (I), (II), (III), (IV), or (V) and the at least one compound selected from the group consisting of the herbicide compound group B and the safener group C in the present composition is within a range of 0.01 to 90% by weight, preferably 1 to 80% b weight.

Hereinafter, when the at least one compound selected from the group consisting of the herbicide compound group B is a salt (for example, glyphosate-potassium salt), the weight of the at least one compound is represented by the acid equivalent.

A mixing ratio of a compound of formula (I), (II), (III), (IV), or (V) to the at least one compound selected from the group consisting of the herbicide compound group B and the safener group C in the present composition is within a range of 1:0.05 to 1:100, preferably 1:0.1 to 1:50 by weight ratio.

A ratio of application rates of a compound of formula (I), (II), (III), (IV), or (V) to the at least one compound selected from the group consisting of the herbicide compound group B and the safener group C in the present method is within a range of 1:0.05 to 1:100, preferably 1:0.1 to 1:50 by weight ratio.

In some variations, the mixing ratio of a compound of formula (I), (II), (III), (IV), or (V) to the at least one compound selected from the group consisting of the herbicide compound group B and the safener group C in the present composition include about 1:0.1, about 1:0.2, about 1:0.3, about 1:0.5, about 1:0.7, about 1:1, about 1:2, about 1:3, about 1:5, about 1:7, about 1:10, about 1:15, about 1:20, about 1:30, and about 1:50 by weight ratio.

In some variations, the ratio of application rates of a compound of formula (I), (II), (III), (IV), or (V) to the at least one compound selected from the group consisting of the herbicide compound group B and the safener group C in the present method include about 1:0.1, about 1:0.2, about 1:0.3, about 1:0.5, about 1:0.6, about 1:0.7, about 1:0.8, about 1:1, about 1:1.2, about 1:1.4, about 1:1.6, about 1:1.8, about 1:2, about 1:2.2, about 1:2.4, about 1:2.6, about 1:2.8, about 1:3, about 1:5, about 1:7, about 1:10, about 1:15, about 1:20, about 1:30, and about 1:50 by weight ratio.

The word ‘about’ in the preceding paragraph means that the specified ratio includes the ratio in the range increased or decreased by 10% by weight relative to the specified ratio. For example, a ratio of about 1:2 includes a range of 1:1.8 to 1:2.2.

In the present composition and the present method, particularly preferable examples of the combination of a compound of formula (I), (II), (III), (IV), or (V) and the at least one compound selected from the group consisting of the herbicide compound group B and the safener group C and the range of weight ratio thereof include, but are not limited to, the following combinations and the ranges:
a combination of a compound of formula (I), (II), (III), (IV) or (V) and pyrithiobac (1:0.1 to 1:20);
a combination of a compound of formula (I), (II), (III), (IV) or (V) and pyrithiobac-sodium salt (1:0.1 to 1:20);
a combination of a compound of formula (I), (II), (III), (IV) or (V) and chlorimuron-ethyl (1:0.1 to 1:20);
a combination of a compound of formula (I), (II), (III), (IV) or (V) and foramsulfuron (1:0.1 to 1:20);
a combination of a compound of formula (I), (II), (III), or (IV) and halosulfuron-methyl (1:0.1 to 1:20);
a combination of a compound of formula (I), (II), (III), (IV) or (V) and nicosulfuron (1:0.1 to 1:20);
a combination of a compound of formula (I), (II), (III), (IV) or (V) and primisulfuron-methyl (1:0.1 to 1:20);
a combination of a compound of formula (I), (II), (III), (IV) or (V) and rimsulfuron (1:0.1 to 1:20);
a combination of a compound of formula (I), (II), (III), (IV) or (V) and trifloxysulfuron-sodium salt (1:0.1 to 1:20);
a combination of a compound of formula (I), (II), (III), (IV) or (V) and chlorsulfuron (1:0.1 to 1:20);
a combination of a compound of formula (I), (II), (III), (IV) or (V) and iodosulfuron-methyl-sodium (1:0.1 to 1:20);
a combination of a compound of formula (I), (II), (III), (IV) or (V) and iofensulfuron-sodium (1:0.1 to 1:20);
a combination of a compound of formula (I), (II), (III), (IV) or (V) and metsulfuron-methyl (1:0.1 to 1:20);
a combination of a compound of formula (I), (II), (III), (IV) or (V) and prosulfuron (1:0.1 to 1:20);
a combination of a compound of formula (I), (II), (III), (IV) or (V) and thifensulfuron-methyl (1:0.1 to 1:20);
a combination of a compound of formula (I), (II), (III), (IV) or (V) and tribenuron-methyl (1:0.1 to 1:20);
a combination of a compound of formula (I), (II), (III), (IV) or (V) and thiencarbazone-methyl (1:0.1 to 1:20);
a combination of a compound of formula (I), (II), (III), (IV) or (V) and cloransulam-methyl (1:0.1 to 1:20);
a combination of a compound of formula (I), (II), (III), (IV) or (V) and flumetsulam (1:0.1 to 1:20);
a combination of a compound of formula (I), (II), (III), (IV) or (V) and imazamethabenz-methyl (1:0.1 to 1:20);
a combination of a compound of formula (I), (II), (III), (IV) or (V) and imazamox-ammonium salt (1:0.1 to 1:20);
a combination of a compound of formula (I), (II), (III), (IV) or (V) and imazapic-ammonium salt (1:0.1 to 1:20);
a combination of a compound of formula (I), (II), (III), (IV) or (V) and imazapyr-isopropylammonium salt (1:0.1 to 1:20);
a combination of a compound of formula (I), (II), (III), (IV) or (V) and imazaquin-ammonium salt (1:0.1 to 1:20);
a combination of a compound of formula (I), (II), (III), (IV) or (V) and imazethapyr-ammonium salt (1:0.1 to 1:20);
a combination of a compound of formula (I), (II), (III), (IV) or (V) and fenoxaprop-ethyl (1:0.1 to 1:20);
a combination of a compound of formula (I), (II), (III), (IV) or (V) and fenoxaprop-P-ethyl (1:0.1 to 1:20), a combination of a compound of formula (I), (II), (III), (IV) or (V) and fluazifop-butyl (1:0.1 to 1:20);
a combination of a compound of formula (I), (II), (III), (IV) or (V) and fluazifop-P-butyl (1:0.1 to 1:20);
a combination of a compound of formula (I), (II), (III), (IV) or (V) and quizalofop-ethyl (1:0.1 to 1:20);
a combination of a compound of formula (I), (II), (III), (IV) or (V) and quizalofop-P-ethyl (1:0.1 to 1:20);
a combination of a compound of formula (I), (II), (III), (IV) or (V) and clethodim (1:0.1 to 1:20);
a combination of a compound of formula (I), (II), (III), (IV) or (V) and sethoxydim (1:0.1 to 1:20);
a combination of a compound of formula (I), (II), (III), (IV) or (V) and carfentrazone-ethyl (1:0.1 to 1:20);
a combination of a compound of formula (I), (II), (III), (IV) or (V) and saflufenacil (1:0.1 to 1:20);
a combination of a compound of formula (I), (II), (III), (IV) or (V) and sulfentrazone (1:0.1 to 1:30);
a combination of a compound of formula (I), (II), (III), (IV) or (V) and pyraflufen-ethyl (1:0.1 to 1:30);
a combination of a compound of formula (I), (II), (III), (IV) or (V) and fluthiacet-methyl (1:0.1 to 1:20);
a combination of a compound of formula (I), (II), (III), (IV) or (V) and flufenpyr-ethyl (1:0.1 to 1:20);
a combination of a compound of formula (I), (II), (III), (IV) or (V) and flumiclorac-pentyl (1:0.1 to 1:20);
a combination of a compound of formula (I), (II), (III), (IV) or (V) and flumioxazin (1:0.1 to 1:20);
a combination of a compound of formula (I), (II), (III), (IV) or (V) and oxyfluorfen (1:0.1 to 1:30);
a combination of a compound of formula (I), (II), (III), (IV) or (V) and acifluorfen-sodium salt (1:0.1 to 1:30);
a combination of a compound of formula (I), (II), (III), (IV) or (V) and fomesafen-sodium salt (1:0.1 to 1:30);
a combination of a compound of formula (I), (II), (III), (IV) or (V) and lactofen (1:0.1 to 1:30);
a combination of a compound of formula (I), (II), (III), (IV) or (V) and tiafenacil (1:0.1 to 1:20);
a combination of a compound of formula (I), (II), (III), (IV) or (V) and ethyl [(3-{2-chloro-4-fluoro-5-[3-methyl-4-(trifluoromethyl)-2,6-dioxo-1,2,3,6-tetrahydropyrimidine-1-yl]phenoxy}pyridin-2-yl)oxy]acetate (1:0.1 to 1:20);
a combination of a compound of formula (I), (II), (III), (IV) or (V) and bicyclopyrone (1:0.1 to 1:20);
a combination of a compound of formula (I), (II), (III), (IV) or (V) and mesotrione (1:0.1 to 1:20);
a combination of a compound of formula (I), (II), (III), (IV) or (V) and tembotrione (1:0.1 to 1:20);
a combination of a compound of formula (I), (II), (III), (IV) or (V) and isoxaflutole (1:0.1 to 1:20);
a combination of a compound of formula (I), (II), (III), (IV) or (V) and fenquinotrione (1:0.1 to 1:20);
a combination of a compound of formula (I), (II), (III), (IV) or (V) and topramezone (1:0.1 to 1:20);
a combination of a compound of formula (I), (II), (III), (IV) or (V) and tolpyralate (1:0.1 to 1:20);
a combination of a compound of formula (I), (II), (III), (IV) or (V) and lancotrione-sodium salt (1:0.1 to 1:20); a combination of a compound of formula (I), (II), (III), (IV), or (V) and 2-methyl-N-(5-methyl-1,3,4-oxadiazol-2-yl)-3-(methylsulfonyl)-4-(trifluoromethyl)benzamide (CAS Registry Number: 1400904-50-8) (1:0.1 to 1:20); a combination of a compound of formula (I), (II), (III), (IV) or (V) and 2-chloro-N-(1-methyl-1H-tetrazol-5-yl)-3-(methylthio)-4-(trifluoromethyl)-benzamide (CAS Registry Number: 1361139-71-0) (1:0.1 to 1:20);
a combination of a compound of formula (I), (II), (III), (IV) or (V) and 4-(4-fluorophenyl)-6-[(2-hydroxy-6-oxo-1-cyclohexene-1-yl)carbonyl]-2-met-hyl-1,2,4-triazine-3,5(2H,4H)-dione (CAS Registry Number: 1353870-34-4) (1:0.1 to 1:20);
a combination of a compound of formula (I), (II), (III), (IV) or (V) and norflurazon (1:0.1 to 1:20);
a combination of a compound of formula (I), (II), (III), (IV) or (V) and fluridone (1:0.1 to 1:20);
a combination of a compound of formula (I), (II), (III), (IV) or (V) and bentazone (1:1 to 1:50);
a combination of a compound of formula (I), (II), (III), or (IV) and bromoxymil octanoate (1:1 to 1:50);
a combination of a compound of formula (I), (II), (III), (IV) or (V) and diuron (1:1 to 1:50); a combination of a compound of formula (I), (II), (III), (IV) or (V) and linuron (1:1 to 1:50); a combination of a compound of formula (I), (II), (III), (IV) or (V) and fluometuron (1:1 to 1:50);
a combination of a compound of formula (I), (II), (III), (IV) or (V) and simazine (1:1 to 1:50);
a combination of a compound of formula (I), (II), (III), (IV) or (V) and atrazine (1:1 to 1:50);
a combination of a compound of formula (I), (II), (III), (IV) or (V) and ametryn (1:1 to 1:50);
a combination of a compound of formula (I), (II), (III), (IV) or (V) and prometryn (1:1 to 1:50);
a combination of a compound of formula (I), (II), (III), (IV) or (V) and metribuzin (1:1 to 1:50);
a combination of a compound of formula (I), (II), (III), (IV) or (V) and alachlor (1:1 to 1:50); a combination of a compound of formula (I), (II), (III), (IV) or (V) and acetochlor (1:1 to 1:50);
a combination of a compound of formula (I), (II), (III), (IV) or (V) and metolachlor (1:1 to 1:50);
a combination of a compound of formula (I), (II), (III), (IV) or (V) and S-metolachlor (1:1 to 1:50);
a combination of a compound of formula (I), (II), (III), (IV) or (V) and dimethenamid (1:1 to 1:50);
a combination of a compound of formula (I), (II), (III), (IV) or (V) and dimethenamid-P (1:1 to 1:50);
a combination of a compound of formula (I), (II), (III), (IV) or (V) and pyroxasulfone (1:0.1 to 1:20);
a combination of a compound of formula (I), (II), (III), (IV) or (V) and flufenacet (1:0.1 to 1:20);
a combination of a compound of formula (I), (II), (III), (IV) or (V) and trifluralin (1:1 to 1:50);
a combination of a compound of formula (I), (II), (III), (IV) or (V) and pendimcthalin (1:1 to 1:50);
a combination of a compound of formula (I), (II), (III), (IV) or (V) and ethalfluralin (1:1 to 1:50);
a combination of a compound of formula (I), (II), (III), (IV) or (V) and 2,4-DB (1:1 to 1:50); a combination of a compound of formula (I), (II), (III), (IV) or (V) and fluroxypyr (1:1 to 1:50);
a combination of a compound of formula (I), (II), (III), (IV) or (V) and fluroxypyr-meptyl (1:1 to 1:50);
a combination of a compound of formula (I), (II), (III), (IV) or (V) and clopyralid-olamine salt (1:1 to 1:50), a combination of a compound of formula (I), (II), (III), (IV) or (V) and clopyralid-potassium salt (1:1 to 1:50);
a combination of a compound of formula (I), (II), (III), (IV) or (V) and clopyralid-triethylammonium salt (1:1 to 1:50);
a combination of a compound of formula (I), (II), (III), (IV) or (V) and halauxifen (1:0.1 to 1:20);
a combination of a compound of formula (I), (II), (III), (IV) or (V) and halauxifen-methyl (1:0.1 to 1:20);
a combination of a compound of formula (I), (II), (III), (IV) or (V) and florpyrauxifen (1:0.1 to 1:20);
a combination of a compound of formula (I), (II), (III), (IV) or (V) and florpyrauxifen-benzyl (1:0.1 to 1:20);
a combination of a compound of formula (I), (II), (III), (IV) or (V) and glyphosate (1:1 to 1:50);
a combination of a compound of formula (I), (II), (III), (IV) or (V) and glyphosate-isopropylammonium salt (1:1 to 1:50);
a combination of a compound of formula (I), (II), (III), (IV) or (V) and glyphosate-ammonium salt (1:1 to 1:50);
a combination of a compound of formula (I), (II), (III), (IV) or (V) and glyphosate-dimethylamine salt (1:1 to 1:50);
a combination of a compound of formula (I), (II), (III), (IV) or (V) and glyphosate-monoethanolamine salt (1:1 to 1:50);
a combination of a compound of formula (I), (II), (III), (IV) or (V) and glyphosate-potassium salt (1:1 to 1:50);
a combination of a compound of formula (I), (II), (III), (IV) or (V) and glyphosate-guanidine salt (1:1 to 1:50);
a combination of a compound of formula (I), (II), (III), (IV) or (V) and glufosinate (1:1 to 1:50);
a combination of a compound of formula (I), (II), (III), (IV) or (V) and glufosinate-ammonium salt (1:1 to 1:50);
a combination of a compound of formula (I), (II), (III), (IV) or (V) and glufosinate-P (1:1 to 1:50);
a combination of a compound of formula (I), (II), (III), (IV) or (V) and glufosinate-P-sodium salt (1:1 to 1:50);
a combination of a compound of formula (I), (II), (III), (IV) or (V) and EPTC (1:1 to 1:50); a combination of a compound of formula (I), (II), (III), (IV) or (V) and diflufenzopyr (1:1 to 1:50);
a combination of a compound of formula (I), (II), (III), (IV) or (V) and diflufenzopyr-sodium salt (1:1 to 1:50);
a combination of a compound of formula (I), (II), (III), (IV) or (V) and clomazone (1:1 to 1:50);
a combination of a compound of formula (I), (II), (III), (IV) or (V) and 2-[(2,4-dichlorophenyl)methyl]-4,4-dimethylisoxazolidin-3-one (CAS Registry Number: 81777-95-9) (1:1 to 1:50);
a combination of a compound of formula (I), (II), (III), (IV) or (V) and (3S,4S)—N-(2-fluorophenyl)-1-methyl-2-oxo-4-[3-(trifluoromethyl)phenyl]-3-pyrrolidinecarboxamide (CAS Registry Number: 2053901-33-8) (1:1 to 1:50);
a combination of a compound of formula (I), (II), (III), (IV) or (V) and cinmethylin (1:1 to 1:50);
a combination of a compound of formula (I), (II), (III), (IV) or (V) and MSMA (1:1 to 1:50); a combination of a compound of formula (I), (II), (III), (IV) or (V) and paraquat (1:1 to 1:50);
a combination of a compound of formula (I), (II), (III), (IV) or (V) and paraquat-dichloride (1:1 to 1:50);
a combination of a compound of formula (I), (II), (III), (IV) or (V) and diquat (1:1 to 1:50); a combination of a compound of formula (I), (II), (III), (IV) or (V) and diquat-dibromide (1:1 to 1:50);
a combination of a compound of formula (I), (II), (III), (IV) or (V) and benoxacor (1:0.1 to 1:20);
a combination of a compound of formula (I), (II), (III), (IV) or (V) and cyprosulfamide (1:0.1 to 1:20); or a combination of a compound of formula (I), (II), (III), (IV) or (V) and isoxadifen-ethyl (1:0.1 to 1:20).

Before, simultaneously with, and/or after sowing a crop seed treated with one or more compounds selected from the group consisting of an insecticide compound, a nematicide compound, a fungicide compound, and the like, the present composition may be applied to the field in which the crop seed have been sown or is to be sown.

In some embodiments, the present composition may be used in combination with another pesticidally-active compound. Examples of the insecticide compound, the nematicide compound, and the fungicide compound which may be used in combination with the present composition include neonicotinoid compounds, diamide compounds, carbamate compounds, organophosphorus compounds, biological nematicide compounds, other insecticide compounds and nematicide compounds, azole compounds, strobilurin compounds, metalaxyl compounds, SDHI compounds, and other fungicide compounds and plant growth regulators.

### EXAMPLES

The presently disclosed subject matter will be better understood by reference to the following Examples, which are provided as exemplary of the invention, and not by way of limitation.

Steps in the following Examples illustrate a procedure for each step in an overall synthetic transformation, and the starting material for each step may not have necessarily been prepared by a procedure described in other Examples or Steps. H-NMR spectra are reported in ppm downfield from tetramethylsilane; “s” means singlet, “d” means doublet, “t” means triplet, “q” means quartet, “m” means multiplet, “dd” means doublet of doublets, “dt” means doublet of triplets, and “br s” means broad singlet. Mass spectra (MS) are reported as the molecular weight of the highest isotopic abundance parent ion (M+1) formed by addition of H⁺ (molecular weight of 1) to the molecule, or (M−1) formed by the loss of H⁺ (molecular weight of 1) from the molecule, observed by using liquid chromatography coupled to a mass spectrometer (LCMS) using either atmospheric pressure chemical ionization (AP+) where “amu” stands for unified atomic mass units or electrospray ionization (ES⁺).

### Example 1. Preparation of methyl 2-(2,2,7-trifluoro-3-oxo-6-(perfluorophenyl)-2,3-dihydro-4H-benzo[b][1,4]oxazin-4-yl)acetate (Compound 1) and 2-(2,2,7-trifluoro-3-oxo-6-(perfluorophenyl)-2,3-dihydro-4H-benzo[b][1,4]oxazin-4-yl)acetic acid (Compound 2)

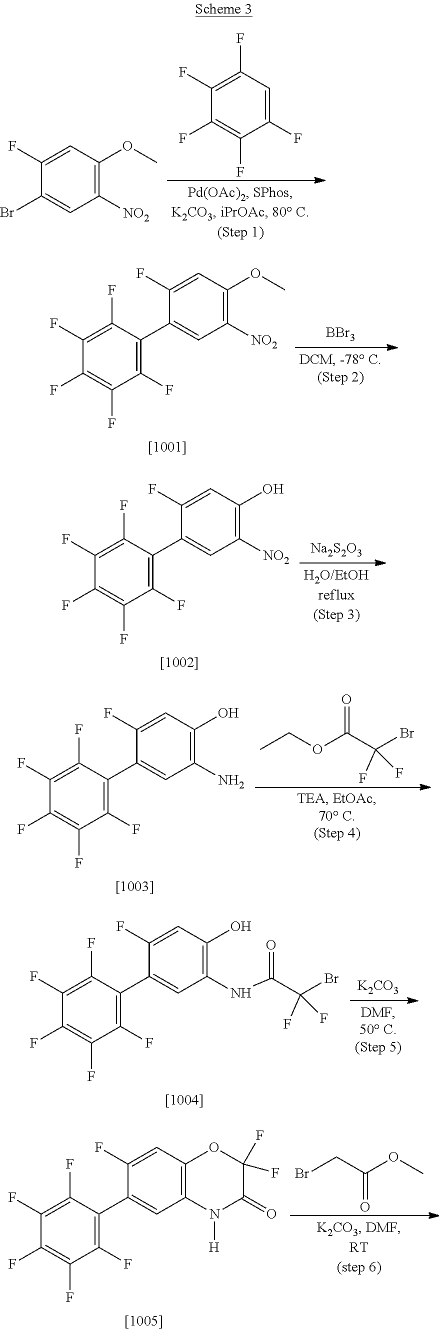

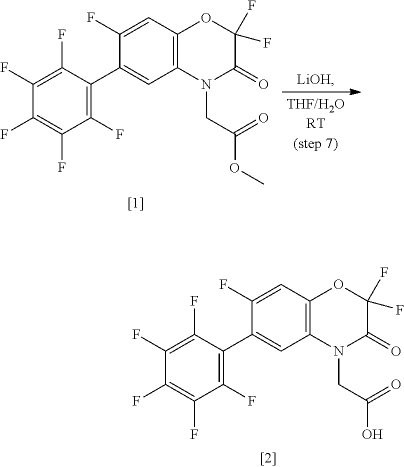 [SMILES: COC(=O)CN1C(=O)C(F)(F)Oc2cc(F)c(-c3c(F)c(F)c(F)c(F)c3F)cc21 ; O=C(O)CN1C(=O)C(F)(F)Oc2cc(F)c(-c3c(F)c(F)c(F)c(F)c3F)cc21]

As shown in Step 1 of Scheme 3, a mixture of 1-bromo-2-fluoro-4-methoxy-5-nitrobenzene (20.0 g, 80.0 mmol), 1,2,3,5-tetrafluorobenzene (36.0 g, 24.0 mmol), K₂PO₄ (33.9 g, 160 mmol), chloro[(diadamantan-1-yl)(n-butyl)phosphino][2-amino-1,1-biphenyl-2-yl]palladium(II) (2.7 g, 4.0 mmol), bis(adamantan-1-yl)(butyl)phosphane (1.4 g, 4.0 mmol) in dioxane (150 mL) was stirred at 90° C. for 16 hours under an atmosphere of nitrogen. The solvent was removed under reduced pressure and the residue purified by reversed-phase flash chromatography (5%-52% acetonitrile in water) to afford 2,2′,3,4,6-pentafluoro-4′-methoxy-5′-nitro-1,1′-biphenyl (Compound 1001, 11.0 g, 38% yield) as a light yellow solid: GCMS calculated for C₁₃H₆F₅NO₃=319.0, found 319.0.

As shown in Step 2 of Scheme 3, to a stirred mixture of 2,2′,3,4,6-pentafluoro-4′-methoxy-5′-nitro-1,1′-biphenyl (1.0 g, 3.13 mmol) in DCM (10 mL) was added boron tribromide (3.9 g, 15.7 mmol) dropwise at 0° C. under an atmosphere of nitrogen. The mixture was stirred at 0° C. for 3 hours, diluted with water, and extracted with dichloromethane. The combined organic layers were washed with brine, dried over anhydrous sodium sulfate, filtered, and concentrated under reduced pressure to afford 2,2′,3′,4′,6′-pentafluoro-5-nitro-[1,1′-biphenyl]-4-ol (Compound 1002, 790 mg, 74% yield) as a brown solid: MS (ESI) calculated for C₁₁H₄F₅NO₃ [M−1]⁻=304.0, found 303.9.

As shown in Step 3 of Scheme 3, to a stirred solution of 2,2′,3′,4′,6′-pentafluoro-5-nitro-[1,1′-biphenyl]-4-ol (790 mg, 2.58 mmol) in water (5 mL) and EtOH (5 mL) was added sodium hyposulfite (2.0 g, 12.94 mmol). The resulting mixture was stirred at 100° C. for 2 hours, cooled to room temperature, diluted with water, and extracted with dichloromethane. The combined organic layers were washed with brine, dried over anhydrous sodium sulfate, filtered, and concentrated under reduced pressure. The residue was purified by flash chromatography (0%-25% ethyl acetate in petroleum ether) to afford 5-amino-2,2′,3′,4′,6′-pentafluoro-[1,1′-biphenyl]-4-ol (Compound 1003, 580.0 mg, 59% yield) as a yellow solid: MS (ESI) calculated for C₁₂H₆F₅NO [M−1]⁻=274.0, found 274.0.

As shown in Step 4 of Scheme 3, to a solution of 5-amino-2,2′,3′,4′,6′-pentafluoro-[1,1′-biphenyl]-4-ol (580 mg, 2.10 mmol) and TEA (427 mg, 4.21 mmol) in EtOAc (5 mL) was added ethyl 2-bromo-2,2-difluoroacetate (856 mg, 4.21 mmol). The resulting mixture was stirred at 80° C. for 2 hours, cooled to room temperature, diluted with water, and extracted with dichloromethane. The combined organic layers were washed with brine, dried over anhydrous sodium sulfate, filtered, and concentrated under reduced pressure to afford 2-bromo-2,2-difluoro-N-(2′,3′,4′,6,6′-pentafluoro-4-hydroxy-[1,1′-biphenyl]-3-yl)acetamide (Compound 1004, 800 mg, 75% yield) as a brown oil: MS (ESI) calculated for C₁₄H₅BrF₇NO₂ [M−1]⁻=429.9, found 430.0.

As shown in Step 5 of Scheme 3, a stirred solution of 2-bromo-2,2-difluoro N-(2′,3′,4′,6,6′-pentafluoro-4-hydroxy-[1,1′-biphenyl]-3-yl)acetamide (870 mg, 2.01 mmol) and K₂CO₃ (417 mg, 3.02 mmol) in DMF (10 mL) was stirred at 50° C. for 2 hours, cooled to room temperature, diluted with water, and extracted with DCM. The combined organic layers were washed with brine, dried over anhydrous sodium sulfate, filtered, and concentrated under reduced pressure. The residue was purified by reversed-phase flash chromatography (5%-80% acetonitrile in water) to afford 2,2,7-trifluoro-6-(2,3,4,6-tetrafluorophenyl)-2H-benzo[b][1,4]oxazin-3(4H)-one (Compound 1005, 250 mg, 31% yield) as a brown solid: MS (ESI) calculated for C₁₄H₄F₇NO₂ [M−1⁻=350.0, found 350.2; ¹H-NMR (400 MHz, DMSO-d₆) δ 12.14 (s, 1H), 7.71-7.65 (m, 1H), 7.62 (d, J=9.6 Hz, 1H), 7.20 (d, J=6.4 Hz, 1H); ¹⁹F-NMR (376 MHz, DMSO-d₆) δ −75.34, −115.87, −116.95, −131.54, −134.11, −164.81.

As shown in Step 6 of Scheme 3, to a solution of 2,2,7-trifluoro-6-(perfluorophenyl)-2H-benzo[b][1,4]oxazin-3(4H)-one 200 mg, 0.54 mmol) in DMF (2 mL) were added K₂CO₃ (112 mg, 0.81 mmol) and methyl 2-bromoacetate (109 mg, 0.81 mmol). The mixture was stirred at room temperature for 4 hours, diluted with water, and extracted with ethyl acetate. The combined organic layers were washed with brine, dried over anhydrous sodium sulfate, filtered, and concentrated under reduced pressure. The residue was purified by reversed-phase flash chromatography (5% to 50% acetonitrile in water) to afford methyl 2-(2,2,7-trifluoro-3-oxo-6-(perfluorophenyl)-2,3-dihydro-4H-benzo[b][1,4]oxazin-4-yl)acetate (Compound 1, 80 mg, 33% yield) as an off-white solid: GCMS calculated for C₁₇H₇F₅NO₄=441.0, found 441.0; ¹H-NMR (400 MHz, DMSO-d₆) δ 7.81-7.79 (m, 2H), 4.89 (s, 2H), 3.72 (s, 3H).

As shown in Step 7 of Scheme 3, to a solution of methyl 2-(2,2,7-trifluoro-3-oxo-6-(perfluorophenyl)-2,3-dihydro-4H-benzo[b][1,4]oxazin-4-yl)acetate (50 mg, 0.11 mmol) in THE (1.5 mL) and water (0.5 mL) was added lithium hydroxide (17 mg, 0.45 mmol). The resulting mixture was stirred at room temperature for 16 hours, acidified with formic acid to pH 2-3, then purified by reversed-phase preparative HPLC (55% to 85% acctonitrile/0.05% aqueous TFA) to afford 2-(2,2,7-trifluoro-3-oxo-6-(perfuorophenyl)-2,3-dihydro-4H-benzo[b][1,4]oxazin-4-yl)acetic acid (Compound 2, 32 mg, 66% yield) as a white solid: MS (ESI) calculated for C₁₆H₃F₅NO₄ [M−1]⁺=427.0, found 425.8, ¹H-NMR (400 MHz, DMSO-d₆) δ 13.74 (br, 1H), 7.79-7.74 (m, 2H), 4.74 (s, 2H).

Using the appropriate alkylating agent in transformations similar to those described in Scheme 3, followed by any subsequent synthetic manipulations or purifications, resulted in the preparation of the following compounds.

Alkylation of Compound 1005 with 4-bromo-2-(bromomethyl)N,N-diethylbenzamide produced 4-bromo-N,N-diethyl-2-((2,2,7-trifluoro-3-oxo-6-(perfluorophenyl)-2,3-dihydro-4H-benzo[b][1,4]oxazin-4-yl)methyl)benzamide (Compound 3): MS (ESI) calculated for C₂₆H₁₇BrF₈N₂O₃ [M+H]⁺=637.03 & 639.06, found 637.05 & 639.05; ¹H-NMR (400 MHz, methanol-d₄) δ 7.66-7.56 (m, 1H), 7.53 (d, J=6.4 Hz, 1H), 7.46-7.36 (m, 2H), 7.26 (d, J=8.0 Hz, 1H), 5.22 (b, 2H), 3.62-3.52 (m, 2H), 3.28-3.24 (m, 2H), 1.30 (t, J=7.2 Hz, 3H), 1.15 (t, J=7.2 Hz, 3H); ¹⁹F-NMR (400 MHz, DMSO-d₆) δ −74.10, −116.69, −142.42, −156.45, −164.98.

Alkylation of Compound 1005 with methyl 2-(bromomethyl)benzoate produced methyl 2-((2,2,7-tri fluoro-3-oxo-6-(perfluorophenyl)-2,3-dihydro-4H-benzo[b][1,4]oxazin-4-yl)methyl)benzoate (Compound 4): GCMS calculated for C₂₃H₁₁F₈NO₄=517.1, found 517.1; ¹H-NMR (400 MHz, DMSO-d₆) δ 8.09-7.99 (m, 1H), 7.85-7.80 (m, 1H), 7.62-7.52 (m, 1H), 7.50-7.40 (m, 2H), 7.10-7.03 (m, 1H), 5.59 (s, 2H), 3.88 (s, 3H); ¹⁹F-NMR (376 MHz, DMSO-d₆) δ −74.27, −115.88, −140.66, −153.34, −162.09; which when treated with with 4M HCl/dioxane at 100° C., subsequently produced 2-((2,2,7-trifluoro-3-oxo-6-(perfluorophenyl)-2,3-dihydro-4H-benzo[b][1,4]oxazin-4-yl)methyl)benzoic acid (Compound 5): MS (ESI) calculated for C₂₂H₉F₈NO₄ [M−1]⁻=502.0, found 502.1; ¹H-NMR (400 MHz, DMSO-d₆) δ 13.27 (s, 1H), 8.06-8.01 (m, 1H), 7.85-7.78 (m, 1H), 7.60-7.32 (m, 3H), 7.08-6.98 (m, 1H), 5.61 (s, 2H); ¹⁹F-NMR (376 MHz, DMSO-d₆) δ −74.35, −115.97, −140.66, −153.52, −162.15.

Alkylation of Compound 1005 with methyl 4-bromobutanoate produced methyl 4-(2,2,7-trifluoro-3-oxo-6-(perfluorophenyl)-2,3-dihydro-4H-benzo[b][1,4]oxazin-4-yl)butanoate (Compound 16): GCMS calculated for C₁₉H₁₁F₈NO₄=469.1, found 469.1; ¹H-NMR (400 MHz, DMSO-d₆) δ 7.80 (d, J=6.4 Hz, 1H), 7.73 (d, J=9.6 Hz, 1H), 4.05 (t, J=7.2 Hz, 2H), 3.55 (s, 3H), 2.44 (t, J=7.2 Hz, 2H), 1.92-1.82 (m, 2H); ¹⁹F-NMR (376 MHz, DMSO-d₆) δ −74.29, −116.41, −140.32, −153.33, −162.08; which when treated with LiOH in THF/H₂O, subsequently produced 4-(2,2,7-trifluoro-3-oxo-6-(perfluorophenyl)-2,3-dihydro-4H-benzo[b][1,4]oxazin-4-yl)butanoic acid (Compound 17): MS (ESI) calculated for C₁₈H₁₉F₈NO₄ [M−1]⁻=454.0, found 453.9; ¹H-NMR (400 MHz, DMSO-d₆) δ 12.20 (s, 1H), 7.82 (d, J=6.4 Hz, 1H), 7.79-7.73 (m, 1H), 4.06-3.99 (m, 2H), 2.39-2.33 (m, 2H), 1.88-1.79 (m, 2H); ¹⁹F-NMR (376 MHz, DMSO-d₆) δ −74.27, −116.44, −140.33, −153.37, −161.96.

Alkylation of Compound 1005 with methyl 2-hydroxy-2-methylpropanoate, under Mitsinobu conditions using triphenyl phosphine and diisopropyl azodicarboxylate, produced methyl-2-methyl-2-(2,2,7-trifluoro-3-oxo-6-(perfluorophenyl)-2,3-dihydro-4H-benzo[b][1,4]oxazin-4-yl)propanoate (Compound 18): GCMS calculated for C₁₉H₁₁F₅NO₄=469.0, found 469.0; which when treated with BBr₃ at 0° C. in DCM, subsequently produced 2-methyl-2-(2,2,7-trifluoro-3-oxo-6-(perfluorophenyl)-2,3-dihydro-4H-benzo[b][1,4]oxazin-4-yl)propanoic acid (Compound 19), isolated as its ring-closed tautomer [4,4,7-trifluoro-3a-hydroxy-1,1-dimethyl-8-(perfluorophenyl)-3a,4-dihydrobenzo[b]oxazolo[3,2-d][1,4]oxazin-2(1H)-one]): MS (ESI) calculated for C₁₈H₉F₈NO₄ [M−1]⁻=454.0, found 454.0; ¹H-NMR (400 MHz, DMSO-d₆) δ 9.47 (d, J=4.0 Hz, 1H), 8.13 (d, J=7.2 Hz, 1H), 7.75-7.65 (m, 1H), 1.64 (s, 3H), 1.42 (s, 3H); ¹⁹F-NMR (376 MHz, DMSO-d₆) δ −82.17, −97.78, −112.87, −141.03, −153.57, −162.13.

Alkylation of Compound 1005 with methyl 2-hydroxy-2-methylpropanoate in DMF at 80° C. produced methyl 1-(2,2,7-trifluoro-3-oxo-6-(perfluorophenyl)-2,3-dihydro-4H-benzo[b][1,4]oxazin-4-yl)cyclopropane-1-carboxylate (Compound 20): GCMS calculated for C₁₉H₉F₅NO₄=467.0, found 467.0; ¹H-NMR (400 MHz, DMSO-d₆) δ 7.76 (d, J=9.6 Hz, 1H), 7.70 (d, J=6.4 Hz, 1H), 3.66 (s, 3H), 1.99-1.82 (m, 2H), 1.76-1.68 (m, 1H), 1.33-1.22 (m, 1H); ¹⁹F-NMR (376 MHz, DMSO-d₆) δ −69.26, −83.76, −115.96, −140.56, −153.91, −162.32.

Alkylation of Compound 1005 with tert-butyl 2,4-dibromobutanoate in DMF at 80° C. produced tert-butyl 1-(2,2,7-trifluoro-3-oxo-6-(perfluorophenyl)-2,3-dihydro-4H-benzo[b][1,4]oxazin-4-yl)cyclopropane-1-carboxylate, which was subsequently treated with trifluoroacetic acid in DCM at 25° C. to afford 1-(2,2,7-trifluo-3-oxo-6-(perfluorophenyl)-2,3-dihydro-4H-benzo[b][1,4]oxazin-4-yl)cyclopropane-1-carboxylic acid (Compound 21): MS (ESI) calculated for C₁₈H₇F₈NO₄ [M−1]⁻=452.0, found 452.0; ¹H-NMR (400 MHz, DMSO-d₆) δ 13.25 (s, 1H), 7.75 (d, J=9.6 Hz, 1H), 7.62 (d, J=6.4 Hz, 1H), 1.91-1.76 (m, 2H), 1.62-1.55 (m, 1H), 1.21-1.14 (m, 1H); ¹⁹F-NMR (376 MHz, DMSO-d₆) δ −69.65, −82.53, −116.27, −140.53, −154.03, −162.30.

Alkylation of Compound 1005 with methyl 3-(bromomethyl)benzoate produced methyl 3-((2,2,7-trifluoro-3-oxo-6-(perfluorophenyl)-2,3-dihydro-4H-benzo[b][1,4]oxazin-4-yl)methyl)benzoate (Compound 24): GCMS (ESI) calculated for C₂₃H₁₁F₈NO₄=517.1, found 517.0; ¹H-NMR (400 MHz, DMSO-d₆), δ 7.99 (s, 1H), 7.90 (d, J=7.6 Hz 1H), 7.79-7.71 (m, 2H), 7.60-7.50 (m, 2H), 5.36 (s, 2H), 3.85 (s, 3H); ¹⁹F-NMR (400 MHz, DMSO-d₆), δ −74.80, −115.66, −140.64, −153.12, −162.06; which when treated with LiOH in THF/H₂O, subsequently produced 3-((2,2,7-trifluoro-3-oxo-6-(perfluorophenyl)-2,3-dihydro-4H-benzo[b][1,4]oxazin-4-yl)methyl)benzoic acid (Compound 25): MS (ESI) calculated for C₂₂H₉F₈NO₄ [M−1]⁻=502.0; found, 502.2; ¹H-NMR (400 MHz, DMSO-d₆), δ 13.09 (s, 1H), 7.96-7.84 (m, 2H), 7.79-7.74 (m, 1H), 7.68 (d, J=6.4 Hz, 1H), 7.50-7.43 (m, 2H), 5.31 (s, 2H); ¹⁹F-NMR (400 MHz, DMSO-d₆), δ −74.86, −115.67, −140.58, −153.17, −161.98.

Alkylation of Compound 1005 with methyl 4-(bromomethyl)benzoate produced methyl 4-((2,2,7-tri fluoro-3-oxo-6-(perfluorophenyl)-2,3-dihydro-4H-benzo[b][1,4]oxazin-4-yl)methyl)benzoate (Compound 26): GCMS calculated for C₂₃H₁₁F₅NO₄=517.0, found 517.0; ¹H-NMR (400 MHz, DMSO-d₆) δ 8.12-7.92 (m, 2H), 7.90-7.76 (m, 1H), 7.60-7.46 (m, 1H), 7.44-7.32 (m, 2H), 5.35 (s, 2H), 3.83 (s, 3H); ¹⁹F-NMR (400 MHz, DMSO-d₆) δ −74.64, −115.66, −140.65, −153.20, −162.01; which when treated with LiOH in THF/H₂O, subsequently produced 4-((2,2,7-trifluoro-3-oxo-6-(perfluorophenyl)-2,3-dihydro-4H-benzo[b][1,4]oxazin-4-yl)methyl)benzoic acid (Compound 27): MS (ESI) calculated for C₂₂H₉F₈NO₄ [M−1]⁻=502.0, found 502.1; ¹H-NMR (400 MHz, DMSO-d₆) δ7.96-7.86 (m, 2H), 7.83-7.73 (m, 1H), 7.64-7.61 (m, 1H), 7.46-7.41 (m, 2H), 5.35 (s, 2H); ¹⁹F-NMR (376 MHz, DMSO-d₆) δ −74.61, −115.65, −140.61, −153.19, −161.98.

Alkylation of Compound 1005 with 4-bromobut-2-yn-1-ol produced 2,2,7-trifluoro-4-(4-hydroxybut-2-yn-1-yl)-6-(perfluorophenyl)-2H-benzo[b][1,4]oxazin-3(4H)-one, which was subsequently reacted with 3-oxo-1λ⁵-benzo[d][1,2]iodaoxole-1,1,1(3H)-triyl triacetate (Dess-Martin reagent) in portions at 20° C. to produce 4-(2,2,7-trifluoro-3-oxo-6-(perfluorophenyl)-2,3-dihydro-4H-benzo[b][1,4]oxazin-4-yl)but-2-ynal, which was subsequently oxidized with NaClO₂ to produce 4-(2,2,7-trifluoro-3-oxo-6-(perfluorophenyl)-2,3-dihydro-4H-benzo[b][1,4]oxazin-4-yl)but-2-ynoic acid (Compound 28); ¹H-NMR (400 MHz, DMSO-d₆) δ 7.80 (d, J=9.6 Hz, 1H), 7.70 (d, J=6.4 Hz, 1H), 4.86 (s, 2H); ¹⁹F-NMR (400 MHz, DMSO-d₆) δ −74.83, −115.66, −140.16, −153.01, −161.86.

Alkylation of Compound 1005 with methyl acrylate, using cesium carbonate as the base in acetonitrile at 80° C., produced methyl 3-(2,2,7-trifluoro-3-oxo-6-(perfluorophenyl)-2,3-dihydro-4H-benzo[b][1,4]oxazin-4-yl)propanoate (Compound 45): GCMS calculated for C₁₈H₉F₈NO₄=455.0, found 455.1; ¹H-NMR (400 MHz, DMSO-d₆) δ 7.84 (d, J=6.4 Hz, 1H), 7.74 (d, J=9.6 Hz, 1H), 4.26 (t, J=7.6 Hz, 2H), 3.59 (s, 3H), 2.70 (d, J=7.6 Hz, 2H); ¹⁹F-NMR (376 MHz, DMSO-d₆) δ −74.53, −116.30, −140.19, −153.28, −162.10; which when treated with trimethylstannanol in dichloroethane at 80° C., subsequently produced 3-(2,2,7-trifluoro-3-oxo-6-perfluorophenyl)-2,3-dihydro-4H-benzo[b][1,4]oxazin-4-yl)propanoic acid (Compound 46): MS (ESI) calculated for C₁₇H₇F₈NO₄ [M−1]⁻=440.0; found, 440.0; ¹H-NMR (400 MHz, DMSO-d₆) δ 7.83 (d, J=6.4 Hz, 1H), 7.71 (d, J=9.6 Hz, 1H), 4.17 (t, J=7.6 Hz, 2H), 2.46 (t, J=7.6 Hz, 2H); ¹⁹F-NMR (377 MHz, DMSO-d₆) δ −74.69, −116.49, −140.31, −153.57, −162.20.

Alkylation of Compound 1005 with methyl 3-bromo-2,2-difluoropropanoate, using cesium carbonate as the base in acetonitrile at 80° C., produced 2,2-difluoro-3-(2,2,7-trifluoro-3-oxo-6-(perfluorophenyl)-2,3-dihydro-4H-benzo[b][1,4]oxazin-4-yl)propanoic acid (Compound 47): MS (ESI) calculated for C₁₇H₅F₁₀NO₄ [M−1]⁻=476.0, found 475.9; ¹H-NMR (400 MHz, methanol-d₄) δ 7.69 (d, J=6.4 Hz, 1H), 7.37 (m, 1H), 4.75-4.66 (m, 2H). ¹⁹F-NMR (377 MHz, methanol-d₄) δ −79.17, −108.24, −117.50, −142.15, −156.59, −165.05; which when treated with methanol and a catalytic amount of sulfuric acid at 80° C., subsequently produced methyl 2,2-difluoro-3-(2,2,7-trifluoro-3-oxo-6-(perfluorophenyl)-2,3-dihydro-4H-benzo[b][1,4]oxazin-4-yl)propanoate (Compound 48): GCMS calculated for C₁₈H₇F₁₀NO₄=491.0, found 491.0; ¹H-NMR (400 MHz, DMSO-d₆) δ 7.88 (d, J=6.4 Hz, 1H), 7.79 (d, J=9.4 Hz, 1H), 4.96-4.83 (m, 2H), 3.85 (s, 3H); ¹⁹F-NMR (377 MHz, DMSO-d₆) δ −75.39, −107.64, −115.36, −140.50, −152.77, −161.80.

Alkylation of Compound 1005 with methyl 3-(bromomethyl)-2-fluorobenzoate produced methyl 2-fluoro-3-((2,2,7-trifluoro-3-oxo-6-(perfluorophenyl)-2,3-dihydro-4H-benzo[b][1,4]oxazin-4-yl)methyl)benzoate (Compound 53): MS (ESI) calculated for C₂₃H₁₀F₉NO₄ [M+1]⁺=536.1, found 536.1; ¹H-NMR (400 MHz, DMSO-d₆) δ 7.85-7.73 (m, 2H), 7.66 (d, J=6.4 Hz, 1H), 7.53-7.47 (m, 1H), 7.29 (d, J=7.6 Hz, 1H), 5.35 (s, 2H), 3.86 (s, 3H); ¹⁹F-NMR (376 MHz, DMSO-d₆) δ −74.55, −114.65, −115.69, −140.58, −153.14, −162.06; which when treated with trimethylstannanol in DCM at 80° C., subsequently produced 2-fluoro-3-((2,2,7-trifluoro-3-oxo-6-(perfluorophenyl)-2,3-dihydro-4H-benzo[b][1,4]oxazin-4-yl)methyl)benzoic acid (Compound 54): MS (ESI) calculated for C₂₂H₇F₉NO₄ [M+1]⁺=521.0, found 521.0; ¹H-NMR (400 MHz, DMSO-d₆) δ 13.35 (br, 1H), 7.81-7.78 (m, 2H), 7.42-7.40 (m, 1H), 7.35-7.30 (m, 1H), 7.25-7.22 (m, 1H), 5.33 (s, 2H); ¹⁹F-NMR (376 MHz, DMSO-d₆) δ −74.61, −115.28, −140.53, −153.21, −162.01.

Arylation of Compound 1005 with methyl 3-bromobenzoate, using Cu(OAc)₂ and triethylamine in pyridine at 80° C., produced methyl 3-(2,2,7-trifluoro-3-oxo-6-(perfluorophenyl)-2,3-dihydro-H-benzo[b][1,4]oxazin-4-yl)benzoate (Compound 55, 60 mg, 11% yield) as a light yellow solid: MS (ESI) calculated for C₂₂H₉F₈NO₄ [M+1]⁺=504.0, found 503.9; ¹H-NMR (400 MHz, DMSO-d₆) δ8.16 (s, 1H), 8.14 (s, 1H), 7.86-7.77 (m, 3H), 6.71 (d, J=6.4 Hz, 1H), 3.88 (s, 3H); ¹⁹F-NMR (400 MHz, DMSO-d₆) δ −72.08, −74.99, −116.16, −140.92, −153.95, −162.36; which, when subsequently treated with 4M HCl/dioxane at 100° C., produced 3-(2,2,7-trifluoro-3-oxo-6-(perfluorophenyl)-2,3-dihydro-4H-benzo[b][1,4]oxazin-4-yl)benzoic acid (Compound 56, 29 mg, 17% yield) as a white solid: MS (ESI) calculated for C₂₁H₇F₈NO₄ [M−1]⁻=487.0, found 487.4; ¹H-NMR (400 MHz, DMSO-d₆) δ 13.38 (s, 1H), 8.29-8.01 (m, 2H), 7.98-7.74 (m, 3H), 6.71 (d, J=6.4 Hz, 1H); ¹⁹F-NMR (400 MHz, DMSO-d₆) δ −74.70, −116.17, −140.98, −153.99, −162.32.

Alkylation of Compound 1005 with 2-bromoacetamide produced 2-(2,2,7-trifluoro-3-oxo-6-(perfluorophenyl)-2,3-dihydro-4H-benzo[b][1,4]oxazin-4-yl)acetamide (Compound 83): MS (ESI) calculated for C₁₆H₆F₈N₂O₃ [M+1]⁺=427.0, found 427.1; ¹H-NMR (400 MHz, DMSO-d₆) δ 7.76 (d, J=9.6 Hz, 2H), 7.55 (d, J=6.4 Hz, 1H), 7.38 (s, 1H), 4.64 (s, 2H); ¹⁹F-NMR (377 MHz, DMSO-d₆) δ −74.38, −116.33, −140.34, −153.27, −162.18.

Alkylation of Compound 1005 with 2-bromo-N,N-dimethylacetamide produced N,N-dimethyl-2-(2,2,7-tri fluoro-3-oxo-6-(perfluorophenyl)-2,3-dihydro-4H-benzo[b][1,4]oxazin-4-yl)acetamide (Compound 90): MS (ESI) calculated for C₁₈H₁₀F₈N₂O₃ [M+1]⁺=454.0, found 454.1; ¹H-NMR (400 MHz, DMSO-d₆) δ 7.77 (d, J=9.6 Hz, 1H), 7.62 (d, J=6.4 Hz, 1H), 4.94 (s, 2H), 3.08 (s, 3H), 2.85 (s, 3H); ¹⁹F-NMR (377 MHz, DMSO-d₆) δ −74.38, −116.33, −140.34, −153.27, −162.18.

Alkylation of Compound 1005 with methyl 2-(bromomethyl)acrylate produced methyl 2-((2,2,7-trifluoro-3-oxo-6-(perfluorophenyl)-2,3-dihydro-4H-benzo[b][1,4]oxazin-4-yl)methyl)acrylate (Compound 100): GCMS calculated for C₁₉H₉F₈NO₄=467.0, found 467.0; ¹H-NMR (400 MHz, DMSO-d₆) δ 7.78 (d, J=9.6 Hz, 1H), 7.64 (d, J=6.4 Hz, 1H), 6.20 (s 1H), 5.53 (s 1H), 4.82 (s, 2H), 3.74 (s, 3H); ¹⁹F-NMR (376 MHz, DMSO-d₆) δ −74.40, −116.05, −140.35, −153.33, −162.05; which after successive treatments of osmium tetroxide and sodium periodate produced methyl 2-oxo-3-(2,2,7-trifluoro-3-oxo-6-(perfluorophenyl)-2,3-dihydro-4H-benzo[b][1,4]oxazin-4-yl)propanoate (Compound 101): MS (ES) calculated for C₁₈H₇F₈NO₅ [M−1]⁻=468.0, found 467.9; ¹H-NMR (400 MHz, DMSO-d₆) a 7.80 (d, J=9.6 Hz, 1H), 7.74 (d, J=6.4 Hz, 1H), 5.35 (s, 2H), 3.87 (s, 3H); ¹⁹F-NMR (377 MHz, DMSO-d₆) δ −75.32, −115.72, −140.12, −152.80, −161.71; which after treatment with trimethylstannanol in DCE at 0° C. produced 2-2-oxo-3-(2,2,7-trifluoro-3-oxo-6-(perfluorophenyl)-2,3-dihydro-4H-benzo[b][1,4]oxazin-4-yl)propanoic acid (Compound 102): MS (ESI) calculated for C₁₇H₅F₈NO₅ [M−1]⁻=454.0, found 453.8; ¹H-NMR (400 MHz, DMSO-d₆) δ 7.79 (d, J=9.6 Hz, 1H), 7.73 (d, J=6.4 Hz, 1H), 5.31 (s, 2H); ¹⁹F-NMR (377 MHz, DMSO-d₆) δ −75.23, −115.87, −140.19, −153.00, −161.93.

Alkylation of Compound 1005 with methyl 2-(2-bromoethyl)benzoate produced methyl 2-(2-(2,2,7-trifluoro-3-oxo-6-(perfluorophenyl)-2,3-dihydro-4H-benzo[b][1,4]oxazin-4-yl)ethyl)benzoate (Compound 105): GCMS calculated for C₂₄H₁₃F₈NO₄=531.1, found 531.1; ¹H-NMR (400 MHz, DMSO-d₆) δ 7.87-7.79 (m, 2H), 7.73 (d, J=9.6 Hz, 1H), 7.53-7.47 (m, 1H), 7.38-7.24 (m, 2H), 4.34-4.24 (m, 2H), 3.74 (s, 3H), 3.30-3.23 (m, 2H); ¹⁹F-NMR (377 MHz, DMSO-d₆) δ −74.83, −116.24, −140.21, −153.06, −162.13; which when treated with 4M HCl/dioxane at 100° C., subsequently produced 2-(2-(2,2,7-trifluoro-3-oxo-6-(perfluorophenyl)-2,3-dihydro-4H-benzo[b][1,4]oxazin-4-yl)ethyl)benzoic acid (Compound 106): MS (ESI) calculated for C₂₃H₁₁F₅NO₄ [M−1]⁻=516.1, found 515.9; ¹H-NMR (400 MHz, methanol-d₄) δ 7.90-7.87 (m, 1H), 7.74-7.73 (m, 1H), 7.33-7.24 (m, 4H), 4.38 (t, J=7.6 Hz, 2H), 3.39-3.37 (m, 2H); ¹⁹F-NMR (377 MHz, methanol-d₄) δ −78.60, −117.64, −142.27, −156.57, −164.86.

Alkylation of Compound 1005 with methyl 4-(2-bromoethyl)benzoate produced methyl 4-(2-(2,2,7-trifluoro-3-oxo-6-(perfluorophenyl)-2,3-dihydro-4H-benzo[b][1,4]oxazin-4-yl)ethyl)benzoate (Compound 128): GCMS calculated for C₂₄H₁₃F₈NO₄=531.1, found, 531.1; ¹H-NMR (400 MHz, DMSO-d₆) δ 7.82 (d, J=8.0 Hz, 2H), 7.71 (d, J=9.6 Hz, 1H), 7.65 (d, J=6.4 Hz, 1H), 7.35 (d, J=4.0 Hz, 2H), 4.35-4.28 (m, 2H), 3.84 (s, 3H), 3.06-3.00 (m, 2H); ¹⁹F-NMR (377 MHz, DMSO-d₆) δ −75.19, −116.19, −140.30, −153.36, −162.09; which when treated with 4M HCl/dioxane at 100° C., subsequently produced 442-(2,2,7-trifluoro-3-oxo-6-(perfluorophenyl)-2,3-dihydro-4H-benzo[b][1,4]oxazin-4-yl)ethyl)benzoic acid (Compound 129): MS (ESI) calculated for C₂₃H₁₁F₈NO₄ [M−1]⁻=516.1, found 516.0; ¹H-NMR (400 MHz, DMSO-d₆) δ 12.82 (s, 1H), 7.86-7.79 (m, 2H), 7.74-7.65 (m, 2H), 7.38-7.28 (m, 2H), 4.31 (t, J=7.2 Hz, 2H), 3.02 (t, J=7.2 Hz, 2H); ¹⁹F-NMR (377 MHz, DMSO-d₆) δ −75.14, −116.18, −140.28, −153.27, −162.01.

Alkylation of Compound 1005 with methyl 2-(3-((tosyloxy)methyl)cyclobutyl)acetate (which in turn was produced by the tosylation of methyl 2-(3-(hydroxymethyl)cyclobutyl)acetate) produced methyl 2-(3-((2,2,7-trifluoro-3-oxo-6-(perfluorophenyl)-2,3-dihydro-4H-benzo[b][1,4]oxazin-4-yl)methyl)cyclobutyl)acetate which was purified by preparative HPLC (10% iPrOH/hexanes) to produce methyl 2-((1r,3r)-3-((2,2,7-trifluoro-3-oxo-6-(perfluorophenyl)-2,3-dihydro-4H-benzo[b][1,4]oxazin-4-yl)methyl)cyclobutyl)acetate (Compound 136): GCMS (ES) calculated for C₂₂H₁₅F₈NO₄=509.0, found 509.1; ¹H-NMR (400 MHz, DMSO-d₆) δ 7.77 (d, J=6.4 Hz, 1H), 7.73 (d, J=9.6 Hz, 1H), 4.06 (d, J=6.8 Hz, 2H), 3.55 (s, 3H), 2.53-2.49 (m, 1H), 2.40-2.28 (m, 3H), 2.16-2.05 (m, 2H), 1.58-1.47 (m, 2H); ¹⁹F-NMR (377 MHz, DMSO-d₆) δ −74.85, −116.34, −140.29, −153.44, −162.14; which when treated with trimethylstannanol in dichloroethane at 65° C., subsequently produced 2-((1 r,3r)-3-((2,2,7-trifluoro-3-oxo-6-(perfluorophenyl)-2,3-dihydro-4H-benzo[b][1,4]oxazin-4-yl)methyl)cyclobutyl)acetic acid (Compound 138): MS (ESI) calculated for C₂₁H₁₃F₅NO₄ [M−1]⁻=494.1, found 494.0; ¹H-NMR (400 MHz, DMSO-d₆) δ 11.85 (b, 1H), 7.79 (d, J=6.4 Hz, 1H), 7.73 (d, J=9.6 Hz, 1H), 4.17 (d, J=7.6 Hz, 2H), 2.75-2.57 (m, 2H), 2.36 (d, J=7.6 Hz, 2H), 1.98-1.84 (m, 2H), 1.80-1.63 (m, 2H); ¹⁹F-NMR (376 MHz, DMSO-d₆) δ −74.75, −116.31, −140.28, −153.48, −162.11; and methyl 2-((1s,3s)-3-((2,2,7-trifluoro-3-oxo-6-(perfluorophenyl)-2,3-dihydro-4H-benzo[b][1,4]oxazin-4-yl)methyl)cyclobutyl)acetate (Compound 137): GCMS (ESI) calculated for C₂₂H₁₅F₈NO₄=509.0, found 509.1; ¹H-NMR (400 MHz, DMSO-d₆) δ 7.77 (d, J=6.4 Hz, 1H), 7.73 (d, J=9.6 Hz, 1H), 4.06 (d, J=6.8 Hz, 2H), 3.55 (s, 3H), 2.53-2.49 (m, 1H), 2.40-2.28 (m, 3H), 2.16-2.05 (m, 2H), 1.58-1.47 (m, 2H); ¹⁹F-NMR (377 MHz, DMSO-d₆) δ −74.78, −116.30, −140.28, −153.49, −162.14, which when treated with trimethylstannanol in dichloroethane at 65° C., subsequently produced 2-((1s,3s)-3-((2,2,7-trifluoro-3-oxo-6-(perfluorophenyl)-2,3-dihydro-4H-benzo[b][1,4]oxazin-4-yl)methyl)cyclobutyl)acetic acid (Compound 139): MS (ESI) calculated for C₂₁H₁₃F₈NO₄ [M−1]⁻=494.1, found 493.9; ¹H-NMR (400 MHz, DMSO-d₆) δ 11.87 (s, 1H), 7.80-7.69 (m, 2H), 4.06 (d, J=6.8 Hz, 2H), 2.41-2.28 (m, 1H), 2.27-2.18 (m, 3H), 2.16-2.05 (m, 2H), 1.57-1.44 (m, 2H); ¹⁹F-NMR (376 MHz, DMSO-d₆) δ −74.86, −116.34, −140.28, −153.44, −162.11.

Alkylation of Compound 1005 with methyl 4-bromo-2,2-dimethylbutanoate produced methyl 2,2-dimethyl-4-(2,2,7-trifluoro-3-oxo-6-(perfluorophenyl)-2,3-dihydro-4H-benzo[b][1,4]oxazin-4-yl)butanoate (Compound 140): GCMS for C₂₁H₁₅F₅NO₄=497.1, found 497.1; ¹H-NMR (400 MHz, DMSO-d₆) δ 7.76 (d, J=9.6 Hz, 1H), 7.63 (d, J=6.4 Hz, 1H), 4.08-3.93 (m, 2H), 3.54 (s, 3H), 1.94-1.73 (m, 2H), 1.20 (s, 6H); ¹⁹F-NMR (376 MHz, DMSO-d₆) δ −74.75, −115.99, −140.67, −153.01, −161.97; which when treated with 4M HCl/dioxane at 100° C., 2,2-dimethyl-4-(2,2,7-trifluoro-3-oxo-6-(perfluorophenyl)-2,3-dihydro-4H-benzo[b][1,4]oxazin-4-yl)butanoic acid (Compound 141): MS (ESI) calculated for C₂₀H₁₃F₈NO₄ [M−1]⁻=482.1, found 481.9; ¹H-NMR (400 MHz, methanol-d₄) δ 7.73 (d, J=6.4 Hz, 1H), 7.38 (d, J=9.2 Hz, 1H), 4.25-3.99 (m, 2H), 1.98-1.80 (m, 2H), 1.30 (s, 6H); ¹⁹F-NMR (377 MHz, methanol-d₄) δ −78.37, −117.33, −142.47, −156.50, −164.75.

Alkylation of Compound 1005 with methyl 3-(2-hydroxyethyl)benzoate, under Mitsinobu conditions using triphenyl phosphine and diisopropyl azodicarboxylate, produced methyl 3-(2-(2,2,7-trifluoro-3-oxo-6-(perfluorophenyl)-2,3-dihydro-4H-benzo[b][1,4]oxazin-4-yl)ethyl)benzoate (Compound 142): MS (ESI) calculated for C₂₄H₁₃F₈NO₄ [M+1]⁺=532.0, found 532.0; ¹H-NMR (400 MHz, DMSO-d₆) δ 7.84-7.66 (m, 4H), 7.48-7.35 (m, 2H), 4.30 (t, J=7.2 Hz, 2H), 3.83 (s, 3H), 3.01 (t, J=7.2 Hz, 2H); ¹⁹F-NMR (400 MHz, DMSO-d₆) δ −75.25, −116.23, −140.20, −153.37, −162.09; which when treated with 4M HCl/dioxane at 100° C., subsequently produced 3-(2-(2,2,7-trifluoro-3-oxo-6-(perfluorophenyl)-2,3-dihydro-4H-benzo[b][1,4]oxazin-4-yl)ethyl)benzoic acid (Compound 143): MS (ESI) calculated for C₂₃H₁₁F₅NO₄ [M−1]⁻=516.1, found 516.0; ¹H-NMR (400 MHz, DMSO-d₆) δ 12.78 (b, 1H), 7.79-7.68 (m, 4H), 7.45-7.32 (m, 2H), 4.30 (t, J=7.2 Hz, 2H), 3.01 (t, J=7.2 Hz, 2H); ¹⁹F-NMR (400 MHz, DMSO-d₆) δ −75.19, −116.25, −140.23, −153.27, −162.01.

Alkylation of Compound 1005 with 3-chloro-2-oxopropyl acetate produced 2-oxo-3-(2,2,7-trifluoro-3-oxo-6-perfluorophenyl)-2,3-dihydro-4H-benzo[b][1,4]oxazin-4-yl)propyl acetate (Compound 144): MS (ESI) calculated for C₁₉H₉F₈NO₅ [M−1]⁻=482.0, found 482.0; ¹H-NMR (400 MHz, DMSO-d₆) δ 7.79 (d, J=9.6 Hz, 1H), 7.53 (d, J=6.4 Hz, 1H), 5.15 (s, 2H), 5.05 (s, 2H), 2.09 (s, 3H); ¹⁹F-NMR (377 MHz, DMSO-d₆) δ −75.04, −115.85, −140.13, −152.83, −161.82.

Alkylation of Compound 1005 with 3-bromo-3,3-difluoroprop-1-ene, after deprotonation of the benzoxazinone amide hydrogen with NaH in DMF at 0° C., produced 4-(3,3-difluoroallyl)-2,2,7-trifluoro-6-(perfluorophenyl)-2H-benzo[b][1,4]oxazin-3(4H)-one: GCMS calculated for C₁₇H₅₁F₁₀NO₂=445.0, found 445.0; ¹H-NMR (400 MHz, DMSO-d₆) δ 7.76 (d, J=9.6 Hz, 1H), 7.65 (d, J=6.4 Hz, 1H), 4.94-4.76 (m, 1H), 4.72-4.63 (m, 2H); ¹⁹F-NMR (376 MHz, DMSO-d₆) δ −74.97, −85.19, −115.92, −140.67, −153.18, −162.08; which was subsequently reacted with OsO₄ and Na₂O₄ to produce 2-hydroxy-3-(2,2,7-trifluoro-3-oxo-6-(perfluorophenyl)-2,3-dihydro-4H-benzo[b][1,4]oxazin-4-yl)propanoic acid (Compound 149): MS (ESI) calculated for C₁₇H₇F₈NO₅ [M−1]⁻=456.0, found 455.9; ¹H-NMR (400 MHz, DMSO-d₆) δ 12.90 (s, 1H), 7.84 (d, J=6.4 Hz, 1H), 7.74 (d, J=9.6 Hz, 1H), 5.81 (s, 1H), 4.35-4.23 (m, 2H), 4.19-4.17 (m, 1H); ¹⁹F-NMR (377 MHz, DMSO-d₆) δ −73.94, −77.07, −116.34, −140.33, −153.26, −162.03.

Alkylation of Compound 1005 with methyl 2-(4-(bromomethyl)phenyl)acetate produced methyl 2-(4-((2,2,7-trifluoro-3-oxo-6-(perfluorophenyl)-2,3-dihydro-4H-benzo[b][1,4]oxazin-4-yl)methyl)phenyl)acetate (Compound 156): GCMS calculated for C₂₄H₁₃F₅NO₄=531.0, found 531.1; ¹H-NMR (400 MHz, DMSO-d₆) δ 7.78-7.68 (m, 2H), 7.31-7.21 (m, 4H), 5.26 (s, 2H), 3.65 (s, 2H), 3.59 (s, 3H); ¹⁹F-NMR (377 MHz, DMSO-d₆) δ −74.86, −115.76, −140.54, −153.20, −162.00; which when treated with 4M HCl/dioxane at 100° C., subsequently produced 2-(4-((2,2,7-trifluoro-3-oxo-6-(perfluorophenyl)-2,3-dihydro-4H-benzo[b][1,4]oxazin-4-yl)methyl)phenyl)acetic acid (Compound 157): MS (ESI) calculated for C₂₃H₁₁F₈NO₄ [M−1]⁻=516.0, found 516.0.1; ¹H-NMR (400 MHz, DMSO-d₆) δ 12.42 (br, 1H), 7.78-7.70 (m, 2H), 7.27-7.22 (m, 4H), 5.31 (s, 2H), 3.63 (s, 2H); ¹⁹F-NMR (377 MHz, DMSO-d₆) δ −74.90, −115.75, −140.50, −153.20, −162.04.

### Example 2. Preparation of methyl (R)-2-(2,2,7-trifluoro-3-oxo-6-(perfluorophenyl)-2,3-dihydro-4H-benzo[b][1,4]oxazin-4-yl)propanoate (Compound 8), methyl (S)-2-(2,2,7-trifluoro-3-oxo-6-(perfluorophenyl)-2,3-dihydro-4H-benzo[b][1,4]oxazin-4-yl)propanoate (Compound 9), (R)-2-(2,2,7-trifluoro-3-oxo-6-(perfluorophenyl)-2,3-dihydro-4H-benzo[b][1,4]oxazin-4-yl)propanoic acid (Compound 10), and (S)-2-(2,2,7-trifluoro-3-oxo-6-(perfluorophenyl)-2,3-dihydro-4H-benzo[b][1,4]oxazin-4-yl)propanoic acid (Compound 11)

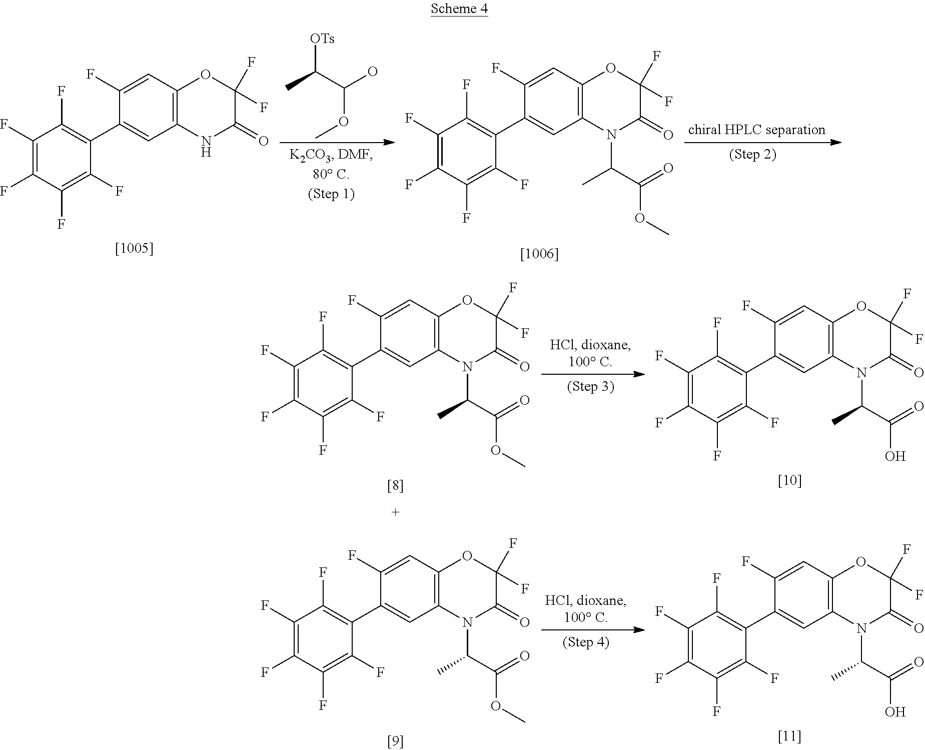

As shown in Step 1 of Scheme 4, a mixture of 2,2,7-trifluoro-6-(2,3,4,5,6-pentafluorophenyl)-4H-1,4-benzoxazin-3-one (Compound 1005, 500 mg, 1.35 mmol), methyl (S)-2-(tosyloxy)propanoate (525 mg, 2.03 mmol), and K₂CO₃ (374 mg, 2.70 mmol) in DMF (5 mL) was stirred at 80° C. for 16 hours, cooled to room temperature, diluted with water, and extracted with ethyl acetate. The combined organics were washed with brine, dried over anhydrous sodium sulfate, filtered, concentrated under reduced pressure, and purified by reversed-phase flash chromatography (5%-60% ACN in water) to afford methyl-2-(2,2,7-trifluoro-3-oxo-6-(perfluorophenyl)-2,3-dihydro-4H-benzo[b][1,4]oxazin-4-yl)propanoate (Compound 1006) as a racemic mixture as indicated by chiral HPLC.

As shown in Step 2 of Scheme 4, racemic Compound 1006 was separated into its respective enantiomers by preparative-Chiral HPLC using the following conditions—column: Chiralpak) AD-H, 2×25 cm, 5 um, eluted with 5% (1:1 EtOH/DCM)/hexane (0.2% diethylamine) to yield methyl (S′)-2-(2,2,7-trifluoro-3-oxo-6-(perfluorophenyl)-2,3-dihydro-4H-benzo[B][1,4]oxazin-4-yl) propanoate (Compound 9, 12 mg, 2% yield) as a yellow oil: GCMS calculated for C₁₈H₉F₈NO₄=455.0, found 455.0; ¹H-NMR (400 MHz, methanol-d₄) δ 7.54 (d, J=6.0 Hz, 1H), 7.46 (d, J=8.8 Hz, 1H), 5.41-5.31 (m, 1H), 3.76 (s, 3H), 1.66 (d, J=6.8 Hz, 3H); ¹⁹F-NMR (376 MHz, methanol-d₄) δ −78.76, −81.33, −116.49, −142.50, −155.12, −164.78. Also collected with a longer retention time was methyl (R)-2-(2,2,7-trifluoro-3-oxo-6-(perfluorophenyl)-2,3-dihydro-4H-benzo[b][1,4]oxazin-4-yl)propanoate (Compound 8) as a yellow oil: GCMS calculated for C₁₈H₉F₅NO₄=455.0, found 455.0; ¹H-NMR (400 MHz, methanol-d₄) δ 7.86-7.76 (m, 2H), 5.51-5.41 (m, 1H), 3.67 (s, 3H), 1.55 (d, J=6.8 Hz, 3H); ¹⁹F-NMR (376 MHz, methanol-d₄) δ −76.05, −115.42, −140.28, −152.93, −161.96.

As shown in Step 3 of Scheme 4, a solution of methyl (R)-2-(2,2,7-trifluoro-3-oxo-6-(perfluorophenyl)-2,3-dihydro-4H-benzo[b][1,4]oxazin-4-yl)propanoate (50 mg, 0.11 mmol) in 4M HCl/dioxane (I mL) was stirred at 100° C. for 48 hours. The mixture was concentrated under reduced pressure and the residue purified by reversed-phase preparative HPLC (40% to 70% acetonitrile/0.5% HCl in water) to afford (R)-2-(2,2,7-trifluoro-3-oxo-6-(perfluorophenyl)-2,3-dihydro-4H-benzo[b][1,4]oxazin-4-yl)propanoic acid (Compound 10, 12 mg, 24% yield) as a white solid: MS (ES) calculated for C₁₇H₇F₈NO₄ [M−1]⁻=440.0, found 440.0; ¹H-NMR (400 MHz, DMSO-d₆) δ 7.87-7.79 (m, 2H), 5.37-5.27 (m, 1H), 1.52 (d, J=7.2 Hz, 3H); ¹⁹F-NMR (376 MHz, DMSO) δ −75.12, −115.76, −140.36, −153.04, −162.02.

As shown in Step 4 of Scheme 4, a solution of methyl (S)-2-(2,2,7-trifluoro-3-oxo-6-(perfluorophenyl)-2,3-dihydro-4H-benzo[b][1,4]oxazin-4-yl)propanoate (70 mg, 0.15 mmol) in 4M HCl/dioxane (1 mL) was stirred at 100° C. for 48 hours. The mixture was concentrated under reduced pressure and the residue purified by reversed-phase preparative HPLC (45% to 75% acetonitrile/0.5% aqueous HCl) to afford (S)-2-(2,2,7-trifluoro-3-oxo-6-(perfluorophenyl)-2,3-dihydro-4H-benzo[b][1,4]oxazin-4-yl)propanoic acid (Compound 11, 19 mg, 28% yield) as a white solid: MS (ESI) calculated for C₁₇H₇F₈NO₄ [M−1]⁻=440.0; found 440.0; ¹H-NMR (400 MHz, DMSO-d₆) δ 7.84-7.76 (m, 2H), 5.33 (m, 1H), 1.53 (d, J=7.2 Hz, 3H); ¹⁹F-NMR (376 MHz, DMSO) δ −76.18, −115.73, −140.03-140.61, −153.03, −162.03.

Using the appropriate alkylating agent in transformations similar to those described in Step 1 of Scheme 4, followed by any subsequent synthetic manipulations or purifications, resulted in the preparation of the following compounds.

Alkylation of Compound 1005 with ethyl 2-bromopropanoate, followed by chiral HPLC purification of the product produced ethyl (S)-2-(2,2,7-trifluoro-3-oxo-6-(perfluorophenyl)-2,3-dihydro-H-benzo[b][1,4]oxazin-4-yl)propanoate (Compound 35): MS (ESI) calculated for C₁₉H₁₁F₈NO [M−1]⁻=468.0, found 468.0; ¹H-NMR (400 MHz, methanol-d₄) δ 7.53 (d, J=6.4 Hz, 1H), 7.45 (d, J=9.2 Hz, 1H), 5.35-5.30 (m, 1H), 4.32-4.13 (m, 2H), 1.66 (d, J=7.2 Hz, 3H), 1.22 (t, J=7.2 Hz, 3H); ¹⁹F-NMR (400 MHz, methanol-d₄) δ −79.75, −116.51, −142.49, −156.12, −164.77; and ethyl (R)-2-(2,2,7-trifluoro-3-oxo-6-(perfuorophenyl)-2,3-dihydro-4H-benzo[b][1,4]oxazin-4-yl)propanoate (Compound 36): MS (ESI) calculated for C₁₉H₁₁F₈NO [M−1]⁻=468.0, found 468.0; ¹H-NMR (400 MHz, methanol-d₄) δ 7.53 (d, J=6.4 Hz, 1H), 7.44 (d, J=9.2 Hz, 1H), 5.35-5.30 (m, 1H), 4.29-4.15 (m, 2H), 1.66 (d, J=7.2 Hz, 3H), 1.23 (t, J=7.2 Hz, 3H); ¹⁹F-NMR (400 MHz, methanol-d₄) δ −79.75, −116.51, −142.48, −156.15, −164.77.

Reaction of racemic Compound 1006 with trimethylstannanol in dichlorocthane at 80° C. to produce 2-(2,2,7-trifluoro-3-oxo-6-(perfluorophenyl)-2,3-dihydro-4H-benzo[b][1,4]oxazin-4-yl)propanoic acid followed by esterification with isopropanol (catalytic H₂SO₄, 80° C.) produced, after chiral HPLC purification, isopropyl (S)-2-(2,2,7-trifluoro-3-oxo-6-(perfluorophenyl)-2,3-dihydro-4H-benzo[b][1,4]oxazin-4-yl)propanoate (Compound 37): MS (ES) calculated for C₂₀H₁₃F₈NO₄ [M−1]⁻=482.1, found 482.0; ¹H-NMR (400 MHz, methanol-d₄) δ 7.53 (d, J=6.0 Hz, 1H), 7.45 (d, J=9.2 Hz, 1H), 5.29-5.17 (m, 1H), 5.13-5.05 (m, 1H), 1.65 (d, J=7.2 Hz, 3H), 1.24 (d, J=6.0 Hz, 3H), 1.17 (d, J=6.0 Hz, 3H); ¹⁹F-NMR (377 MHz, methanol-d₄) δ −80.05, −116.55, −142.65, −156.10, −164.76, and isopropyl (R)-2-(2,2,7-trifluoro-3-oxo-6-(perfluorophenyl)-2,3-dihydro-4H-benzo[b][1,4]oxazin-4-yl)propanoate (Compound 38): MS (ESI) calculated for C₂₀H₁₃F₈NO₄ [M−1]⁻=482.1, found 482.0; ¹H-NMR (400 MHz, methanol-d₄) δ 7.53 (d, J=6.0 Hz, 1H), 7.45 (d, J=9.2 Hz, 1H), 5.29-5.17 (m, 1H), 5.13-5.05 (m, 1H), 1.65 (d, J=7.2 Hz, 3H), 1.24 (d, J=6.0 Hz, 3H), 1.17 (d, J=6.0 Hz, 3H); ¹⁹F-NMR (377 MHz, methanol-d₄) δ −80.05, −116.55, −142.65, −156.10, −164.76.

Alkylation of Compound 1005 with methyl 2-bromobutanoate, followed by chiral HPLC purification produced methyl (S)-2-(2,2,7-trifluoro-3-oxo-6-(perfluorophenyl)-2,3-dihydro-4H-benzo[b][1,4]oxazin-4-yl)butanoate (Compound 49): MS (ES) calculated for C₁₉H₁₁F₈NO₄ [M+1]⁺=469.0, found 469.1; ¹H-NMR (400 MHz, DMSO-d₆) δ 7.85 (t, J=8.6 Hz, 2H), 5.43-5.40 (m, 1H), 3.67 (s, 3H), 2.26-2.15 (m, 1H), 2.14-2.04 (m, 1H), 0.86 (t, J=7.5 Hz, 3H); ¹⁹F-NMR (400 MHz, DMSO-d₆) δ −76.90, −115.11, −140.85, −152.99, −162.09; which with treatment with trimethylstannanol in dichloroethane at 65° C., subsequently produced (S)-2-(2,2,7-trifluoro-3-oxo-6-(perfluorophenyl)-2,3-dihydro-4H-benzo[b][1,4]oxazin-4-yl)butanoic acid (Compound 51): MS (ESI) calculated for C₁₈H₉F₅NO₄ [M−1]⁻=454.0, found 454.3; ¹H-NMR (400 MHz, DMSO-d₆) δ 7.74-7.70 (m, 1H), 7.60 (s, 1H), 5.09-5.05 (m, 1H), 2.24-2.13 (m, 1H), 2.02-1.90 (m, 1H), 0.79 (t, J=7.2 Hz, 3H); ¹⁹F-NMR (376 MHz, DMSO-d₆) δ −72.17, −79.32, −116.12, −140.75, −153.18, −161.97; and methyl (R)-2-(2,2,7-trifluoro-3-oxo-6-(perfluorophenyl)-2,3-dihydro-4H-benzo[b][1,4]oxazin-4-yl)butanoate (Compound 50): GCMS calculated for C₁₉H₁₁F₅NO₄=469.0, found 469.1; ¹H-NMR (400 MHz, DMSO-d₆) δ 7.87-7.83 (m, 2H), 5.43-5.40 (m, 1H), 3.67 (s, 3H), 2.26-2.016 (m, 1H), 2.14-2.04 (m, 1H), 0.86 (t, J=7.6 Hz, 3H); ¹⁹F-NMR (400 MHz, DMSO-d₆) δ −76.90, −115.11, −140.85, −152.99, −162.09; which with treatment with trimethylstannanol in dichloroethane at 65° C., subsequently produced (R)-2-(2,2,7-trifluoro-3-oxo-6-(perfluorophenyl)-2,3-dihydro-4H-benzo[b][1,4]oxazin-4-yl)butanoic acid (Compound 52): MS (ESI) calculated for C₁₈H₉F₅NO₄ [M−1]⁻=454.0, found 454.2; ¹H-NMR (400 MHz, DMSO-d₆) δ 7.71 (d, J=9.6 Hz, 1H), 7.44 (d, J=6.4 Hz, 1H), 5.02-4.87 (m, 1H), 2.29-2.12 (m, 1H), 1.93-1.73 (m, 1H), 0.86-0.66 (m, 3H); ¹⁹F-NMR (376 MHz, DMSO-d₆) δ −72.92, −79.32, −116.12, −140.75, −153.18, −161.97.

Alkylation of Compound 1005 with methyl 2-bromopentanoate produced racemic methyl 2-(2,2,7-trifluoro-3-oxo-6-(perfluorophenyl)-2,3-dihydro-4H-benzo[b][1,4]oxazin-4-yl)pentanoate, which was separated into its respective enantiomers methyl (S)-2-(2,2,7-trifluoro-3-oxo-6-(perfluorophenyl)-2,3-dihydro-4H-benzo[b][1,4]oxazin-4-yl)pentanoate (Compound 115): MS (ESI) calculated for C₂₀H₁₃F₈NO₄ [M−1]⁻=482.0, found 481.9; ¹H-NMR (400 MHz, CD₃OD) δ 7.50-7.44 (m, 2H), 5.44-5.41 (m, 1H), 3.50 (s, 3H), 2.29-2.12 (m, 2H), 1.42-1.27 (m, 2H), 0.94 (d, J=6.4 Hz, 3H); ¹⁹F-NMR (376 MHz, CD₃OD) δ −80.95, −116.29, −143.04, −156.11, −164.74; and methyl (R)-2-(2,2,7-trifluoro-3-oxo-6-(perfluorophenyl)-2,3-dihydro-4H-benzo[b][1,4]oxazin-4-yl)pentanoate (Compound 116): MS (ESI) calculated for C₂₀H₁₃F₅NO₄ [M−1]⁻=482.0, found 481.9; ¹H-NMR (400 MHz, CD₃OD) δ 7.50-7.44 (m, 2H), 5.44-5.41 (m, 1H), 3.50 (s, 3H), 2.29-2.12 (m, 2H), 1.42-1.27 (m, 2H), 0.94 (d, J=6.4 Hz, 3H); ¹⁹F-NMR (376 MHz, CD₃OD) δ −80.95, −116.29, −143.04, −156.11, −164.74.

Hydrolysis of the enantiomers of racemic methyl 2-(2,2,7-trifluoro-3-oxo-6-(perfluorophenyl)-2,3-dihydro-4H-benzo[b][1,4]oxazin-4-yl)pentanoate, followed by chiral HPLC purification, produced (S)-2-(2,2,7-trifluoro-3-oxo-6-(perfluorophenyl)-2,3-dihydro-4H-benzo[b][1,4]oxazin-4-yl)pentanoic acid (Compound 117): MS (ESI) calculated for C₁₉H₁₁F₈NO₄ [M−1]⁻=468.0, found 467.9; ¹H-NMR (400 MHz, CD₃OD) δ 7.48-7.43 (m, 2H), 5.43-5.40 (m, 1H), 2.31-2.05 (m, 2H), 1.44-1.25 (m, 2H), 0.95 (d, J=6.4 Hz, 3H); ¹⁹F-NMR (376 MHz, CD₃OD) δ −79.22, −81.60, −116.59, −142.52, −156.17, −164.77; and (R)-2-(2,2,7-trifluoro-3-oxo-6-(perfluorophenyl)-2,3-dihydro-4H-benzo[b][1,4]oxazin-4-yl)pentanoic acid (Compound 118): MS (ESI) calculated for C₁₉H₁₁F₅NO₄ [M−1]⁻=468.0, found 467.9; ¹H-NMR (400 MHz, CD₃OD) δ 7.48-7.43 (m, 2H), 5.43-5.40 (m, 1H), 2.31-2.05 (m, 2H), 1.44-1.25 (m, 2H), 0.95 (d, J=6.4 Hz, 3H); ¹⁹F-NMR (376 MHz, CD₃OD) δ −78.75, −81.50, −116.62, −142.65, −156.18, −164.77.

Alkylation of Compound 1005 with methyl 2-bromo-4-methylpentanoate, followed by HPLC chiral separation, produced methyl (S)-4-methyl-2-(2,2,7-trifluoro-3-oxo-6-(perfluorophenyl)-2,3-dihydro-4H-benzo[b][1,4]oxazin-4-yl)pentanoate (Compound 122): MS (ESI) calculated for C₂₁H₁₁F₈NO₄ [M−1]⁻=496.0, found 496.0; ¹H-NMR (400 MHz, DMSO-d₆) δ 7.89-7.81 (m, 2H), 5.55-5.47 (m, 1H), 3.68 (s, 3H), 2.11-1.99 (m, 2H), 1.53-1.38 (m, 1H), 0.85 (d, J=6.4 Hz, 6H); ¹⁹F-NMR (376 MHz, DMSO-d₆) δ −77.14, −114.97, −140.69, −152.91, −161.91; which with treatment with trimethylstannanol in dichloroethane at 65° C., subsequently produced (S)-4-methyl-2-(2,2,7-trifluoro-3-oxo-6-(perfluorophenyl)-2,3-dihydro-4H-benzo[b][1,4]oxazin-4-yl)pentanoic acid (Compound 124): MS (ESI) calculated for C₂₀H₁₃F₈NO₄ [M−1]⁻=482.0, found 482.0; ¹H-NMR (400 MHz, DMSO-d₆) δ 13.35 (s, 1H), 7.84-7.80 (m, 2H), 5.42-5.35 (m, 1H), 2.12-1.95 (m, 2H), 1.50-1.38 (m, 1H), 0.88-0.80 (m, 6H); ¹⁹F-NMR (400 MHz, DMSO-d₆) δ −76.19, −115.09, −140.83, −152.98, −161.91; and methyl (R)-4-methyl-2-(2,2,7-trifluoro-3-oxo-6-(perfluorophenyl)-2,3-dihydro-4H-benzo[b][1,4]oxazin-4-yl)pentanoate (Compound 123): MS (ESI) calculated for C₂₁H₁₅F₈NO₄ [M−1]⁻=496.0, found 496.0; ¹H-NMR (400 MHz, DMSO-d₆) δ 7.92-7.81 (m, 2H), 5.52-5.49 (m, 1H), 3.68 (s, 3H), 2.12-1.99 (m, 2H), 1.52-1.39 (m, 1H), 0.84 (d, J=6.4 Hz, 6H); ¹⁹F-NMR (376 MHz, DMSO-d₆) δ −77.14, −114.97, −140.69, −152.92, −161.92; which with treatment with trimethylstannanol in dichloroethane at 65° C., subsequently produced (R)-4-methyl-2-(2,2,7-trifluoro-3-oxo-6-(perfluorophenyl)-2,3-dihydro-4H-benzo[b][1,4]oxazin-4-yl)pentanoic acid (Compound 125): MS (ESI) calculated for C₂₀H₁₃F₅NO₄ [M−1]⁻=482.0, found 482.0; ¹H-NMR (400 MHz, DMSO-d₆) δ 13.38 (b, 1H), 7.92-7.82 (m, 2H), 5.43-5.39 (m, 1H), 2.10-1.99 (m, 2H), 1.47-1.38 (m, 1H), 0.84 (d, J=1.6 Hz, 6H); ¹⁹F-NMR (400 MHz, DMSO-d₆) δ −75.90, −115.35, −140.72, −153.06, −162.00.

Alkylation of Compound 1005 with methyl 2-hydroxy-3-methylbutanoate under Mitsunobu conditions (PPh₃, diisopropyl azodicarboxylate, 0° C.) produced racemic methyl 3-methyl-2-(2,2,7-trifluoro-3-oxo-6-(perfluorophenyl)-2,3-dihydro-4H-benzo[b][1,4]oxazin-4-yl)butanoate which was hydrolyzed by treatment with trimethylstannanol (dichloroethane, 65° C.), then separated by chiral HPLC, to produce (S)-3-methyl-2-(2,2,7-trifluoro-3-oxo-6-(perfluorophenyl)-2,3-dihydro-4H-benzo[b][1,4]oxazin-4-yl)butanoic acid (Compound 150): MS (ESI) calculated for C₁₉H₁₁F₈NO₄ [M−1]⁻=468.0, found 467.9; ¹H-NMR (400 MHz, DMSO-d₆) δ 13.32 (s, 1H), 7.90-7.80 (m, 2H), 4.93 (d, J=9.6 Hz, 1H), 2.67-2.65 (m, 1H), 1.25-1.15 (m, 3H), 0.75 (d, J=6.8 Hz, 3H); ¹⁹F-NMR (377 MHz, DMSO-d) δ −77.29, −153.20, −140.73, −153.13, −161.92; and (R)-3-methyl-2-(2,2,7-trifluoro-3-oxo-6-(perfluorophenyl)-2,3-dihydro-4H-benzo[b][1,4]oxazin-4-yl)butanoic acid (Compound 151): MS (ESI) calculated for C₁₉H₁₁F₈NO₄ [M−1]⁻=468.0, found 468.0; ¹H-NMR (400 MHz, DMSO-d₆) δ 13.34 (s, 1H), 7.88-7.78 (m, 2H), 4.92 (d, J=9.6 Hz, 1H), 2.69-2.59 (m, 1H), 1.25-1.15 (m, 3H), 0.79-0.69 (m, 3H); ¹⁹F-NMR (377 MHz, DMSO-d₆) δ −77.28, −115.20, −140.73, −153.16, −162.93.

Esterification of Compound 150 and Compound 151 with trimethylsilyldiazomethane produced, respectively, methyl (S)-3-methyl-2-(2,2,7-trifluoro-3-oxo-6-(perfluorophenyl)-2,3-dihydro-4H-benzo[b][1,4]oxazin-4-yl)butanoate (Compound 152): MS (ESI) calculated for C₂₀H₁₃F₈NO₄ [M−1]⁻⁰=482.1 found 481.9; ¹H-NMR (400 MHz, DMSO-d₆) δ 7.90-7.72 (m, 2H), 5.09 (d, J=9.6 Hz, 1H), 3.64 (s, 3H), 2.68 (t, J=8.8 Hz, 1H), 1.20 (d, J=6.4 Hz, 3H), 0.77 (d, J=6.8 Hz, 3H); ¹⁹F-NMR (377 MHz, DMSO-d₆) δ −76.66, −114.86, −140.50, −153.03, −161.95; and methyl (R)-3-methyl-2-(2,2,7-trifluoro-3-oxo-6-(perfluorophenyl)-2,3-dihydro-4H-benzo[b][1,4]oxazin-4-yl)butanoate (Compound 153): MS (ESI) calculated for C₂₀H₁₃F₈NO₄ [M−1]⁻=482.1, found 482.0; ¹H-NMR (400 MHz, DMSO-d₆) δ 8.02-7.84 (m, 2H), 5.09 (d, J=9.2 Hz, 1H), 3.64 (s, 3H), 2.77-2.60 (m, 1H), 1.20 (d, J=6.4 Hz, 3H), 0.77 (d, J=6.8 Hz, 3H); ¹⁹F-NMR (377 MHz, DMSO-d₆) δ −77.33, −114.86, −140.04, −153.05, −161.95.

Alkylation of Compound 1005 with methyl 2-bromo-2-methoxyacetate produced racemic methyl 2-methoxy-2-(2,2,7-trifluoro-3-oxo-6-(perfluorophenyl)-2,3-dihydro-4H-benzo[b][1,4]oxazin-4-yl)acetate which was separated into its enantiomers by chiral HPLC to produce methyl (S)-2-methoxy-2-(2,2,7-trifluoro-3-oxo-6-(perfluorophenyl)-2,3-dihydro-4H-benzo[b][1,4]oxazin-4-yl)acetate (Compound 185): GCMS calculated for C₁₈H₉F₈NO₅=471.0, found 471.0, ¹H-NMR (400 MHz, DMSO-d₆) δ 7.85 (d, J=9.4 Hz, 1H), 7.48 (d, J=6.4 Hz, 1H), 7.11 (s, 1H), 6.63 (s, 1H), 3.69 (s, 3H), 3.47 (s, 3H); ¹⁹F-NMR (376 MHz, DMSO-d₆) δ −74.79, −114.28, −140.38, −142.19, −152.80, −161.54; and methyl (R)-2-methoxy-2-(2,2,7-trifluoro-3-oxo-6-(perfluorophenyl)-2,3-dihydro-4H-benzo[b][1,4]oxazin-4-yl)acetate (Compound 186): GCMS calculated for C₁₈H₉F₈NO₅=471.0, found 471.0; ¹H-NMR (400 MHz, DMSO-d₆) δ 7.85 (d, J=9.4 Hz, 1H), 7.48 (d, J=6.4 Hz, 1H), 6.63 (s, 1H), 3.69 (s, 3H), 3.47 (s, 3H); ¹⁹F-NMR (376 MHz, DMSO-d₆) δ −74.79, −114.28, −140.38, −142.25, −152.80, −161.54.

Each of Compounds 185 and 186 were treated with trimethylstannanol in DCE at 65° C. to provide, respectively, (S)-2-methoxy-2-(2,2,7-trifluoro-3-oxo-6-(perfluorophenyl)-2,3-dihydro-4H-benzo[b][1,4]oxazin-4-yl)acetic acid (Compound 187): MS (ESI) calculated for C₁₇H₇F₈NO₅ [M−1]⁻=456.0, found 455.9; ¹H-NMR (400 MHz, DMSO-d₆) δ 14.05 (br, 1H), 7.84 (d, J=9.6 Hz, 1H), 7.55 (d, J=6.4 Hz, 1H), 6.47 (s, 1H), 3.45 (s, 3H); ¹⁹F-NMR (377 MHz, DMSO-d₆) δ −76.92, −114.47, −140.61, −141.55, −152.69, −161.67; and (R)-2-methoxy-2-(2,2,7-trifluoro-3-oxo-6-(perfluorophenyl)-2,3-dihydro-4H-benzo[b][1,4]oxazin-4-yl)acetic acid (Compound 188): MS (ES) calculated for C₁₇H₇F₈NO₅ [M−1]⁻=456.0, found 455.9; ¹H-NMR (400 MHz, DMSO-d₆) δ 14.05 (br, 1H), 7.84 (d, J=9.6 Hz, 1H), 7.55 (d, J=6.4 Hz, 1H), 6.47 (s, 1H), 3.45 (s, 3H); ¹⁹F-NMR (377 MHz, DMSO-d₆) δ −76.92, −114.47, −140.61, −141.55, −152.69, −161.67.

### Example 3. Preparation of Preparation of 2-(2,2,7-trifluoro-3-oxo-6-(perfluorophenyl)-2,3-dihydro-4H-benzo[b][1,4]oxazin-4-yl)N-((trifluoromethyl)sulfonyl)acetamide (Compound 12)

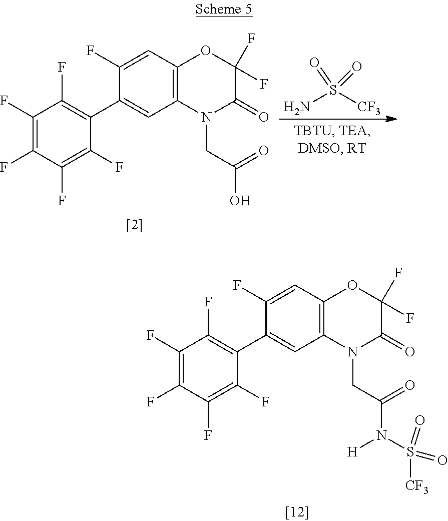

As shown in Scheme 5, to a solution of 2-(2,2,7-trifluoro-3-oxo-6-(perfluorophenyl)-2,3-dihydro-4H-benzo[b][1,4]oxazin-4-yl)acetic acid (Compound 2, 70 mg, 0.16 mmol) in DMSO (1 mL) were added triethylamine (50 mg, 0.49 mmol), trifluoromethanesulfonamide (29 mg, 0.20 mmol), and O-(benzotriazol-1-yl)-N,N,N′,N′-tetramethyluronium tetrafluoroborate (TBTU, 79 mg, 0.25 mmol). The mixture was stirred at room temperature for 2 hours then purified by reversed-phase preparative HPLC (40%-60% acetonitrile/10 mM aqueous NH₄HCO₃) to afford 2-(2,2,7-trifluoro-3-oxo-6-(perfluorophenyl)-2,3-dihydro-4H-benzo[b][1,4]oxazin-4-yl)-N-((trifluoromethyl)sulfonyl)-aeetamide (Compound 12, 25 mg, 25% yield) as a white solid: MS (ESI) calculated for (C₁₇H₅F₁₁N₂O₅S) [M−1]⁻=557.0, found 556.8; ¹H-NMR (400 MHz, DMSO-d₆) δ 7.70 (d, J=9.4 Hz, 1H), 7.40 (d, J=6.4 Hz, 1H), 4.54 (s, 2H); ¹⁹F-NMR (376 MHz, DMSO-d₆) δ −75.41, −78.01, −116.34, −140.37, −153.14, −162.06.

Reaction of Compound 2 with the appropriate amine, as shown in Scheme 5, followed by any subsequent synthetic manipulations or purifications, resulted in the preparation of the following compounds.

Condensation of Compound 2, after TBTU activation, with methanesulfonamide produced N-(methylsulfonyl)-2-(2,2,7-trifluoro-3-oxo-6-(perfluorophenyl)-2,3-dihydro-4H-benzo[b][1,4]oxazin-4-yl)acetamide (Compound 13): MS (ESI) calculated for C₁₇H₈F₈N₂O₅S [M−1]⁻=503.0, found 502.9; ¹H-NMR (400 MHz, methanol-d₄) δ 7.61-7.18 (m, 2H), 4.69 (d, J=4.0 Hz, 2H), 2.99-3.15 (m, 3H); ¹⁹F-NMR (376 MHz, methanol-d₄) δ −77.87, −117.62, −142.25, −156.47, −164.96.

Condensation of Compound 2, after 1-ethyl-3-(3-dimethylaminopropyl)carbodiimidc (EDCI) activation, with isopropyl(methyl)sulfamoylamine produced N-(N-isopropyl-N-methylsulfamoyl)-2-(2,2,7-trifluoro-3-oxo-6-(perfluorophenyl)-2,3-dihydro-4H-benzo[b][1,4]oxazin-4-yl)acetamide (Compound 14): MS (ESI) calculated for C₂₀H₁₅F₈N₃O₅S [M−1]⁻=560.1, found 559.9; ¹H-NMR (400 MHz, methanol-d₄) δ 7.54-7.23 (m, 2H), 4.73 (s, 2H), 4.10 (s, 1H), 2.73 (s, 3H), 1.08 (d, J=6.8 Hz, 61-1); ¹⁹F-NMR (400 MHz, methanol-d₄) δ −78.39, −117.29, −142.09, −156.27, −164.90.

Condensation of Compound 2, after EDCT activation, with dimethyl(sulfamoyl)amine produced N-(N,N-dimethylsulfamoyl)-2-(2,2,7-trifluoro-3-oxo-6-(perfluorophenyl)-2,3-dihydro-4H-benzo[b][1,4]oxazin-4-yl)acetamide (Compound 15): MS (ESI) calculated for C₁₈H₁₁F₈N₃O₅S [M−1]⁻=532.0, found 531.9; ¹H-NMR (400 MHz, DMSO-d₆) δ 11.94 (s, 1H), 7.78 (d, J=9.6 Hz, 1H), 7.72 (d, J=6.4 Hz, 1H), 4.80 (s, 2H), 2.75 (s, 6H); ¹⁹F-NMR (376 MHz, DMSO-d₆) δ −74.89, −115.95, −140.29, −152.91, −161.94.

Condensation of Compound 2, after EDCI activation, with 1,1-dimethylhydrazine produced N,N-dimethyl-2-(2,2,7-trifluoro-3-oxo-6-(perfluorophenyl)-2,3-dihydro-4H-benzo[b][1,4]oxazin-4-yl)acetohydrazide (Compound 57): MS (ESI) calculated for C₁₉H₉F₈N₃O₂ [M+1]⁺=470.1, found 470.1; ¹H-NMR (400 MHz, methanol-d₄) δ 7.52-7.25 (m, 2H), 5.09 (s, 1H), 4.70 (s, 1H), 2.64 (s, 3H), 2.55 (s, 3H); ¹⁹F-NMR (376 MHz, methanol-d₄) δ −78.50, −117.05, −142.39, −156.33, −164.99.

Condensation of Compound 2, after activation With carbonyldiimidazole (CDI), with O-methylhydroxylamine produced N-methoxy-2-(2,2,7-trifluoro-3-oxo-6-(perfluorophenyl)-2,3-dihydro-H-benzo[b][1,4]oxazin-4-yl)acetamide (Compound 58): MS (ESI) calculated for C₁₇H₈F₈N₂O₄ [M−1]⁻=455.0, found 455.0; ¹H-NMR (400 MHz, DMSO-d₆) δ 11.54 (s, 1H), 7.78 (d, J=9.2 Hz, 1H), 7.63 (d, J=6.4 Hz, 1H), 4.62 (s, 2H), 3.59 (s, 3H); ¹⁹F-NMR (400 MHz, DMSO-d₆) δ−74.85, −115.97, −140.39, −153.02, −161.95.

Condensation of Compound 2, after EDCI activation, with tert-butyl 1-methylhydrazine-1-carboxylate produced tert-butyl 1-methyl-2-(2-(2,2,7-trifluoro-3-oxo-6-(perfluorophenyl)-2,3-dihydro-4H-benzo[b][1,4]oxazin-4-yl)acetyl)hydrazine-1-carboxylate: MS (ESI) calculated for C₂₂H₁₇F₈N₃O₅ [M−1]⁻=554.1, found 554.0; which, after treatment with trifluoroacetic acid in DCM at 0° C., produced N′-methyl-2-(2,2,7-trifluoro-3-oxo-6-(perfluorophenyl)-2,3-dihydro-4H-benzo[b][1,4]oxazin-4-yl)acetohydrazide (Compound 59): MS (ESI) calculated for C₁₇H₉F₈N₃O; [M+1]⁻=456.0, found 456.1; ¹H-NMR (400 MHz, DMSO-d₆) δ9.70 and 8.96 (s, 1H), 7.75 (d, J=1.2 Hz, 1H), 7.57-7.23 (m, 1H), 4.90 and 4.64 (s, 3H), 2.52-2.50 (m, 1H), 2.41 (s, 2H); ¹⁹F-NMR (400 MHz, DMSO-d₆) δ −74.18, −116.29, −140.43, −153.15, −162.00.

Condensation of Compound 2, after EDCI activation, with O-benzylhydroxylamine produced N-(benzyloxy)-2-(2,2,7-trifluoro-3-oxo-6-(perfluorophenyl)-2,3-dihydro-4H-benzo[b][1,4]oxazin-4-yl)acetamide (Compound 70): MS (ESI) calculated for C₂₃H₁₂F₈N₂O₄=533.0, found 533.0; ¹H-NMR (400 MHz, CDCl₃) δ 8.08 & 8.55 (br, 1H), 7.36-7.34 (m, 6H), 7.19-7.14 (m, 1H), 4.94 (s, 2H), 4.80-4.42 (m, 2H); ¹⁹F-NMR (400 MHz, DMSO-d₆) δ −76.88, −113.22, −139.62, −152.10, −160.91; which, after treatment with hydrogen and Pd/C, produced N-hydroxy-2-(2,2,7-trifluoro-3-oxo-6-(perfluorophenyl)-2,3-dihydro-4H-benzo[b][1,4]oxazin-4-yl)acetamide (Compound 71): MS (ESI) calculated for C₁₆H₆F₈N₂O₄=443.0, found 443.0; ¹H-NMR (400 MHz, methanol-d₄) δ 7.42-7.18 (m, 2H), 4.72 (s, 2H); ¹⁹F-NMR (400 MHz, methanol-d₄) δ −78.20, −117.13, −142.27, −156.25, −164.85.

Condensation of Compound 2, after EDCI activation, with methyl (2S)-pyrrolidine-2-carboxylate, hydrochloride produced methyl (2-(2,2,7-trifluoro-3-oxo-6-(perfluomphenyl)-2,3-dihydro-4H-benzo[b][1,4]oxazin-4-yl)acetyl)-D-prolinate (Compound 72): MS (ESI) calculated for (C₂₂H₁₄F₈N₂O₅) [M+1]⁺, 539.0; found, 539.1; ¹H-NMR (400 MHz, methanol-d₄) δ 7.43-7.30 (m, 2H), 5.22-5.11 (m, 1H), 4.95-4.74 (m, 1H), 4.58-4.46 (m, 1H), 3.89-3.42 (m, 5H), 2.45-2.22 (m, 1H), 2.16-1.92 (m, 3H); ¹⁹F-NMR (377 MHz, DMSO-d₆) δ −78.12, −117.40, −142.50, −156.38, −165.11; which, after treatment with 4M HCl in dioxane at 100° C., produced (2-(2,2,7-trifluoro-3-oxo-6-(perfluorophenyl)-2,3-dihydro-4H-benzo[b][1,4]oxazin-4-yl)acetyl)-D-proline (Compound 73): MS (ESI) calculated for C₂₂H₁₂F₈N₂O₅ [M+1]⁺=525.0, found 525.0; ¹H-NMR (400 MHz, methanol-d₄) δ 7.52-7.44 (m, 1H), 7.40-7.34 (m, 1H), 5.18-5.07 (m, 1H), 4.64-4.43 (m, 2H), 3.86-3.68 (m, 1H), 3.63-3.45 (m, 1H), 2.39-2.00 (m, 3H), 1.95-1.81 (m, 1H); ¹⁹F-NMR (376 MHz, methanol-d₄) δ −78.99, −117.57, −141.27, −156.71, −165.08.

Condensation of Compound 2, after activation as the acyl chloride via oxalyl chloride treatment at 0° C., with methyl L-prolinate produced methyl (2-(2,2,7-trifluoro-3-oxo-6-(perfluorophenyl)-2,3-dihydro-4H-benzo[b][1,4]oxazin-4-yl)acetyl)-L-prolinate (Compound 74): MS (ESI) calculated for C₂₂H₁₄F₈N₂O₅ [M+1]⁺=539.0, found 539.1; ¹H-NMR (400 MHz, methanol-d₄) δ 7.42-7.37 (m, 1H), 7.34 (d, J=10.4 Hz, 1H), 5.20-5.13 (m, 1H), 4.89-4.75 (m, 1H), 4.60-4.46 (m, 1H), 3.89-3.50 (m, 5H), 2.42-2.23 (m, 1H), 2.15-1.73 (m, 3H); ¹⁹F-NMR (400 MHz, DMSO-d₆) δ −77.91, −117.40, −142.37, −156.44, −165.11; which, after treatment with 4M HCl in dioxane at 100° C., produced (2-(2,2,7-trifluoro-3-oxo-6-(perfluorophenyl)-2,3-dihydro-4H-benzo[b II 1,4]oxazin-4-yl)acetyl)-L-proline (Compound 75): MS (ESI) calculated for C₂₁H₁₂F₈N₂O₅ [M+1]⁺=525.0, found 525.1; ¹H-NMR (400 MHz, methanol-d₄) δ 7.46-7.29 (m, 2H), 5.20-5.08 (m, 1H), 4.65-4.44 (m, 1H), 3.89-3.71 (m, 1H), 3.64-3.45 (m, 2H), 2.42-2.24 (m, 1H), 2.17-1.78 (m, 3H); ¹⁹F-NMR (400 MHz, DMSO-d₆) δ −77.95, −117.37, −142.35, −156.61, −164.96.

Condensation of Compound 2, after activation with n-propanephosphonic acid anhydride (T₃P) and DIEA, with methyl 3-(methylamino)propanoate produced methyl 3-(N-methyl-2-(2,2,7-trifluoro-3-oxo-6-(perfluorophenyl)-2,3-dihydro-4H-benzo[b][1,4]oxazin-4-yl)acetamido)propanoate (Compound 80): MS (ESI) calculated for C₂₁H₁₄F₈N₂O₅ [M+1]⁺=527.0, found 527.1; ¹H-NMR (400 MHz, CDCl₃) δ 7.39-6.84 (m, 2H), 5.17 & 4.77 (s, 2H), 3.71-3.65 (m, 2H), 3.58 (s, 3H), 3.18 & 2.92 (s, 3H), 2.82-2.49 (m, 2H); ¹⁹F-NMR (376 MHz, CDCl₃) δ −76.84, −114.40, −139.43, −152.94, −161.57; which, after treatment with trimethylstannanol in DCE at 80° C., produced 3-(N-methyl-2-(2,2,7-trifluoro-3-oxo-6-(perfluorophenyl)-2,3-dihydro-H-benzo[b][1,4]oxazin-4-yl)acetamido)propanoic acid (Compound 81): MS (ESI) calculated for C₂₀H₁₂F₈N₂O₅ [M−1]⁻=511.0, found 511.0; ¹H-NMR (400 MHz, CDCl₃) δ 7.21-6.83 (m, 2H), 5.05 & 4.79 (s, 2H), 3.75-3.62 (m, 2H), 3.20 & 2.95 (s, 3H), 2.80-2.55 (m, 2H); ¹⁹F-NMR (376 MHz, CDCl₃) 5-76.94, −114.12, −139.69, −152.73, −161.42.

Condensation of Compound 2, after 1-[bis(dimethylamino)methylene]-1H-1,2,3-triazolo[4,5-b]pyridinium 3-oxid hexafluorophosphate (HATU) and DIEA activation, with methanamine hydrochloride produced N-methyl-2-(2,2,7-trifluoro-3-oxo-6-(perfluorophenyl)-2,3-dihydro-4H-benzo[b][1,4]oxazin-4-yl)acetamide (Compound 82): MS (ESI) calculated for C₁₇H₈F₈N₂O₃ [M−1]⁻=439.0, found, 438.9; ¹H-NMR (400 MHz, DMSO-d₆) δ 8.22 (d, J=5.2 Hz, 1H), 7.77-7.74 (d, J=9.6 Hz, 1H), 7.57-7.55 (d, J=6.4 Hz, 1H), 4.65 (s, 2H), 2.63-2.60 (d, J=4.4 Hz, 3H); ¹⁹F-NMR (377 MHz, DMSO-d₆) δ −74.32, −116.21, −140.44, −153.18, −162.01.

Condensation of Compound 2, after EDCI activation, with methyl 2-(methylamino) acetate, hydrochloride produced methyl N-methyl-N-(2-(2,2,7-trifluoro-3-oxo-6-(perfluorophenyl)-2,3-dihydro-4H-benzo[b][1,4]oxazin-4-yl)acetyl)glycinate (Compound 84): MS (ESI) calculated for C₂₀H₁₂F₈N₂O₅ [M+1]⁺=513.1, found 513.1; ¹H-NMR (400 MHz, methanol-d₄) δ 7.42-7.37 (m, 1H), 7.32 (d, J=6.4 Hz, 1H), 5.09 (s, 2H), 4.37 and 4.18 (s, 2H), 3.79 and 3.69 (s, 3H), 3.24 (s, 2H), 2.97 (s, 1H); ¹⁹F-NMR (377 MHz, methanol-d₄) δ −78.11, −117.40, −141.93, −156.57, −165.08; which, after treatment with trimethylstannanol in DCE at 80° C., produced N-methyl-N-(2-(2,2,7-trifluoro-3-oxo-6-(perfluorophenyl)-2,3-dihydro-4H-benzo[b][1,4]oxazin-4-yl)acetyl)glycine (Compound 85): MS (ESI) calculated for (C₁₉H₁₀F₈N₂O₅) [M−1]⁻=497.0, found 497.0; ¹H-NMR (400 MHz, methanol-d₄) δ 7.54-7.46 (m, 1H), 7.38-7.35 (m, 1H), 5.06 & 4.95 (s, 2H), 4.11 (s, 2H), 3.32 & 2.98 (s, 3H); ¹⁹F-NMR (377 MHz, DMSO-d₆) δ −78.25, −117.82, −142.18, −156.93, −165.11.

Condensation of Compound 2, after activation as the acyl chloride via oxalyl chloride treatment at 0° C., with methyl azetidine-3-carboxylate produced methyl 1-(2-(2,2,7-trifluoro-3-oxo-6-(perfluorophenyl)-2,3-dihydro-4H-benzo[b][1,4]oxazin-4-yl)acetyl)azetidine-3-carboxylate (Compound 86): MS (ESI) calculated for (C₂₁H₂F₈N₂O₅ [M+1]⁺=525.2, found 525.1; ¹H-NMR (400 MHz, DMSO-d₆) δ 7.77 (d, J=9.2 Hz, 1H), 7.60 (d, J=6.4 Hz, 1H), 4.74 (s, 2H), 4.52-4.47 (m, 2H), 4.09-3.95 (m, 2H), 3.68 (s, 3H), 3.62-3.48 (m, 1H); ¹⁹F-NMR (376 MHz, DMSO-d₆) δ −75.15, −115.98, −140.28, −152.95, −161.81; which, after treatment with trimethylstannanol in DCE at 80° C., produced 1-(2-(2,2,7-trifluoro-3-oxo-6-(perfluorophenyl)-2,3-dihydro-4H-benzo[b][1,4]oxazin-4-yl)acetyl)azetidine-3-carboxylic acid (Compound 87): MS (ESI) calculated for C₂₀H₁₀F₈N₂O₅ [M+1]⁺=511.0, found 511.1; ¹H-NMR (400 MHz, DMSO-d₆) δ 12.82 (s, 1H), 7.77 (d, J=9.6 Hz, 1H), 7.61 (d, J=6.4 Hz, 1H), 4.74 (s, 2H), 4.51-4.42 (m, 1H), 4.42-4.34 (m, 1H), 4.13-4.03 (m, 1H), 3.98-3.90 (m, 1H), 3.56-3.44 (m, 1H); ¹⁹F-NMR (376 MHz, DMSO-d₆) δ −75.13, −116.03, −140.29, −152.92, −161.81.

Condensation of Compound 2, after HATU and DIEA activation, with methyl piperidine-4-carboxylate produced methyl 1-(2-(2,2,7-trifluoro-3-oxo-6-(perfluorophenyl)-2,3-dihydro-4H-benzo[b][1,4]oxazin-4-yl)acetyl)piperidine-4-carboxylate (Compound 88): MS (ESI) calculated for C₂₃H₁₆F₈N₂O₅ [M+1]⁺=553.0, found 553.1; ¹H-NMR (400 MHz, DMSO-d₆) δ 7.78 (d, J=9.6 Hz, 1H), 7.57 (d, J=6.4 Hz, 1H), 5.08-4.93 (m, 2H), 4.16-4.13 (m, 1H), 3.89-3.85 (m, 1H), 3.63 (s, 3H), 3.23-3.19 (m, 1H), 2.82-2.80 (m, 1H), 2.73-2.61 (m, 1H), 1.96-1.82 (m, 2H), 1.70-1.56 (m, 1H), 1.43-1.32 (m, 1H); ¹⁹F-NMR (376 MHz, DMSO-d₆) δ −75.65, −116.09, −140.56, −153.04, −161.82; which, after treatment with 4M HCl/dioxane at 90° C., produced 1-(2-(2,2,7-trifluoro-3-oxo-6-(perfluorophenyl)-2,3-dihydro-4H-benzo[b][1,4]oxazin-4-yl)acetyl)piperidine-4-carboxylic acid (Compound 89): MS (ESI) calculated for C₂₂H₁₄F₈N₂O₅ [M+1]⁺=539.1, found 539.1; ¹H-NMR (400 MHz, DMSO-d₆) δ 12.29 (s, 1H), 7.78 (d, J=9.6 Hz, 1H), 7.57 (d, J=6.4 Hz, 1H), 5.09-4.92 (m, 2H), 4.18-4.08 (m, 1H), 3.85 (t, J=10.2 Hz, 1H), 3.24-3.15 (m, 1H), 2.84-2.76 (m, 1H), 2.59-2.53 (m, 1H), 1.94-1.80 (m, 2H), 1.66-1.55 (m, 1H), 1.42-1.32 (m, 1H); ¹⁹F-NMR (376 MHz, DMSO-d₆) δ −75.00, −116.11, −140.56, −152.93, −161.76.

Condensation of Compound 2, after HATU and DIEA activation, with methyl 2-aminoacetate produced methyl (2-(2,2,7-trifluoro-3-oxo-6-(perfluorophenyl)-2,3-dihydro-4H-benzo[b][1,4]oxazin-4-yl)acetyl)glycinate (Compound 91): MS (ESI) calculated for C₁₉H₁₀F₈N₂O₅ [M+1]⁺=499.0, found 499.1; ¹H-NMR (400 MHz, DMSO-d₆) δ8.86-8.78 (m, 1H), 7.77 (d, J=9.6 Hz, 1H), 7.48 (d, J=6.4 Hz, 1H), 4.76 (s, 2H), 3.90 (d, J=6.0 Hz, 2H), 3.59 (s, 3H); ¹⁹F-NMR (376 MHz, DMSO-d₆) δ 74.63, −116.16, −140.22, −153.24, −162.14; which, after treatment with 4M HCl/dioxane at 100° C., produced (2-(2,2,7-trifluoro-3-oxo-6-(perfluorophenyl)-2,3-dihydro-4H-benzo[b][1,4]oxazin-4-yl)acetyl)glycine (Compound 92): MS (ESI) calculated for C₁₈H₈F₈N₂O₅ [M+1]⁺=485.0, found 485.1; ¹H-NMR (400 MHz, DMSO-d₆) δ 12.56 (s, 1H), 8.75-8.68 (m, 1H), 7.77 (d, J=9.6 Hz, 1H), 7.48 (d, J=6.4 Hz, 1H), 4.74 (s, 2H), 3.80 (d, J=5.6 Hz, 2H); ¹⁹F-NMR (376 MHz, DMSO-d₆) δ −74.59, −116.10, −140.33, −153.20, −161.96.

Condensation of Compound 2, after HATU and DIEA activation, with methyl 3-aminopropanoate produced methyl 3-(2-(2,2,7-trifluoro-3-oxo-6-(perfluorophenyl)-2,3-dihydro-4H-benzo[b][1,4]oxazin-4-yl)acetamido)propanoate (Compound 94): MS (ESI) calculated for C₂₀H₁₂F₈N₂O₅ [M+1]⁺=513.0, found 513.2; ¹H-NMR (400 MHz, DMSO-d₆) δ 8.46-8.40 (m, 1H), 7.77 (d, J=9.6 Hz, 1H), 7.49 (d, J=6.4 Hz, 2H), 4.65 (s, 3H), 3.56 (s, 3H), 2.48-2.43 (m, 2H); ¹⁹F-NMR (376 MHz, DMSO-d₆) δ −74.55, −116.14, −140.40, −153.21, −162.03; which, after treatment with trimethylstannanol in DCE at 80° C., produced 3-(2-(2,2,7-trifluoro-3-oxo-6-(perfluorophenyl)-2,3-dihydro-4H-benzo[b][1,4]oxazin-4-yl)acetamido)propanoic acid (Compound 95): MS (ESI) calculated for C₁₉H₁₀FN₂O₅ [M+1]⁺=499.0, found 499.1, ¹H-NMR (400 MHz, DMSO-d₆) δ8.44-8.41 (m, 1H), 7.77 (d, J=9.6 Hz, 1H), 7.50 (d, J=6.4 Hz, 1H), 4.65 (s, 2H), 3.28-3.24 (m, 2H), 2.31 (t, J=6.8 Hz, 2H); ¹⁹F-NMR (377 MHz, DMSO-d₆) δ −74.57, −119.18, −140.45, −153.10, −161.96.

Condensation of Compound 2, after HATU and DIEA activation, with N-methyl-1-phenylmethanamine produced N-benzyl-N-methyl-2-(2,2,7-trifluoro-3-oxo-6-(perfluorophenyl)-2,3-dihydro-4H-benzo[b][1,4]oxazin-4-yl)acetamide (Compound 96): MS (ESI) calculated for C₂₄H₁₄ClF₈N₂O₃ [M+1]⁺=531.0, found 531.1; ¹H-NMR (400 MHz, DMSO-d₆) δ 7.79 (d, J=9.2 Hz, 1H), 7.67 (d, J=6.4 Hz, 1H), 7.47-7.36 (m, 1H), 7.36-7.26 (m, 3H), 7.22 (d, J=7.2 Hz, 1H), 5.12-4.95 (m, 2H), 4.73-4.47 (m, 2H), 3.04 (s, 2H), 2.85 (s, 1H); ¹⁹F-NMR (376 MHz, DMSO-d₆) δ −75.09, −116.12, −140.28, −152.90, −161.84.

Condensation of Compound 2, after HATU and DIEA activation, with N,O-dimethylhydroxylamine, hydrochloride produced N-methoxy-N-methyl-2-(2,2,7-trifluoro-3-oxo-6-(perfluorophenyl)-2,3-dihydro-4H-benzo[b][1,4]oxazin-4-yl)acetamide (Compound 97: MS (ESI) calculated for C₁₈H₁₀F₈N₂O₄ [M+1]⁺=471.0, found, 471.0; ¹H-NMR (400 MHz, DMSO-d₆) δ 7.78 (d, J=9.6 Hz, 1H), 7.70 (d, J=6.4 Hz, 1H), 4.99 (s, 2H), 3.82 (s, 3H), 3.15 (s, 3H); ¹⁹F-NMR (377 MHz, DMSO-d₆) δ −75.46, −116.06, −140.24, −153.20, −161.99.

Condensation of Compound 2, after propanephosphonic acid anhydride (T₃P) and triethylamine activation, with N-methylhydroxylamine produced N-hydroxy-N-methyl-2-(2,2,7-trifluoro-3-oxo-6-(perfluorophenyl)-2,3-dihydro-4H-benzo[b][1,4]oxazin-4-yl)acetamide (Compound 98, 30 mg, 29% yield) as a white solid: MS (ESI) calculated for C₁₇H₈F₈N₂O₄ [M−1]⁻=455.0, found 454.9; ¹H-NMR (400 MHz, DMSO-d₆) δ 10.24 (s, 1H), 7.77 (d, J=9.6 Hz, 1H), 7.54 (d, J=6.4 Hz, 1H), 4.93 (s, 2H), 3.14 (s, 3H); ¹⁹F-NMR (400 MHz, DMSO-d₆) δ −75.23, −116.16, −140.27, −153.23, −162.02.

Condensation of Compound 2, after HATU and DIEA activation, with phenylmethanamine produced N-benzyl-2-(2,2,7-trifluoro-3-oxo-6-(perfluorophenyl)-2,3-dihydro-4H-benzo[b][1,4]oxazin-4-yl)acetamide (Compound 99): MS (ESI) calculated for C₂₃H₁₂ClFN₂O₃ [M+1]⁺=517.1, found 517.1; ¹H-NMR (400 MHz, DMSO-d₆) δ 8.80 (s, 1H), 7.78 (d, J=9.6 Hz, 1H), 7.57 (d, J=6.4 Hz, 1H), 7.36-7.21 (m, 5H), 4.71 (s, 2H), 4.30 (d, J=5.6 Hz, 3H). ¹⁹F-NMR (376 MHz, DMSO-d₆) δ −74.26, −116.15, −140.28, −153.09, −161.98;

condensation of Compound 2, after HATU and DIEA activation, with methyl (R)-thiomorpholine-3-carboxylate produced methyl (R)-4-(2-(2,2,7-trifluoro-3-oxo-6-(perfluorophenyl)-2,3-dihydro-4H-benzo[b][1,4]oxazin-4-yl)acetyl)thiomorpholine-3-carboxylate (Compound 107): MS (ESI) calculated for C₂₂H₁₄F₈N₂O₅S [M−1]⁻=569.0, found 568.9; ¹H-NMR (400 MHz, DMSO-d₆) δ 7.84-7.74 (m, 1H), 7.46-7.34 (m, 1H), 5.47-5.26 (m, 2H), 5.04-4.88 (m, 1H), 4.59-4.16 (m, 1H), 3.68 (s, 3H), 3.16-2.98 (m, 1H), 2.93-2.73 (m, 2H), 2.70-2.53 (m, 2H); ¹⁹F-NMR (376 MHz, DMSO-d₆) δ −74.04, −76.77, −115.88, −140.29, −152.70, −161.85; which, after treatment with trimethylstannanol in DCE at 80° C., produced (R)-4-(2-(2,2,7-trifluoro-3-oxo-6-(perfluorophenyl)-2,3-dihydro-4H-benzo[b][1,4]oxazin-4-yl)acetyl)thiomorpholine-3-carboxylic acid (Compound 108): MS (ESI) calculated for C₂₁H₁₂F₈N₂O₅S [M−1]⁻=555.0, found 554.9; ¹H-NMR (400 MHz, DMSO-d₆) δ 13.10 (s, 1H), 7.82-7.74 (m, 1H), 7.46-7.35 (m, 1H), 5.34-5.05 (m, 2H), 4.95-4.55 (m, 1H), 4.18-3.44 (m, 2H), 3.08-2.77 (m, 3H), 2.66-2.55 (m, 1H); ¹⁹F-NMR (377 MHz, DMSO-d₆) δ −74.17, −76.76, −115.89, −140.52, −152.96, −161.69.

Condensation of Compound 2, after HATU and DIEA activation, with methyl (5)-piperidine-2-carboxylate produced methyl (S)-1-(2-(2,2,7-trifluoro-3-oxo-6-(perfluorophenyl)-2,3-dihydro-4H-benzo[b][1,4]oxazin-4-yl)acetyl)piperidine-2-carboxylate (Compound 109): MS (ESI) calculated for (C₂₃H₁₆F₈N₂O₅) [M+1]⁺=553.0, found 553.1; ¹H-NMR (400 MHz, DMSO-d₆) δ 7.79 (d, J=8.4 Hz, 1H), 7.42-7.37 (m, 1H), 5.28-4.90 (m, 3H), 4.91-4.75 (m, 1H), 4.27-3.79 (m, 1H), 3.64 (s, 3H), 3.28-3.12 (m, 1H), 2.26-2.15 (m, 1H), 1.73-1.48 (m, 4H); ¹⁹F-NMR (376 MHz, DMSO-d₆) δ −73.49, −77.37, −115.96, −140.28, −152.73, −161.93; which, after treatment with trimethylstannanol in DCE at 80° C., produced (S)-1-(2-(2,2,7-trifluoro-3-oxo-6-(perfluorophenyl)-2,3-dihydro-4H-benzo[b][1,4]oxazin-4-yl)acetyl)piperidine-2-carboxylic acid (Compound 110): MS (ESI) calculated for (C₂₂H₁₄F₈N₂O₅) [M+1]⁺=539.0, found 539.1; ¹H-NMR (400 MHz, DMSO-d₆) δ13.02 (s, 1H), 7.78 (d, J=9.6 Hz, 1H), 7.45-7.30 (m, 1H), 5.31-5.13 (m, 1H), 5.06-4.63 (m, 2H), 4.33-3.79 (m, 1H), 3.29-3.16 (m, 1H), 2.24-2.07 (m, 1H), 1.69-1.66 (m, 2H), 1.61-1.45 (m, 1H), 1.40-1.17 (m, 2H); ¹⁹F-NMR (376 MHz, DMSO-d₆) δ −74.96, −77.26, −116.01, −140.54, −153.04, −161.65.

Condensation of Compound 2, after HATU and DIEA activation, with methyl (R)-thiomorpholine-3-carboxylate 1,1-dioxide produced methyl (R)-4-(2-(2,2,7-trifluoro-3-oxo-6-(perfluorophenyl)-2,3-dihydro-4H-benzo[b][1,4]oxazin-4-yl)acetyl)thiomorpholine-3-carboxylate 1,1-dioxide (Compound 119): MS (ESI) calculated for C₂₂H₁₄F₈N₂O₇S [M−1]⁻=601.0, found 600.9; ¹H-NMR (400 MHz, DMSO-d₆) δ7.83-7.73 (m, 1H), 7.51-7.25 (m, 1H), 5.82-5.46 (m, 1H), 5.46-5.11 (m, 2H), 5.10-4.70 (m, 1H), 4.59-3.78 (m, 2H), 3.73-3.64 (m, 3H), 3.64-3.50 (m, 2H), 3.22-3.20 (m, 1H); ¹⁹F-NMR (376 MHz, DMSO-d₆) δ −74.24, −76.79, −115.78, −140.24, −152.63, −161.86; which, after treatment with trimethylstannanol in DCE at 80° C., produced, after chiral preparative HPLC. (R)-4-(2-(2,2,7-trifluoro-3-oxo-6-(perfluorophenyl)-2,3-dihydro-4H-benzo[b][1,4]oxazin-4-yl)acetyl)thiomorpholine-3-carboxylic acid 1,1-dioxide (Compound 120): MS (ES) calculated for C₂₁H₁₂F₈N₂O₇S [M−1]⁻=587.0, found 587.0; ¹H-NMR (400 MHz, DMSO-d₆) δ 7.80-7.77 (m, 1H), 7.51-7.21 (m, 1H), 5.50-5.06 (m, 2H), 4.73-4.69 (m, 2H), 4.59-3.33 (m, 4H), 3.23-3.13 (m, 2H); ¹⁹F-NMR (376 MHz, DMSO-d₆) δ −74.20, −76.69, −116.10, −140.40, −153.10, −161.79; and (S)-4-(2-(2,2,7-trifluoro-3-oxo-6-(perfluorophenyl)-2,3-dihydro-4H-benzo[b][1,4]oxazin-4-yl)acetyl)thiomorpholine-3-carboxylic acid 1,1-dioxide (Compound 121): MS (ESI) calculated for (C₂₁H₁₂F₈N₂O₇S [M−1]⁻=587.0, found 587.0; ¹H-NMR (400 MHz, DMSO-d₆) δ7.80-7.77 (m, 1H), 7.51-7.21 (m, 1H), 5.50-5.06 (m, 2H), 4.73-4.69 (m, 2H), 4.59-3.33 (m, 4H), 3.23-3.13 (m, 2H); ¹⁹F-NMR (376 MHz, DMSO-d₆) δ −74.20, −76.70, −115.84, −140.48, −153.10, −161.99.

Condensation of Compound 2, after HATU and DIEA activation, with methyl (S)-morpholine-3-carboxylate produced methyl (S)-4-(2-(2,2,7-trifluoro-3-oxo-6-(perfluorophenyl)-2,3-dihydro-4H-benzo[b][1,4]oxazin-4-yl)acetyl)morpholine-3-carboxylate (Compound 126): MS (ESI) calculated for C₂₂H₁₄F₈N₂O₆ [M−1]⁻=553.0, found 552.9; ¹H-NMR (400 MHz, DMSO-d₆) δ 7.85-7.71 (m, 1H), 7.46-7.25 (m, 1H), 5.40-5.13 (m, 1H), 4.96-4.69 (m, 2H), 4.37-4.16 (m, 1H), 4.04-3.84 (m, 1H), 3.84-3.71 (m, 1H), 4.27-3.66 (m, 4H), 3.62-3.32 (m, 2H); ¹⁹F-NMR (377 MHz, DMSO-d₆) δ −73.29, −77.14, −115.91, −140.40, −152.76, −161.87; which, after treatment with trimethylstannanol in DCE at 80° C., produced (S)-4-(2-(2,2,7-trifluoro-3-oxo-6-(perfluorophenyl)-2,3-dihydro-4H-benzo[b][1,4]oxazin-4-yl)acetyl)morpholine-3-carboxylic acid (Compound 127): MS (ESI) calculated for C₂₁H₂F₈N₂O₆ [M−1]⁻=538.9, found 538.9; ¹H-NMR (400 MHz, methanol-d₄) δ 7.56-7.27 (m, 2H), 5.38-5.28 (m, 1H), 4.82-4.75 (m, 1H), 4.63-4.45 (m, 1H), 4.43-4.40 (m, 1H), 3.99-3.82 (m, 1H), 3.79-3.48 (m, 3H), 3.10-3.04 (m, 1H); ¹⁹F-NMR (377 MHz, methanol-d₄) δ −76.19, −79.90, −117.49, −142.25, −156.58, −164.92.

Condensation of Compound 2, after HATU and DIEA activation, with 1-(tert-butyl) 3-methyl (S)-piperazine-1,3-dicarboxylate produced 1-(tert-butyl) 3-methyl (S)-4-(2-(2,2,7-trifluoro-3-oxo-6-(perfluorophenyl)-2,3-dihydro-4H-benzo[b][1,4]oxazin-4-yl)acetyl)piperazine-1,3-dicarboxylate; LCMS calculated for C₂₇H₂₃F₈NiO₇, [M+1]=654.1, found 654, which, after treatment with TFA in DCM, produced methyl (S)-1-(2-(2,2,7-trifluoro-3-oxo-6-(perfluorophenyl)-2,3-dihydro-4H-benzo[b][1,4]oxazin-4-yl)acetyl)piperazine-2-carboxylate (Compound 145): MS (ESI) calculated for C₂H₁₅F₈N₂O₅ [M+1]⁺=554.1, found 554.1; ¹H-NMR (400 MHz, DMSO-d₆) δ7.79-7.77 (m, 1H), 7.38-7.33 (m, 1H), 5.32-5.23 (m, 1H), 4.88-4.82 (m, 2H), 4.02-3.99 (m, 1H), 3.75-3.72 (m, 1H), 3.66-3.60 (m, 2H), 3.44-3.41 (m, 1H), 3.26-3.25 (m, 2H), 2.88-2.85 (m, 1H), 2.75-2.71 (m, 1H), 2.68-2.60 (m, 1H); ¹⁹F-NMR (400 MHz, DMSO-d₆) δ −76.9, −115.9, −140.63, −153.06, −161.90; which, after treatment with trimethylstannanol in DCE at 80° C., produced (0.5)-1-(2-(2,2,7-trifluoro-3-oxo-6-(perfuorophenyl)-2,3-dihydro-4H-benzo[b][1,4]oxazin-4-yl)acetyl)piperazine-2-carboxylic acid (Compound 148): MS (ESI) calculated for C₂₁H₁₃F₈N₃O₅ [M+1]⁺=540.0, found 540.0; ¹H-NMR (400 MHz, DMSO-d₆) δ 7.90-7.88 (m, 1H), 7.78-7.76 (m, 1H), 5.26-5.22 (m, 1H), 4.89-4.84 (m, 1H), 4.50-4.46 (m, 1H), 4.05-4.03 (m, 1H), 3.74-3.72 (m, 1H), 3.56-3.52 (m, 1H), 2.97-2.92 (m, 2H), 2.87-2.83 (m, 2H); ¹⁹F-NMR (400 MHz, DMSO-d₆) δ −70.99, −78.37, −116.32, −140.23, −153.41, −162.0; or, after treatment of Compound 145 with acetyl chloride and triethylamine in DCM, produced methyl (0.5)-4-acetyl-1-(2-(2,2,7-trifluoro-3-oxo-6-(perfluorophenyl)-2,3-dihydro-4H-benzo[b][1,4]oxazin-4-yl)acetyl)piperazine-2-carboxylate (Compound 146): MS (ES) calculated for C₂₄H₁₇F₈N₃O₆ [M+1]⁺=596.1, found 596.1; ¹H-NMR (400 MHz, DMSO-d₆) δ 7.79 (d, J=9.6 Hz, 1H), 7.47-7.28 (m, 1H), 5.34-5.24 (m, 1H), 5.12-4.79 (m, 2H), 4.43-4.05 (m, 2H), 3.99-3.76 (m, 1H), 3.65 (d, J=9.6 Hz, 2H), 3.59-3.47 (m, 2H), 3.10-2.78 (m, 1H), 2.71-2.53 (m, 1H), 2.07-2.00 (m, 3H); ¹⁹F-NMR (377 MHz, DMSO-d₆) δ −73.82, −76.98, −115.87, −140.14, −152.73, −161.87; which, after treatment with trimethylstannanol in DCE at 80° C., produced (S)-4-acetyl-1-(2-(2,2,7-trifluoro-3-oxo-6-(perfluorophenyl)-2,3-dihydro-4H-benzo[b][1,4]oxazin-4-yl)acetyl)piperazine-2-carboxylic acid (Compound 147): MS (ES) calculated for C₂₃H₁₅F₈N₃O₆ [M−1]⁻=580.1, found 580.0; H-NMR (400 MHz, DMSO-d₆) δ 13.08 (b, 1H), 7.78 (d, J=9.6 Hz, 1H), 7.46-7.20 (m, 1H), 5.46-5.18 (m, 1H), 5.04-4.55 (m, 2H), 4.40-4.03 (m, 2H), 3.98-3.73 (m, 1H), 3.64-3.43 (m, 1H), 3.15-2.85 (m, 1H), 2.71-2.53 (m, 1H), 2.08-1.93 (m, 3H); ¹⁹F-NMR (400 MHz, DMSO-d₆) δ −73.81, −76.52, −115.94, −140.60, −153.06, −161.50.

### Example 4. Preparation of methyl (S)-3-(2-(2,2,7-trifluoro-3-oxo-6-(perfluomphenyl)-2,3-dihydro-4H-benzo[b][1,4]oxazin-4-yl)propanamido)propanoate (Compound 130) and methyl (R)-3-(2-(2,2,7-trifluoro-3-oxo-6-(perfluorophenyl)-2,3-dihydro-4H-benzo[b][1,4]oxazin-4-yl)propanamido)propanoate (Compound 131)

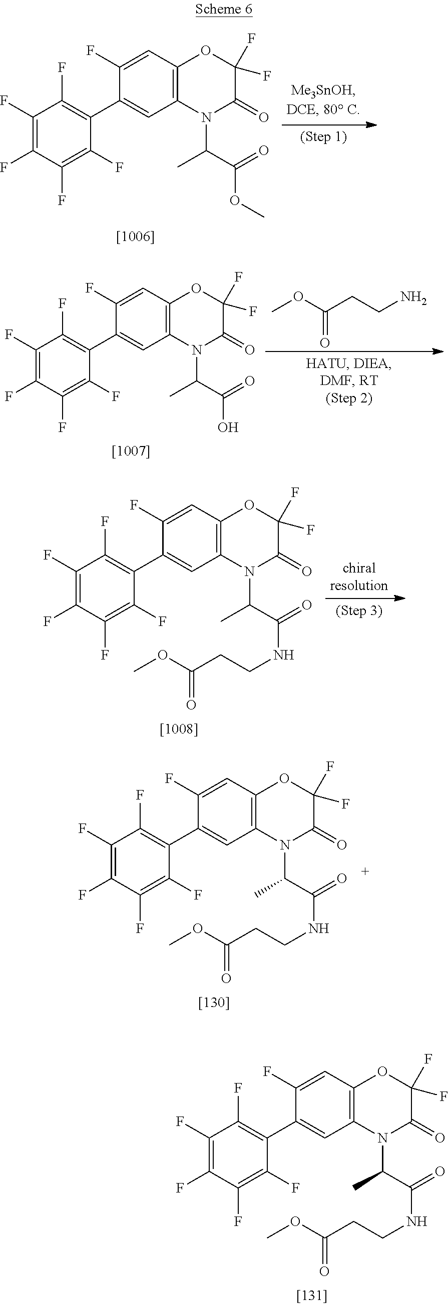

As shown in Step 1 of Scheme 6, a mixture of methyl 2-(2,2,7-trifluoro-3-oxo-6-(perfluorophenyl)-2,3-dihydro-4H-benzo[b][1,4]oxazin-4-yl)propanoate (Compound 1006, 720 mg, 1.58 mmol) and trimethylstannyl (141 mg, 1.90 mmol) in dichloroethane (8 mL) was stirred at 80° C. for 16 hours, cooled to room temperature, diluted with ethyl acetate, washed with brine, dried over anhydrous sodium sulfate, filtered, and concentrated under reduced pressure. The residue was purified by flash chromatography (0%-60% ethyl acetate in petroleum ether) to afford racemic 2-(2,2,7-trifluoro-3-oxo-6-(perfluorophenyl)-2,3-dihydro-4H-benzo[b][1,4]oxazin-4-yl)propanoic acid (Compound 1007, 103 mg, 14% yield) as a white solid: MS (ESI) calculated for C₁₇H₇F₈NO₄ [M+1]⁺=440.0, found 439.4; ¹H-NMR (400 MHz, DMSO-d₆) δ 13.26 (s, 1H), 7.79 (t, J=6.8 Hz, 2H), 5.33 (d, J=7.2 Hz, 1H), 1.53 (d, J=7.2 Hz, 3H); ¹⁹F-NMR (400 MHz, DMSO-d₆H) δ −75.96, −115.72, −140.33, −153.07, −162.04.

As shown in Step 2 of Scheme 6, to a solution of 2-[2,2,7-trifluoro-3-oxo-6-(2,3,4,5,6-pentafluorophenyl)-1,4-benzoxazin-4-yl]propanoic acid (300 mg, 0.68 mmol) in DMF (6 mL) methyl 3-aminopropanoate (18 mg, 0.17 mmol), DIEA (44 mg, 0.34 mmol) and HATU (54 mg, 0.14 mmol). The resulting mixture was stirred at room temperature for 2 hours under a nitrogen atmosphere, diluted with water, and extracted with ethyl acetate. The combined organic layers were washed with brine, dried over anhydrous sodium sulfate, filtered, and concentrated under reduced pressure. The residue was purified by reversed-phase flash chromatography (5%-69% acetonitrile in water) to afford racemic methyl 3-(2-(2,2,7-trifluoro-3-oxo-6-(perfluorophenyl)-2,3-dihydro-4H-benzo[B][1,4]oxazin-4-yl)propanamido)propanoate (Compound 1008, 21 mg, 33% yield) as a white solid: MS (ESI) calculated for C₂₁H₁₄F₈N₂O₅ [M−1]⁻=525.1, found 524.9. ¹H-NMR (400 MHz, methanol-d₄) δ 7.44-7.39 (m, 2H), 5.46 (d, J=7.2 Hz, 1H), 3.61 (s, 3H), 3.45-3.42 (m, 2H), 2.47 (t, J=2.4 Hz, 2H), 1.63 (d, J=7.2 Hz, 3H), ¹⁹F-NMR (376 MHz, methanol-d₄) δ −78.99, −116.76, −142.50, −156.28, −164.78.

As shown in Step 3 of Scheme 6, racemic methyl 3-(2-(2,2,7-trifluoro-3-oxo-6-(perfluorophenyl)-2,3-dihydro-4H-benzo[b][1,4]oxazin-4-yl)propanamido)propanoate (70 mg) was separated by preparative chiral HPLC using the following conditions—column: CHIRAL ART Cellulose-SC, 2×25 cm, 5 μm, eluted with 50% iPrOH/hexanes to afford methyl (R)-3-(2-(2,2,7-trifluoro-3-oxo-6-(perfluorophenyl)-2,3-dihydro-4H-benzo[b][1,4]oxazin-4-yl)propanamido)propanoate (Compound 131, 28 mg) as a white solid: MS (ESI) calculated for C₂₁H₁₄F₈N₂O₅ [M+1]⁺=527.1, found 527.0; ¹H-NMR (400 MHz, DMSO-d₆) δ 8.31 (t, J=6.4 Hz, 1H), 7.77 (d, J=8.8 Hz, 1H), 7.46 (d, J=6.4 Hz, 1H), 5.32-5.30 (m, 1H), 3.53 (s, 3H), 3.27-3.24 (m, 2H), 2.40-2.35 (m, 2H), 1.50 (d, J=7.2 Hz, 3H); ¹⁹F-NMR (376 MHz, DMSO-d₆) δ −74.53, −115.94, −140.73, −153.20, −162.14. Also recovered with a longer retention time was methyl (S)-3-(2-(2,2,7-trifluoro-3-oxo-6-(perfluorophenyl)-2,3-dihydro-4H-benzo[b][1,4]oxazin-4-yl)propanamido)propanoate (Compound 130, 28 mg) as a white solid: MS (ESI) calculated for C₂₁H₁₄F₈N₂O₅ [M+1]⁺=527.1, found 527.0; ¹H-NMR (400 MHz, DMSO-d₆) δ 8.31 (t, J=6.4 Hz, 1H), 7.77 (d, J=8.8 Hz, 1H), 7.46 (d, J=6.4 Hz, 1H), 5.32-5.30 (m, 1H), 3.53 (s, 3H), 3.27-3.24 (m, 2H), 2.40-2.35 (m, 2H), 1.50 (d, J=7.2 Hz, 3H); ¹⁹F-NMR (376 MHz, DMSO-d₆) δ −74.53, −115.94, −140.73, −153.20, −162.04.

Reaction of Compound 1007 with methyl 3-(methylamino)propanoate, under the HATU coupling conditions of Step 2 of Scheme 6, produced racemic methyl 3-(N-methyl-2-(2,2,7-trifluoro-3-oxo-6-(perfluorophenyl)-2,3-dihydro-4H-benzo[B][1,4]oxazin-4-yl)propanamido)propanoate: MS (ESI) calculated for C₂₂H₁₆F₈N₂O₅ [M+1]⁺=541.1, found 541.0; which when separated into its enantiomers by chiral HPLC produced methyl (S)-3-(N-methyl-2-(2,2,7-trifluoro-3-oxo-6-(perfluorophenyl)-2,3-dihydro-4H-benzo[b][1,4]oxazin-4-yl)propanamido)propanoate (Compound 132): MS (ESI) calculated for C₂₂H₁₆F₈N₂O₅ [M+1]⁺ =541.1, found 541.0; ¹H-NMR (400 MHz, DMSO-d₆) δ 7.82-7.74 (m, 2H), 5.73-5.65 (m, 1H), 3.67-3.58 (m, 1H), 3.55 and 3.49 (s, 3H), 3.38 (t, J=6.8 Hz, 1H), 2.83 and 2.78 (s, 3H), 2.47-2.30 (m, 2H), 1.52-1.42 (m, 3H); ¹⁹F-NMR (377 MHz, DMSO-d₆) δ −74.09, −77.52, −15.08, −140.85, −152.78, −161.78; which was subsequently hydrolyzed using trimethylstannanol in DCE at 80° C. to produce (S)-3-(N-methyl-2-(2,2,7-trifluoro-3-oxo-6-(perfluorophenyl)-2,3-dihydro-4H-benzo[b][1,4]oxazin-4-yl)propanamido)propanoic acid (Compound 134): MS (ESI) calculated for C₂₁H₁₄F₈N₂O₅ [M+1]⁺=527.1, found 527.0; ¹H-NMR (400 MHz, DMSO-d₆) δ 12.30 (b, 1H), 7.88-7.72 (m, 2H), 6.03-5.57 (m, 1H), 3.62-3.36 (m, 2H), 2.84 and 2.78 (s, 3H), 2.60-2.51 (m, 1H), 2.38-2.25 (m, 1H), 1.49-1.24 (m, 3H); ¹⁹F-NMR (377 MHz, DMSO-d₆) δ −73.87, −76.68, −115.09, −140.82, −152.76, −161.74; and also produced methyl (R)-3-(N-methyl-2-(2,2,7-trifluoro-3-oxo-6-(perfluorophenyl)-2,3-dihydro-4H-benzo[b][1,4]oxazin-4-yl)propanamido)propanoate (Compound 133): MS (ESI) calculated for C₂₂H₁₆F₈N₂O₅ [M+1]⁺=541.1, found 541.0; ¹H-NMR (400 MHz, DMSO-d₆) δ 7.82-7.72 (m, 2H), 5.75-5.65 (m, 1H), 3.66-3.56 (m, 1H), 3.55-3.52 (m, 1H), 3.50-3.43 (m, 2H), 3.42-3.34 (m, 1H), 2.83 and 2.78 (s, 3H), 2.48-2.29 (m, 2H), 1.52-1.42 (m, 3H); ¹⁹F-NMR (377 MHz, DMSO-d₆) −74.11, −77.52, −115.10, −140.85, −152.79, −161.78; which was subsequently hydrolyzed using trimethylstannanol in DCE at 80° C. to produce (R)-3-(N-methyl-2-(2,2,7-trifluoro-3-oxo-6-(perfluorophenyl)-2,3-dihydro-4H-benzo[b][1,4]oxazin-4-yl)propanamido)propanoic acid (Compound 135): MS (ESI) calculated for C₂₁H₁₄F₈N₂O₅ [M+1]⁺=527.1, found 527.0; ¹H-NMR (400 MHz, DMSO-d₆) δ 12.23 (b, 1H), 7.83-7.72 (m, 2H), 5.90-5.67 (m, 1H), 3.57-3.51 (m, 1H), 3.49-3.34 (m, 1H), 2.84 and 2.78 (s, 3H), 2.43-2.22 (m, 2H), 1.52-1.42 (m, 3H); ¹⁹F-NMR (377 MHz, DMSO-d₆) δ −74.19, −77.39, −115.07, −140.82, −152.84, −161.78.

### Example 5. Preparation of methyl 2-(2,2,7-trifluoro-3-oxo-6-(2,3,4,6-tetrafluorophenyl)-2,3-dihydro-4H-benzo[b][1,4]oxazin-4-yl)acetate (Compound 64) and 2-(2,2,7-trifluoro-3-oxo-6-(2,3,4,6-tetrafluorophenyl)-2,3-dihydro-4H-benzo[b][1,4]oxazin-4-yl)acetic acid (Compound 65)

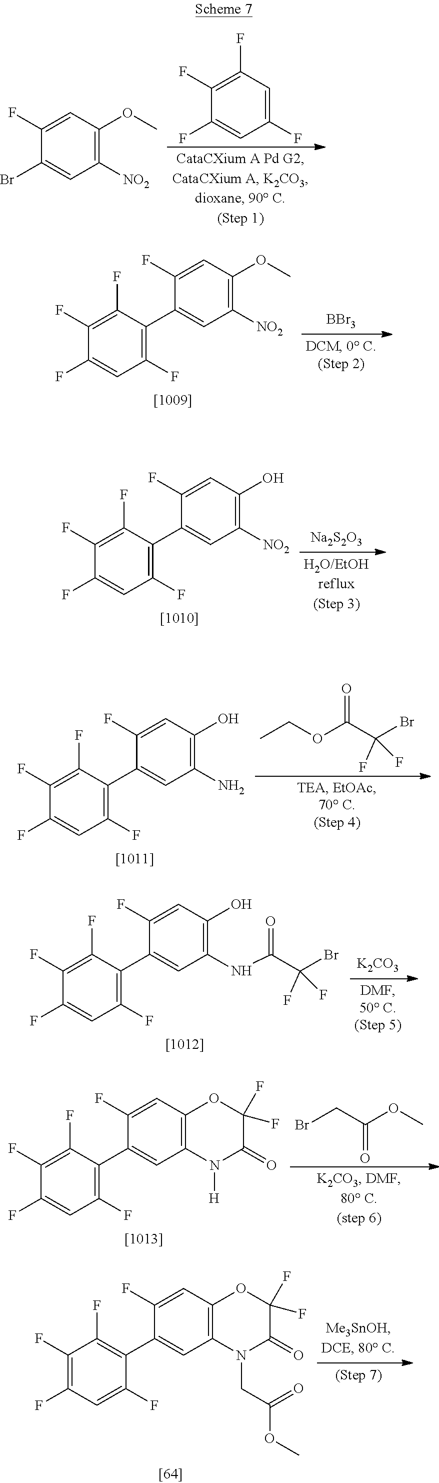

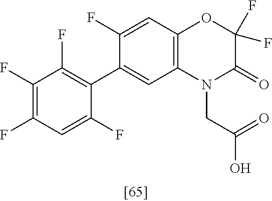 [SMILES: O=C(O)CN1C(=O)C(F)(F)Oc2cc(F)c(-c3c(F)cc(F)c(F)c3F)cc21]

As shown in Step 1 of Scheme 7, a solution of 1-bromo-2-fluoro-4-methoxy-5-nitrobenzene (20.0 g, 80.0 mmol), 1,2,3,5-tetrafluorobenzene (36.0 g, 24.0 mmol), K₂PO₄ (33.9 g, 160 mmol), chloro[(diadamantan-1-yl)(n-butyl)phosphino][2-amino-1,1-biphenyl-2-yl]palladium(II) (2.7 g, 4.0 mmol), bis(adamantan-1-vi)(butyl)phosphane (1.4 g, 4.0 mmol) in dioxane (150 mL) was stirred at 90° C. for 16 hours under an atmosphere of nitrogen. The solvent was removed under reduced pressure and the residue purified by reversed-phase flash chromatography (5%-52% acetonitrile in water) to afford 2,2′,3,4,6-pentafluoro-4′-methoxy-5′-nitro-1,1′-biphenyl (Compound 1009, 11.0 g, 38% yield) as a light yellow solid: GCMS calculated for C₁₃H₆F₅NO₃=319.0, found 319.0.

As shown in Step 2 of Scheme 7, to a stirred mixture of 2,2′,3,4,6-pentafluoro-4′-methoxy-5′-nitro-1,1′-biphenyl (1.0 g, 3.13 mmol) in DCM (10 mL) was added boron tribromide (3.9 g, 15.7 mmol) dropwise at 0° C. under an atmosphere of nitrogen. The mixture was stirred at 0° C. for 3 hours, diluted with water, and extracted with dichloromethane. The combined organic layers were washed with brine, dried over anhydrous sodium sulfate, filtered, and concentrated under reduced pressure to afford 2,2′,3′,4′,6′-pentafluoro-5-nitro-[1,1′-biphenyl]-4-ol (Compound 1010, 790 mg, 74% yield) as a brown solid: MS (ESI) calculated for C₁₂H₄F₅NO₃ [M−1]⁻=304.0, found 303.9.

As shown in Step 3 of Scheme 7, to a stirred solution of 2,2′,3′,4′,6′-pentafluoro-5-nitro-[1,1′-biphenyl]-4-ol (790 mg, 2.58 mmol) in water (5 mL) and EtOH (5 mL) was added sodium hyposulfite (2.0 g, 12.94 mmol). The resulting mixture was stirred at 100° C. for 2 hours, cooled to room temperature, diluted with water, and extracted with dichloromethane. The combined organic layers were washed with brine, dried over anhydrous sodium sulfate, filtered, and concentrated under reduced pressure. The residue was purified by flash chromatography (0%-25% ethyl acetate in petroleum ether) to afford 5-amino-2,2′,3′,4′,6′-pentafluoro-[1,1′-biphenyl]-4-ol (Compound 1011, 580 mg, 59% yield) as a yellow solid: MS (ESI) calculated for C₁₂H₆F₅NO [M−1]⁻=274.0, found 274.0.

As shown in Step 4 of Scheme 7, to a solution of 5-amino-2,2′,3′,4′,6′-pentafluoro-[1,1′-biphenyl]-4-ol (580 mg, 2.10 mmol) and triethylamine (427 mg, 4.21 mmol) in EtOAc (5 mL) was added ethyl 2-bromo-2,2-difluoroacetate (856 mg, 4.21 mmol). The resulting mixture was stirred at 80° C. for 2 hours, cooled to room temperature, diluted with water, and extracted with dichloromethane. The combined organic layers were washed with brine, dried over anhydrous sodium sulfate, filtered, and concentrated under reduced pressure to afford 2-bromo-2,2-difluoro-N-(2′,3′,4′,6,6′-pentafluoro-4-hydroxy-[1,1-biphenyl]-3-yl)acetamide (Compound 1012, 800 mg, 75% yield) as a brown oil: MS (ESI) calculated for C₁₄H₅BrF₇NO₂ [M−1]⁻=429.9, found 430.0.

As shown in Step 5 of Scheme 7, a stirred solution of 2-bromo-2,2-difluoro-N-(2′,3′,4′,6,6′-pentafluoro-4-hydroxy-[1,1′-biphenyl]-3-yl)acetamide (870 mg, 2.01 mmol) and K₂CO₃ (417 mg, 3.02 mmol) in DMF (10 mL) was stirred at 50° C. for 2 hours, cooled to room temperature, diluted with water, and extracted with dichloromethane. The combined organic layers were washed with brine, dried over anhydrous sodium sulfate, filtered, and concentrated under reduced pressure. The residue was purified by reversed-phase flash chromatography (5%-80% acetonitrile in water) to afford 2,2,7-trifluoro-6-(2,3,4,6-tetrafluorophenyl)-2H-benzo[b][1,4]oxazin-3(4H)-one (Compound 1013, 250 mg, 31% yield) as a brown solid: MS (ESI) calculated for C₁₄H₄F₇NO₂ [M−1]⁻=350.0, found 350.2; ¹H-NMR (400 MHz, DMSO-d₆) δ 12.14 (s, 1H), 7.71-7.65 (m, 1H), 7.62 (d, J=9.6 Hz, 1H), 7.20 (d, J=6.4 Hz, 1H); ¹⁹F-NMR (376 MHz, DMSO-d₆) δ −75.34, −115.87, −116.95, −131.54, −134.11, −164.81.

As shown in Step 6 of Scheme 7, to a stirred mixture of 2,2,7-trifluoro-6-(2,3,4,6-tetrafluorophenyl)-2H-benzo[b][1,4]oxazin-3(4H)-one (450 mg, 1.28 mmol) in DMF (3 mL) were added K₂CO₃ (354 mg, 2.56 mmol) and methyl 2-bromoacetate (294 mg, 1.92 mmol). The resulting mixture was stirred at 25° C. under nitrogen atmosphere for 2 hours, diluted with water, and extracted with ethyl acetate. The combined organic layers were washed with brine, dried over anhydrous sodium sulfate, filtered, and concentrated under reduced pressure. The residue was purified by flash chromatography (0%-50% ethyl acetate in petroleum ether) to afford methyl 2-(2,2,7-trifluoro-3-oxo-6-(2,3,4,6-tetrafluorophenyl)-2,3-dihydro-4H-benzo[b][1,4]oxazin-4-yl)acetate (Compound 64, 54 mg, 9% yield) as a white solid: GCMS calculated for C₁₇H₈F₇NO₄=423.0, found 423.0; ¹H-NMR (400 MHz, methanol-d₄) δ 7.45-7.35 (m, 2H), 7.39-7.21 (m, 1H), 4.90 (s, 2H), 3.79 (s, 3H); ¹⁹F-NMR (377 MHz, methanol-d₄) δ −79.00, −117.03, −133.42, −135.32, −167.69.

As shown in Step 7 of Scheme 7, to a stirred mixture of methyl 2-(2,2,7-trifluoro-3-oxo-6-(2,3,4,6-tetrafluorophenyl)-2,3-dihydro-4H-benzo[b][1,4]oxazin-4-yl)acetate (340 mg, 0.80 mmol) in DCE (2 mL) was added trimethylstannanol (292 mg, 1.60 mmol). The resulting mixture was stirred at 65° C. for 16 hours under a nitrogen atmosphere, cooled to room temperature, diluted with water, acidified to pH 4 to 5 with formic acid, and extracted with ethyl acetate. The combined organic layers were washed with brine, dried over anhydrous sodium sulfate, filtered, and concentrated under reduced pressure. The residue was purified by reversed-phase flash chromatography (5% to 50% acetonitrile in water) to afford 2-(2,2,7-trifluoro-3-oxo-6-(2,3,4,6-tetrafluorophenyl)-2,3-dihydro-4H-benzo[b][1,4]oxazin-4-yl)acetic acid (Compound 65, 52 mg, 15% yield) as a yellow semi-solid: MS (ESI) calculated for C₁₆H₆F₇NO₄ [M−1]⁻=408.0, found 407.9; ¹H-NMR (400 MHz, DMSO-d₆) δ 7.80-7.68 (m, 3H), 4.72 (s, 2H); ¹⁹F-NMR (376 MHz, DMSO-d₆) δ −75.44, −115.12, −116.31, −131.44, −133.46, −165.01.

Alkylation of Compound 1013 with methyl 2-bromopropanoate, using cesium carbonate in acetonitrile at 65° C., produced racemic methyl 2-(2,2,7-trifluoro-3-oxo-6-(2,3,4,6-tetrafluorophenyl)-2,3-dihydro-4H-benzo[b][1,4]oxazin-4-yl)propanoate, which was subsequently separated into its enantiomers methyl (2S)-2-(2,2,7-trifluoro-3-oxo-6-(2,3,4,6-tetrafluorophenyl)-2,3-dihydro-4H-benzo[b][1,4]oxazin-4-yl)propanoate (Compound 76): GCMS calculated for C₁₈H₁₀F₇NO₄=437.0, found 437.0; ¹H-NMR (400 MHz, methanol-d₄) δ 7.49 (d, J=6.4 Hz, 1H), 7.39 (d, J=9.2 Hz, 1H), 7.33-7.26 (m, 1H), 5.37 (q, J=7.2 Hz, 1H), 3.76 (s, 3H), 1.65 (d, J=7.2 Hz, 3H); ¹⁹F-NMR (376 MHz, methanol-d₄) δ −78.57, −81.67, −116.96, −117.45, −133.25, −135.67, −167.60; and methyl (2R)-2-(2,2,7-trifluoro-3-oxo-6-(2,3,4,6-tetrafluorophenyl)-2,3-dihydro-4H-benzo[b][1,4]oxazin-4-yl)propanoate (Compound 77): GCMS calculated for C₁₈H₁₀F₇NO₄=437.0, found 437.0; ¹H-NMR (400 MHz, methanol-d₄) δ 7.48 (d, J=6.4 Hz, 1H), 7.40 (d, J=9.6 Hz, 1H), 7.33-7.26 (m, 1H), 5.37 (q, J=7.2 Hz, 1H), 3.76 (s, 3H), 1.65 (d, J=7.2 Hz, 3H); ¹⁹F-NMR (376 MHz, methanol-d₄) δ −78.55, −81.68, −116.62, −117.45, −133.21, −135.64, −167.60; which were each subsequently treated with trimethylstannanol in DCE at 65° C. to provide, respectively, (2S)-2-(2,2,7-trifluoro-3-oxo-6-(2,3,4,6-tetrafluorophenyl)-2,3-dihydro-4H-benzo[b][1,4]oxazin-4-yl)propanoic acid (Compound 78): MS (ESI) calculated for C₁₇H₈F₇NO₄ [M−1]⁻=422.0, found 422.1; ¹H-NMR (400 MHz, DMSO-d₆) δ 13.18 (s, 1H), 7.81-7.73 (m, 3H), 5.33 (d, J=6.8 Hz, 1H), 1.53 (d, J=7.2 Hz, 3H); ¹⁹F-NMR (376 MHz, DMSO-d₆) δ −75.96, −115.12, −115.90, −131.26, −133.49, −164.94; and (2R)-2-(2,2,7-trifluoro-3-oxo-6-(2,3,4,6-tetrafluorophenyl)-2,3-dihydro-H-benzo[b][1,4]oxazin-4-yl)propanoic acid (Compound 79): MS (ESI) calculated for (C₁₇H₈F₇NO₄) [M−1]⁻=422.0, found 421.8; ¹H-NMR (400 MHz, methanol-d₄) δ 7.46 (d, J=6.4 Hz, 1H), 7.39 (d, J=8.8 Hz, 1H), 7.31-7.25 (m, 2H), 5.41-5.35 (m, 1H), 1.65 (d, J=7.2 Hz, 3H); ¹⁹F-NMR (376 MHz, methanol-d₄) δ −75.52, −82.02, −116.89, −133.30, −135.38, −167.62.

Condensation of Compound 65, after activation using EDC and triethylamine, with dimethyl(sulfamoyl)amine produced N-(N,N-dimethylsulfamoyl)-2-(2,2,7-trifluoro-3-oxo-6-(2,3,4,6-tetrafluorophenyl)-2,3-dihydro-4H-benzo[b][1,4]oxazin-4-yl)acetamide (Compound 93): MS (ESI) calculated for C₁₈H₁₂F₇N₃O₅S [M−1]⁻=514.0, found 514.0; ¹H-NMR (400 MHz, methanol-d₄) δ 7.43-7.38 (m, 2H), 7.33-7.23 (m, 1H), 4.84 (s, 2H), 2.89 (s, 6H); ¹⁹F NMR (400 MHz, methanol-d₄) δ −78.71, −116.65, −117.03, −133.31, −135.13, −167.65.

Condensation of Compound 65, after activation using HATU and DEA, with methyl 3-(methylamino)propanoate produced methyl 3-(N-methyl-2-(2,2,7-trifluoro-3-oxo-6-(2,3,4,6-tetrafluorophenyl)-2,3-dihydro-4H-benzo[b][1,4]oxazin-4-yl)acetamido)propanoate (Compound 162): MS (ESI) calculated for C₂₁H₁₅F₇N₂₀₅ [M+1]⁺=509.1, found, 509.0; ¹H-NMR (400 MHz, DMSO-d₆) δ 7.79-7.69 (m, 2H), 7.57-7.47 (m, 1H), 5.07 (s, 1H), 4.93 (s, 1H), 3.64 (t, J=6.4 Hz, 1H), 3.54 (d, J=6.4 Hz, 4H), 3.08 (s, 2H), 2.80 (s, 1H), 2.75 (t, J=6.4 Hz, 1H), 2.49 (d, J=7.6 Hz, 1H); ¹⁹F-NMR (377 MHz, DMSO-d₆) δ −75.69, −115.46, −116.02, −131.50, −133.71, −164.87; which after treatment with trimethylstannanol in DCE at 65° C., produced 3-(N-methyl-2-(2,2,7-trifluoro-3-oxo-6-(2,3,4,6-tetrafluorophenyl)-2,3-dihydro-4H-benzo[b][1,4]oxazin-4-yl)acetamido)propanoic acid (Compound 163): MS (ESI) calculated for C₂₀H₁₃F₇N₂O₅ [M+1]⁺=495.1, found 495.0; ¹H-NMR (400 MHz, DMSO-d₆) δ 12.29 (s, 1H), 7.78-7.68 (m, 2H), 7.58-7.48 (m, 1H), 5.08 (s, 1H), 4.93 (s, 1H), 3.64-3.56 (m, 1H), 3.47-3.46 (m, 1H), 3.09 & 2.82 (s, 3H), 2.65-2.54 (m, 1H), 2.48-2.45 (m, 1H); ¹⁹F-NMR (377 MHz, DMSO-d₆) δ −75.29, −115.15, −116.47, −131.33, −133.65, −164.78.

Condensation of Compound 65, after activation using HATU and DIEA, with methyl azetidine-3-carboxylate produced methyl 1-(2-(2,2,7-trifluoro-3-oxo-6-(2,3,4,6-tetrafluorophenyl)-2,3-dihydro-4H-benzo[b][1,4]oxazin-4-yl)acetyl)azetidine-3-carboxylate (Compound 164): MS (ESI) calculated for C₂₁H₁₃F₇N₂O₅ [M+1]⁺=507.1, found 507.0; ¹H NMR (400 MHz, DMSO-d₆) δ 7.82-7.68 (m, 2H), 7.59 (s, 1H), 4.74 (s, 2H), 4.44-4.54 (m, 1H), 4.30-4.43 (m, 1H), 4.06-4.16 (m, 1H), 3.92-4.05 (m, 1H), 3.69 (s, 3H), 3.57-3.68 (m, 1H); ¹⁹F-NMR (377 MHz, DMSO-d₆) δ −74.75, −115.01, −116.27, −131.22, −133.50, −164.76; which after treatment with trimethylstannanol in DCE at 65° C., produced 1-(2-(2,2,7-trifluoro-3-oxo-6-(2,3,4,6-tetrafluorophenyl)-2,3-dihydro-4H-benzo[b][1,4]oxazin-4-yl)acetyl)azetidine-3-carboxylic acid (Compound 165): MS (ESI) calculated for C₂₀H₁₁F₇N₂O₅ [M−1]⁻1=491.1, found 491.0; ¹H-NMR (400 MHz, DMSO-d₆) δ 12.83 (b, 1H), 7.68-7.84 (m, 2H), 7.60 (s, 1H), 4.74 (s, 2H), 4.46-4.54 (m, 1H), 4.35-4.53 (m, 1H), 4.07-4.17 (m, 1H), 3.82-3.98 (m, 1H), 3.48-3.63 (m, 1H); ¹⁹F-NMR (377 MHz, DMSO-d₆) δ −75.23, −115.04, −116.29, −131.18, −133.49, −164.75.

### Example 6. Preparation of methyl 2-(2,2,7-trifluoro-3-oxo-6-(2,3,5,6-tetrafluorophenyl)-2,3-dihydro-4H-benzo[b][1,4]oxazin-4-yl)acetate (Compound 6) and 2-(2,2,7-trifluoro-3-oxo-6-(2,3,5,6-tetrafluorophenyl)-2,3-dihydro-4H-benzo[b][1,4]oxazin-4-yl)acetic acid (Compound 7)

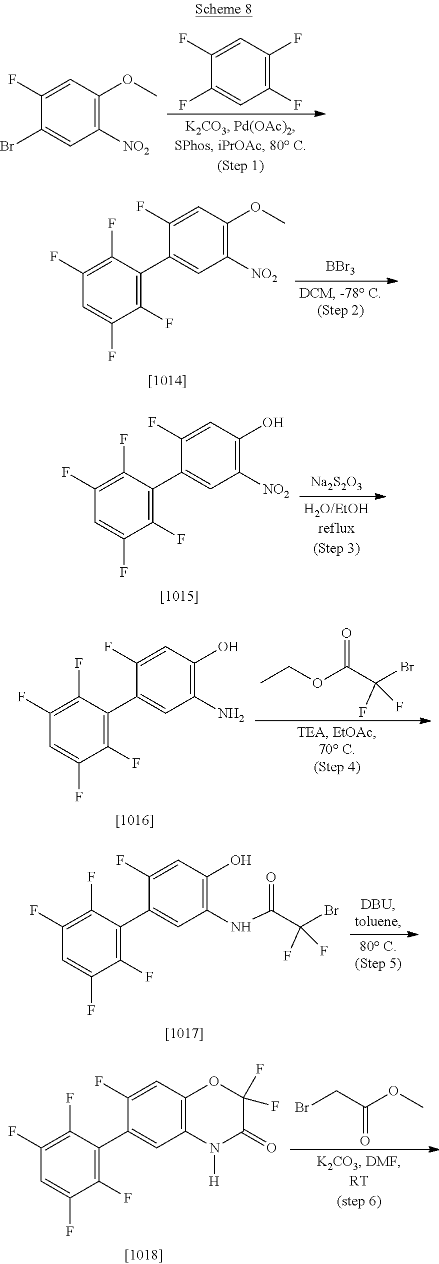

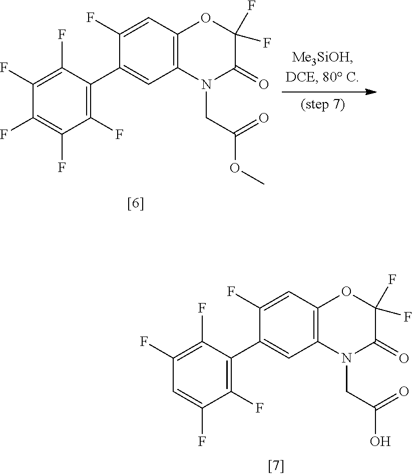 [SMILES: COC(=O)CN1C(=O)C(F)(F)Oc2cc(F)c(-c3c(F)c(F)c(F)c(F)c3F)cc21 ; O=C(O)CN1C(=O)C(F)(F)Oc2cc(F)c(-c3c(F)c(F)cc(F)c3F)cc21]

As shown in Step 1 of Scheme 8, to a stirred mixture of dicyclohexyl(2′,6′-dimethoxy[1,1′-biphenyl]-2-yl)phosphane (Sphos, 990 mg, 2.40 mmol). Pd(OAc)₂ (270 mg, 1.20 mmol) and K₂CO₃ (3.3 g, 24.0 mmol) in isopropyl acetate (20 mL) was added 1,2,4,5-tetrafluorobenzene (3.6 g, 24.0 mmol) at room temperature under an atmosphere of nitrogen. The resulting mixture was stirred at room temperature for 10 minutes under nitrogen and 1-bromo-2-fluoro-4-methoxy-5-nitrobenzene (3.0 g, 12.0 mmol) in isopropyl acetate (10 mL) was added dropwise over 0.5 hours at 80° C. The resulting mixture was stirred at 80° C. for additional 2 hours, the solvent removed under reduced pressure, and the residue purified by flash chromatography (0%-40% EtOAc in petroleum ether) to afford 2,2′,3,5,6-pentafluoro-4′-methoxy-5′-nitro-1,1′-biphenyl (Compound 1014, 1.5 g, 35% yield) as a purple solid: GCMS calculated for C₁₃H₆F₅NO₃=319.0, found, 319.0.

As shown in Step 2 of Scheme 8, to a mixture of 2,2′,3,5,6-pentafluoro-4′-methoxy-5′-nitro-1,1′-biphenyl (1.5 g, 4.69 mmol) in DCM (15 mL) at −78° C. was added BBr, (5.8 g, 23.5 mmol) dropwise. The resulting mixture was stirred at −78° C. for 3 hours under an atmosphere of nitrogen, diluted with water, and extracted with CH₂Cl₂. The combined organic solution was dried over sodium sulfate, filtered, and concentrated to about 20% volume under reduced pressure, filtered, and the filtrate concentrated under reduced pressure to afford 2,2′,3′,5′,6′-pentafluoro-5-nitro-[1,1′-biphenyl]-4-ol (Compound 1015, 1.3 g, crude) as a brown solid: MS (ESI) calculated for C₁₂H₄F₅NO₃ [M−1]⁻=304.0, found 304.0. This material was used in subsequent steps without further purification.

As shown in Step 3 of Scheme 8, to a stirred solution of 2,2′,3′,5′,6′-pentafluoro-5-nitro-[1,1′-biphenyl]-4-ol (1.4 g, 4.58 mmol) in EtOH (10 mL) and H₂O (3 mL) was added sodium hyposulfite (3.9 g, 22.91 mmol) in portions. The resulting mixture was stirred at 100° C. for 1 hour under an atmosphere of nitrogen, cooled to room temperature, diluted with water, and extracted with ethyl acetate. The combined organics dried over sodium sulfate, filtered, and concentrated under reduced pressure to afford 5-amino-2,2′,3′,5′,6′-pentafluoro-[1,1′-biphenyl]-4-ol (Compound 1016, 1.2 g) as a yellow solid: MS (ESI) calculated for C₁₂H₅F₆NO [M−1]⁻=292.1, found 292.1. This material was used as is in subsequent reactions.

As shown in Step 4 of Scheme 8, to a solution of 5-amino-2,2′,3′,5′,6′-pentafluoro-[1,1′-biphenyl]-4-ol (500 mg, 1.81 mmol) and triethylamine (184 mg, 1.81 mmol) in EtOAc (5 mL) was added ethyl 2-bromo-2,2-difluoroacetate (369 mg, 1.81 mmol). The resulting mixture was stirred at 80° C. for 5 hours under an atmosphere of nitrogen, cooled to room temperature, diluted with water, and extracted with ethyl acetate. The combined organic layers were washed with brine, dried over anhydrous sodium sulfate, filtered, and concentrated under reduced pressure. The residue was purified by flash chromatography (0%-38% EtOAc in petroleum ether) to afford 2-bromo-2,2-difluoro-N-(2′,3′,5′,6,6′-pentafluoro-4-hydroxy-[1,1′-biphenyl]-3-yl) acetamide (Compound 1017, 300 mg, 38% yield) as a brown solid: MS (ES) calculated for C₁₄H₅BrF₇NO₂ [M+1]⁺=431.9, found 431.9.

As shown in Step 5 of Scheme 8, a solution of 2-bromo-2,2-difluoro-N-[2′,3′,5′,6,6′-pentafluoro-4-hydroxy-[1,1′-biphenyl]-3-yl]acetamide (500 mg, 1.15 mmol) and 1,8-diazabicyclo[5.4.0]undec-7-ene (DBU, 178 mg, 1.16 mmol) in toluene (5 ml) was stirred at 80° C. for 2 hours under an atmosphere of nitrogen. The reaction solution was diluted with water and extracted with ethyl acetate. The combined organic layers were washed with brine, dried over anhydrous sodium sulfate, filtered, and concentrated under reduced pressure. The residue was purified by flash chromatography (0%-30% ethyl acetate in petroleum ether) to afford 2,2,7-trifluoro-6-(2,3,5,6-tetrafluorophenyl)-2H-benzo[b][1,4]oxazin-3(4H)-one (Compound 1018, 230 mg, 55% yield) as a brown solid: MS (ESI) calculated for C₁₄H₄F₇NO₂ [M−1]⁻=350.0, found 350.0; ¹H-NMR (400 MHz, DMSO-d₆) δ 12.20 (s, 1H), 8.07 (m, 1H), 7.70-7.62 (m, 1H), 7.24 (d, J=6.4 Hz, 1H); ¹⁹F-NMR (376 MHz, DMSO-dh) δ −75.21, −116.80, −138.87, −141.58.

As shown in Step 6 of Scheme 8, to a solution of 2,2,7-trifluoro-6-(2,3,5,6-tetrafluorophenyl)-2H-benzo[B][1,4]oxazin-3(4H)-one (50 mg, 0.14 mmol) in DMF (1 mL) were added K₂CO₃ (59 mg, 0.42 mmol) and methyl 2-bromoacetate (26 mg, 0.17 mmol). The mixture was stirred at room temperature for 16 hours, diluted with water, and extracted with ethyl acetate. The combined organic layers were washed with brine, dried over anhydrous sodium sulfate, filtered, and concentrated under reduced pressure. The residue was purified by reversed-phase preparative HPLC [55% to 68% acetonitrile/water (10 mM NH₄HCO₃)] to afford 2-(2,2,7-trifluoro-3-oxo-6-(2,3,5,6-tetrafluorophenyl)-2,3-dihydro-4H-benzo[b][1,4]oxazin-4-yl)acetate (Compound 6, 10 mg, 16% yield) as an off-white solid: GCMS calculated for C₁₇H₈F₇NO₄=423.0, found 423.0; ¹H-NMR (400 MHz, DMSO-d₆) δ 8.11 (s, 1H), 7.86-7.75 (m, 2H), 4.88 (s, 2H), 3.71 (s, 3H); ¹⁹F-NMR (376 MHz, CDCl) δ −75.03, −112.96, −137.17, −139.25.

As shown in Step 7 of Scheme 8, to a solution of methyl 2-(2,2,7-trifluoro-3-oxo-6-(perfluorophenyl)-2,3-dihydro-4H-benzo[b][1,4]oxazin-4-yl)acetate (460 mg, 1.08 mmol) in DCE (5 mL) was added trimethystannanol (394 mg, 2.17 mmol) in portions at 20° C. under a nitrogen atmosphere. The resulting mixture was stirred at 80° C. under a nitrogen atmosphere for 2 hours, cooled to room temperature, diluted with water, acidified to pH 4-5 with formic acid, and extracted with ethyl acetate. The combined organic layers were washed with brine, dried over anhydrous sodium sulfate, filtered, and concentrated under reduced pressure. The residue purified by reversed-phase flash chromatography (5%-70% acetonitrile in water) to afford 2-(2,2,7-trifluoro-3-oxo-6-(2,3,5,6-tetrafluorophenyl)-2,3-dihydro-4H-benzo[b][1,4]oxazin-4-yl)acetic acid (Compound 7, 228 mg, 51% yield) as a white solid: MS (ESI) calculated for C₁₆H₆F₇NO₄ [M−1]⁻=408.0, found 408.0; ¹H-NMR (400 MHz, DMSO-d₆) δ 13.41 (s, 1H), 8.17-8.03 (m, 1H), 7.90-7.63 (m, 2H), 4.77 (s, 2H); ¹⁹F-NMR (376 MHz, DMSO-d₆) δ −75.44, −116.08, −139.00, −140.86.

Alkylation of Compound 1018 with methyl 2-bromopropanoate, using potassium carbonate in DMF at 25° C., produced racemic methyl 2-(2,2,7-trifluoro-3-oxo-6-(2,3,5,6-tetrafluorophenyl)-2,3-dihydro-4H-benzo[b][1,4]oxazin-4-yl)propanoate, which was subsequently separated into its enantiomers methyl (S)-2-(2,2,7-trifluoro-3-oxo-6-(2,3,5,6-tetrafluorophenyl)-2,3-dihydro-4H-benzo[b][1,4]oxazin-4-yl)propanoate (Compound 66): GCMS calculated for C₁₈H₁₀F₇NO₄=437.0, found 437.0; ¹H-NMR (400 MHz, methanol-d₄) δ 7.67-7.54 (m, 2H), 7.44 (d, J=9.2 Hz, 1H), 5.42-5.32 (m, 1H), 3.76 (s, 3H), 1.66 (d, J=7.2 Hz, 3H); ¹⁹F-NMR (377 MHz, methanol-d₄) δ −78.72, −81.35, −116.49, −140.98; and methyl (R)-2-(2,2,7-trifluoro-3-oxo-6-(2,3,5,6-tetrafluorophenyl)-2,3-dihydro-4H-benzo[b][1,4]oxazin-4-yl)propanoate (Compound 67): GCMS calculated for C₁₈H₁₀F₇NO₄=437.0, found 437.0; ¹H-NMR (400 MHz, methanol-d₄) δ 7.67-7.54 (m, 2H), 7.44 (d, J=9.2 Hz, 1H), 5.42-5.32 (m, 1H), 3.76 (s, 3H), 1.66 (d, J=7.2 Hz, 3H); ¹⁹F-NMR (377 MHz, methanol-d₄) δ −78.72, −81.35, −116.49, −140.98; which were each subsequently treated with trimethylstannanol in DCE at 65° C. to provide, respectively, (S)-2-(2,2,7-trifluoro-3-oxo-6-(2,3,5,6-tetrafluorophenyl)-2,3-dihydro-4H-benzo[b][1,4]oxazin-4-yl)propanoic acid (Compound 68): MS (ESI) calculated for C₁₇H₈F₇NO₄ [M−1]⁻=422.0, found 421.9; ¹H-NMR (400 MHz, DMSO-d₆) δ8.14-8.05 (m, 1H), 7.72 (d, J=9.6 Hz, 1H), 7.61-7.43 (m, 1H), 5.19-5.12 (m, 1H), 1.45 (d, J=7.2 Hz, 3H); ¹⁹F-NMR (376 MHz, DMSO-dh) δ −72.60, −78.91, −116.65, −138.72, −141.95; and (R)-2-(2,2,7-trifluoro-3-oxo-6-(2,3,5,6-tetrafluorophenyl)-2,3-dihydro-4H-benzo[b][1,4]oxazin-4-yl)propanoic acid (Compound 69): MS (ESI) calculated for C₁₇H₈F₇NO₄ [M−1]⁻=422.0, found 421.9; ¹H-NMR (400 MHz, DMSO-d₆) δ 8.16-8.02 (m, 1H), 7.70 (d, J=9.6 Hz, 1H), 7.43 (d, J=6.4 Hz, 1H), 5.11 (d, J=7.6 Hz, 1H), 1.44 (d, J=7.2 Hz, 3H); ¹⁹F-NMR (376 MHz, DMSO-d₆) δ −71.89, −79.60, −116.89, −138.64, −142.29.

### Example 7. Preparation of methyl 2-(2,2,7-trifluoro-3-oxo-6-(2,3,4,5-tetrafluorophenyl)-2,3-dihydro-4H-benzo[b][1,4]oxazin-4-yl)acetate (Compound 158) and 2-(2,2,7-trifluoro-3-oxo-6-(2,3,4,5-tetrafluorophenyl)-2,3-dihydro-4H-benzo[b][1,4]oxazin-4-yl)acetic acid (Compound 159)

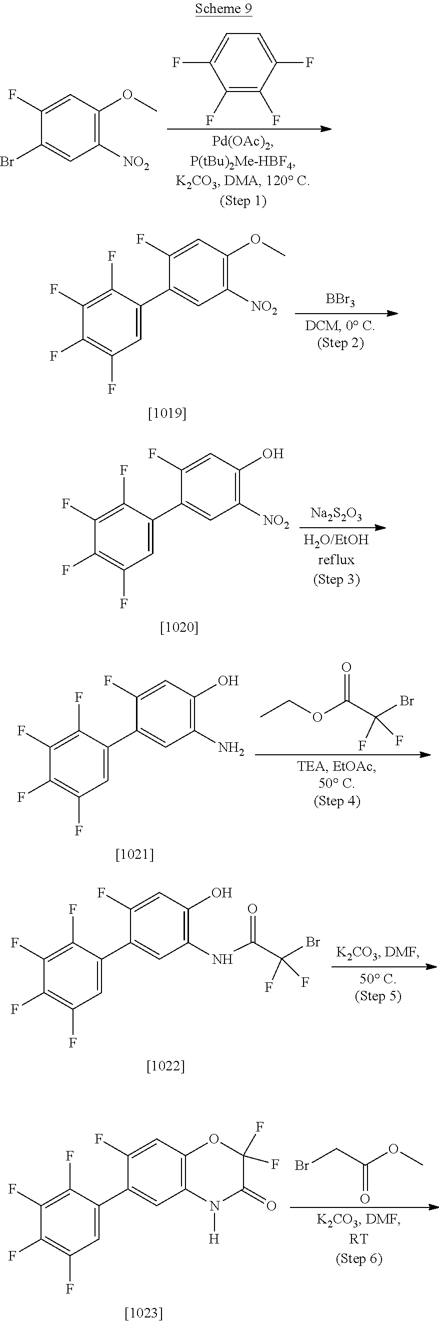

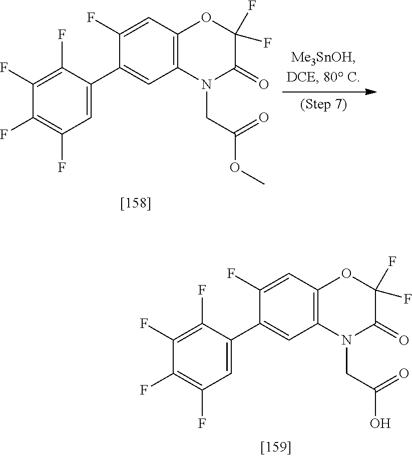 [SMILES: COC(=O)CN1C(=O)C(F)(F)Oc2cc(F)c(-c3cc(F)c(F)c(F)c3F)cc21 ; O=C(O)CN1C(=O)C(F)(F)Oc2cc(F)c(-c3cc(F)c(F)c(F)c3F)cc21]

As shown in Step 1 of Scheme 9, a mixture of 1,2,3,4-tetrafluorobenzyne (3 equiv.), 1-bromo-2-fluoro-4-methoxy-5-nitrobenzene (1 equiv.), Pd(OAc₂) (0.1 equiv.), di-tert-butylmethylphosphine (0.1 equiv.), and K₂CO₃ (1 equiv.) in dioxane are heated to 90° C. for 16 hours, cooled to room temperature, diluted with water, and extracted with EtOAc. The combined extracts are washed with brine, dried over anhydrous sodium sulfate, filtered, and concentrated under reduced pressure. The residue is purified by reversed-phase flash chromatography to yield 2,2′,3,4,5-pentafluoro-4′-methoxy-5′-nitro-1,1′-biphenyl (Compound 1019).

As shown in Step 2 of Scheme 9, to a stirred mixture of 2,2′,3,4,5-pentafluoro-4′-methoxy-5′-nitro-1,1′-biphenyl (1 equiv.) in DCM is added boron tribromide (5 equiv.) dropwise at 0° C. under an atmosphere of nitrogen. The mixture is stirred at 0° C. for 3 hours, diluted with water, and extracted with dichloromethane. The combined organic layers are washed with brine, dried over anhydrous sodium sulfate, filtered, and concentrated under reduced pressure to afford 2,2′,3′,4′,5′-pentafluoro-5-nitro-[1,1′-biphcnyl]-4-ol (Compound 1020).

As shown in Step 3 of Scheme 9, to a stirred solution of 2,2′,3′,4′,5′-pentafluoro-5-nitro-[1,1′-biphenyl]-4-ol (1 equiv) in 1:1 water/EtOH is added sodium hyposulfite (5 equiv.). The resulting mixture is stirred at 100° C. for 2 hours, cooled to room temperature, diluted with water, and extracted with dichloromethane. The combined organic layers are washed with brine, dried over anhydrous sodium sulfate, filtered, and concentrated under reduced pressure. The residue is purified by flash chromatography to afford 5-amino-2,2′,3′,4′,5′-pentafluoro-[1,1′-biphenyl]-4-ol (Compound 1021).

As shown in Step 4 of Scheme 9, to a stirred solution of 5-amino-2,2′,3′,4′,5′-pentafluoro-[1,1′-biphenyl]-4-ol (1 equiv.) and TEA (2 equiv.) in EtOAc is added and ethyl 2-bromo-2,2-difluoroacetate (2 equiv.) in portions at 20° C. The resulting mixture is stirred at 50° C. for 16 hours under nitrogen, cooled to room temperature, diluted with water, and extracted with ethyl acetate. The combined organic layers are washed with brine, dried over anhydrous sodium sulfate, filtered, concentrated under vacuum, and purified by reversed-phase flash chromatography to afford 2-bromo-2,2-difluoro-N-(2′,3′,4′,5′,6-pentafluoro-4-hydroxy-[1,1′-biphenyl]-3-yl)acetamide (Compound 1022).

As shown in Step 5 of Scheme 9, to a stirred solution of 2-bromo-2,2-difluoro-N-(2′,3′,4′,5′,6-pentafluoro-4-hydroxy-[1,1′-biphenyl]-3-yl)acetamide (1 equiv.) in DMF is added K₂CO₃ (2 equiv.) in portions at 20° C. The resulting mixture is stirred at 50° C. for 2 hours under nitrogen, diluted with water, and extracted with ethyl acetate. The combined organic layers are washed with brine, dried over anhydrous sodium sulfate, filtered, concentrated under reduced pressure, and purified by flash chromatography to afford 2,2,7-trifluoro-6-(2,3,4,5-tetrafluorophenyl)-2H-benzo[b][1,4]oxazin-3(4H)-one (Compound 1023).

As shown in Step 6 of Scheme 9, to a stirred solution of 2,2,7-trifluoro-6-(2,3,4,5-tetrafluorophenyl)-2H-benzo[b][1,4]oxazin-3(4H)-one (1 equiv.) and methyl 2-bromoacetate (1.2 equiv.) in DMF is added K₂CO₃ (2 equiv.) at room temperature under a nitrogen atmosphere. The mixture is stirred at room temperature for 2 hours, diluted with water, and extracted with ethyl acetate. The combined organic layers are washed with brine, dried over anhydrous sodium sulfate, filtered, concentrated under reduced pressure, and purified by reversed-phase preparative HPLC to afford methyl 2-(2,2,7-trifluoro-3-oxo-6-(2,3,4,5-tetrafluorophenyl)-2,3-dihydro-4H-benzo[b][1,4]oxazin-4-yl)acetate (Compound 158): GCMS calculated for C₁₇H₈F₇NO₄=423.0; found 423.0; ¹H-NMR (400 MHz, DMSO-d₆) δ 7.81-7.63 (m, 3H), 4.96 (s, 2H), 3.73 (s, 3H); ¹⁹F-NMR (376 MHz, DMSO-d₆) δ −76.04, −82.16, −116.48, −139.33, −155.52, −156.00.

As shown in Step 7 of Scheme 9, to a stirred mixture of methyl 2-(2,2,7-trifluoro-3-oxo-6-(2,3,4,5-tetrafluorophenyl)-2,3-dihydro-4H-benzo[b][1,4]oxazin-4-yl)acetate (1 equiv.) in DCE is added trimethylstannanol (2 equiv.). The resulting mixture is stirred at 65° C. for 16 hours under a nitrogen atmosphere, cooled to room temperature, diluted with water, acidified to pH 4 to 5 with formic acid, and extracted with ethyl acetate. The combined organic layers are washed with brine, dried over anhydrous sodium sulfate, filtered, and concentrated under reduced pressure. The residue is purified by reversed-phase flash chromatography to afford 2-(2,2,7-trifluoro-3-oxo-6-(2,3,4,5-tetrafluorophenyl)-2,3-dihydro-4H-benzo[b][1,4]oxazin-4-yl)acetic acid (Compound 159): MS (ESI) calculated for C₁₆H₆F₇NO₄ [M−1]⁻=408.0, found 407.9; ¹H-NMR (400 MHz, DMSO-d₆) δ 13.40 (b, 1H), 7.78-7.60 (m, 3H), 4.83 (s, 2H); ¹⁹F-NMR (377 MHz, DMSO-d₆) δ −75.88, −82.09, −116.77, −139.39, −155.63, −156.00.

Condensation of Compound 159, after activation using N,N,N′,N′-tetramethylchlorofomiamidinium hexafluorophosphate (TCFH) and N-methylimidazole (NMI), with methyl 3-(methylamino)propanoate produced methyl 3-(N-methyl-2-(2,2,7-trifluoro-3-oxo-6-(2,3,4,5-tetrafluorophenyl)-2,3-dihydro-4H-benzo[b][1,4]oxazin-4-yl)acetamido)propanoate (Compound 166): MS (ESI) calculated for C₂₁H₁₅F₇N₂O₅ [M+1]⁺=509.1, found 509.1, ¹H-NMR (400 MHz, DMSO-d₆) δ 7.74-7.59 (m, 2H), 7.48-7.38 (m, 1H), 5.12 (s, 1H), 4.99 (s, 1H), 3.65 (t, J=6.4 Hz, 1H), 3.58-3.57 (m, 1H), 3.38 (s, 3H), 3.09 & 2.78 (s, 3H), 2.75-2.65 (m, 1H), 2.48-2.46 (m, 1H); ¹⁹F-NMR (377 MHz, DMSO-d₆) δ −75.68, −117.21, −139.43, −155.16, −156.00; which after treatment with trimethylstannanol in DCE at 65° C., produced 3-(N-methyl-2-(2,2,7-trifluoro-3-oxo-6-(2,3,4,5-tetrafluorophenyl)-2,3-dihydro-4H-benzo[b][1,4]oxazin-4-yl)acetamido)propanoic acid (Compound 167): MS (ESI) calculated for C₂₀H₁₃F₇N₂O₅ [M+1]⁺=495.0, found 494.9; ¹H-NMR (400 MHz, DMSO-d₆) δ 12.36 (s, 1H), 7.73-7.59 (m, 2H), 7.44 (t, J=6.4 Hz, 1H), 5.13 (s, 1H), 4.98 (s, 1H), 3.68-3.58 (m, 1H), 3.49 (t, J=7.2 Hz, 1H), 3.10 (s, 2H), 2.82 (s, 1H), 2.66-2.62 (m, 1H), 2.43-2.38 (m, 1H), ¹⁹F-NMR (377 MHz, DMSO-d₆) δ −75.66, −117.23, −139.40, −155.33, −155.88.

Condensation of Compound 159, after activation using N,N,N′,N′-tetramethylchloroformamidinium hexafluorophosphate (TCFH) and N-methylimidazole (NMI), with methyl azetidine-3-carboxylate produced methyl 1-(2-(2,2,7-trifluoro-3-oxo-6-(2,3,4,5-tetrafluorophenyl)-2,3-dihydro-4H-benzo[b][1,4]oxazin-4-yl)acetyl)azetidine-3-carboxylate (Compound 168): MS (ESI) calculated for C₂₁H₁₃F₇N₂O₅ [M+1]⁺=507.1, found 507.1; ¹H-NMR (400 MHz, DMSO-d₆) δ 7.68 (m, 2H), 7.49 (d, J=6.4 Hz, 1H), 4.80 (s, 2H), 4.50 (t, J=8.8 Hz, 1H), 4.46-4.37 (m, 1H), 4.12 (t, J=9.2 Hz, 1H), 3.97-3.95 (m, 1H), 3.69 (s, 3H), 3.62-3.60 (m, 1H); ¹⁹F-NMR (377 MHz, DMSO-d₆) δ −75.63, −116.95, −139.36, −155.19, −155.74, −155.81; which aftertreatment with trimethylstannanol in DCE at 80° C. produced 1-(2-(2,2,7-trifluo-3-oxo-6-(2,3,4,5-tetrafluorophenyl)-2,3-dihydro-4H-benzo[b][1,4]oxazin-4-yl)acetyl)azetidine-3-carboxylic acid (Compound 169): MS (ESI) calculated for C₂₀H₁₁F₇N₂O₅ [M+1]⁺=493.1, found 493.0, ¹H-NMR (400 MHz, DMSO-d₆) δ 12.78 (br, 1H), 7.70-7.64 (m, 2H), 7.50 (d, J=6.4 Hz, 1H), 4.80 (s, 2H), 4.47 (d, J=8.4 Hz, 1H), 4.40-4.36 (m, 1H), 4.08 (t, J=9.6 Hz, 1H), 3.96-3.92 (m, 1H), 3.48-3.41 (m, 1H); ¹⁹F-NMR (377 MHz, DMSO-d₆) δ −75.61, −116.98, −139.37, −155.18, −155.70, −155.81.

### Example 8. Preparation of methyl 2-(2,2,7-trifluoro-3-oxo-6-(2,3,5,6-tetrafluoro-methoxyphenyl)-2,3-dihydro-4H-benzo[b][1,4]oxazin-4-yl)acetate (Compound 103) and 2-(2,2,7-trifluoro-3-oxo-6-(2,3,5,6-tetrafluoro-4-methoxyphenyl)-2,3-dihydro-4H-benzo[b][1,4]oxazin-4-yl)acetic acid (Compound 104)

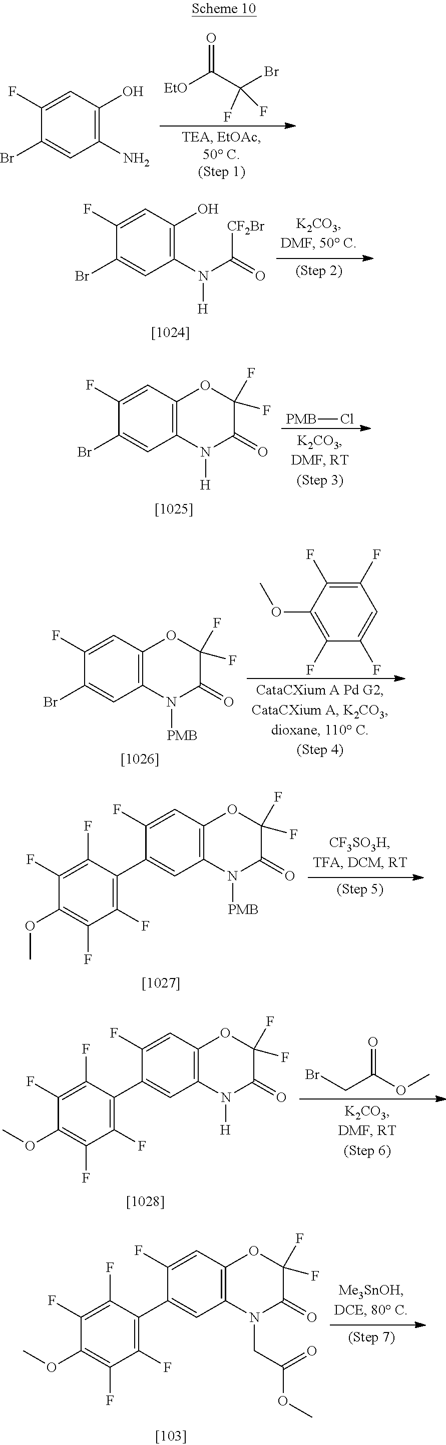

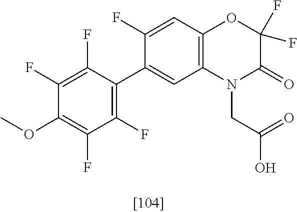 [SMILES: COc1c(F)c(F)c(-c2cc3c(cc2F)OC(F)(F)C(=O)N3CC(=O)O)c(F)c1F]

As shown in Step 1 of Scheme 10, to a solution of 2-amino-4-bromo-5-fluorophenol (18.0 g, 87.4 mmol) and methyl 2-bromo-2,2-difluoroacetate (550 mg, 2.91 mmol) in EtOAc (180 mL) was added triethylamine (17.6 g, 175 mmol) at 20° C. The resulting solution was then stirred at 50° C. for 2 hours, cooled to room temperature, diluted with water, and extracted with ethyl acetate. The combined organic layers were washed with brine, dried over anhydrous sodium sulfate, filtered, and concentrated under reduced pressure. The residue was purified by flash chromatography (0%-40% ethyl acetate in petroleum ether) to afford 2-bromo N-(5-bromo-4-fluoro-2-hydroxyphenyl)-2,2-difluoroacetamide (Compound 1024, 11.0 g, 31% yield) as a brown solid: MS (ESI) calculated for C₈H₄Br₂F₃NO₂ [M−1]⁻=360.0, found 360.0.

As shown in Step 2 of Scheme 10, to a solution of 2-bromo-N-(5-bromo-4-fluoro-2-hydroxyphenyl)-2,2-difluoroacetamide (7.0 g, 19.3 mmol) in DMF (70 mL) was added K₂CO₃ (5.3 g, 38.6 mmol) at 20° C. The resulting solution was stirred at 50° C. for 16 hours, cooled to room temperature, diluted with water, and extracted with ethyl acetate. The combined organic layers were washed with brine, dried over anhydrous sodium sulfate, filtered, and concentrated under reduced pressure. The residue was purified by flash chromatography (0%-30% ethyl acetate in petroleum ether) to afford 6-bromo-2,2,7-trifluoro-4H-1,4-benzoxazin-3-one (Compound 1025, 4.0 g, 66% yield) as a brown solid: MS (ESI) calculated for C₈H₃BrF₃NO₂ [M−1]⁻=280.0, found 280.0.

As shown in Step 3 of Scheme 10, to a solution of 6-bromo-2,2,7-trifluoro-4H-1,4-benzoxazin-3-one (2.0 g, 7.09 mmol) in DMF (20 mL) were added p-methoxybenzyl chloride (1.6 g, 10.7 mmol) and K₂CO₃ (1.9 g, 14.2 mmol) at 20° C. The resulting solution was stirred at 20° C. for 16 hours, diluted with water, and extracted with ethyl acetate. The combined organic layers were washed with brine, dried over anhydrous sodium sulfate, filtered, and concentrated under reduced pressure. The residue was purified by flash chromatography (0%-30% ethyl acetate in petroleum ether) to afford 6-bromo-2,2,7-trifluoro-4-(4-methoxybenzyl)-2H-benzo[b][1,4]oxazin-3(4H)-one (Compound 1026, 2.5 g, 78% yield) as a yellow solid: GCMS calculated for C₁₆H₁₁BrF₃NO₃=401.0; found=401.0.

As shown in Step 4 of Scheme 10, to a solution of 6-bromo-2,2,7-trifluoro-4-[(4-methoxyphenyl)methyl]-1,4-benzoxazin-3-one (Compound 1021, 2.0 g, 4.97 mmol) and 2,3,5,6-tetrafluoroanisole (1.3 g, 7.44 mmol) in dioxane (10 mL) were added chloro[(diadamantan-1-yl)(n-butyl)phosphino][2-amino-1,1-biphenyl-2-yl]palladium (II) (CataCXium A Pd G2, 300 mg, 0.50 mmol), bis(adamantan-1-yl)(butyl)phosphane (CataCXium A, 200 mg, 0.50 mmol) and K₂CO₃ (1.4 g, 9.95 mmol). The mixture was stirred at 110° C. for 16 hours under nitrogen, cooled to room temperature, diluted with water, and extracted with ethyl acetate. The combined organic layers were washed with brine, dried over anhydrous sodium sulfate, filtered, and concentrated under reduced pressure. The residue was purified by reversed-phase flash chromatography (5% to 60% acetonitrile in water) to afford 2,2,7-tri fluoro-4-(4-methoxybenzyl)-6-(2,3,5,6-tetrafluoro-4-methoxyphenyl)-2H-benzo[b][1,4]oxazin-3(4H)-one (Compound 1027, 680 mg, 25% yield) as a yellow solid: GCMS calculated for C₂₃H₁₄F₇NO₄=501.1, found 501.0.

As shown in Step 5 of Scheme 10, to a solution of 2,2,7-trifluoro-4-(4-methoxybenzyl)-6-(2,3,5,6-tetrafluoro-4-methoxyphenyl)-2H-benzo[b][1,4]oxazin-3(4H)-one (640 mg, 1.28 mmol) in DCM (10 mL) were added trifluoromethanesulfonic acid (1.9 g, 12.77 mmol) and trifluoroacetic acid (1.4 g, 12.77 mmol). The mixture was stirred at 25° C. for 2 hours, diluted with water, and extracted with ethyl acetate. The combined organic layers were washed with brine, dried over anhydrous sodium sulfate, filtered, and concentrated under reduced pressure. The residue was purified by flash chromatography (0%-20% ethyl acetate in petroleum ether) to afford 2,2,7-trifluoro-6-(2,3,5,6-tetrafluoro-4-methoxyphenyl)-2H-benzo[b][1,4]oxazin-3(4H)-one (Compound 1028, 420 mg, 78% yield) as a colorless oil: GCMS calculated for C₁₅H₆F₇NO₃=381.0, found 381.0.

As shown in Step 6 of Scheme 10, to a stirred solution of 2,2,7-trifluoro-6-(2,3,5,6-tetrafluoro-4-methoxyphenyl)-2H-benzo[b][1,4]oxazin-3(4H)-one (Compound 1028, 100 mg, 0.26 mmol) and DMF (2 mL) were added methyl 2-bromoacetate (60.2 mg, 0.39 mmol) and K₂CO₃ (73 mg, 0.52 mmol). The mixture was stirred at 20° C. for 2 hours, diluted with water, and extracted with ethyl acetate. The combined organic layers were washed with brine, dried over anhydrous sodium sulfate, filtered, and concentrated under reduced pressure. The residue was purified by reversed-phase flash chromatography (5%-40% acetonitrile in water) to afford methyl 2-(2,2,7-trifluoro-3-oxo-6-(2,3,5,6-tetrafluoro-4-methoxyphenyl)-2,3-dihydro-4H-benzo[b][1,4]oxazin-4-yl)acetate (Compound 103, 100 mg, 84% yield) as a white solid: GCMS calculated for C₁₈H₁₀F₇NO₅=453.0, found 453.0. ¹H-NMR (400 MHz, DMSO-d₆) δ 7.85-7.69 (m, 2H), 4.88 (s, 2H), 4.13 (s, 3H), 3.72 (s, 3H); ¹⁹F-NMR (376 MHz, DMSO-d₆) δ −75.60, −115.73, −142.07, −157.69.

As shown in Step 7 of Scheme 10, to a stirred solution of methyl 2-(2,2,7-trifluoro-3-oxo-6-(2,3,5,6-tetrafluoro-4-methoxyphenyl)-2,3-dihydro-4H-benzo[b][1,4]oxazin-4-yl)acetate (60 mg, 0.13 mmol) in 1,2-dichloroethane (2 mL) was added trimethystannanol (48 mg, 0.26 mmol). The mixture was stirred at 80° C. for 16 hours under a nitrogen atmosphere, diluted with water, acidified to pH=4 with 1M HCl, and extracted with ethyl acetate. The combined organic layers were washed with brine, dried over anhydrous sodium sulfate, filtered, and concentrated under reduced pressure. The residue was purified by reversed-phase flash chromatography with (5%-50% acetonitrile in water) to afford 2-(2,2,7-trifluoro-3-oxo-6-(2,3,5,6-tetrafluoro-4-methoxyphenyl)-2,3-dihydro-4H-benzo[b][1,4]oxazin-4-yl)acetic acid (Compound 104, 30 mg, 52% yield) as a white solid: MS (ES) calculated for C₁₇H₈F₇NO₅ [M−1]⁻=438.0, found 438.0; ¹H-NMR (400 MHz, methanol-d₄) δ 7.48-7.27 (m, 2H), 4.85 (s, 2H), 4.16 (s, 3H); ¹⁹F-NMR (376 MHz, methanol-d₄) δ −78.85, −117.01, −144.43, −160.22.

Alkylation of Compound 1028 with methyl 2-bromopropanoate, followed by separation of the resulting enantiomers by chiral HPLC, produced methyl (S)-2-(2,2,7-trifluoro-3-oxo-6-2,3,5,6-tetrafluoro-4-methoxyphenyl)-2,3-dihydro-4H-benzo[b][1,4]oxazin-4-yl)propanoate (Compound 111): GCMS calculated for C₁₉H₁₂F₇NO₅=467.0, found 467.1; ¹H-NMR (400 MHz, chloroform-d) δ 7.21-7.12 (m, 1H), 6.91 (d, J=6.0 Hz, 1H), 5.54-5.45 (m, 1H), 4.21-4.11 (m, 3H), 3.77 (s, 3H), 1.71 (d, J=7.2 Hz, 3H); ¹⁹F-NMR (376 MHz, chloroform-d) δ −76.23, −79.97, −113.45, −142.28, −157.23; and methyl (R)-2-(2,2,7-trifluoro-3-oxo-6-(2,3,5,6-tetrafluoro-4-methoxyphenyl)-2,3-dihydro-4H-benzo[b][1,4]oxazin-4-yl)propanoate (Compound 112): GCMS calculated for C₁₉H₁₀F₇NO₅=467.0, found 467.1; ¹H-NMR (400 MHz, chloroform-d) δ 7.21-7.12 (m, 1H), 6.91 (d, J=6.4 Hz, 1H), 5.55-5.45 (m, 1H), 4.23-4.09 (m, 3H), 3.77 (s, 3H), 1.71 (d, J=7.2 Hz, 3H); ¹⁹F-NMR (376 MHz, chloroform-d) 5-76.23, −79.97, −113.45, −142.28, −157.23. Each of Compounds 111 and 112 was treated with trimethylstannanol in DCE at 80° C. to produce, respectively, (S)-2-(2,2,7-trifluoro-3-oxo-6-(2,3,5,6-tetrafluoro-4-methoxyphenyl)-2,3-dihydro-4H-benzo[b][1,4]oxazin-4-yl)propanoic acid (Compound 113): MS (ESI) calculated for C₁₈H₁₀F₇NO₅ [M−1]⁻=452.0, found 451.9; ¹H-NMR (400 MHz, DMSO-d₆) δ 13.22 (s, 1H), 7.80-7.72 (m, 2H), 5.38-5.28 (m, 1H), 4.14 (s, 3H), 1.53 (s, 3H); ¹⁹F-NMR (376 MHz, DMSO-d₆) δ −75.98, −118.78, −142.14, −151.73; and (R)-2-(2,2,7-trifluoro-3-oxo-6-(2,3,5,6-tetrafluoro-4-methoxyphenyl)-2,3-dihydro-4H-benzo[b][1,4]oxazin-4-yl)propanoic acid (Compound 114): MS (ESI) calculated for C₁₈H₁₀F₇NO₅ [M−1]⁻=452.0, found 451.9; ¹H-NMR (400 MHz, DMSO-d₆) δ7.80-7.72 (m, 2H), 5.38-5.28 (m, 1H), 4.14 (s, 3H), 1.65-1.33 (m, 3H); ¹⁹F-NMR (376 MHz, DMSO-d₆) δ −75.70, −115.85, −142.19, −157.72.

### Example 9. Preparation of methyl 2-(2,2,7-trifluoro-3-oxo-6-(2,3,4,6-tetrafluoro-5-methylphenyl)-2,3-dihydro-4H-benzo[b][1,4]oxazin-4-yl)acetate (Compound 60) and 2 (2,2,7-tri fluoro-3-oxo-6-(2,3,4,6-tetrafluoro-5-methylphenyl)-2,3-dihydro-4H-benzo[b][1,4]oxazin-4-yl)acetic acid (Compound 61)

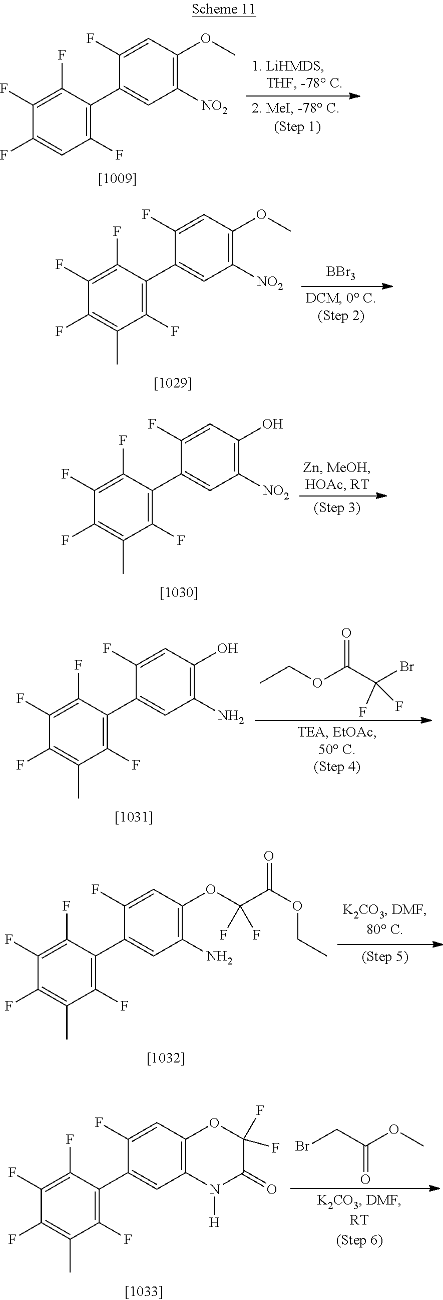

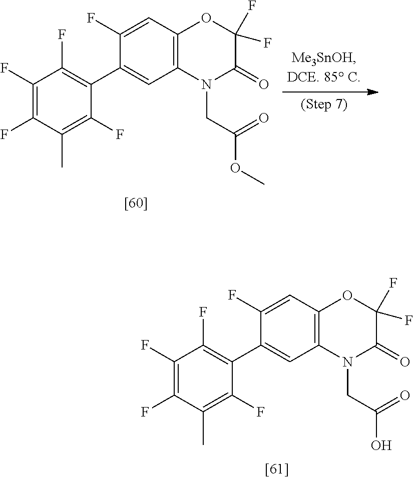 [SMILES: COC(=O)CN1C(=O)C(F)(F)Oc2cc(F)c(-c3c(F)c(C)c(F)c(F)c3F)cc21 ; Cc1c(F)c(F)c(F)c(-c2cc3c(cc2F)OC(F)(F)C(=O)N3CC(=O)O)c1F]

As shown in Step 1 of Scheme 11, to a stirred mixture of 2,2′,3,4,6-pentafluoro-4′-methoxy-5′-nitro-1,1′-biphenyl (Compound 1009, 1.0 g, 3.13 mmol) in THE (20 mL) was added lithium bis(trimethylsilyl)amide (LiHMDS, 6.27 mmol, 1M in THF) dropwise at −78° C. under an atmosphere of nitrogen. The resulting mixture was stirred at −78° C. for 30 minutes and methyl iodide (0.7 g, 4.70 mmol) was added at −78° C. under an atmosphere of nitrogen. The resulting mixture was stirred at −78° C. for 4 hours under nitrogen, diluted with water, and extracted with ethyl acetate. The combined organic layers were washed with brine, dried over anhydrous sodium sulfate, filtered, and concentrated under reduced pressure. The residue was purified by flash chromatography (0%-50% ethyl acetate in petroleum ether) to afford 2,2′,3,4,6-pentafluoro-4′-methoxy-5-methyl-5′-nitro-1,1′-biphenyl (Compound 1029, 800 mg, 77% yield) as a yellow solid: GCMS calculated for C₁₄H₈F₅NO₃=333.0, found 333.0.

As shown in Step 2 of Scheme 11, to a stirred mixture of 2,2′,3,4,6-pentafluoro-4′-methoxy-5-methyl-5′-nitro-1,1′-biphenyl (800 mg, 2.40 mmol) in DCM (20 mL) was added boron tribromide (3.7 g, 12.00 mmol) dropwise at 0° C. under an atmosphere of nitrogen. The resulting mixture was stirred at 0° C. for 2 hours, diluted with water, and extracted with ethyl acetate. The combined organic layers were washed with brine, dried over anhydrous sodium sulfate, filtered, and concentrated under reduced pressure. The residue was purified by flash chromatography (0%-50% ethyl acetate in petroleum ether) to afford methyl 2-(6-amino-2,2,7-trifluoro-3-oxo-2,3-dihydro-4H-benzo[b][1,4]oxazin-4-yl)acetate (Compound 1030, 710 mg, 93% yield) as a yellow oil: MS (ESI) calculated for C₁₃H₆F₅NO₃ [M−1]⁻=318.0, found 318.0.

As shown in Step 3 of Scheme 11, to a stirred mixture of 2,2′,3′,4′,6′-pentafluoro-5′-methyl-5-nitro-[1,1′-biphenyl]-4-ol (710 mg, 2.22 mmol) in acetic acid (0.2 mL) and MeOH (20 mL) was added zinc powder (727 mg, 11.12 mmol). The resulting mixture was stirred at room temperature for 2 hours under an atmosphere of nitrogen. The solids were filtered out and the resulting solution diluted with water. The aqueous solution was extracted with ethyl acetate and the combined organic layers washed with NaHCO₃, dried over anhydrous sodium sulfate, filtered, and concentrated under reduced pressure. The residue was purified by flash chromatography (0%-50% ethyl acetate in petroleum ether) to afford 5-amino-2,2′,3′,4′,6′-pentafluoro-5′-methyl-[1,1′-biphenyl]-4-ol (Compound 1031, 560 mg, 87% yield) as a brown solid: MS (ESI) calculated for C₁₃H₈F₅NO [M−1]⁻=288.0, found 288.0.

As shown in Step 4 of Scheme 11, to a stirred mixture of 5-amino-2,2′,3′,4′,6′-pentafluoro-5′-methyl-[1,1′-biphenyl]-4-ol (600 mg, 2.08 mmol) in MeOH (15 mL) were added ethyl 2-bromo-2,2-difluoroacetate (632 mg, 3.11 mmol) and triethylamine (420 mg, 4.15 mmol). The resulting mixture was stirred at 50° C. for 16 hours under an atmosphere of nitrogen then diluted with water. The aqueous solution was extracted with ethyl acetate and the combined organic layers washed with brine, dried over anhydrous sodium sulfate, filtered, and concentrated under reduced pressure. The residue was purified by flash chromatography (0%-50% ethyl acetate in petroleum ether) to afford 2-bromo-2,2-difluoro-N-(2′,3′,4′,6,6′-pentafluoro-4-hydroxy-5′-methyl-[1,1′-biphenyl]-3-yl)acetamide (Compound 1032, 550 mg, 59% yield) as a brown solid: MS (ESI) calculated for C₁₅H₇BrF₇NO₂ [M−1]⁻=444.0, found 444.0.

As shown in Step 5 of Scheme 11, to a stirred mixture of 2-bromo-2,2-difluoro-N-(2′,3′,4′,6,6′-pentafluoro-4-hydroxy-5′-methyl-[1,1′-biphenyl]-3-yl)acetamide (550 mg, 1.23 mmol) in DMF (10 mL) was added K₂CO₃ (511 mg, 3.70 mmol). The resulting mixture was stirred at 80° C. for 2 hours under an atmosphere of nitrogen, cooled to room temperature, diluted with water, and extracted with ethyl acetate. The combined organic layers were washed with brine, dried over anhydrous sodium sulfate, filtered, and concentrated under reduced pressure. The residue was purified by flash chromatography (0%-50% ethyl acetate in petroleum ether) to afford 2,2,7-trifluoro-6-(2,3,4,6-tetrafluoro-5-methylphenyl)-2H-benzo[b][1,4]oxazin-3(4H)-one (Compound 1033, 360 mg, 80% yield) as a brown solid; MS (ESI) calculated for C₁₅H₆F₇NO₂ [M−1]⁻=364.0, found 364.0, ¹H-NMR (400 MHz, DMSO-d₆) δ 12.17 (s, 1H), 7.64 (d, J=9.6 Hz, 1H), 7.18 (d, J=6.4 Hz, 1H), 2.24 (s, 3H); ¹⁹F-NMR (376 MHz, DMSO-d₆) δ −75.25, −116.88, −120.24, −135.31, −138.86, −165.49.

As shown in Step 6 of Scheme 11, to a solution of 2,2,7-trifluoro-6-(2,3,4,6-tetrafluoro-5-methylphenyl)-2H-benzo[b][1,4]oxazin-3(4H)-one (40 mg, 0.11 mmol) in DMF (2 mL) were added methyl 2-bromoacetate (25 mg, 0.16 mmol) and K₂CO₃ (45 mg, 0.33 mmol) at 20° C. under a nitrogen atmosphere. The resulting solution was stirred at 20° C. for 2 hours, diluted with water, and extracted with ethyl acetate. The combined organic layers were washed with brine, dried over anhydrous sodium sulfate, filtered, concentrated under reduced pressure, and purified by reversed-phase flash chromatography (10%-50% acetonitrile in water) to afford methyl 2-(2,2,7-trifluoro-3-oxo-6-(2,3,4,6-tetrafluoro-5-methylphenyl)-2,3-dihydro-4H-benzo[b][1,4]oxazin-4-yl)acetate (Compound 60, 30 mg, 75% yield) as a colorless oil: GCMS calculated for C₁₈H₁₀F₇NO₄=437.0, found 437.0; ¹H-NMR (400 MHz, methanol-d₄) δ 7.42-7.35 (m, 2H), 4.90 (s, 2H), 3.78 (s, 3H), 2.28 (s, 3H); ¹⁹F-NMR (377 MHz, methanol-d₄) δ −79.05, −116.98, −121.84, −137.66, −140.46, −168.48.

As shown in Step 7 of Scheme 11, to a stirred solution of methyl 2-(2,2,7-trifluoro-3-oxo-6-(2,3,4,6-tetrafluoro-5-methylphenyl)-2,3-dihydro-4H-benzo[b][1,4]oxazin-4-yl)acetate (90 mg, 0.21 mmol) in DCE (5 mL) was added trimethylstannanol (74 mg, 0.41 mmol). The resulting mixture was stirred at 65° C. under a nitrogen atmosphere for 16 hours, cooled to room temperature, diluted with water, acidified to pH 4-5 with formic acid, and extracted with ethyl acetate. The combined organic layers were washed with brine, dried over anhydrous sodium sulfate, filtered, and concentrated under reduced pressure. The residue was purified by reversed-phase flash chromatography (10%-50% acetonitrile in water) to afford 2-(2,2, 7-tri fluoro-3-oxo-6-(2,3,4,6-tetrafluoro-5-methylphenyl)-2,3-dihydro-4H-benzo[b][1,4]oxazin-4-yl)acetic acid (Compound 61, 25 mg, 28% yield) as a white solid: MS (ES) calculated for C₁₇H₈F₇NO₄ [M−1]⁻=422.0, found 421.9; ¹H-NMR (400 MHz, methanol-d₆) δ 7.30 (d, J=9.2 Hz, 1H), 7.24 (d, J=6.4 Hz, 1H), 4.58 (s, 2H), 2.27 (s, 3H); ¹⁹F-NMR (377 MHz, methanol-d₄) δ −77.44, −118.12, −121.58, −137.89, −140.14, −168.62.

### Example 10. Preparation of methyl 2-(2,2,7-trifluoro-3-oxo-6-(2,3,5,6-tetrafluoro-4-(trifluoromethyl)phenyl)-2,3-dihydro-4H-benzo[b][1,4]oxazin-4-yl)acetate (Compound 62) and 2-(2,2,7-trifluoro-3-oxo-6-(2,3,5,6-tetrafluoro-4-(trifluoromethyl)phenyl)-2,3-dihydro-4H-benzo[b][1,4]oxazin-4-yl)acetic acid (Compound 63)

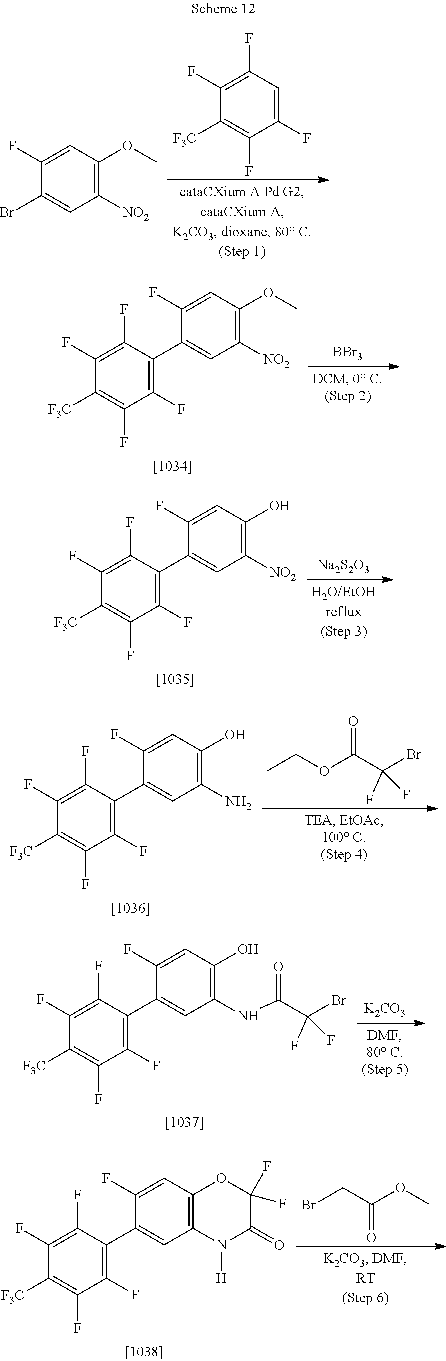

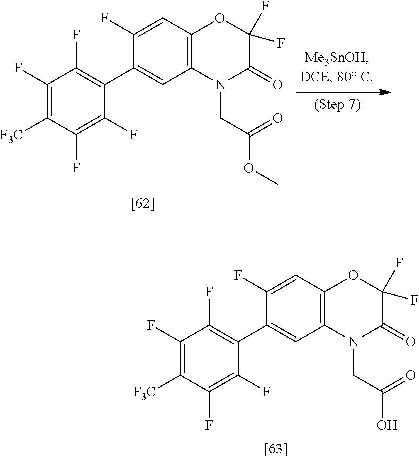 [SMILES: COC(=O)CN1C(=O)C(F)(F)Oc2cc(F)c(-c3c(F)c(F)c(C(F)(F)F)c(F)c3F)cc21 ; O=C(O)CN1C(=O)C(F)(F)Oc2cc(F)c(-c3c(F)c(F)c(C(F)(F)F)c(F)c3F)cc21]

As shown in Step 1 of Scheme 12, to a solution of 1-bromo-2-fluoro-4-methoxy-5-nitrobenzene (3.0 g, 11.99 mmol) and 1,2,4,5-tetrafluoro-3-(trifluoromethyl)benzene (3.9 g, 17.99 mmol) in dioxane (70 mL) were added chloro[(di(1-adamantyl)-N-butylphosphine)-2-(2-aminobiphenyl)]palladium(11) (cataCXium A Pd G2, 400 mg, 0.60 mmol), di(1-adamantyl)-n-butylphosphine (cataCXium A, 220 mg, 0.60 mmol) and K₂CO₃ (3.3 g, 23.99 mmol). The resulting mixture was stirred at 90° C. for 16 hours under a nitrogen atmosphere then concentrated under reduced pressure. The residue was purified by flash chromatography (0%-13% ethyl acetate in petroleum ether) to afford 2,2′,3,5,6-pentafluoro-4′-methoxy-5′-nitro-4-(trifluoromethyl)-1,1′-biphenyl (Compound 1034, 3.3 g, 63% yield) as a yellow oil: GCMS calculated for C₁₄H₅F₈NO₃=387.0, found 387.0.

As shown in Step 2 of Scheme 12, to a solution of 2,2′,3,5,6-pentafluoro-4′-methoxy-5′-nitro-4-(trifluoromethyl)-1,1′-biphenyl (3.1 g, 8.00 mmol) in DCM (20 mL) was added BBr₃ (10.0 g, 40.03 mmol) in portions at 0° C. The resulting mixture was stirred at 0° C. for 3 hours under a nitrogen atmosphere. The resulting mixture was diluted with water and the aqueous layer extracted with ethyl acetate. The combined organic solution was dried over sodium sulfate, filtered, and concentrated under reduced pressure. The residue was purified by flash chromatography (0%-19% ethyl acetate in petroleum ether) to afford 2,2′,3′,5′,6′-pentafluoro-5-nitro-4′-(trifluoromethyl)-[1,1′-biphenyl]-4-ol (Compound 1035, 1.9 g, 54% yield) as a black solid: MS (ESI) calculated for C₁₃H₃F₅NO₃ [M−1]⁻=372.0, found 372.1.

As shown in Step 3 of Scheme 12, to a solution of 2,2′,3′,5′,6′-pentafluoro-5-nitro-4′-(trifluoromethyl)-[1,1′-biphenyl]-4-ol (1.8 g, 4.82 mmol) in H₂O (10 mL) and EtOH (10 mL) was added sodium hypophosphite (2.1 g, 24.12 mmol). The resulting mixture was stirred at 100° C. for 2 hours under a nitrogen atmosphere, cooled to room temperature, diluted with water, and extracted with ethyl acetate. The combined organic solution was dried over sodium sulfate, filtered, and concentrated under reduced pressure. The residue was purified by flash chromatography (0%-18% ethyl acetate in petroleum ether) to afford 5-amino-2,2′,3′,5′,6′-pentafluoro-4′-(trifluoromethyl)-[1,1′-biphenyl]-4-ol (Compound 1036, 1.4 g, 78% yield) as a brown solid: MS (ESI) calculated for C₁₃H₃F₈NO₃ [M−1]⁻=342.0, found 342.0.

As shown in Step 4 of Scheme 12, to a solution of 5-amino-2,2′,3′,5′,6′-pentafluoro-4′-(trifluoromethyl)-[1,1′-biphenyl]-4-ol (1.1 g, 3.40 mmol) and triethylamine (690 mg, 6.81 mmol) in T₁₄F (25 mL) was added bromodifluoroacetyl chloride (790 mg, 4.09 mmol) dropwise at room temperature. The resulting mixture was stirred at 20° C. for 2 hours under a nitrogen atmosphere, diluted with water, and extracted with ethyl acetate. The combined organic solution was dried over sodium sulfate, filtered, and concentrated under reduced pressure to afford 2-bromo-2,2-difluoro-N [2′,3′,5′,6,6′-pentafluoro-4-hydroxy-4′-(trifluoromethyl)-[1,1′-biphenyl]-3-yl]acetamide (Compound 1037, 1.0 g, 54% yield) as a brown oil: MS (ES) calculated for C₁₅H₄BrF₁₀NO₂ [M−1]⁻=497.9, found 498.0.

As shown in Step 5 of Scheme 12, a mixture of 2-bromo-2,2-difluoro-N-[2′,3′,5′,6,6′-pentafluoro-4-hydroxy-4′-(trifluoromethyl)-[1,1′-biphenyl]-3-yl]acetamide (1.7 g, 3.39 mmol) and K₂CO₃ (940 mg, 6.79 mmol) in DMF (10 mL) was stirred at 80° C. for 3 hours under a nitrogen atmosphere. The resulting mixture was diluted with water and the aqueous layer extracted with ethyl acetate. The combined organic solution was dried over sodium sulfate, filtered, and concentrated under reduced pressure. The residue was purified by reversed-phase flash chromatography (5%-36% acetonitrile in water) to afford 2,2,7-trifluoro-6-(2,3,5,6-tetrafluoro-4-(trifluoromethyl)phenyl)-2H-benzo[b][1,4]oxazin-3(4H)-one (Compound 1038, 600 mg, 41% yield) as a white solid: MS (ESI) calculated for C₁₅H₃F₁₀NO₂ [M−1]⁻=418.0, found 418.2; ¹H-NMR (400 MHz, DMSO-d₆) δ 12.24 (s, 1H), 7.72-7.70 (d, J=9.6 Hz, 1H), 7.30-7.28 (d, J=6.8 Hz, 1H); ¹⁹F-NMR (376 MHz, DMSO-d₆) δ −55.60, −74.99, −116.42, −139.04, −141.63.

As shown in Step 6 of Scheme 12, to a stirred solution of 2,2,7-trifluoro-6-(2,3,5,6-tetrafluoro-4-(trifluoromethyl)phenyl)-2H-benzo[b][1,4]oxazin-3(4H)-one (200 mg, 0.48 mmol) in DMF (4 mL) were added methyl 2-bromoacetate (110 mg, 0.72 mmol) and K₂CO₃ (132 mg, 0.95 mmol). The resulting mixture was stirred at 80° C. for 16 hours under a nitrogen atmosphere, cooled to room temperature, diluted with water, and extracted with ethyl acetate. The combined organic solution was dried over sodium sulfate, filtered, and concentrated under reduced pressure. The residue was purified by flash chromatography (0%-30% ethyl acetate in petroleum ether) to afford methyl 2-(2,2,7-trifluoro-3-oxo-6-(2,3,5,6-tetrafluoro-4-(trifluoromethyl)phenyl)-2,3-dihydro-4H-benzo[b][1,4]oxazin-4-yl)acetate (Compound 62, 200 mg, 85% yield) as a colorless oil: GCMS calculated for C₁₈H₇F₁₀NO₄=491.0, found 491.0.

As shown in Step 7 of Scheme 12, to a stirred solution of methyl 2-(2,2,7-trifluoro-3-oxo-6-(2,3,5,6-tetrafluoro-4-(trifluoromethyl)phenyl)-2,3-dihydro-4H-benzo[b][1,4]oxazin-4-yl)acetate (180 mg, 0.37 mmol) in DCE (4 mL) was added trimethylstannanol (133 mg, 0.73 mmol). The resulting mixture was stirred at 65° C. for 16 hours under a nitrogen atmosphere, cooled to room temperature, diluted with water, acidified to pH 4-5 with formic acid, extracted with ethyl acetate. The combined organics were dried over sodium sulfate, filtered, and concentrated under reduced pressure. The residue was purified by flash chromatography (0%-30% ethyl acetate in petroleum ether) to afford 2-(2,2,7-trifluoro-3-oxo-6-(2,3,5,6-tetrafluoro-4-(trifluoromethyl)phenyl)-2,3-dihydro-4H-benzo[b][1,4]oxazin-4-yl)acetic acid (Compound 63, 80 mg, 46% yield) as a white solid: MS (ESI) calculated for C₁₇H₅F₁₀NO₄ [M−1]⁻=476.0, found 476.3; ¹H-NMR (400 MHz, DMSO-d₆) δ 7.82 (d, J=9.6 Hz, 2H), 4.74 (s, 2H); ¹⁹F-NMR (376 MHz, DMSO-d) δ −55.64, −75.33, −115.46, −138.07, −141.59.

### Example 11. Preparation of methyl 2-(3-oxo-6-(perfuorophenyl)-2,3-dihydro-4H-benzo[b][1,4]oxazin-4-yl)acetate (Compound 39) and 2-(3-oxo-6-(perfuorophenyl)-2,3-dihydro-4H-benzo[b][1,4]oxazin-4-yl)acetic acid (Compound 40)

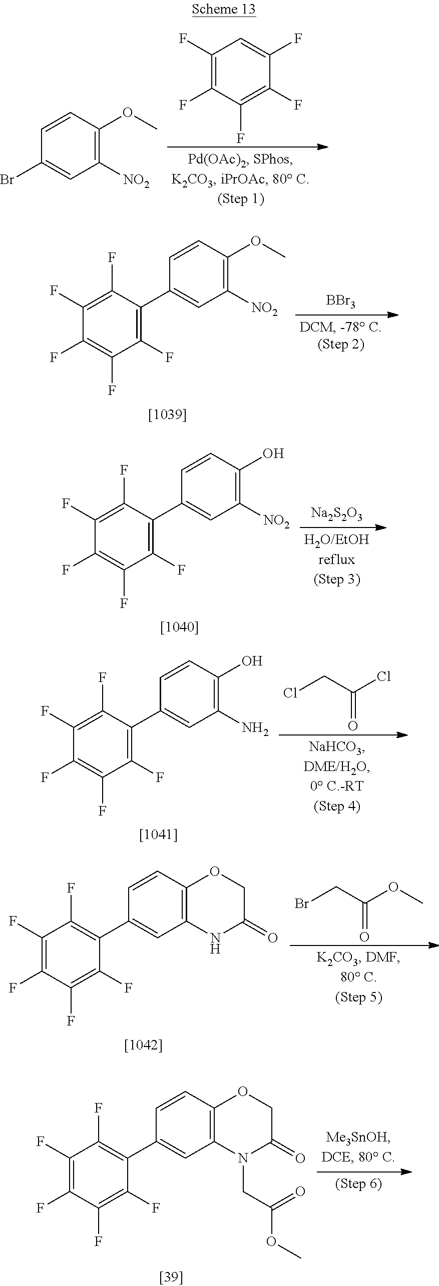

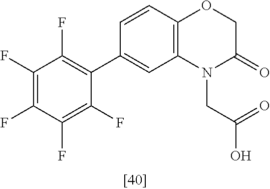 [SMILES: O=C(O)CN1C(=O)COc2ccc(-c3c(F)c(F)c(F)c(F)c3F)cc21]

As shown in Step 1 of Scheme 13, to a degassed mixture of 4-bromo-1-methoxy-2-nitrobenzene (2.0 g, 8.62 mmol) in isopropyl acetate (10 mL) were added Pd(OAc)₂ (190 mg, 0.86 mmol), Sphos (700 mg, 1.72 mmol), and K₂CO₃ (2.4 g, 17.2 mmol) under a nitrogen atmosphere. The mixture was stirred at room temperature for 5 minutes and a solution of pentafluorobenzene (2.9 g, 17.24 mmol) in isopropyl acetate (10 mL) was added. The resulting mixture was stirred at 80° C. for 16 hours under nitrogen, cooled to room temperature, filtered, and concentrated under reduced pressure. The residue was purified by flash chromatography (0%-45% ethyl acetate in petroleum ether) to afford 2,3,4,5,6-pentafluoro-4′-methoxy-3′-nitro-1,1′-biphenyl (Compound 1039, 1.7 g, 54% yield) as a brown solid: GCMS calculated for C₁₃H₆F₅NO₃=319.0, found 319.0.

As shown in Step 2 of Scheme 13, to a stirred solution of 2,3,4,5,6-pentafluoro-4-methoxy-3-nitro-1,1-biphenyl (1.3 g, 4.1 mmol) in DCM (10 mL) was added boron tribromide (5.1 g, 20.4 mmol) dropwise at −78° C. under a nitrogen atmosphere. The resulting mixture was stirred at −78° C. for 2 hours, then warmed to room temperature and stirred for an additional 16 hours. The reaction diluted with water, extracted with DCM, and the combined organics washed with brine, dried over anhydrous sodium sulfate, filtered, and concentrated under reduced pressure to afford 2′,3′,4′,5′,6′-pentafluoro-3-nitro-[1,1′-biphenyl]-4-ol (Compound 1040, 1.2 g, crude) as a yellow solid: MS (ESI) calculated for C₁₂H₄F₅NO₃ [M−1]⁻=304.0, found 303.8. This material was used in subsequent reactions as is.

As shown in Step 3 of Scheme 13, to a stirred solution of 2′,3′,4′,5′,6′-pentafluoro-3-nitro-[1,1′-biphenyl]-4-ol (1.3 g, 4.2 mmol) in EtOH (6 mL) was added a solution of sodium hyposulfite (3.6 g, 20.8 mmol) in water (6 mL). The mixture was stirred at reflux for 1 hour, cooled to room temperature, and the volatiles removed under reduced pressure. The resulting mixture was diluted with water and extracted with ethyl acetate. The combined organic layers were washed with brine, dried over anhydrous sodium sulfate, filtered, and concentrated under reduced pressure to afford 3-amino-2′,3′,4′,5′,6′-pentafluoro-[1,1′-biphenyl]-4-ol (Compound 1041, 400 mg, crude) as a yellow solid: MS (ESI) calculated for C₁₂H₆F₅NO [M−1]⁻=274.0, found 274.1. This material was used in subsequent reactions as is.

As shown in Step 4 of Scheme 13, to a stirred solution of 3-amino-2′,3′,4′,5′,6′-pentafluoro-[1,1′-biphenyl]-4-ol (5.0 g, 18.2 mmol) and NaHCO₃ (4.5 g, 54.5 mmol) in DME (25 mL) and H₂O (25 mL) was added chloroacetyl chloride (3.1 g, 27.25 mmol) dropwise at 0° C. under a nitrogen atmosphere. The resulting solution was stirred for 16 hours at room temperature under a nitrogen atmosphere, diluted with water, and extracted with ethyl acetate. The combined organic layers were washed with brine, dried over anhydrous sodium sulfate, and concentrated under reduced pressure. The residue was purified by flash chromatography (10%-60% ethyl acetate/petroleum ether) to afford 6-(perfluorophenyl)-2H-benzo[b][1,4]oxazin-3(4H)-one (Compound 1042, 3.4 g, 57% yield) as a brown solid: MS (ESI) calculated for C₁₄H₆F₅NO₂ [M−1]⁻=315.2, found 314.0; ¹H-NMR (400 MHz, DMSO-d₆) δ 10.89 (s, 1H), 7.13 (d, J=8.4 Hz, 1H), 7.06 (d, J=8.4 Hz, 1H), 7.00 (s, 1H), 4.67 (s, 2H); ¹⁹F-NMR (400 MHz, DMSO-d₆) δ −143.63, −156.62, −162.75.

As shown in Step 5 of Scheme 13, a solution of 6-(perfluorophenyl)-2H-benzo[b][1,4]oxazin-3(4H)-one (200 mg, 0.63 mmol) and K₂CO₃ (105 mg, 0.76 mmol) in DMF (4.0 mL) was stirred at 80° C. for 2 hours under a nitrogen atmosphere, cooled to room temperature, diluted with water, and extracted with ethyl acetate. The combined organic layers were washed with brine, dried over anhydrous sodium sulfate, filtered, and concentrated under reduced pressure. The residue was purified by flash chromatography (0%-10% methanol in dichloromethane) to afford methyl 2-(3-oxo-6-(perfluorophenyl)-2,3-dihydro-4H-benzo[b][1,4]oxazin-4-yl)acetate (Compound 39, 29 mg, 11% yield) as a red solid: MS (ESI) calculated for C₁₇H₁₀F₅NO₄ [M+1]⁺=388.0, found 388.1; ¹H-NMR (400 MHz, DMSO-d₆) δ7.31 (s, 1H), 7.24-7.13 (m, 2H), 4.83 (s, 2H), 4.76 (s, 2H), 3.69 (s, 3H); ¹⁹F-NMR (376 MHz, DMSO-d₆) δ −142.89, −156.29, −162.85.

As shown in Step 6 of Scheme 13, to a solution of methyl 2-(3-oxo-6-(perfluorophenyl)-2,3-dihydro-H-benzo[b][1,4]oxazin-4-yl)acetate (110 mg, 0.28 mmol) in DCE (3 mL) was added trimethylstannanol (103 mg, 0.56 mmol). The resulting solution was stirred at 80° C. for 2 hours, cooled to room temperature, diluted with water, and acidified to a pH of 4-5 with formic acid, and extracted with ethyl acetate. The combined organic layers were washed with brine, dried over anhydrous sodium sulfate, filtered, and concentrated under reduced pressure. The residue was purified by reversed-phase flash chromatography (5%-50% acetonitrile in water) to afford 2-(3-oxo-6-(perfluorophenyl)-2,3-dihydro-4H-benzo[b][1,4]oxazin-4-yl)acetic acid (Compound 40, 75 mg, 69% yield) as a white solid: MS (ESI) calculated for C₁₄H₈F₅NO₄ [M−1]⁻=372.2, found 372.2; ¹H-NMR (400 MHz, DMSO-d₆) δ 7.15 (d, J=2.4 Hz, 2H), 7.09 (d, J=2.4 Hz, 2H), 4.69 (s, 2H), 4.07 (s, 2H); ¹⁹F-NMR (377 MHz, DMSO-d₆) δ −143.12, −156.42, −162.72.

Alkylation of Compound 1042 with methyl 2-bromopropanoate, followed by separation of the resulting enantiomers by chiral HPLC, produced methyl (S)-2-(3-oxo-6-(perfluorophenyl)-2,3-dihydro-4H-benzo[b][1,4]oxazin-4-yl)propanoate (Compound 41): MS (ES) calculated for C₁₈H₁₂F₅NO₄ [M+1]⁺=401.0, found 402.0; ¹H-NMR (400 MHz, methanol-d₄) δ 7.22-7.15 (m, 3H), 5.31-5.23 (m, 1H), 4.83 (s, 1H), 4.68 (d, J=1.2 Hz, 1H), 3.74 (s, 3H), 1.62 (d, J=7.2 Hz, 3H); ¹⁹F-NMR (377 MHz, methanol-d₄) δ −145.36, −158.85, −165.37; and methyl (R)-2-(3-oxo-6-(perfluorophenyl)-2,3-dihydro-4H-benzo[b][1,4]oxazin-4-yl)propanoate (Compound 42): MS (ES) calculated for (C₁₈H₁₂F₅NO₄ [M+1]⁺=401.0, found 402.0; ¹H-NMR (400 MHz, methanol-d₄) δ 7.24-7.14 (m, 3H), 5.30-5.23 (m, 1H), 4.83 (s, 1H), 4.69 (d, J=1.2 Hz, 1H), 3.74 (s, 3H), 1.62 (d, J=7.0 Hz, 3H); ¹⁹F-NMR (377 MHz, methanol-d₄) δ −145.37, −158.86, −165.38. Each of Compounds 41 and 42 were treated with trimethylstannanol in DCE at 80° C. to produce, respectively, (S)-2-(3-oxo-6-(perfluorophenyl)-2,3-dihydro-4H-benzo[b][1,4]oxazin-4-yl)propanoic acid (Compound 43) as a white solid: MS (ESI) calculated for C₁₇H₁₀F₅NO₄ [M−1]⁻=386.0, found 386.1; ¹H-NMR (400 MHz, DMSO-d₆) δ7.14-7.03 (m, 3H), 5.15-5.09 (m, 1H), 4.71-4.60 (m, 2H), 1.36 (d, J=7.2 Hz, 3H); ¹⁹F-NMR (377 MHz, DMSO-d₆) δ −143.52, −156.23, −162.57; and (R)-2-(3-oxo-6-(perfluorophenyl)-2,3-dihydro-4H-benzo[b][1,4]oxazin-4-yl)propanoic acid (Compound 44): MS (ESI) calculated for C₁₇H₁₀F₅NO₄ [M+1]⁺=388.1, found 388.0; ¹H-NMR (400 MHz, DMSO-d₆) δ 7.29-7.03 (m, 3H), 5.15-5.11 (m, 1H), 4.70 (s, 2H), 1.42 (d, J=7.2 Hz, 3H); ¹⁹F-NMR (377 MHz, DMSO-d₆) δ −143.24, −156.23, −162.67.

Reaction of Compound 1041 with ethyl 2-bromo-2,2-difluoroacetate and subsequently cyclization, similar to the procedure shown in Scheme 3, produced 2,2-difluoro-6-(perfluorophenyl)-2H-benzo[b][1,4]oxazin-3(4H)-one. Alkylation of this material with methyl 2-bromoacetate produced methyl 2-(2,2-difluoro-3-oxo-6-(perfluorophenyl)-2,3-dihydro-4H-benzo[b][1,4]oxazin-4-yl)acetate (Compound 29): GCMS calculated for C₁₇H₈F₇NO₄=423.0, found 423.0; ¹H-NMR (400 MHz, DMSO-d₆) δ 7.68 (s, 1H), 7.62 (d, J=8.4 Hz, 1H), 7.42 (d, J=8.4 Hz, 1H), 4.92 (s, 2H), 3.72 (s, 3H); ¹⁹F-NMR (400 MHz, DMSO-d₆) δ −75.61, −142.53, −155.30, −162.56; which after treatment with 4M HCl in dioxane at 100° C., produced 2-(2,2-difluoro-3-oxo-6-(perfluorophenyl)-2,3-dihydro-4H-benzo[b][1,4]oxazin-4-yl)acetic acid (Compound 30): MS (ESI) calculated for C₁₆H₆F₇NO₄ [M−1]⁻=408.0, found 408.1; ¹H-NMR (400 MHz, DMSO-d₆) δ 13.34 (br, 1H), 7.62-7.59 (m, 2H), 7.41-7.39 (d, J=8.0 Hz 1H), 4.77 (s, 2H); ¹⁹F-NMR (377 MHz, DMSO-d₆) δ −75.45, −142.51, −155.43, −162.65.

### Example 12. Preparation of methyl 2-(2,2,5,7-tetrafluoro-3-oxo-6-(perfluorophenyl)-2,3-dihydro-4H-benzo[b][1,4]oxazin-4-yl)acetate (Compound 154) and 2-(2,2,5,7-tetrafluoro-3-oxo-6-(perfluorophenyl)-2,3-dihydro-4H-benzo[b][1,4]oxazin-4-yl)acetic acid (Compound 155)

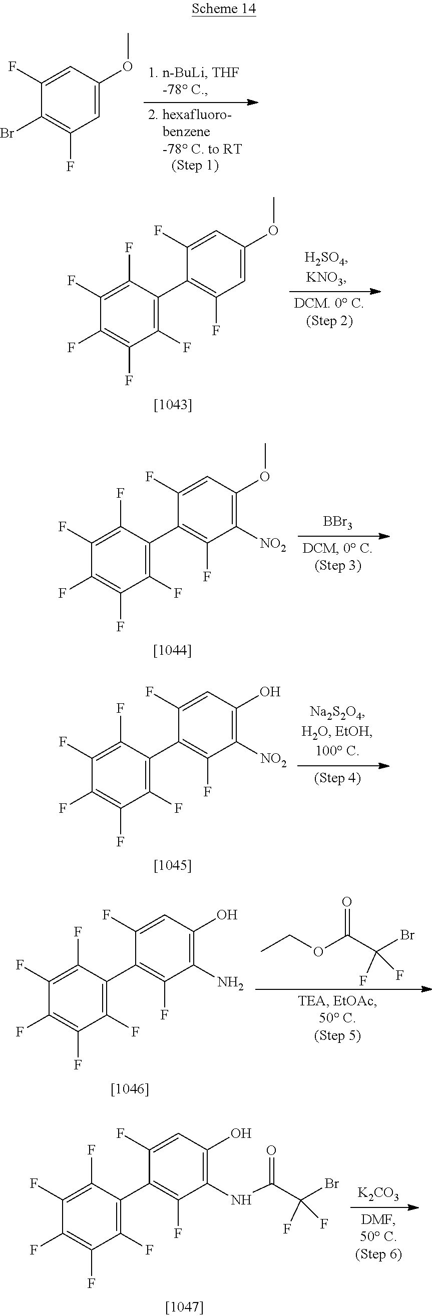

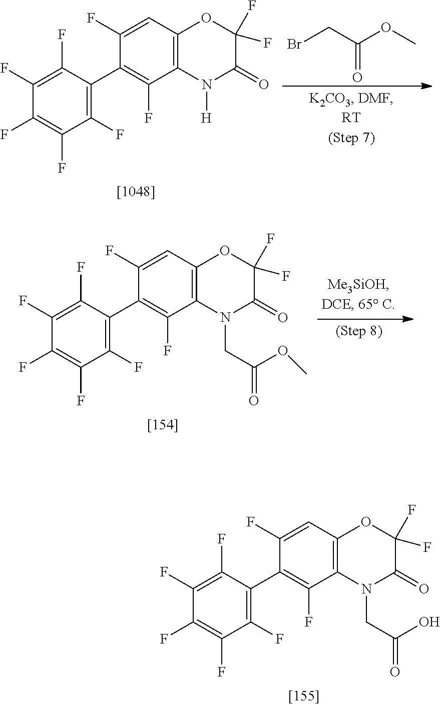

As shown in Step 1 of Scheme 14, to a stirred solution of 2-bromo-1,3-difluoro-5-methoxybenzene (20.0 g, 90.1 mmol) in anhydrous THE (200 mL) under an atmosphere of nitrogen was added n-BuLi (2.5 M in hexane, 39.6 mL, 99.1 mmol) dropwise at −78° C. After addition was complete, stirring was continued at −78° C. for 15 minutes and hexafluorobenzene (25.1 g, 135.1 mmol) was added dropwise at −78° C. The resulting mixture was warmed to room temperature, stirred for 2 hours under nitrogen, diluted with water, and extracted with ethyl acetate. The combined organic layers were washed with brine, dried over anhydrous sodium sulfate, filtered, concentrated under reduced pressure, and purified by flash chromatography (0%-10% ethyl acetate in petroleum ether) within to afford 2,2′,3,4,5,6,6′-heptafluoro-4′-methoxy-1,1′-biphenyl (Compound 1043, 9.0 g, 26% yield) as a white solid: GCMS calculated for C₁₃H₅F₇O=310.0, found 310.0.

As shown in Step 2 of Scheme 14, to a stirred solution of 2,2′,3,4,5,6,6′-heptafluoro-4′-methoxy-1,1′-biphenyl (8.0 g, 25.8 mmol) in DCM (20 mL) and concentrated H₂SO₄ (80 mL) was added KNO₃ (2.6 g, 25.8 mmol) in portions at 0° C. The resulting solution was stirred at 20° C. for 16 hours under nitrogen, diluted with water, and extracted with ethyl acetate. The combined organic layers were washed with brine, dried over anhydrous sodium sulfate, filtered, concentrated under reduced pressure, and purified by flash chromatography (0%-25% ethyl acetate in petroleum ether) to afford 2,2′,3,4,5,6,6′-heptafluoro-4′-methoxy-3′-nitro-1,1′-biphenyl (Compound 1044, 5.5 g, 54% yield) as a yellow solid: GCMS (ESI) calculated for C₁₃H₄F₇NO₃=355.0, found 355.0.

As shown in Step 3 of Scheme 14, to a stirred solution of 2,2′,3,4,5,6,6′-heptafluoro-4′-methoxy-3′-nitro-1,1′-biphenyl (5.5 g, 15.5 mmol) in DCM (70 mL) under a nitrogen atmosphere was added BBr₃ (19.4 g, 77.4 mmol) dropwise at 0° C. The solution was stirred at 0° C. for 2 hours under nitrogen, diluted with water, and extracted with ethyl acetate. The combined organic layers were washed with brine, dried over anhydrous sodium sulfate, filtered, and concentrated under reduced pressure to afford 2,2′,3′,4′,5′,6,6′-heptafluoro-3-nitro-[1,1′-biphenyl]-4-ol (Compound 1045, 4.9 g, crude) as yellow oil: MS (ESI) calculated for C₁₂H₂F₇NO₃ [M−1]⁻=339.9, found 339.9. This material was used as is in subsequent reactions.

As shown in Step 4 of Scheme 14, to a stirred solution of 2,2′,3′,4′,5′,6,6′-heptafluoro-3-nitro-[1,1′-biphenyl]-4-ol (4.9 g, 14.4 mmol) in EtOH (30 mL) and H₂O (30 mL) was added Na₂S₂O₄ (12.5 g, 71.8 mmol) in portions at 20° C. The resulting mixture was stirred at 100° C. for 2 hours under nitrogen, diluted with water, and extracted with ethyl acetate. The combined organic layers were washed with brine, dried over anhydrous sodium sulfate, filtered, concentrated under reduced pressure, and purified by flash chromatography (0%-30% ethyl acetate in petroleum ether) to afford 3-amino-2,2′,3′,4′,5′,6,6′-heptafluoro-[1,1′-biphenyl]-4-ol (Compound 1046, 3.9 g, 78% yield) as a yellow solid: MS (ESI) calculated for C₁₂H₄F₇NO [M+1]⁺=312.0, found 311.9.

As shown in Step 5 of Scheme 14, to a stirred solution of 3-amino-2,2′,3′,4′,5′,6,6′-heptafluoro-[1,1′-biphenyl]-4-ol (3.9 g, 12.5 mmol) and TEA (2.5 g, 25.1 mmol) in EtOAc (50 mL) was added and ethyl 2-bromo-2,2-difluoroacetate (5.1 g, 25.1 mmol) in portions at 20° C. The resulting mixture was stirred at 50° C. for 16 hours under nitrogen, cooled to room temperature, diluted with water, and extracted with ethyl acetate. The combined organic lavers were washed with brine, dried over anhydrous sodium sulfate, filtered, concentrated under vacuum, and purified by reversed-phase flash chromatography (5%-70% acetonitrile in water) to afford 2-bromo-2,2-difluoro-N-(2,2′,3′,4′,5′,6,6′-heptafluoro-4-hydroxy-[1,1′-biphenyl]-3-yl)acetamide (Compound 1047, 1.5 g, 17% yield): MS (ESI) calculated for C₁₄H₃BrF₉NO [M−1]⁻=465.9, found 465.8.

As shown in Step 6 of Scheme 14, to a stirred solution of 2-bromo-2,2-difluoro-N-(2,2′,3′,4′,5′,6,6′-heptafluoro-4-hydroxy-[1,1′-biphenyl]-3-yl)acetamide (1.0 g, 2.1 mmol) in DMF (10 mL) was added K₂CO₃ (591 mg, 4.27 mmol) in portions at 20° C. The resulting mixture was stirred at 50° C. for 2 hours under nitrogen, diluted with water, and extracted with ethyl acetate. The combined organic layers were washed with brine, dried over anhydrous sodium sulfate, filtered, concentrated under reduced pressure, and purified by flash chromatography (0%-20% ethyl acetate in petroleum ether) to afford 2,2,5,7-tetrafluoro-6-(perfluorophenyl)-2H-benzo[b][1,4]oxazin-3(4H)-one (Compound 1048, 590 mg, 71% yield) as a yellow solid: MS (ESI) calculated for C₁₄H₂F₉NO₂ [M−1]⁻=385.9, found 385.9 ¹H-NMR (400 MHz, DMSO-d₆) δ 12.53 (s, 1H), 7.63 (d, J=9.6 Hz, 1H); ¹⁹F-NMR (376 MHz, DMSO-d₆) δ −76.31, −116.25, −124.01, −139.00, −151.21, −161.25.

As shown in Step 7 of Scheme 14, to a stirred solution of 2,2,5,7-tetrafluoro-6-(perfluorophenyl)-2H-benzo[b][1,4]oxazin-3(4H)-one (200 mg, 0.52 mmol) and methyl 2-bromoacetate (95 mg, 0.62 mmol) in DMF (2 mL) was added K₂CO₃ (143 mg, 1.03 mmol) at room temperature under a nitrogen atmosphere. The mixture was stirred at room temperature for 2 hours, diluted with water, and extracted with ethyl acetate. The combined organic layers were washed with brine, dried over anhydrous sodium sulfate, filtered, concentrated under reduced pressure, and purified by reversed-phase preparative HPLC using the following conditions—Column: XBridge Prep Phenyl OBD Column, 19×250 mm, 5 μm; gradient: 55% to 75% acetonitrile/10 mM aq. NH₄HCO₃, to afford methyl 2-(2,2,5,7-tetrafluoro-3-oxo-6-(perfluorophenyl)-2,3-dihydro-4H-benzo[b][1,4]oxazin-4-yl)acetate (Compound 154, 179 mg, 74% yield) as brown oil: GCMS calculated for C₁₇H₆F₉NO₄=459.0, found 459.0, ¹H-NMR (400 MHz, DMSO-d₆) δ 7.83 (d, J=9.2 Hz, 1H), 4.88 (d, J=5.2 Hz, 2H), 3.72 (s, 3H); ¹⁹F-NMR (376 MHz, DMSO-d₆) δ −76.32, −112.93, −120.76, −138.80, −150.69, −161.11.

As shown in Step 8 of Scheme 14, to a stirred solution of methyl 2-(2,2,5,7-tetrafluoro-3-oxo-6-(perfluorophenyl)-2,3-dihydro-4H-benzo[b][1,4]oxazin-4-yl)acetate (32 mg, 0.07 mmol) in DCE (1 mL) was added trimethylstannanol (25 mg, 0.14 mmol) at room temperature under a nitrogen atmosphere. The resulting mixture was stirred at 65° C. for overnight under nitrogen atmosphere, cooled to room temperature, diluted with water, acidified to pH 4-5 with 2M HCl, and extracted with ethyl acetate. The combined organic layers were washed with brine, dried over anhydrous Na₂SO₄, concentrated under reduced pressure, and purified by reversed-phase preparative-HPLC using the following conditions—Column: Xselect CSH C18 OBD Column 30×150 mm 5 μm; gradient: 54% to 64% B acetonitrile/0.1% aq. formic acid to afford 2-(2,2,5,7-tetrafluoro-3-oxo-6-(perfluorophenyl)-2,3-dihydro-4H-benzo[b][1,4]oxazin-4-yl)acetic acid (Compound 155, 9.2 mg, 31% yield) as a white solid: MS (ESI) calculated for C₁₆H₄F₉NO₄ [M−1]⁻=444.0, found 443.8; ¹H-NMR (400 MHz, DMSO-d₆) δ 13.46 (s, 1H), 7.84-7.76 (m, 1H), 4.72 (d, J=5.2 Hz, 2H); ¹⁹F-NMR (376 MHz, DMSO-d₆) δ −76.10, −113.39, −120.19, −138.82, −150.82, −161.17.

Alkenylation of Compound 1048 with propiolic acid methyl ester, using PPh₃, HOAc, in toluene at 110° C., produced methyl 2-(2,2,5,7-tetrafluoro-3-oxo-6-(perfluorophenyl)-2,3-dihydro-4H-benzo[b][1,4]oxazin-4-yl)acrylate (Compound 178): MS (ESI) calculated for C₁₈F₉NO₄ [M−1]⁻=458.0, found 458.0; ¹H-NMR (400 MHz, DMSO-d₆) δ 7.89-7.82 (m, 1H), 6.83-6.77 (m, 1H), 6.52 (d, J=1.6 Hz, 1H), 3.79 (s, 3H); ¹⁹F-NMR (377 MHz, DMSO-d₆) δ −77.37, −112.97, −118.18, −138.91, −150.93, −161.12.

Hydrogenation of Compound 178 using Pd(OH)₂/C as the catalyst, followed by separation of the resulting enantiomeric mixture by chiral HPLC produced methyl (S)-2-(2,2,5,7-tetrafluoro-3-oxo-6-(perfuorophenyl)-2,3-dihydro-4H-benzo[b][1,4]oxazin-4-yl)propanoate (Compound 174): GCMS calculated for C₁₈H₈F₉NO₄=473.0, found 473.0; ¹H-NMR (400 MHz, DMSO-d₆) δ 7.85 (d, J=9.6 Hz, 1H), 5.39-5.51 (m, 1H), 3.67 (s, 3H), 1.60 (d, J=7.2 Hz, 3H); ¹⁹F-NMR (377 MHz, DMSO-d₆) δ −75.01, −84.05, −112.32, −116.94, −138.74, −150.65, −161.11; and methyl (R)-2-(2,2,5,7-tetrafluoro-3-oxo-6-(perfluorophenyl)-2,3-dihydro-H-benzo[b][1,4]oxazin-4-yl)propanoate (Compound 175): GCMS calculated for C₁₈H₈F₉NO₄=473.0, found 473.0; ¹H-NMR (400 MHz, DMSO-d₆) δ 7.86 (d, J=9.6 Hz, 1H), 5.40-5.50 (m, 1H), 3.67 (s, 3H), 1.60 (d, J=7.2 Hz, 3H); ¹⁹F-NMR (377 MHz, DMSO-d₆) δ −75.01, −84.05, −112.32, −116.93, −138.74, −150.65, −161.11. Hydrolysis of Compounds 174 and 175 with trimethylstannanol in DCE at 80° C. produced, respectively, (S)-2-(2,2,5,7-tetrafluoro-3-oxo-6-(perfluorophenyl)-2,3-dihydro-4H-benzo[b][1,4]oxazin-4-yl)propanoic acid (Compound 177): MS (ESI) calculated for C₁₇H₆F₉NO₄ [M−1]⁻=458.0, found 458.0; ¹H-NMR (400 MHz, DMSO-d₆) δ 13.26 (b, 1H), 7.84 (d, J=9.2 Hz, 1H), 5.35-5.48 (m, 1H), 1.58 (d, J=7.2 Hz, 3H); ¹⁹F-NMR (377 MHz, DMSO-d₆) δ −84.14, −12.85, −115.94, −138.71, −150.67, −161.12, and (R)-2-(2,2,5,7-tetrafluoro-3-oxo-6-(perfluorophenyl)-2,3-dihydro-4H-benzo[b][1,4]oxazin-4-yl)propanoic acid (Compound 176): MS (ESI) calculated for C₁₇H₆F₉NO₄ [M−1]⁻=458.0, found 458.0; ¹H-NMR (400 MHz, DMSO-d₆) δ 13.26 (s, 1H), 7.87-7.80 (m, 1H), 5.41-5.31 (m, 1H), 1.61-1.55 (m, 3H); ¹⁹F-NMR (377 MHz, DMSO-d₆) δ −84.14, −112.85, −115.94, −138.71, −150.67, −161.12.

### Example 13. Preparation of methyl 2-(7-chloro-2,2-difluoro-3-oxo-6-(perfluorophenyl)-2,3-dihydro-4H-benzo[b][1,4]oxazin-4-yl)acetate (Compound 160) and 2-(7-chloro-2,2-difluoro-3-oxo-6-(perfluorophenyl)-2,3-dihydro-4H-benzo[b][1,4]oxazin-4-yl)acetic acid (Compound 161)

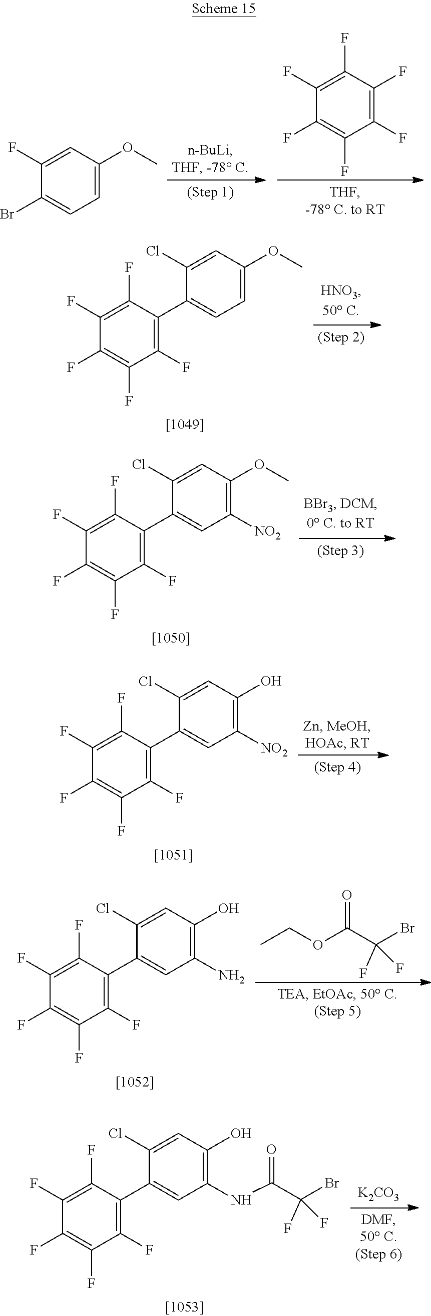

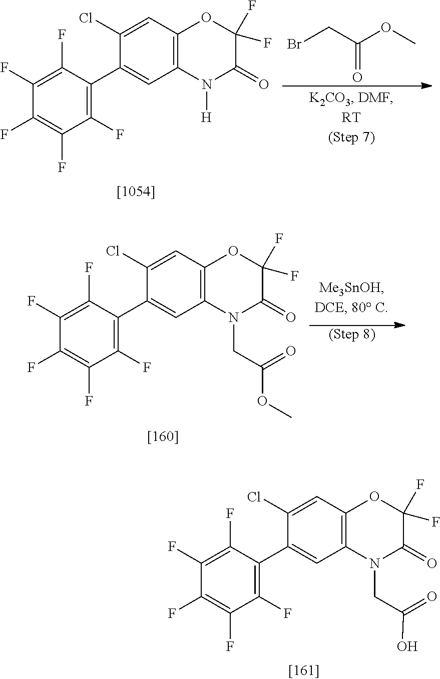

As shown in Step 1 of Scheme 15, to a stirred solution of 1-bromo-2-chloro-4-methoxybenzyne in anhydrous THF under an atmosphere of nitrogen was added n-BuLi (2.5 M in hexane) dropwise at −78° C. After addition was complete, stirring was continued at −78° C. for 15 minutes and hexafluorobenzene was added dropwise at −78° C. The resulting mixture was warmed to room temperature, stirred for 2 hours under nitrogen, diluted with water, and extracted with ethyl acetate. The combined organic layers were washed with brine, dried over anhydrous sodium sulfate, filtered, concentrated under reduced pressure, and purified by flash chromatography (0%-10% ethyl acetate in petroleum ether) within to afford 2′-chloro-2,3,4,5,6-pentafluoro-4′-methoxy-1,1′-biphenyl (Compound 1049).

As shown in Step 2 of Scheme 15, to a stirred solution of 2′-chloro-2,3,4,5,6-pentafluoro-4′-methoxy-1,1′-biphenyl in DCM (20 mL) and concentrated H₂SO₄ (80 mL) was added KNO₃ in portions at 0° C. The resulting solution was stirred at 20° C. for 16 hours under nitrogen, diluted with water, and extracted with ethyl acetate. The combined organic layers were washed with brine, dried over anhydrous sodium sulfate, filtered, concentrated under reduced pressure, and purified by flash chromatography (0%-25% ethyl acetate in petroleum ether) to afford 2′-chloro-2,3,4,5,6-pentafluoro-4′-methoxy-5′-nitro-1,1′-biphenyl (Compound 1050).

As shown in Step 3 of Scheme 15, to a stirred solution of 2′-chloro-2,3,4,5,6-pentafluoro-4′-methoxy-5′-nitro-1,1′-biphenyl in DCM under a nitrogen atmosphere was added BBr₃ dropwise at 0° C. The solution was stinted at 0° C. for 2 hours under nitrogen, diluted with water, and extracted with ethyl acetate. The combined organic layers were washed with brine, dried over anhydrous sodium sulfate, filtered, and concentrated under reduced pressure to afford 2-chloro-2′,3′,4′,5′,6′-pentafluoro-5-nitro-[1,1′-biphenyl]-4-ol (Compound 1051). This material was used as is in subsequent reactions.

As shown in Step 4 of Scheme 15, to a stirred solution of 2-chloro-2′,3′,4′,5′,6′-pentafluoro-5-nitro-[1,1′-biphenyl]-4-ol in MeOH/HOAc was added Zn dust in portions. The resulting mixture was stirred at 25° C. under nitrogen, diluted with water, and extracted with ethyl acetate. The combined organic layers were washed with brine, dried over anhydrous sodium sulfate, filtered, concentrated under reduced pressure, and purified by flash chromatography (0%-30% ethyl acetate in petroleum ether) to afford 5-amino-2-chloro-2′,3′,4′,5′,6′-pentafluoro-[1,1′-biphenyl]-4-ol (Compound 1052).

As shown in Step 5 of Scheme 15, to a stirred solution of 5-amino-2-chloro-2′,3′,4′,5′,6′-pentafluoro-[1,1′-biphenyl]-4-ol and TEA in EtOAc was added and ethyl 2-bromo-2,2-difluoroacetate in portions at 20° C. The resulting mixture was stirred at 50° C. for 16 hours under nitrogen, cooled to room temperature, diluted with water, and extracted with ethyl acetate. The combined organic layers were washed with brine, dried over anhydrous sodium sulfate, filtered, concentrated under vacuum, and purified by reversed-phase flash chromatography to afford 2-bromo-N-(6-chloro-2′,3′,4′,5′,6′-pentafluoro-4-hydroxy-[1,1′-biphenyl]-3-yl)-2,2-difluoroacetamide (Compound 1047).

As shown in Step 6 of Scheme 15, to a stirred solution of 2-bromo-N-(6-chloro-2′,3′,4′,5′,6′-pentafluoro-4-hydroxy-[1,1′-biphenyl]-3-yl)-2,2-difluoroacetamide in DMF was added K₂CO₃ in portions at 20° C. The resulting mixture was stirred at 50° C. for 2 hours under nitrogen, diluted with water, and extracted with ethyl acetate. The combined organic layers were washed with brine, dried over anhydrous sodium sulfate, filtered, concentrated under reduced pressure, and purified by flash chromatography (0%-20% ethyl acetate in petroleum ether) to afford 7-chloro-2,2-difluoro-6-(perfluorophenyl)-2H-benzo[B][1,4]oxazin-3(4H)-one (Compound 1054).

As shown in Step 7 of Scheme 14, to a stirred solution of 7-chloro-2,2-difluoro-6-(perfluorophenyl)-2H-benzo[b][1,4]oxazin-3(4H)-one (200 mg, 0.51 mmol) and methyl 2-bromoacetate (95 mg, 0.62 mmol) in DMF (5 mL) was added K₂CO₃ (143 mg, 1.0 mmol) at room temperature under a nitrogen atmosphere. The mixture was stirred at room temperature for 3 hours, diluted with water, and extracted with ethyl acetate. The combined organic layers were washed with brine, dried over anhydrous sodium sulfate, filtered, concentrated under reduced pressure, and purified by reversed-phase flash chromatography (5% to 90% acetonitrile water), to afford methyl 2-(7-chloro-2,2-difluoro-3-oxo-6-(perfluorophenyl)-2,3-dihydro-4H-benzo[b][1,4]oxazin-4-yl)acetate (Compound 160, 38 mg, 16% yield): GCMS calculated for C₁₇H₇ClF₇NO₄=457.0, found 457.0, ¹H-NMR (400 MHz, DMSO-d₆) δ 7.97 (s, 1H), 7.81 (s, 1H), 4.88 (s, 2H), 3.72 (s, 3H); ¹⁹F-NMR (377 MHz, DMSO-d₆) δ −75.31, −139.72, −153.03, −162.02.

As shown in Step 8 of Scheme 14, to a stirred solution of methyl 2-(7-chloro-2,2-difluoro-3-oxo-6-(perfluorophenyl)-2,3-dihydro-4H-benzo[b][1,4]oxazin-4-yl)acetate (50 mg, 0.10 mmol) in DCE (2 mL) was added trimethylstannanol (40 mg, 0.21 mmol) at room temperature under a nitrogen atmosphere. The resulting mixture was stirred at 80° C. for 1 hour under nitrogen atmosphere, cooled to room temperature, diluted with water, acidified to pH 4-5 with formic acid, extracted with ethyl acetate. The combined organic layers were washed with brine, dried over anhydrous Na₂SO₄, concentrated under reduced pressure, and purified by reversed-phase flash chromatography (5% to 95% acetonitrile water), to afford 2-(7-chloro-2,2-difluoro-3-oxo-6-(perfluorophenyl)-2,3-dihydro-4H-benzo[b][1,4]oxazin-4-yl)acetic acid (Compound 161, 21 mg, 43% yield): MS (ESI) calculated for C₁₆H₅ClF₇NO₄ [M−1]⁻=442.0, found, 441.9; ¹H-NMR (400 MHz, DMSO-d₆) δ 13.57 (b, 1H), 7.94 (s, 1H), 7.76 (s, 1H), 4.72 (s, 2H); ¹⁹F-NMR (377 MHz, DMSO-d₆) δ −75.12, −139.78, −153.25, −162.17.

Alkylation of 7-chloro-2,2-difluoro-6-(perfluorophenyl)-2H-benzo[b][1,4]oxazin-3(4H)-one (Compound 1054) with methyl 2-bromopropanoate followed by separation of the resulting enantiomeric mixture by chiral HPLC produced methyl (S)-2-(7-chloro-2,2-difluoro-3-oxo-6-(perfluorophenyl)-2,3-dihydro-4H-benzo[b][1,4]oxazin-4-yl)propanoate (Compound 170): GCMS calculated for C₁₈H₉ClF₇NO₄=471.0, found 471.0; ¹H-NMR (400 MHz, DMSO-d₆) δ 7.97 (s, 1H), 7.82 (s, 1H), 5.35-5.54 (m, 1H), 3.67 (s, 3H), 1.55 (d, J=6.8 Hz, 3H), ¹⁹F-NMR (376 MHz, DMSO-d₆) δ −75.67, −139.73, −153.01, −162.08; and methyl (R)-2-(7-chloro-2,2-difluoro-3-oxo-6-(perfluorophenyl)-2,3-dihydro-4H-benzo[b][1,4]oxazin-4-yl)propanoate (Compound 171): GCMS calculated for C₁₈H₉ClF₇NO₄=471.0, found 471.0, ¹H-NMR (400 MHz, DMSO-d₆) δ7.98 (s, 1H), 7.82 (s, 1H), 5.34-5.52 (m, 1H), 3.67 (s, 3H), 1.55 (d, J=6.8 Hz, 3H); ¹⁹F-NMR (376 MHz, DMSO-d₆) δ −75.67, −139.73, −153.01, −162.07. Hydrolysis of Compounds 170 and 171 with trimethylstannanol in DCE at 80° C. produced, respectively. (S)-2-(7-chloro-2,2-difluoro-3-oxo-6-(perfluorophenyl)-2,3-dihydro-4H-benzo[b][1,4]oxazin-4-yl)propanoic acid (Compound 172): MS (ESI) calculated for C₁₇H₇ClF₇NO₄ [M−1]⁻=456.0, found 455.9; ¹H-NMR (400 MHz, DMSO-d₆) δ 13.22 (s, 1H), 7.96 (s, 1H), 7.81 (s, 1H), 5.36-5.26 (m, 1H), 1.54 (d, J=6.8 Hz, 3H); ¹⁹F-NMR (376 MHz, DMSO-d₆) δ −77.12, −139.78, −153.10, −162.17; and (R)-2-(7-chloro-2,2-difluoro-3-oxo-6-(perfluorophenyl)-2,3-dihydro-4H-benzo[b][1,4]oxazin-4-yl)propanoic acid (Compound 173): MS (ES) calculated for C₁₇H₇ClF₇NO₄ [M−1]⁻=456.0, found 455.9; ¹H-NMR (400 MHz, DMSO-d₆) δ 13.26 (b, 1H), 7.96 (s, 1H), 7.81 (s, 1H), 5.32-5.50 (m, 1H), 1.54 (d, J=6.8 Hz, 3H); ¹⁹F-NMR (376 MHz, DMSO-d₆) δ −73.90, −77.66, −139.78, −153.10, −162.17.

### Example 14. Preparation of methyl 2-(2,2-difluoro-7-methyl-3-oxo-6-(perfluorophenyl)-2,3-dihydro-4H-benzo[b][1,4]oxazin-4-yl)acetate (Compound 160) and 2-(2,2-difluoro-7-methyl-3-oxo-6-(perfluorophenyl)-2,3-dihydro-4H-benzo[b][1,4]oxazin-4-yl)acetic acid (Compound 161)

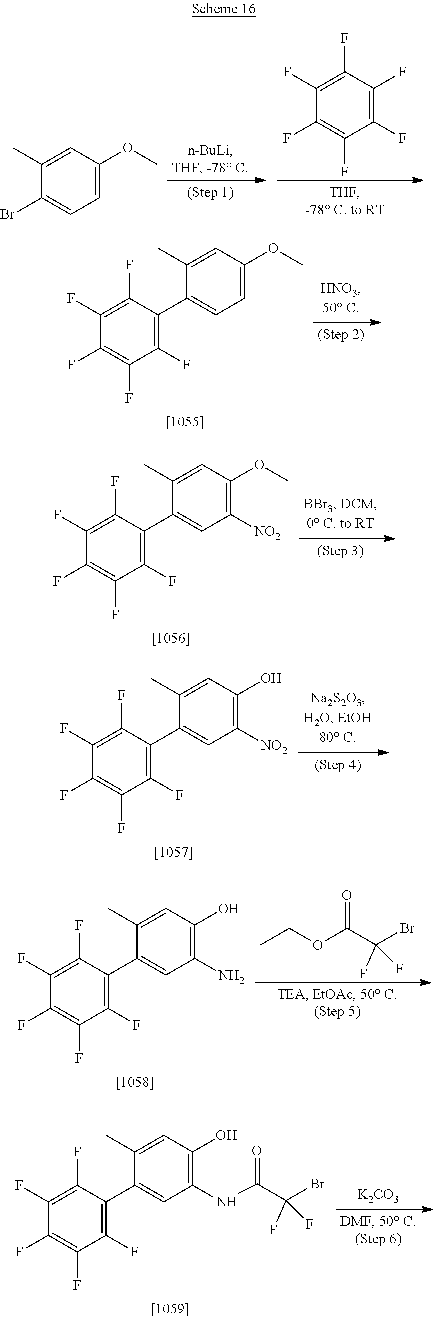

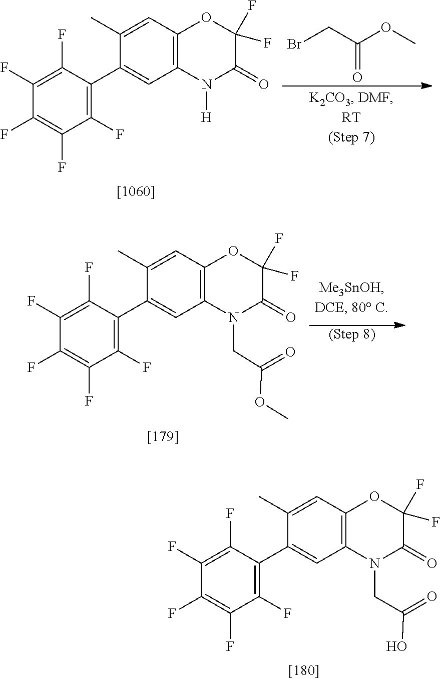

As shown in Step 1 of Scheme 16, to a stirred solution of 1-bromo-4-methoxy-2-methylbenzene in anhydrous THE under an atmosphere of nitrogen was added n-BuLi (2.5 M in hexane) dropwise at −78° C. After addition was complete, stirring was continued at −78° C. for 15 minutes and hexafluorobenzene was added dropwise at −78° C. The resulting mixture was warmed to room temperature, stirred for 2 hours under nitrogen, diluted with water, and extracted with ethyl acetate. The combined organic layers were washed with brine, dried over anhydrous sodium sulfate, filtered, concentrated under reduced pressure, and purified by flash chromatography (0%-10% ethyl acetate in petroleum ether) within to afford 2,3,4,5,6-pentafluoro-4′-methoxy-2′-methyl-1,1′-biphenyl (Compound 1055).

As shown in Step 2 of Scheme 16, to a stirred solution of 2,3,4,5,6-pentafluoro-4′-methoxy-2′-methyl-1,1′-biphenyl in DCM (20 mL) and concentrated H₂SO₄ (80 mL) was added KNO₃ in portions at 0° C. The resulting solution was stirred at 20° C. for 16 hours under nitrogen, diluted with water, and extracted with ethyl acetate. The combined organic layers were washed with brine, dried over anhydrous sodium sulfate, filtered, concentrated under reduced pressure, and purified by flash chromatography (0%-25% ethyl acetate in petroleum ether) to afford 2,3,4,5,6-pentafluoro-4′-methoxy-2′-methyl-5′-nitro-1,1′-biphenyl (Compound 1056).

As shown in Step 3 of Scheme 16, to a stirred solution of 2,3,4,5,6-pentafluoro-4′-methoxy-2′-methyl-5′-nitro-1,1′-biphenyl in DCM (70 mL) under a nitrogen atmosphere was added BBr₃ dropwise at 0° C. The solution was stirred at 0° C. for 2 hours under nitrogen, diluted with water, and extracted with ethyl acetate. The combined organic layers were washed with brine, dried over anhydrous sodium sulfate, filtered, and concentrated under reduced pressure to afford 2′,3′,4′,5′,6′-pentafluoro-2-methyl-5-nitro-[1,1′-biphenyl]-4-ol (Compound 1057). This material was used as is in subsequent reactions.

As shown in Step 4 of Scheme 16, to a stirred solution of 2′,3′,4′,5′,6′-pentafluoro-2-methyl-5-nitro-[1,1′-biphenyl]-4-ol in EtOH and H₂O was added Na₂S₂O₄ in portions at 20° C. The resulting mixture was stirred at 100° C. for 2 hours under nitrogen, diluted with water, and extracted with ethyl acetate. The combined organic layers were washed with brine, dried over anhydrous sodium sulfate, filtered, concentrated under reduced pressure, and purified by flash chromatography (0%-30% ethyl acetate in petroleum ether) to afford 5-amino-2′,3′,4′,5′,6′-pentafluoro-2-methyl-[1,1′-biphenyl]-4-ol (Compound 1058).

As shown in Step 5 of Scheme 16, to a stirred solution of 5-amino-2′,3′,4′,5′,6′-pentafluoro-2-methyl-[1,1′-biphenyl]-4-ol and TEA in EtOAc was added and ethyl 2-bromo-2,2-difluoroacetate in portions at 20° C. The resulting mixture was stirred at 50° C. for 16 hours under nitrogen, cooled to room temperature, diluted with water, and extracted with ethyl acetate. The combined organic layers were washed with brine, dried over anhydrous sodium sulfate, filtered, concentrated under vacuum, and purified by reversed-phase flash chromatography to afford 2-bromo-2,2-difluoro N-(2′,3′,4′,5′,6′-pentafluoro-4-hydroxy-6-methyl-[1,1′-biphenyl]-3-yl)acetamide (Compound 1059).

As shown in Step 6 of Scheme 16, to a stirred solution of 2-bromo-2,2-difluoro-N-(2′,3′,4′,5′,6′-pentafluoro-4-hydroxy-6-methyl-[1,1′-biphenyl]-3-yl)acetamide) in DMF was added K₂CO₃ in portions at 20° C. The resulting mixture was stirred at 50° C. for 2 hours under nitrogen, diluted with water, and extracted with ethyl acetate. The combined organic layers were washed with brine, dried over anhydrous sodium sulfate, filtered, concentrated under reduced pressure, and purified by flash chromatography (0%-20% ethyl acetate in petroleum ether) to afford 2,2-difluoro-7-methyl-6-(perfluorophenyl)-2H-benzo[b][1,4]oxazin-3(4H)-one (Compound 1060).

As shown in Step 7 of Scheme 16, to a stirred solution of 2,2-difluoro-7-methyl-6-(perfluorophenyl)-2H-benzo[b][1,4]oxazin-3(4H)-one (100 mg, 0.24 mmol) and methyl 2-bromoacetate (50 mg, 0.32 mmol) in DMF (2.5 mL) was added K₂CO₃ (57 mg, 0.41 mmol) at room temperature under a nitrogen atmosphere. The mixture was stirred at room temperature for 2 hours, diluted with water, and extracted with ethyl acetate. The combined organic layers were washed with brine, dried over anhydrous sodium sulfate, filtered, concentrated under reduced pressure, and purified by reversed-phase flash chromatography (5% to 78% acetonitrile/water) to afford methyl 2-(2,2-difluoro-7-methyl-3-oxo-6-(perfluorophenyl)-2,3-dihydro-4H-benzo[b][1,4]oxazin-4-yl)acetate (Compound 179, 38 mg, 30% yield): GCMS calculated for C₁₈H₁₀F₇NO₄=437.0, found 437.0; ¹H-NMR (400 MHz, DMSO-d₆) δ 7.52 (d, J=4.8 Hz, 2H), 4.86 (s, 2H), 3.70 (s, 3H), 2.16 (s, 3H); ¹⁹F-NMR (377 MHz, DMSO-d₆) δ −75.60, −140.51, −155.08, −162.39.

As shown in Step 8 of Scheme 16, to a stirred solution of methyl 2-(7-chloro-2,2-difluoro-3-oxo-6-(perfluorophenyl)-2,3-dihydro-4H-benzo[b][1,4]oxazin-4-yl)acetate (100 mg, 0.22 mmol) in DCE (3 mL) was added trimethyltin chloride (91 mg, 0.48 mmol) at room temperature under a nitrogen atmosphere. The resulting mixture was stirred at 65° C. for 5 hours under a nitrogen atmosphere, cooled to room temperature, diluted with water, acidified to pH 4-5 with formic acid, and extracted with ethyl acetate. The combined organic layers were washed with brine, dried over anhydrous Na₂SO₄, concentrated under reduced pressure, and purified by reversed-phase flash chromatography (5% to 53% acetonitrile/water) to afford 2-(2,2-difluoro-7-methyl-3-oxo-6-(perfluorophenyl)-2,3-dihydro-4H-benzo[b][1,4]oxazin-4-yl)acetic acid (Compound 180, 52 mg, 52% yield): MS (ESI) calculated for C₁₇H₈F₇NO₄ [M−1]⁻=422.0, found 421.9; ¹H-NMR (400 MHz, DMSO-d₆) δ 13.36 (s, 1H), 7.49 (d, J=2.7 Hz, 2H), 4.74 (s, 2H), 2.16 (s, 3H); ¹⁹F-NMR (377 MHz, DMSO-d₆) δ −75.44, −140.54, −155.25, −162.50.

Alkylation of 2,2-difluoro-7-methyl-6-(perfluorophenyl)-2H-benzo[b][1,4]oxazin-3(4H)-one (Compound 1060) with methyl 2-bromopropanoate followed by separation of the resulting enantiomeric mixture by chiral HPLC produced methyl (S)-2-(2,2-difluoro-7-methyl-3-oxo-6-(perfluorophenyl)-2,3-dihydro-4H-benzo[b][1,4]oxazin-4-yl)propanoate (Compound 181): MS (ESI) calculated for C₁₉H₁₂F₇NO₄ [M+1]⁺=452.1, found 452.0; ¹H-NMR (400 MHz, DMSO-d₆) δ 7.54 (d, J=8.0 Hz, 2H), 5.41 (d, J=7.2 Hz, 1H), 3.66 (s, 3H), 2.17 (s, 3H), 1.54 (d, J=6.8 Hz, 3H); ¹⁹F-NMR (377 MHz, DMSO-d₆) δ −76.52, −140.40, −155.04, −162.44; and methyl (R)-2-(2,2-difluoro-7-methyl-3-oxo-6-(perfluorophenyl)-2,3-dihydro-4H-benzo[b][1,4]oxazin-4-yl)propanoate (Compound 182): MS (ESI) calculated for C₁₉H₁₂F₇NO₄ [M+1]=452.1, found 452.0; ¹H-NMR (400 MHz, DMSO-d₆) δ 7.54 (d, J=8.0 Hz, 2H), 5.46-5.38 (m, 1H), 3.66 (d, J=2.4 Hz, 3H), 2.17 (s, 3H), 1.59-1.49 (m, 3H); ¹⁹F-NMR (377 MHz, DMSO-d₆) δ −76.33, −140.52, −154.99, −162.38. Hydrolysis of Compounds 181 and 182 with trimethylstannanol in DCE at 80° C. produced, respectively, (S)-2-(2,2-difluoro-7-methyl-3-oxo-6-(perfluorophenyl)-2,3-dihydro-4H-benzo[b][1,4]oxazin-4-yl)propanoic acid (Compound 183): MS (ESI) calculated for C₁₈H₁₀F₇NO₄ [M−1]⁻=436.0, found 436.0; ¹H-NMR (400 MHz, DMSO-dh) δ 13.21 (b, 1H), 7.58-7.40 (m, 2H), 5.32-5.50 (m, 1H), 2.16 (s, 3H), 1.51 (d, J=6.8 Hz, 3H); ¹⁹F-NMR (377 MHz, DMSO-d) δ −75.62, −77.45, −140.51, −155.05, −162.41; and (R)-2-(2,2-difluoro-7-methyl-3-oxo-6-(perfluorophenyl)-2,3-dihydro-4H-benzo[b][1,4]oxazin-4-yl)propanoic acid (Compound 184): MS (ESI) calculated for C₁₈H₁₀F₇NO₄ [M−1]⁻=436.0, found 436.0; ¹H-NMR (400 MHz, DMSO-d₆) δ 13.22 (s, 1H), 7.52 (d, J=9.6 Hz, 2H), 5.33-5.23 (m, 1H), 2.16 (s, 3H), 1.51 (d, J=6.8 Hz, 3H); ¹⁹F-NMR (377 MHz, DMSO-d₆) δ −76.51, −77.69, −140.49, −155.05, −162.41.

### Example 15. Preparation of methyl 2-(7-fluoro-3-oxo-6-(perfluorophenyl)spiro[benzo[b][1,4]oxazine-2,1′-cyclopropan]-4(3H)-yl)acetate (Compound 22) and 2-(7-fluoro-3-oxo-6-(perfluorophenyl)spiro[benzo[b][1,4]oxazine-2,1′-cyclopropan]-4(3H)-yl)acetic acid (Compound 23)

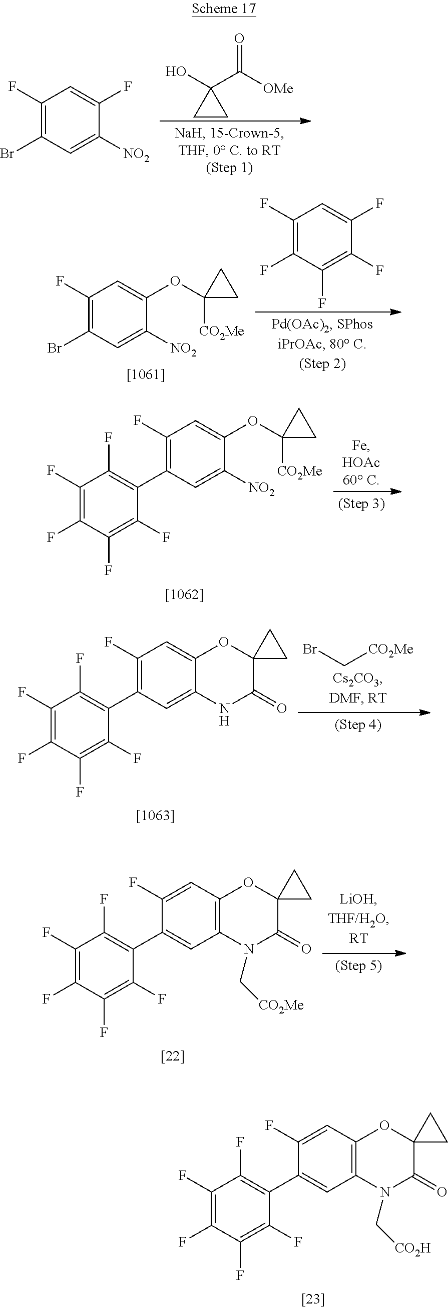

As shown in Step 1 of Scheme 17, to a solution of 1-bromo-2,4-difluoro-5-nitrobenzene (102 mg, 4.30 mmol) in THE (5 mL) at 0° C. was added NaH (60% oil dispersion, 206 mg, 5.16 mmol) in portions under an atmosphere in nitrogen. The mixture was stirred at room temperature for 10 minutes, followed by the addition of 15-crown-5 (94 mg, 0.43 mmol) and methyl 1-hydroxycyclopropane-1-carboxylate (500 mg, 4.30 mmol). The resulting mixture was stirred at room temperature for 16 hours. The reaction was diluted by the addition of water at 0° C. and extracted with ethyl acetate. The combined organics were washed with brine, dried over anhydrous sodium sulfate, filtered, and concentrated under reduced pressure. The residue was purified by flash chromatography (0-35% ethyl acetate in petroleum ether) to afford methyl 1-(4-bromo-5-fluoro-2-nitrophenoxy)cyclopropane-1-carboxylate (Compound 1061, 500 mg, 33% yield) as a yellow solid: GCMS calculated for C₁₁H₉BrFNO₅=332.9, found 332.9.

As shown in Step 2 of Scheme 17, to a solution of 1,2,3,4,5-pentafluorobenzene (503 mg, 2.99 mmol) in isopropyl acetate (5 mL) under an atmosphere of nitrogen were added K₂CO₃ (413 mg, 2.99 mmol), 2-dicyclohexylphosphino-2′,6′-dimethoxybiphenyl (SPhos, 122 mg, 0.29 mmol), and Pd(OAc)₂ (33 mg, 0.15 mmol). The mixture was stirred at room temperature for 5 minutes and methyl 1-(4-bromo-5-fluoro-2-nitrophenoxy)cyclopropane-1-carboxylate (500 mg, 1.49 mmol) was added. The resulting mixture was stirred at 80° C. for 12 hours under an atmosphere of nitrogen, cooled to room temperature, diluted with water, and extracted with ethyl acetate. The combined organics were washed with brine, dried over anhydrous sodium sulfate, filtered, and concentrated under reduced pressure. The residue was purified by flash chromatography (0-20% ethyl acetate in petroleum ether) to afford methyl 1-((2,2′,3′,4′,5′,6′-hexafluoro-5-nitro-[1,1′-biphenyl]-4-yl)oxy)cyclopropane-1-carboxylate (Compound 1062, 400 mg, 71% yield) as a brown oil: GCMS calculated for C₁₇H₉F₆NO_{5=421.0}, found 421.0.

As shown in Step 3 of Scheme 17, to a solution of methyl 1-((2,2′,3′,4′,5′,6′-hexafluoro-5-nitro-[1,1′-biphenyl]-4-yl)oxy)cyclopropane-1-carboxylate (200 mg, 0.47 mmol) in acetic acid (1 mL) was added iron powder (132 mg, 2.37 mmol). The mixture was stirred at 60° C. for 12 hours, filtered, and the filtrate concentrated under reduced pressure. The residue was purified by preparative reversed-phase HPLC using the following conditions—Waters XBridge C₁₈ column (30 mm×150 mm, 5 μm, 130 angstrom); mobile phase A: water (10 mM NH₄HCO₃); mobile phase B=acetonitrile, gradient; 55% B/A to 66% B/A, to afford 7-fluoro-6-(perfluorophenyl)spiro[benzo[b]-[1,4]oxazine-2,1′-cyclopropan]-3(4H)-one (Compound 1063, 100 mg, 58% yield) as a white solid: MS (ESI) calculated for C₁₆H₇F₆NO₂ [M−1]⁻=358.0, found 358.0; ¹H-NMR (400 MHz, DMSO-d₆) δ 11.02 (s, 1H), 7.12 (d, J=10.4 Hz, 1H), 6.99 (d, J=6.8 Hz, 1H), 1.35-1.29 (m, 2H), 1.29-1.23 (m, 2H); ¹⁹F-NMR (400 MHz, DMSO-d₆) δ −118.92, −141.27, −154.15, −162.24.

As shown in Step 4 of Scheme 17, to a solution of 7-fluoro-6-(perfluorophenyl)spiro-[benzo[b][1,4]oxazine-2,1′-cyclopropan]-3(4H)-one (150 mg, 0.42 mmol) in DMF (2 mL) were added Cs₂CO₃ (272 mg, 0.83 mmol) and methyl 2-bromoacetate (95 mg, 0.63 mmol). The resulting mixture was stirred at room temperature for 3 hours, then purified by reversed-phase flash chromatography (20% to 60% acetonitrile in water gradient) to afford methyl 2-(7-fluoro-3-oxo-6-(perfluorophenyl)-spiro[benzo[b][1,4]oxazine-2,1′-cyclopropan]-4(3H)-yl)acetate (Compound 22, 60 mg, 33% yield) as a white solid: GCMS calculated for C₁₉H₁₁F₆NO₄=431.1, found 431.1; ¹H-NMR (400 MHz, DMSO-d₆) δ 7.41 (d, J=6.4 Hz, 1H), 7.20 (d, J=9.6 Hz, 1H), 4.73 (s, 2H), 3.68 (s, 3H), 1.38-1.30 (m, 4H); ¹⁹F-NMR (376 MHz, DMSO-d₆) δ −118.29, −140.44, −154.09, −162.30.

As shown in Step 5 of Scheme 17, to a mixture of methyl 2-(7-fluoro-3-oxo-6-(perfluorophenyl)spiro[benzo[b][1,4]oxazine-2,1′-cyclopropan]-4(3H)-yl)acetate (40 mg, 0.09 mmol) in THF (1 mL) and H₂O (0.3 mL) was added LiOH (5.5 mg, 0.13 mmol). The resulting mixture was stirred at 25° C. for 16 hours under nitrogen, concentrated under reduced pressure, and purified by reversed-phase flash chromatography (5% to 80% ACN in water) to afford 2-(7-fluoro-3-oxo-6-(perfluorophenyl)spiro[benzo[b][1,4]oxazine-2,1′-cyclopropan]-4(3H)-yl)acetic acid (Compound 23, 20 mg, 51% yield) as a white solid: MS (ESI) calculated for C₁₈H₉F₆NO₄ [M−1]⁻=416.0, found 431.1; ¹H-NMR (400 MHz, DMSO-d₆) δ 13.07 (s, 1H), 7.35 (d, J=6.4 Hz, 1H), 7.19 (d, J=9.6 Hz, 1H), 4.61 (s, 2H), 1.37-1.30 (m, 4H); ¹⁹F-NMR (376 MHz, DMSO-d₆) δ 118.61, −140.44, −154.09, −162.36.

Alkylation of Compound 1063 with methyl 2-bromopropanoate, followed by separation of the resulting enantiomers by chiral HPLC, produced methyl (S)-2-(7-fluoro-3-oxo-6-(perfluorophenyl)spiro[benzo[b][1,4]oxazine-2,1′-cyclopropan]-4(3H)-yl)propanoate (Compound 31): GCMS calculated for C₂₀H₁₃F₆NO₄=445.1, found 445.1; ¹H-NMR (400 MHz, DMSO-d₆) δ 7.45 (d, J=6.4 Hz, 1H), 7.23 (d, J=9.6 Hz, 1H), 5.19-5.15 (m, 1H), 3.64 (s, 3H), 1.51 (d, J=6.8 Hz, 3H), 1.38-1.23 (m, 4H); ¹⁹F-NMR (377 MHz, DMSO-d₆) δ −118.02, −140.44, −153.91, −162.31; and methyl (R)-2-(7-fluoro-3-oxo-6-(perfluorophenyl)spiro[benzo[b][1,4]oxazine-2,1′-cyclopropan]-4(3H)-yl)propanoate (Compound 32): GCMS calculated for C₂₀H₁₃F₆NO₄=445.1, found 445.1; ¹H-NMR (400 MHz, DMSO-d₆) δ 7.45 (d, J=6.4 Hz, 1H), 7.23 (d, J=9.6 Hz, 1H), 5.19-5.15 (m, 1H), 3.64 (s, 3H), 1.51 (d, J=6.8 Hz, 3H), 1.38-1.23 (m, 4H); ¹⁹F-NMR (377 MHz, DMSO-d₆) δ −118.02, −140.44, −153.91, −162.31. Each of Compounds 31 and 32 was treated with 4M HCl in dioxane at 100° C. to produce, respectively, (S)-2-(7-fluoro-3-oxo-6-(perfluorophenyl)spiro[benzo[b][1,4]oxazine-2,1′-cyclopropan]-4(3H)-yl)propanoic acid (Compound 33): MS (ESI) calculated for C₁₉H₁₁F₆NO₄ [M−1]⁻=430.1, found 430.0; ¹H-NMR (400 MHz, DMSO-d₆) δ 12.89 (s, 1H), 7.40 (d, J=6.4 Hz, 1H), 7.21 (d, J=9.6 Hz, 1H), 5.08 (m, 1H), 1.48 (d, J=7.2 Hz, 3H), 1.38-1.21 (m, 4H); ¹⁹F-NMR (376 MHz, DMSO-d₆) δ −118.36, −140.51, −153.95, −162.30; and (R)-2-(7-fluoro-3-oxo-6-(perfluorophenyl)spiro-[benzo[b][1,4]oxazine-2,1′-cyclopropan]-4(311)-yl)propanoic acid (Compound 34): MS (ESI) calculated for C₁₉H₁₁F₆NO₄ [M−1]⁻=430.1, found 430.0; ¹H-NMR (400 MHz, DMSO-d₆) δ 12.88 (s, 1H), 7.40 (d, J=6.4 Hz, 1H), 7.21 (d, J=9.6 Hz, 1H), 5.10-5.06 (m, 1H), 1.49 (d, J=6.8 Hz, 3H), 1.38-1.22 (m, 41T); ¹⁹F-NMR (377 MHz, DMSO-d₆) δ −118.32, −140.49, −153.98, −162.32.

### BIOLOGICAL EXAMPLES

### Example 16. Amaranthus tuberculantum Protoporphyrinogen Oxidase (AmPPO) Expression and Purification

The coding sequence of AmPPO was optimized for E. coli expression and assembled from synthetic oligonucleotides. Synthetic fragments were introduced into a pET28b vector (Novagen) using restriction-less “Hot Fusion” cloning process (Fu C., et al., ‘Hot Fusion: An Efficient Method to Clone Multiple DNA Fragments as Well as Inverted Repeats without Ligase’, PLoS One (2014) Vol. 9(12), page e115318). The resulting DNA was sequence-verified. Construct encoding mutant version (Δ210-AmPPO) of the enzyme was produced via PCR-based mutagenesis using Q5 mutagenesis kit (NEB).

Lysogeny broth (LB media, 10 mL) supplemented with 100 μg/mL kanamyci was inoculated with a single colony of BL21(DE3) competent E. coli transformed with pET28b_PPO_CHis. [Is pET28b_PPO_CHis. The culture was grown at 37° C. with shaking at 230 rpm overnight. This culture was then used to inoculate 1 L of autoinduction media (AIM) prepared by the method of Fox, B. G., & Blommel, P. G. (2009). Autoinduction of protein expression, ‘Current Protocols in Protein Science,’Chapter 5, Unit-5.23. The resulting culture was grown at 37° C. with shaking at 230 rpm for 4 to 6 hours and an additional 40 to 48 hours at 18° C. The culture was collected and centrifuged. The resulting AmPPO enzyme-containing cell pellets were frozen and stored at −80° C. for future use.

The same procedure used to produce AmPPO was used to produce mutant ΔG210-AmPPO (a PPO mutant in which the glycine at position 210 is absent), except E. coli used was transformed with pET28b_ΔG210 PPO_Chis.

A detergent solution was prepared by mixing together the following: 175 mL of B-PER Thermo Scientific); 75 mL of Y-PER (Thermo Scientific); 15 mL of 1M TRIS buffer, pH 9.0, 15 mL of 5M NaCl; 50 mL of glycerol; 2.5 mL of Triton-X 100; and 1 mg of Flavin Adenine Dinucleotide (FAD). A portion of this solution (about 80 mL-100 mL) is set aside and supplemented with imidazole to a final concentration of 10 mM and a pH of pH 8.0. The remainder of detergent solution was supplemented with Hen Egg White Lysozyme (Gold Bio, 1 mg/mL) and Serratia endonuclease (produced in house) and added to about 45 g of frozen enzyme-containing cell pellets, which were allowed to thaw in the lysis solution with vigorous stirring for 30 minutes at room temperature, then briefly sonicated (30 seconds on 50% power using a VNN R brand sonic disruptor). Incubation was continued with stirring for additional 15-30 minutes at 4° C. The lysate was clarified for 35 minutes by centrifugation at 14,000 RPM. The resulting clarified lysate was incubated for 1 hour at 4° C. with gentle stirring with His-SELECT® resin (Sigma, 20 mL of 50% slurry in 20% ethanol, washed 2× with 30 mM TRIS pH 8.1, 10% glycerol, 220 mM NaCl). The resin slurry was transferred to a disposable plastic column and washed with 10 mM Imidazole, 250 mM NaCl, 30 mM TRIS pH 8.5, 10% glycerol until the bound protein was deemed sufficiently washed away from lysate components (about 6-8 column volumes). The resin was then washed thoroughly (about 3 column volumes) with the previously set-aside detergent 1-10 final buffer, followed by elution with the same buffer supplemented with 250 mM imidazole, pH 8.1. Enzyme-containing fractions were collected and pooled based on SDS-PAGE analysis. Pooled fractions were diluted with pure glycerol to final concentration of 50% and the AmPPO enzyme or mutant ΔG210-AmPPO was stored at −20° C. in liquid form.

### Example 17a. PPO In Vitro Assay

Protoporphyrinogen IX (PPGIX) is prepared by reduction of protoporphyrin IX (PPIX) with a sodium amalgam as described by Jacobs and Jacobs, Enyzme 28: 206 (1982). Once prepared, the PPGIX solution is kept in the dark and all subsequent manipulations of it are performed in the dark.

The Base Buffer for the assay was 50 mM TRIS pH 8.5, 160 mM NaCl, 2 mM DTT, 0.01% Triton X-100. An antifoam solution was prepared by two serial 1 to 10 dilutions of Antifoam B Emulsion (SigmaAldrich) with Milli-Q water. Buffer A was freshly prepared by diluting AmPPO or mutant ΔG210-AmPPO in Base Buffer to 3-8 ug/ml concentration of enzyme. Buffer B was prepared by adding 2 ml of reduced 2 mM PPIX to 60 ml of Base Buffer and adjusting the pH back to 8.5 using glacial acetic acid. Finally, antifoam B (Sigma) was added to 0.01% final concentration. Buffer B was protected from light and used within 3 hours of its preparation.

A 384 well, clear bottom plate was used for the assay. Each test compound was dissolved in DMSO to a concentration of 30 mM. The test compounds, tested in triplicate, a butafenacil control, and a DMSO control were dispensed as 1.2 μL drops into a well of the plate. The wells were diluted with 60 μL of Buffer A and serially diluted 1 volume to 3 volumes over 7 dilutions by removing 20 μL from the first well, mixing well with 40 μL of Buffer A in a second well, removing 20 μL from the second well, and continuing the dilutions in this manner until there were 8 test wells. To initiate the reaction, 40 μL of Buffer B was added to each well and the wells gently mixed at least 2 times. The plate was centrifuged at 2000 rpm for 1 minute and the absorbance or fluorescence were read at ambient temperature using a plate reader. IC₅₀'s were calculated using a nonlinear regression Sigmoidal dose-response model (GraphPad Prism, variable slope) with curve bottoms constrained to zero and curve tops constrained to plate-specific V_{average}.

Each of Compounds 1 to 6, 8 to 22, 24 to 33, 35 to 39, 41 to 42, 45 to 64, 66 to 67, and 70 to 148 had an IC₅₀ of less than 100 nM in the PPO in vitro assay.

### Example 17b. PPO In Vitro Assay

Protoporphyrinogen IX (PPGIX) is prepared by reduction of protoporphyrin IX (PPIX) with a sodium amalgam as described by Jacobs and Jacobs, Enyzme 28: 206 (1982). Once prepared, the PPGIX solution is kept in the dark and all subsequent manipulations of it are performed in the dark.

The Base Buffer for the assay was 50 mM TRIS pH 8.5, 160 mM NaCl, 2 mM DTT, 0.01% Triton X-100. An antifoam solution was prepared by two serial 1 to 10 dilutions of Antifoam B Emulsion (SigmaAldrich) with Milli-Q water. Buffer A was freshly prepared by diluting AmPPO or mutant ΔG210-AmPPO in Base Buffer to 3-8 ug/ml concentration of enzyme. Buffer B was prepared by adding 2 ml of reduced 2 mM PPIX to 60 ml of Base Buffer and adjusting the pH back to 8.5 using glacial acetic acid. Finally, antifoam B (Sigma) was added to 0.01% final concentration. Buffer B was protected from light and used within 3 hours of its preparation.

A 384 well, clear bottom plate was used for the assay. Each test compound was dissolved in DMSO to a concentration of 30 mM. The test compounds, tested in triplicate, a butafenacil control, and a DMSO control were dispensed as 1.2 μL drops into a well of the plate. The wells were diluted with 60 μL of Buffer A and serially diluted 1 volume to 3 volumes over 7 dilutions by removing 20 μL from the first well, mixing well with 40 μL of Buffer A in a second well, removing 20 μL from the second well, and continuing the dilutions in this manner until there were 8 test wells. To initiate the reaction, 40 μL of Buffer B was added to each well and the wells gently mixed at least 2 times. The plate was centrifuged at 2000 rpm for 1 minute and the absorbance or fluorescence were read at ambient temperature using a plate reader. IC₅₀s were calculated using a nonlinear regression Sigmoidal dose-response model (GraphPad Prism, variable slope) with curve bottoms constrained to zero and curve tops constrained to plate-specific Vera.

Each of Compounds 1 to 6, 8 to 22, 24 to 33, 35 to 39, 41 to 42, 45 to 64, 66 to 67, 70 to 154, 156 to 160, 162 to 171, 173 to 175, 177 to 179, 181 to 182, and 185 to 187 had an IC₅₀ of less than 100 nM in the PPO in vitro assay. Each of Compounds 23, 34, 43, 65, 68, 155, 161, 172, 176, 180, and 188 had an IC₅₀ of less than 1 μM in this assay.

Each of Compounds 8, 12, 15, 26 to 29, 31 to 32, 35 to 38, 45, 47 to 50, 53, 56, 62, 64, 66, 70, 76 to 77, 80, 84, 86, 88 to 89, 91, 93 to 94, 96 to 100, 103, 105 to 107, 109, 111 to 113, 115 to 116, 121 to 146, 152 to 154, 157, 162, 164, 174 to 175, 178, and 185 to 186 had an IC₅₀ of less than 100 nM in the AG210 PPO in vitro assay. Each of Compounds 11, 13 to 14, 21, 33, 39, 41 to 42, 46, 51, 54 to 55, 57 to 60, 63, 67 to 68, 71 to 75, 78 to 79, 81 to 83, 85, 87, 90, 92, 95, 101 to 102, 104, 108, 110, 114, 117 to 120, 147 to 151, 156, 158, 160, 163, 165 to 166, and 168 to 171 had an IC₅₀ of less than 1 μM in this assay.

### Example 18a. Testing the Post-Emergence Herbicidal Activity of Compounds of the Invention

Selected compounds of the invention were screened at 100 PPM against Amaranthus retroflexus (AMARE), Setaria italica (SETIT), and Echinochloa crus-galli (ECHCG).

Accordingly, PPO susceptible weed seeds were sown in 5″×5″ pots by quadrant containing Miracle-Gro potting mix (Scotts Miracle-Gro Company, Marysville, OH. USA) and grown in a Conviron growth chamber with appropriate growth conditions (temperature of 26/22° C. with photoperiod 16/8 h light day/night and light intensity of 300 μmol m⁻² s⁻¹ supplemented by LED lamps). Relative humidity in the growth chamber was maintained at around 65%. Plants were grown until 2-4 leaf stage and thinned to 5-8 plants per quadrant per species.

Compounds were formulated in 25% Acetone, 1% Crop oil concentrate (COC-Agridex), 0.1% Tween-20, and 2.5% Ammonium sulphate (AMS). Three replicate pots were treated with each compound. Treatment consisting of the above formulation excluding active compound was applied as a treatment control (TC). Plants were treated with the test compound solution in a laboratory spray chamber fitted with 8003 flat fan nozzles calibrated to deliver 187-200 L ha⁻¹ at 269 kPa. Plants were placed back in the growth chamber and evaluated for % visual injury compared to TC 7 days after treatment (DAT). The data presented in Table 3 indicate a percentage control, where 100% control indicates complete inhibition of growth.

Representative Compounds 8, 11, 74, 77, 78, 80, 85, 130, 131, and 132 showed herbicidal activity against weed species at a concentration of 100 parts per million (PPM), as shown in Table 3a.

TABLE 3a
Post-emergence herbicidal activity of selected compounds
of the invention 7 days after the compound application

| Cmpd. | Post-emergence % control (100 PPM) | | |
|---|---|---|---|
| No. | AMARE | SETIT | ECHCG |
| 8 | 80 | 75 | 45 |
| 11 | 95 | 96 | 83 |
| 74 | 100 | 100 | 33 |
| 77 | 100 | 98 | 88 |
| 78 | 100 | 100 | 70 |
| 80 | 100 | 93 | 75 |
| 85 | 100 | 88 | 25 |
| 130 | 100 | 100 | 92 |
| 131 | 100 | 83 | 15 |
| 132 | 100 | 100 | 70 |

### Example 18b. Testing the Post-Emergence Herbicidal Activity of Compounds of the Invention

Selected compounds of the invention were screened at 100 PPM against Amaranthus retroflexus (AMARE), Echinochloa crus-galli (ECHCG), Kochia scoparia (KCHSC), and Setaria italica (SETIT).

Accordingly, PPO susceptible weed seeds were sown in 5″ 5″ pots by quadrant containing Miracle-Gro potting mix (Scotts Miracle-Gro Company, Marysville, OH, USA) and grown in a Conviron growth chamber with appropriate growth conditions (temperature of 26/22° C. with photoperiod 16/8 h light day/night and light intensity of 300 μmol m² s⁻¹ supplemented by LED lamps). Relative humidity in the growth chamber was maintained at around 65%. Plants were grown until 2-4 leaf stage and thinned to 5-8 plants per quadrant per species.

Compounds were formulated in 25% Acetone, 1% Crop oil concentrate (COC-Agridex), 0.1% Tween-20, and 2.5% Ammonium sulphate (AMS). Three replicate pots were treated with each compound. Treatment consisting of the above formulation excluding active compound was applied as a treatment control (TC). Plants were treated with the test compound solution in a laboratory spray chamber fitted with 8003 flat fan nozzles calibrated to deliver 187-200 L ha⁻¹ at 269 kPa. Plants were placed back in the growth chamber and evaluated for % visual injury compared to TC 7 days after treatment (DAT). The data presented in Table 3 indicate a percentage control, where 100% control indicates complete inhibition of growth.

TABLE 3b
Post-emergence herbicidal activity of selected compounds
of the invention 7 days after the compound application

| | |
|---|---|
| Cmpd. | Post-emergence (100 ppm) |

| No. | AMARE | KCHSC | SETIT | ECHCG |
|---|---|---|---|---|
| 77 | 100% | 100% | 98% | 88% |
| 78 | 100% | 93% | 100% | 70% |
| 80 | 100% | 20% | 93% | 75% |
| 86 | 100% | 11% | 100% | 69% |
| 185 | 100% | 100% | 100% | 50% |

### Example 19. Testing the Pm-Emergence Herbicidal Activity of Compounds of the Invention

Selected compounds of the invention were screened at 200 PPM against Amaranthus retroflexus (AMARE). Echinochloa crus-galli (ECHCG). Kochia scoparia (KCHSC), and Selaria italica (SETIT).

Accordingly, PPO susceptible weed seeds were planted in 5″×5″ pots by quadrant containing custom field soil mix (Sandy loam with 4.7% OM, pH 7.0) and covered with a fine layer of the same soil. Compounds were formulated in 25% Acetone, 1% Crop oil concentrate (COC-Agridex), 0.1% Tween-20, and 2.5% Ammonium sulphate (AMS). Three replicate pots were treated with each compound. Treatment consisting of the above formulation excluding active compound was applied as a treatment control (TC). Pots were treated with the test compound solution in a laboratory spray chamber fitted with 8003 flat fan nozzles calibrated to deliver 187-200 L ha⁻¹ at 269 kPa. Compound was incorporated into the soil by simulating rainfall equivalent to 0.2 mm using the same track sprayer. Subsequently, pots were irrigated from the bottom until assessed for % growth and germination inhibition. Pots were placed back in the growth chamber and evaluated for % growth and germination inhibition compared to TC 7 days after treatment (DAT). Growth conditions are similar to the ones mentioned in POST emergence assay.

The data presented in Table 4 indicate a percentage control, where 100% control indicates complete inhibition of growth.

TABLE 4
Pre-emergence herbicidal activity of selected compounds
of the invention 7 days after the compound application

| | |
|---|---|
| Cmpd. | Pre-emergence (200 ppm) |

| No. | AMARE | KCHSC | SETIT | ECHCG |
|---|---|---|---|---|
| 77 | 100% | 0% | 70% | 3% |
| 78 | 100% | 60% | 18% | 5% |
| 80 | 98% | 0% | 3% | 0% |
| 86 | 68% | 27% | 7% | 5% |
| 185 | 100% | 83% | 100% | 43% |

1. A compound of formula (I): 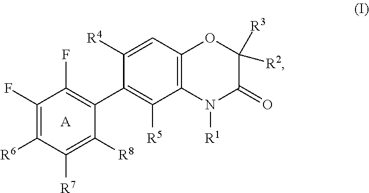
or salt thereof, wherein:
R¹ is C₁₋₆alkyl, C₃₋₄alkenyl, C₃₋₄alkynyl, cyclopropyl, CH₂C₃₋₆cycloalkyl, phenyl or C₁₋₂alkyl-phenyl, each substituted with C(O)Ria or CH₂C(O)R^{1a} and each optionally substituted with up to 3 F or Cl atoms, wherein each C₁₋₆alkyl is also optionally substituted with —OR^{1b};
R^{1a} is OR^{1b}, CH₂OC(O)C₁₋₄alkyl, C(O)OR^{1b}, N(R^{1b})(R^{1c}), ON(R^{1b})(R^{1c}), NHN(R^{1b})(R^{1c}), NHS(O)₂N(R^{1b})₂, NHS(O)₂C₁₋₄alkyl, or NHOR^{1b};
each R^{1b} is, independently, H, C₃₋₆cycloalkyl, CH₂phenyl, or C₁₋₄alkyl optionally substituted with up to 3 F or Cl atoms;
R^{1c} is H or C₁₋₄alkyl optionally substituted with C(O)OR^{1b} or R^{1b} and R^{1c} together with an intervening nitrogen atom form a 4 to 6 membered heterocyclic ring, optionally containing an additional atom or group selected from N, O, S, S(O)₂ and optionally substituted with one or more groups selected from —C(O)OR^{1b} and —C(O)R^{1b};
each of R² and R³ is H, F, or R² and R³ together with the intervening carbon is cyclopropyl;
R⁴ is H, F, CH₃, or Cl;
R⁵ is H or F;
each of R⁶ and R⁷ is, independently, F, H, CH₃, CF₃, or OCH₃;
R⁸ is H or F; and
wherein Ring A contains at least 4 F atom substituents.

2. The compound according to claim 1, or a salt thereof, wherein each of R², R³, and R⁴ is F.

3. The compound according to claim 1, or a salt thereof, wherein each of R² and R³ is H and R⁴ is F.

4. The compound according to claim 1, or a salt thereof, wherein R¹ is C₁₋₆alkyl substituted with —C(O)R^{1a}, wherein R^{1a} is —OR^{1b} or —N(R^{1b})(R^{1c}).

5. The compound according to claim 1 having formula (II): 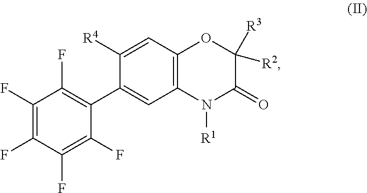
or salt thereof.

6. The compound according to claim 5, or a salt thereof, wherein each of R², R³, and R⁴ is F.

7. The compound according to claim 5, or a salt thereof, wherein R¹ is C₁₋₆alkyl substituted with —C(O)R^{1a}, wherein R^{1a} is —OR^{1b} or —N(R^{1b})(R^{1c}).

8. The compound according to claim 1 having formula (III): 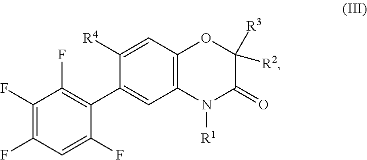
or salt thereof.

9. The compound according to claim 8, or a salt thereof, wherein each of R², R³, and R⁴ is F.

10. The compound according to claim 8, or a salt thereof, wherein R¹ is C₁₋₆alkyl substituted with —C(O)R^{1a}, wherein R^{1a} is —OR^{1b} or —N(R^{1b})(R^{1c}).

11. The compound according to claim 1 having formula (IV): 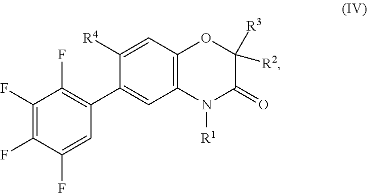
or salt thereof.

12. The compound according to claim 11, or a salt thereof, wherein each of R², R³, and R⁴ is F.

13. The compound according to claim 11, or a salt thereof, wherein R¹ is C₁₋₆alkyl substituted with —C(O)R^{1a}, wherein R^{1a} is —OR^{1b} or —N(R^{1b})(R^{1c}).

14. The compound according to claim 1 having formula (V): 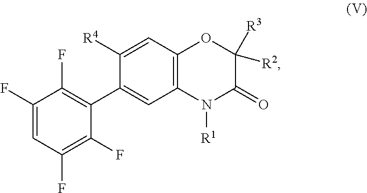
or salt thereof.

15. The compound according to claim 14, or a salt thereof, wherein each of R², R³, and R⁴ is F.

16. The compound according to claim 14, or a salt thereof, wherein R¹ is C₁₋₆ alkyl substituted with —C(O)R^{1a}, wherein R^{1a} is —OR^{1b} or —N(R^{1b})(R^{1c}).

17. The compound according to claim 1, or a salt thereof, wherein the compound of formula (I) is selected from the compounds listed in Table 1, or a salt thereof.

18. An agricultural composition comprising a compound of claim 1, or a salt thereof, and at least one additional component that serves as a carrier.

19. The composition of claim 18, wherein at least one additional component is a surfactant or a diluent.

20. The composition of claim 18, wherein the composition is an herbicidal composition.

21. A method of controlling undesired vegetation, said method comprising contacting said vegetation or its environment with an herbicidally effective amount of a compound of claim 1, or a salt thereof.

22. The method of claim 21, wherein the undesired vegetation comprises weeds.

23. The method of claim 21, wherein the undesired vegetation comprises protoporphyrinogen IX oxidase (PPO) inhibitor-resistant weeds.

24. The method of claim 23, wherein the PPO inhibitor-resistant weeds have a dG210 mutation.

25. The method of claim 21, wherein the compound or composition is applied at a rate of 1 to 100 g per 10,000 m².

26. The method of claim 21, wherein contacting the undesired vegetation or its environment with the compound or composition leads to postemergence control of the undesired vegetation.

27. The method of claim 21, wherein contacting the undesired vegetation or its environment with the compound or composition leads to preemergence control of the undesired vegetation.

28. The method of claim 21, wherein the undesired vegetation is at least 60% controlled.
